(19) United States
(12) Patent Application Publication
(10) Pub. No.: US 2021/0319887 A1
(43) Pub. Date: Oct. 14, 2021

(54) METHOD OF TREATING DIABETES INFORMED BY SOCIAL DETERMINANTS OF HEALTH

(71) Applicant: AMUSENEERING TECHNOLOGIES, LLC, Chicago, IL (US)

(72) Inventors: William Alfred DERRICK, JR., Chicago, IL (US); Dianne Belmear DERRICK, Chicago, IL (US); Bradley Alexander DERRICK, Chicago, IL (US); William Alfred DERRICK, III, Los Angeles, CA (US); Christopher Belmear DERRICK, Los Angeles, CA (US)

(21) Appl. No.: 17/238,905

(22) Filed: Apr. 23, 2021

Related U.S. Application Data
(63) Continuation-in-part of application No. 15/161,188, filed on May 20, 2016, now abandoned.
(60) Provisional application No. 62/164,018, filed on May 20, 2015.
(60) Provisional application No. 63/017,168, filed on Apr. 29, 2020.

(51) Int. Cl. G16H 40/20; G16H 50/30; G16H 10/60; G16H 50/20; G16H 10/20; G16H 10/40; G16H 50/70; A61B 5/00

(52) U.S. Cl. CPC G16H 40/20; G16H 50/30; G16H 10/60; G16H 50/20; G16H 50/80; G16H 10/40; G16H 50/70; A61B 5/7275; A61B 5/4833; G16H 10/20

(57) ABSTRACT

Disclosed herein is an invention that is a medical treatment method for diabetes, its comorbidities and its complications where their treatment requires lifestyle modification. The invention changes or alters lifestyle by identifying what is valuable or harmful to the health-related determinants of the pattern-of-life of a person, modifying the such determinants of health of the person as the person navigates their pattern of life, applying the resulting insights to modify the lifestyle of the person, promoting and improving therapy adherence and compliance and thereby improving the health of the person. The method is intended to prevent diabetes, to increase the early detection of diabetes, to diagnose diabetes, to delay the progression of diabetes, to reduce the severity of diabetes and to operationalize the insights from lifestyle modification through disease risk assessment, medical decision-making, comprehensive care plan management and patient outreach, engagement and retention in the care plan.

### FIELD OF THE INVENTION

Disclosed herein is a method of treating, including diagnosing, delaying the onset of, preventing and reducing the severity of, diabetes, by which novel lifestyle modification treatment methods and risk factors are informed by changes or alterations to a patient's modifiable social determinants of health.

### CROSS-REFERENCE TO RELATED APPLICATIONS

This application is a continuation-in-part of U.S. patent application Ser. No. 15/161,188, filed May 20, 2016, which claims priority to U.S. Provisional Application Ser. No. 62/164,018, filed May 20, 2015, the entire contents of which are hereby incorporated by reference in their entirety. This application also claims priority to U.S. Provisional Application Ser. No. 63/017,168, filed Apr. 29, 2020, the entire contents of which is hereby incorporated by reference in its entirety.

### BACKGROUND

### Field of the Invention

The inventive subject matter relates generally to patient adherence to therapy programs.

### Description of Related Art

The related art extends to public health, clinical healthcare practice, and other research and publications in the field of patient adherence. The relevance of the prior art to the inventive subject matter is that there has been a long-felt, but unresolved, need to take into account the relevant features of human patterns-of-life that determine health as an integral part of therapy programs. The context of the long-felt need is complex and includes the absence of a standard or valid method of predicting therapy adherence, the definitions of “adherence” and “compliance,” the healthcare provider's perspective of the adherence problem, the patient's perspective of the problem, the scope of the social determinants of health, the root causes or the “causes of causes” of the social determinants of health, the approaches to assembling evidence for addressing the problem of therapy adherence, key research gaps, the data points in everyday life, comorbidities, therapy algorithms and guidelines, the systemic burden of non-adherence, and the emergence of medical informatics.

Context Of The Problem—Standardization: The above complexities interconnected with the social determinants of health have prevented the development of a gold standard method of measurement or prediction of therapy adherence or compliance. The lack of a standard or valid method for measuring or predicting per se is a major barrier to treatment adherence and compliance research, and in the case of chronic disease, a hurdle to effective long-term interventions. Because of the difficulties in measuring therapy adherence or compliance, no estimate of treatment adherence, non-adherence, compliance or non-compliance, or predictors thereof, can be generalized.

Context Of The Problem—Definitions Of Adherence & Compliance: In approaching the inventive subjective matter, “adherence” and “compliance” must be distinguished from each other and defined. For purposes of the inventive subject matter, the term “adherence” is referred to as the ability and the willingness of a patient to follow medical advice, such as the acceptance by a patient of the notification by the healthcare professional that a medical problem exists and the commitment made to the healthcare professional by the patient to accept and follow the prescribed or recommended medical and treatment therapies. In participating in a prescribed or recommended therapy, the patient generally will be considered adherent when the patient agrees with and accepts the diagnosis and the prescribed or recommended therapeutic activity. For example, as part of a therapy for a particular disease or condition, a healthcare professional may prescribe or recommend to a patient that the patient perform certain activities (such as, for example in the case of a diabetic, the daily recording in a journal of nutrition information or the regular testing and, in response, taking of an insulin dose) and/or change in a personal daily-life activity (such as, stop smoking, or eat four small meals a day, or stop skipping breakfast).

The term “compliance” is referred to for purposes of the inventive subject matter as the patient's behavior in following medical advice, such as the failure to take or inadequate taking of medication, the failure to execute or inadequate execution of prescribed lifestyle changes, smoking cessation, or following a diet. The patient generally will be considered compliant during the period in which the patient performs the therapeutic activity in the manner as prescribed or recommended. When the patient does not perform such activity or does not perform such activity in the manner as prescribed or recommended (e.g., the number of times per day and/or at the appropriate times per day), the patient generally may be deemed to be not adherent or not compliant.

As such, adherence and compliance embrace acceptance, adaptability, and persistence. Acceptance for purposes of the inventive subject matter may be referred to as the initial decision of the patient to agree to the medical advice, recommendation, or prescribed therapy (collectively referred to hereinafter as the “prescribed therapy”), such as the utilization of counseling, and taking medications. Adaptability to the prescribed therapy for purposes of the inventive subject matter may be referred to as taking or performing treatment in accord with facets impacting the patient's daily pattern-of-life activities, such as: following protocols for changing behavior (such as, modifying diet, increasing physical activity, quitting smoking, self-surveilling and/or self-monitoring of symptoms, safe food handling, dental hygiene, safer sex behaviors, and safer injection practices); health-seeking or health-accessing behaviors (such as, appointment-keeping); medication use (such as, use of appropriate agents, correct dosing, and timing, filling, and refilling prescriptions, consistency of use, and duration of use); and obtaining inoculations. Persistence for purposes of the inventive subject matter may be referred to as continued or sustained or regular performance of the activities required by the prescribed therapy.

Context Of The Problem The Healthcare Provider's Perspective: According to the United States Centers for Medicare & Medicaid Services (“CMS”), there is emerging evidence that addressing health-related social needs can improve health outcomes and reduce costs. CMS reports that many of the largest drivers of health care costs fall outside the clinical care environment—40% of the modifiable variation in health outcomes is due to social determinants of health, whereas only 20% is due to clinical care, 30% to health behaviors, and 10% to the physical environment.

Health-related social needs increase the risk of developing chronic conditions and reduce individuals' ability to manage these conditions. These conditions also are associated with increased emergency department visits and inpatient hospital admissions and re-admissions. Some 500,000 hospitalizations could be averted annually if the rate of preventable hospitalizations were the same for residents of low-income neighborhoods as for those of high-income neighborhoods, and research familiar to one of ordinary skill in the art indicates that unmet health-related social needs may play a significant role in that disparity.

The management of non-medical drivers of health has significant implications for health care utilization. Medicaid invests over $69 billion in home and community based services, as well as investments in countless supports services available through other service delivery systems. Research familiar to one of ordinary skill in the art suggests that services that address health-related social needs have the potential to reduce health care utilization and costs.

Historically, however, patients' health-related social needs have not been addressed in traditional healthcare delivery systems. Many health systems lack the infrastructure and incentives to develop systematic screening and referral protocols or build relationships with existing community service providers.

Generally, data analytics from the healthcare provider's side has focused on clinical data, administrative data, insurance claims data, prescription drug data, and other similar healthcare-provider-focused data. A missing critical factor has been the absence of data analysis and insights from the patient's perspective—pattern-of-life activities within the context of the social determinants of health and their root causes.

Context Of The Problem—The Patient's Perspective: In practice, research indicates that patients define adherence and compliance in terms of good health as perceived by the patient. This definition leads patients to seek treatment approaches that in the patients' view are manageable, tolerable, and effective. From the patients' view, especially patients with chronic disease, illness, or medical condition (interchangeably referred to hereinafter for convenience as “disease,” or “illness,” or “medical condition,” or “condition” as the context may warrant and as known to one of ordinary skill in the art), concerns that take precedence over prescribed therapies include controlling symptoms, preventing medical crises, enjoying a quality lifestyle and/or maintaining financial comfort. As a result, patients do not view all prescribed therapies as necessary for patients' best interests. A patient may believe that the patient has a right to non-adherence or non-compliance with a prescribed therapy, where “intelligent non-compliance” is viewed as the clinical situation where a prescribed medication intentionally is not taken or intentionally inadequately taken or a prescribed activity intentionally is not performed or intentionally inadequately performed, and the patient's reason for non-adherence or non-compliance appears rational to the patient when analyzed dispassionately. Some examples of intelligent non-compliance are: the impact on adherence or compliance of managing everyday life; the discrepancies between doctor's and patient's perceptions of risks and benefits; the patient's perceived remedial effect of medicine; patient experiences with adverse reactions or side-effects that were undisclosed when the medication or treatment originally was prescribed; the patient with a chronic condition becomes aware that the disease has changed; misdiagnosis by the healthcare provider; and inappropriate prescribing.

Context Of The Problem—Social Determinants Of Health And Health Equity: Inequality and equality are dimensional concepts referring to measurable quantities. Inequity and equity, on the other hand, also are political concepts, expressing a moral commitment to social justice. Health inequality is the generic term often used to designate differences, variations, and disparities in the health achievements of individuals and groups. Health inequity often refers to those inequalities in health that are deemed to be unfair or stemming from some form of injustice. The crux of the distinction between equality and equity is that the identification of health inequities entails normative judgment premised upon (a) one's theories of justice; (b) one's theories of society; and (c) one's reasoning underlying the genesis of health inequalities. Because identifying health inequities involves normative judgment, science alone cannot determine which inequalities are also inequitable, nor what proportion of an observed inequality is unjust or unfair. There are many dimensions along which health inequalities could be described, including: gender and race, as well as political power (household authority, work place control, legislative authority), cultural assets (privileged lifestyles, high status consumption practices), social assets (access to social networks, ties, associations), honorific status (prestige, respect), and human resources (skills, expertise, training). The empirical inquiry into health inequalities has only begun to scratch this surface with respect to the social determinants of health.

The United Nations, through its World Health Organization, established the Commission On social determinants of health (“Commission”) in order to address globally the issue of health equity and the significant impact thereon of the global economic and political system. The Commission was created to marshal the evidence on what can be done to promote health equity and to foster a global movement to achieve it. The Commission takes a holistic view that: globally, the poor-health of the poor, the social gradient in health within countries, and the marked health inequities between countries are caused by the unequal distribution of power, income, goods, and services. The consequence of such holistic view, from a national perspective, impacts unfairness in the immediate visible circumstances of peoples' lives, through their access to health care, schools, and education, their conditions of work and leisure, and their homes, communities, towns, or cities, and as a result, their chances of leading a flourishing life. The Commission's positon is that such unequal distribution of health-damaging experiences is the result of a structural combination of poor social policies and programs, unfair economic arrangements, and bad politics, and that together, the structural determinants and conditions of daily life constitute the social determinants of health and are responsible for a major part of health inequities between and within countries.

The focus of the Commission is on the “causes of the causes”—the fundamental global and national structures of social hierarchy and the resulting socially-determined conditions in which people grow, live, work, and age. Globally, the Commission recognizes that it is now understood better than at any moment in history how social factors affect health and health equity. By linking the global understanding of poverty and the social gradient, the Commission asserts the common issues underlying health inequity. Below is the Commission's conceptual framework for addressing the social determinants of health.

Such framework suggests that interventions can be aimed at taking action on: (a) the circumstances of daily life (for example, differential exposures to disease-causing influences in early life, the social and physical environments, and work, associated with social stratification. Depending on the nature of these influences, different groups will have different experiences of material conditions, psychosocial support, and behavioral options, which make individuals and populations more or less vulnerable to poor health; health-care responses to health promotion, disease prevention, and treatment of illness); and (b) the structural drivers (for example, the nature and degree of social stratification in society—the magnitude of inequity along the dimensions listed; biases, norms, and values within society; global and national economic and social policy; processes of governance at the global, national, and local level).

The Commission recognizes that, by their nature many, of the social determinants are relatively distant, spatially and temporally, from individuals and health experience. This is challenging, both conceptually and empirically, when trying to attribute causality and demonstrate effectiveness of action on health equity. In the Commission's choosing of the range of social determinants on which to focus, the selection was based on coherence in the global evidence base; that is, a mixture of conceptual plausibility, availability of supporting empirical evidence, and consistency of relationship between and among populations, together with the demonstration that these determinants were amenable to intervention. In addition, a few determinants were identified that, while they had a strong plausible relationship with health inequities, still lacked evidence on what could be done to effect change.

The Commission's conceptual framework recognizes that there needs to be evidence on what can be done and what is likely to work in practice to improve health and reduce health inequities. As to what constitutes evidence when it comes to the social determinants of health, the Commission recognizes two linked problems: the nature of the intervention and the lack of evidence in areas where it matters. The Commission takes a broad view of what constitutes evidence of the social determinants of health, and includes evidence that comes from observational studies (including natural experiments and cross-country studies), case studies, and field visits, from expert and lay knowledge, and from community intervention trials where available. There are gaps inevitably, particularly in low- and middle-income countries, possibly because the information does not exist, was not published in an accessible manner, or is not available in English (the working language of the Commission).

Recognizing the evidentiary challenges integral to the social determinants of health, and underpinned by the conceptual framework, a knowledge work stream has been established by the Commission primarily around nine Knowledge Networks whose themes incorporate global issues, health systems level issues, and a life-course approach to health. The Knowledge Networks focus on early child development, employment conditions, urban settings, social exclusion, women and gender equity, globalization, health systems, priority public health conditions, and measurement and evidence. Gender issues have been systematically considered in each of the other themes. Other issues, including food and nutrition, rural factors, violence and crime, and climate change, do not have a dedicated Knowledge Network but are recognized as important factors for health equity.

Although the Commission recognizes the social determinants of health and their impact on individual and public health globally, the Commission reports that key research gaps identify the most pressing continuing research needs required to generate new understanding and to disseminate that understanding to individuals and institutions in practical accessible ways. New methodologies are needed, including: (a) developing and testing of social determinants of health indicators and evaluation of the impact of interventions; (b) recognizing and utilizing a range of types of evidence; (c) recognizing and utilizing the added value of globally-expanded Knowledge Networks and communities; (d) expanding the scope of evidence across the thematic areas reflected in the nine Knowledge Networks and across country contexts; (e) recognizing that the social and economic drivers of health inequities are dynamic, changing over time.

Other overarching research needs identified by the Commission include: (a) the determinants of health inequities in addition to the determinants of average population health (for example, understanding reasons for the relationship between social stratification and health outcomes, understanding the interaction between aspects of stratification [for example, gender, ethnicity, and income] and health inequities; and quantifying the impact of supra-national political, economic, and social systems on health and health inequities within and between countries; (b) monitoring and measurement (for example, developing new methodologies for measuring and monitoring health inequities, and for assessing the impact of population-level interventions); (c) interventions, global to local, to address the social determinants of health and health equity (for example, evaluating the impact of societal-level action [policies and programs] on health inequities and research on the social, economic, and health costs and benefits of reducing health inequities); (d) policy analysis (for example (analyzing policy processes towards health equity-related interventions, understanding contextual barriers and enablers to intersectional action and coherence in national and local governance and policymaking; and (e) identifying current good practice and developing tools for intersectional action;

In addition to the Commission, additional research is needed to address key questions pertinent to health inequalities, such as: What is the distinction between health inequality and health inequity? Should we assess health inequalities themselves, or social group inequalities in health? Do health inequalities mainly reflect the effects of poverty, or are they generated by the socioeconomic gradient? Are health inequalities mediated by material deprivation or by psychosocial mechanisms? Is there an effect of relative income on health, separate from the effects of absolute income? Do health inequalities between places simply reflect health inequalities between social groups or, more significantly, do they suggest a contextual effect of place? What is the contribution of the life-course to health inequalities? What kinds of inequality should we study?

Context Of The Problem—Data Points In Everyday-Life; Determinants Of Health: The inventive subject matter embraces the complex data points of everyday patterns-of-life. Individuals are unlikely to be able to directly control many of the determinants of health. These determinants as reported by the World Health Organization include the social and economic environment, the physical environment, and the person's individual characteristics and behaviors, as well as many other determinants such as: income and social status (higher income and social status are linked to better health, and the greater the gap between the richest and poorest people, the greater the differences in health); education (low education levels are linked with poor health, more stress and lower self-confidence); physical environment (safe water and clean air, healthy workplaces, safe houses, communities and roads all contribute to good health); employment and working conditions (people in employment are healthier, particularly those who have more control over their working conditions); social support networks (greater support from families, friends and communities is linked to better health); culture (customs and traditions, and the beliefs of the family and community all affect health); genetics (inheritance plays a part in determining lifespan, healthiness, and the likelihood of developing certain illnesses); personal behavior and coping skills (balanced eating, keeping active, smoking, drinking, and how we deal with life's stresses and challenges all affect health); health services (access and use of services that prevent and treat disease influences health); and gender (men and women suffer from different types of diseases at different ages).

The evidence of health-impacts often is not available, because of the long causal pathway between the implementation of a health project, program, or policy and any potential impact on population and individual health, and because of the many confounding factors that make the determination of a causal pathway link difficult. In addition, providing a comprehensive review of the evidence base is not simple. However, the World Health Organization reports that there are examples where the best available evidence has been documented, and in some cases summarized.

Context Of The Problem—Chronic Disease: Long-term adherence and compliance are critical success factors to today's health policies, where the burden of disease in the population has shifted toward chronic diseases and preventive health. Chronic disease is a long-term condition, and adherence and compliance over the long-term are critical to achieving optimal outcomes in disease management and in prevention.

The daily treatment-related demands associated with the social determinants of health may be seen as significantly impacting, if not driving, adherence and compliance. Long-term successful adherence and compliance, such as required in the management of chronic illness, may be achievable only where an individual successfully manages those social determinants specific to the individual, particularly as such determinants are impacted by the root causes of chronic illness.

Non-adherence and non-compliance are likely with respect to chronic diseases in every situation in which patients are required to administer their own treatment, since almost everyone has difficulty adhering to and complying with medical recommendations, especially when the advice entails self-administered care. Further, chronic diseases are burdened with the risk that poor adherence and compliance increases with the duration and complexity of treatment regimens, together with the long duration (typically lifetime) of the chronic disease.

Context Of The Problem—Comorbidities: Comorbidity deals with people having two or more multiple chronic diseases, multiple chronic illnesses or multiple chronic conditions (interchangeably referred to as “MCC” as the context may require). Examples of MCC include, but are not limited to, the simultaneous presence of one or more of: arthritis, asthma, chronic respiratory conditions, diabetes, heart disease, human immunodeficiency virus infection, obesity/overweight, and hypertension HHS defines chronic illnesses as “conditions that last a year or more and require ongoing medical attention and/or limit activities of daily living”. Although presently there is no standard definition of comorbidity, the term “comorbid” generally is understood: (a) to indicate a medical condition existing simultaneously but independently with another condition in a patient and (b) to indicate a medical condition in a patient that causes, is caused by, or is otherwise related to another condition in the same patient. In medicine, comorbidity is the presence of one or more disorders or diseases in addition to a primary disease or disorder, or the effect of such additional disorders or diseases. Also in medicine, comorbidity describes the effect of all other diseases an individual patient might have other than the primary disease of interest. In addition to comprising physical medical conditions, chronic conditions also include problems such as substance use and addiction disorders, mental illnesses, dementia and other cognitive impairment disorders, and developmental disabilities.

Commonly, MCC are analyzed by the following major sociodemographic factors—gender; age; race/ethnicity; health insurance (physician visits, prescription medicine). However, standard analysis of MCC does not yet include the host of factors represented by the social determinants of health. The absence of social determinants in the analysis of MCC further complicates the many complex issues that dovetail with the challenges of defining MCC. As a result, defining a chronic condition and MCC requires careful consideration. In the HHS MCC Strategic Framework, a chronic condition is defined as a condition lasting 12 or more months and requiring ongoing medical care. But how should remittent diseases such as asthma, certain mental illnesses, or multiple sclerosis be considered? What about late recurrences of tumors thought to be controlled? There are no clear standard answers to these questions, and as a result, period prevalence rates are not sufficient to define MCC.

Looking at an “individual” disease, is it one or many? Many diseases that are regarded as single entities exhibit diverse organ involvement, and over time, special and distinct clinical manifestations and sequellae. (In ordinary language, a sequellae may be described as a further condition that is different from, but a consequence of, the first condition, for example: chronic kidney disease is sometimes a sequela of diabetes and diabetes is often a sequela of obesity or overweight). Additionally, diabetes mellitus is clearly associated with coronary heart disease, renal insufficiency, retinal disease, skin abnormalities, and other important clinical problems. Should each of these be considered separately in the multiplicity of MCC, or as part of one condition for analytical purposes? Again, it depends on the question being addressed.

How should the “secondary outcomes” of a variety of biologically unrelated chronic conditions be considered and counted? Many chronic conditions clearly lead to a variety of common and functional outcomes that are not necessarily related to the underlying causes of the primary disease, including falls, cognitive impairment, anemia, malnutrition, polypharmacy, sleep disorders, and sexual dysfunction. Often, statistically significant associations between various primary index illnesses and these secondary outcomes are present, even if the latter are not biologically related to the primary condition. The complex downstream pathways for additional chronic illnesses, whether they are biologically related or less specific secondary conditions, all may be clinically important.

As a result of the complexities in describing a chronic condition, preventive interventions are as important as managing the primary condition.

Chronic conditions are an increasing concern in the United States, as MCC affect nearly half of the adult population and their prevalence has increased in recent years. More than one in four Americans have two or more concurrent chronic conditions, including for example, arthritis, asthma, chronic respiratory conditions, diabetes, heart disease, human immunodeficiency virus infection, obesity/overweight, and hypertension. MCC result in numerous adverse health outcomes, increased health care needs, and subsequently higher medical costs.

The prevalence of multiple chronic conditions among individuals increases with age and is substantial among older adults, even though many Americans with MCC are under the age of 65 years.

As the number of chronic conditions in an individual increases, the risks of the following outcomes also increase: mortality, poor functional status, unnecessary hospitalizations, adverse drug events, duplicative tests, and conflicting medical advice. This picture is even more complex as some combinations of conditions, or clusters, have synergistic interactions, but others do not. For example, the poor health outcomes of individuals with serious mental illnesses and other behavioral health problems warrants special attention because of the co-occurrences of those conditions with other chronic conditions.

Managing MCC is quite complex. How does one deal with complex clinical manifestations of conditions, such as signs (visually observable patient abnormalities), symptoms (abnormal perceptions of illness that only the patients can report, such as pain, itching, fatigue, depressive feelings), and syndromes (clusters of signs, symptoms, and other clinical phenomena that may or may not be indicative of a specific underlying disease)? Do these signs, symptoms, and syndromes belong in the study of MCC? These signs, symptoms, and syndromes must be carefully and systematically addressed, since many never reach the level of a specific diagnosable “disease” with an ICD code, therapy cost reimbursement codes known by one of ordinary skill in the art. Although specific ICD codes may not have been assigned to such signs symptoms and syndromes, they nevertheless can cause considerable suffering and require health care.

In the past, MMC management strategies have focused on preventing and ameliorating a single disease at a time. However, the large percentage of people with MCC has added a layer of complexity to developing prevention and intervention strategies. As a result, HHS has developed a MCC Strategic Framework to address multiple chronic conditions. The HHS MCC Strategic Framework for managing has four overarching goals: foster health care and public health system changes to improve the health of individuals with multiple chronic conditions; maximize the use of proven self-care management and other services by individuals with multiple chronic conditions; provide better tools and information to health care, public health, and social services workers who deliver care to individuals with multiple chronic conditions; facilitate research to fill knowledge gaps about, and interventions and systems to benefit, individuals with multiple chronic conditions. Strategies of the framework include the stimulation of epidemiologic research to determine the most common MCC dyads and triads (terms known by one of ordinary skill in the art) and to explain more clearly the differences in MCC and the opportunities for prevention and treatment among various sociodemographic groups.

Another issue is whether to consider many infectious diseases as chronic conditions. Research indicates that important chronic infections exist (such as, tuberculosis and hepatitis B and C) that impact the definition of MCC. These conditions, and their co-occurring illnesses, encumber all of the management challenges of important noninfectious diseases such as coronary heart disease, cancer, diabetes, or stroke-related disability.

In counting diseases and conditions, at least two other issues remain. First, how should adverse effects of therapy be counted? They can be costly and deadly. Second, how should disease risk factors such as elevated blood pressure, elevated cholesterol, and the physiological changes of aging (such as, osteopenia or sarcopenia) be treated? These “non-diseases” may require further consideration as MCC.

A related consideration for the population of persons with MCC is disparities in access to health care, public health, and other services. The resource implications for addressing MMC are immense—66% of total health care spending is directed toward care for the approximately 27% of Americans with MCC. Increased spending on chronic diseases among Medicare beneficiaries is a key factor driving the overall growth in spending in the traditional Medicare program. Individuals with MCC have faced substantial challenges related to the out-of-pocket costs of their care, including higher costs for prescription drugs and total out-of-pocket health care. MCC can contribute to frailty and disability; conversely, many older persons who are frail or disabled have MCC. The confluence of MCC and functional limitations, especially the need for assistance with activities of daily living, produces high levels of spending. Functional limitations can often complicate access to health care, interfere with self-management, and necessitate reliance on caregivers.

Research by the Institute of Medicine (“TOM”) indicates that patients receiving care for one chronic condition may not receive care for other, unrelated conditions. As a result, there is a challenge of designing care around specific conditions so as to avoid defining patients solely by their disease or condition. The IOM Chronic Care Model further elucidates the elements required to improve chronic illness care, including systems requirements for healthcare organization, community resources, self-management support, delivery design, decision support, and clinical information. This seminal model represents a conceptual foundation for innovative approaches to addressing MCC.

Developing means for determining homogeneous subgroups among the heterogeneous MCC population is viewed as an important step in the effort to improve the health status of the total population and only recently is beginning to be addressed by researchers. Identifying such subgroups will assist in more effectively developing and targeting interventions.

The combined effects of increasing life expectancy and the aging of the population will dramatically increase the challenges of managing MCC among the burgeoning population of older persons. Nevertheless, the delivery of community health and health care services generally continues to be centered on individual chronic diseases. In addition, insufficient attention has been paid to the services and support required to meet longer-term needs of those with MCC to enable them to live as well as possible in community settings.

Context Of The Problem—Therapy Algorithms & Guidelines: The nature of adherence and compliance is recognized as a complex behavioral process. As such, adherence and compliance strategies seek to address the type of patient behavior, an acceptable frequency of behavior, consistency of behavior, and behavior intensity and/or accuracy.

Interacting factors impacting adherence, and compliance include attributes of the patient influenced by the social determinants of health, such as poor health literacy, lack of comprehension of treatment benefits, the cost of prescription medicine, the complexity of modern medication regimens, poor communication between the patient and the individual's healthcare provider, the occurrence of undiscussed side effects, and the lack of trust between the patient and the patient's healthcare provider. In addition to patient attributes, the patient's daily-living attributes also are impacted by the social determinants of health, such as the availability of social supports, the availability and accessibility of healthcare resources, functioning of the healthcare team, and characteristics of the healthcare system.

Research familiar to one of ordinary skill in the art has identified certain correlates and predictors of adherence and non-adherence. They include socioeconomic variables embedded in the social determinants of health, as well as interaction between healthcare practitioner and patient, aspects of the complexity and duration of treatment, characteristics of the disease, illness, or medical condition, iatrogenic effects of treatment or advice (such as, a complication following a surgical procedure, complex drug interactions, side effects of a treatment, chance, medical error, negligence, unexamined instrument design, anxiety or annoyance in the treatment provider in relation to medical procedures or treatments, and unnecessary treatment for profit), costs of treatment, and characteristics of health service delivery.

Research familiar to one of ordinary skill in the art suggests that four interdependent factors operate on adherence and compliance and are impacted by the social determinants of health. The first factor is patient knowledge and skills about: the health problem, self-regulation of the required patient behaviors, the mechanisms of patient action, and the importance of adherence. The second factor is patient beliefs, such as: perceived severity and susceptibility or relevance to the patient, self-efficacy, outcome expectations, and response costs. The third factor is patient motivation, that is: value and reinforcement where internal attribution of success with positive outcomes is seen as reinforcing, while negative results are seen not as failure, but rather as an indication to reflect on and modify behavior. The fourth factor is patient action, which is stimulated by relevant cues driven by information recall, evaluation, selection of behavioral options, and available resources.

Context Of The Problem—Systemic Burden: Treatment non-compliance has a dramatic impact on the United States healthcare system. Research familiar to one of ordinary skill in the art indicates that poor compliance is to be expected in approximately 30-50% of all patients, irrespective of disease, prognosis, or setting. Such research indicates that the estimated compliance rate of long-term medication therapies is 40%-50%, and compliance rate for short-term medication therapy is 70%-80%, while the compliance with therapy such as lifestyle changes is the lowest at 20%-30%. For the management of diabetes, such research indicates that the rate of compliance to diet varied from 25% to 65%, and insulin administration compliance was about 20%, while compliance with oral medication for type 2 diabetes mellitus ranged from 65% to 85%. Such research further indicates that hospital readmission rates for chronic conditions were 23%, compared to 19% for acute conditions in Medicare patients over 65. For Medicaid patients age 18 to 44, the readmission rate was 26% for chronic conditions versus 19% for acute conditions—about one-third higher. Moreover, 33%-69% of all chronic disease-related hospital admissions in the United States were due to poor adherence to and/or compliance with instructions for self-management of chronic disease. Poor or non-adherence or compliance contribute to annual indirect costs exceeding $1.5 billion in lost earning, and $50 billion in lost productivity.

Public health is linked through the application of technology to population health, community health and personalized health. Public health, perhaps the seminal discipline in the healthcare field, is the most interdisciplinary, and therefore, the most challenging health profession. To practice public health well requires an integrated knowledge of disciplines including: molecular biology, the basic medical sciences, all the clinical disciplines, epidemiology, statistics, environmental sciences, psychology, sociology, anthropology, economics, administration and management, law, politics and policy and ethics, as well as engineering, urban planning, education, architecture and social work all figure into public health interventions or procedures.

Context Of The Problem—Medical Informatics: The inventive subject matter relates generally to Medical Informatics (also called Healthcare Informatics, Health Informatics, and Clinical Informatics). Medical Informatics is a discipline at the intersection of information science, computer science, and health care. The focus of Medical Informatics is on the diagnosis and/or treatment of patients and/or diseases. Medical Informatics deals with the resources, devices, and methods required to optimize the acquisition, storage, retrieval, and use of information in healthcare. Medical Informatics tools include not only computers, but also clinical guidelines, formal medical terminologies, and information and communication systems. Medical Informatics is applied to the areas of public health, clinical care, nursing, dentistry, pharmacy, occupational therapy, and related research. The application of Medical Informatics faces several challenges, including the context of the social determinants of health, the sources of data, the types of data to be used as measures of health, the quantification of social determinants data, and limitations of the analytic tools.

Medical Informatics approaches to analytics, particularly predictive analytics, are challenged by the interweaving into the fabric of social determinants of health data evidencing the context of such determinants. A patient must cope with treatment-related demands that are characterized by the requirement to learn new behaviors, alter daily routines, and tolerate discomforts and inconveniences, as well as persist in doing so while trying to function effectively in a patient's various life-roles, together with the broad, interdisciplinary, professional health expertise and integrated knowledge required to effectively analyze pattern-of-life activities and to operationalize the likelihood of adherence to, compliance with, and cessation of therapy programs. There is much controversy around the theoretical and analytical framework of group and individual data and the outcomes associated with patterns-of-life. Much of such controversy is associated with terms such as health disparity, health inequality, socioeconomic inequality in health, and socioeconomic health differentials.

A social structure and individual personality perspective of patterns-of-life data provide a theoretical and analytical framework through Medical Informatics for the association among health disparities, socioeconomic status, and health outcomes. Research suggests that psychosocial factors, such as health behaviors, stress, social ties, and attitudinal orientations, are critical links between social structure and health status. Research further suggests that psychosocial factors are linked more strongly to health status than is medical care and are related systematically to socioeconomic status. The social distributions of psychosocial factors represent the patterned response of social groups to the conditions imposed on them by social structure. Accordingly, the elimination of inequalities in health status ultimately may require changes not only in psychosocial factors or health care delivery, but also in socioeconomic conditions.

The application of Medical Informatics to patterns-of-life data from an empirical analysis perspective may deal with health disparities and health inequalities through two distinct approaches. One examines overall inequalities in health and proceeds in much the same way as the literature on measuring income inequality. In this approach, sometimes referred to as the univariate approach, all inequalities in health are measured, irrespective of the other characteristics of the individuals involved. The second approach looks at a subset of health inequalities, namely those occurring across the distribution of some measure of socioeconomic status, sometimes referred to as the bivariate approach. Under both approaches, empirical measurement methodologies developed for both individual-level data and grouped data compare overall health inequality and socioeconomic status health inequality, by arriving at coefficients or scores utilizing statistical techniques and processes.

Research is needed to address long-felt but unresolved needs: that will identify the critical features of patterns-of-life associated with health disparities or socioeconomic status that determine health; that delineate the mechanisms and processes whereby social stratification produces disease; and that specify the psychological and interpersonal processes that can intensify or mitigate the effects of social structure.

Another challenge for the application of Medical Informatics is that data evidencing patterns-of-life and their context must be located, assembled, and analyzed. Large-scale surveys commonly are used in performing healthcare analysis. Inappropriate inferences or bias can be drawn from large-scale survey data, which can lead to faulty “representativeness” of the survey data and its impact on conclusions about the wider population. All statistical surveys, whether based on samples or attempted complete enumerations, are subject to potential inaccuracies. These risks include errors in conceptual formulation, ambiguities in definition and in the questionnaire, faulty classification, interviewer variability and bias, respondent bias and variability, biases from nonresponse or incomplete coverage, mistakes in editing, and tabulation errors. The manner in which the survey sample is selected, the manner of the sample design, the implications of the selection process, and the way the survey is implemented may be sources of bias. The survey analysis may require adjustments, such as stratification or multistage sampling, for departures from simple random sampling which may lead to bias. Additional sampling bias can arise from the practice of “convenience sampling” aimed at avoiding remote or inaccessible population areas or from the use of an inaccurate or inappropriate sampling frame. There also are potential sampling biases that arise in the process of survey implementation, such as nonresponse or measurement errors related systematically with target variables and errors in recording or data entry. In addition, large-scale surveys may result in bias when converted to outcomes for individuals. Moreover, the scope, focus, and measurement approaches with large surveys vary across surveys and over time, limiting the scope for comparisons. These surveys are expensive to conduct and tend to be implemented only periodically.

Living standards studies, typically developed from large-scale surveys, often are the basis from which healthcare analysis is performed. The construction of different measures of living standards is a source of sampling bias. There are conceptual, as well as practical, differences among different measures of living standards making it difficult, if not impossible, to establish the “best” living standards measure. Often, there is a preference to assess living standards by reference to long-term command over assets, tangible and intangible, or by reference to consumption. Asset and consumption variables can be proxied by an asset index or a consumption index. Consumption data as a measure of living standards, like large-scale surveys, also are expensive to collect and are susceptible to measurement error. Arguably, income as a measure of living standards is an inferior measure, not only because of measurement challenges, but also because for most households the fluctuation in income over time does not imply commensurate changes in living standards. If a household suffers a temporary negative income shock due to illness, but is able to maintain consumption through savings, insurance, or some other resource, it may be a source of sampling bias to rank the household based on income or to express out-of-pocket payments as a share of income.

Asset data, particularly housing data, are easier to collect and potentially less susceptible to measurement error than consumption measures. However, results have been shown to be sensitive to the choice of assets and household characteristics that are included in an asset index. Although asset indices are often poor predictors of consumption, asset indices continue to be used in testing the hypothesis of whether consumption is a significant determinant of health outcomes, particularly where sample sizes are large and there is a great deal of variation in consumption.

Software and computer capabilities have advanced the application of statistical techniques to the quantification of socioeconomic data. Approaches to quantifying such data are challenged initially by having been based on data sourced from large-scale household surveys. Approaches to quantify socioeconomic data have focused on measuring and explaining inequality in health service delivery to support public spending decisions. Such approaches apply statistical techniques to establish coefficients for applications as proxies for measuring income related to inequality of healthcare use and to determine the separate contributions of patient need, income and other non-need factors to such indices. The empirical analyses of socioeconomic inequalities issues are further challenged by being based on different measures of socioeconomic status, including continuous variables, such as income and consumption, and categorical variables such as social class, occupational group, educational attainment, and ethnicity. Such work also is challenged by its concern with horizontal equity in the delivery of health care in the case of more-developed countries, while in the case of lesser-developing countries, concern has been on the narrower issue of equality. Health equity requires that people in equal need of treatment receive the same treatment irrespective of their income. Accordingly, if illness incidence is unequally distributed along income lines, equity requires that utilization of services related to that specific illness be similarly distributed. In contrast, equality is concerned only with the distribution of the service itself. Once healthcare use is standardized for consumer need in nonlinear settings, inequality is explained by decomposing the concentration index, with adjustments made for standard errors for the contributions to the concentration index decomposition. The perspective of health equity does not require a control for consumer need;

as a result, data requirements often are relaxed considerably. In the absence of service-specific unit cost estimates, many studies have restricted their attention to binary indicators of whether a person used a particular healthcare service or not.

In addition, Medical Informatics approaches to the social determinants of health must manage statistical and other analytics techniques such as: incomplete or conflicting techniques of analyzing pattern-of-life data; a necessity for multiple adjustments to the analytical process; measurement methods that fail to gather valid information on the extent of patient adherence or compliance; the failure of qualitative methods (for example, questionnaires, a popular method) used to gather subjective intonation (such as, the social and the historical context or unbiased interpretation of medication use or personalized experiences in a person's own words); the sheer multitude of determinant, component, and indicator data; the confounding that results from the application of statistical analysis methods to the wide number and variation of patient pattern-of-life activities; the identification of determinants data; the determination and the categorization of relevance determinants data; the analysis and preparation of actionable insights from determinants data and the reporting of such insights to patients and to healthcare providers; the correlation of relevant determinants data with therapy programs; the sourcing of relevant determinants data within privacy and technical limitations; the creation of new and the improvement of existing therapies and their treatment and medication algorithms and guidelines based on determinants data; the creation of new patient outreach, encounter, intervention and/or retention environments based on such actionable insights; the application of new or improved preventive health strategies based on such actionable insights from determinants data for patient outreach, encounter, intervention, and retention within the regulatory limitations on healthcare marketing and the promotion of preventive health; the measurement and validation of patient adherence and compliance; and the frequent interchangeable use of “adherence” and “compliance”.

In practice, researchers have relied on morbidity variables (such as, self-assessed health, presence of chronic conditions, activity limitations, insurance claims data, clinical data, administrative data, etc.) and have ignored or not have had available the root causes of illness, such as the social determinants of health and health status, as measures in diagnosing healthcare need, in prescribing or recommending healthcare therapy, and in managing adherence and compliance.

Conclusion Of Background: Thus, there remains a substantial and compelling need for methods and systems that can reasonably and efficiently analyze and quantify the social determinants of health and operationalize the resulting actionable insights to improve health through therapy adherence and compliance, to develop new or improved healthcare benefits plans featuring adherence and compliance performance, and to promote preventive health through therapy adherence and compliance.

The diabetes global epidemic challenges healthcare providers to develop novel strategies to prevent and treat this life-long disease. City dwellers are at especially high risk, because they tend to be less physically active and are more likely to be obese as compared to their rural counterparts. Notwithstanding this epidemic, there is abundant evidence that diabetes can be prevented, its onset can be delayed, its severity can be reduced and its complications can be avoided.

In the majority of cases, type 2 diabetes is considered to be one component within a group of disorders referred to as the metabolic syndrome characterized by a group or combination of recognizable, complex, correlated or coexisting Symptoms and Signs or physical findings that, together, represent the diabetes process or a metabolic condition for which a direct cause is not necessarily understood. Factors characteristic of metabolic syndrome are a cluster of risk factors for diabetes and cardiovascular disease (CVD), including abdominal obesity, dyslipidemia, hyperglycemia and hypertension.

The presence of additional coexisting chronic conditions has a significant impact on the diagnosis, prevention, delay of the onset, treatment and management of diabetes and premature death. CVD is the leading cause of death for all people with diabetes. The Centers for Medicare and Medicaid (CMS) reports that its Medicare beneficiaries account for 80+ commonly coexisting triads of diabetes and other chronic diseases, of which the top 20 triads, their prevalence among all Medicare beneficiaries and the per capita Medicare spending, respectively, were: (1) diabetes & hyperlipidemia & hypertension, 29.2%, $20,071; (2) diabetes & chronic kidney disease & hypertension, 24.2%, $24,494; (3) diabetes & ischemic heart disease & hypertension, 19.9%, $25,680; (4) diabetes & chronic kidney disease & hyperlipidemia, 18.7%, $25,787; (5) diabetes & arthritis & hypertension, 18.4%, $22,920; (6) diabetes & ischemic heart disease & hyperlipidemia, 16.0%, $26,992; (7) diabetes & arthritis & hyperlipidemia, 14.13%, $24,470; (8) diabetes & chronic kidney disease & ischemic heart disease, 14.1%, $30,923; (9) diabetes & chronic kidney disease & arthritis, 12.1%, $28,308; (10) diabetes & heart failure & hypertension, 11.9%, $34,995; (11) diabetes & depression & hypertension, 10.4%, $30,710; (12) diabetes & ischemic heart disease & arthritis, 10.3%, $29,242; (13) diabetes & heart failure & chronic kidney disease, 9.7%, $38,880; (14) diabetes & heart failure & ischemic heart disease, 9.6%, $36,620; (15) diabetes & heart failure & hyperlipidemia, 9.1%, $37,502; (16) diabetes & depression & hyperlipidemia, 8.1%, $32,439; (17) COPD & diabetes & hypertension, 8.0%, $34,186; (18) diabetes & chronic kidney disease & depression, 7.3%, $36,847; (19) Alzheimer's disease/dementia & diabetes & hypertension, 6.8%, $34,489; and (20) diabetes & heart failure & arthritis, 6.5%, $37,923.

Complications commonly associated with diabetes include a significantly higher prevalence of lower-extremity diseases, such as peripheral arterial disease, neuropathy, foot ulcer and amputation. Diabetes is associated with a significant excess risk of disabling conditions, such as: about 2-fold for depression; 1.2 to 1.7-fold for cognitive decline; 1.6-fold for dementia; 1.7-fold for hip fracture; and 2 to 3-fold for physical disability.

People with diabetes together with multiple chronic conditions report a number of barriers to self-care, such as physical limitations, lack of knowledge, financial constraints, logistics in obtaining care and the need for social and emotional support. The specific combination of comorbidities in diabetes patients has been found to impact their ability to prioritize and manage the disease. Patients with conditions often considered unrelated to diabetes may need additional support in making decisions about care priorities and self-management activities. While the presence of diabetes-“concordant” conditions (such as sharing the same management goals), tends to be positively associated with quality of care, certain “discordant” comorbidities (such as depression and arthritis) impact diagnosis and treatment options, posing barriers to lifestyle changes or alterations and self-care behaviors recommended for diabetes management.

The Burden Of Diabetes The current epidemic of obesity and physical inactivity also has led to an increased prevalence of the metabolic syndrome. There is ongoing interest, research and debate around the definition of metabolic syndrome and its diagnosis and clinical utility. Error! Bookmark not defined. Notwithstanding the controversy, international diabetes organizations allow the inclusion of patients with diabetes, a CVD risk equivalent, and share the common goal of identifying individuals at increased risk for developing CVD. Such publications stress the importance of lifestyle modification, including weight loss and increased physical activity. Behavior modification interventions produced identical 58% reductions in progression to diabetes demonstrating that lifestyle modification is effective in delaying the onset of or preventing the development of both the metabolic syndrome and diabetes.

Impact of Ethnicity on the Diabetes Burden

It is well-documented that race/ethnic minorities have a higher prevalence of diabetes and shoulder a higher disease burden than nonminority individuals. Multiple factors contribute to the disease disparities, including social, health system, biological and clinical factors.

The importance of social factors is evidenced in the Final Rule of the Office of Inspector General of the U.S. Department of Health and Human Services, “Revisions to the Safe Harbors Under the Anti-Kickback Statute and Civil Monetary Penalty Rules Regarding Beneficiary Inducements.” This Final Rule is part of HHS's Regulatory Sprint to Coordinated Care, which aims to reduce regulatory barriers to care coordination and accelerate the transformation of the health care system into one that better pays for value and promotes care coordination. HHS has identified the broad reach of the Federal anti-kickback statute, 42 U.S.C. § 1320a-7b(b), and the civil monetary penalty law provision prohibiting inducements to beneficiaries, 42 U.S.C. § 1320a-7a(a)(5), as potentially inhibiting beneficial arrangements that would advance the transition to value-based care and improve the coordination of patient care across care settings in both the Federal health care programs and commercial sector. The safe-harbor to the Anti-Kickback laws permits referrals of patients in connection with value-based arrangements for the provision of certain patient engagement tools and support provided to patients. The safe-harbor applies specifically to services to identify and address a patient's social determinants of health used to directly advance specified goals, such as improving patient adherence to certain treatment regimens and improving evidence-based health outcomes for a target patient population

In a nationally representative survey of US adults from 2011 to 2016, Prevalence of Diabetes by Race and Ethnicity in the United States, the prevalence of diabetes and undiagnosed diabetes varied by race/ethnicity and among subgroups identified within the Hispanic and non-Hispanic Asian populations. According to the study, the weighted age- and sex-adjusted prevalence of total diabetes was 12.1% for non-Hispanic white, 20.4% for non-Hispanic black, 22.1% for Hispanic and 19.1% for non-Hispanic Asian adults. Among Hispanic adults, the prevalence of total diabetes was 24.6% for Mexican, 21.7% for Puerto Rican, 20.5% for Cuban/Dominican, 19.3% for Central American and 12.3% for South American subgroups. Among non-Hispanic Asian adults, the prevalence of total diabetes was 14.0% for East Asian, 23.3% for South Asian and 22.4% for Southeast Asian subgroups. The prevalence of undiagnosed diabetes was 3.9% for non-Hispanic white, 5.2% for non-Hispanic black, 7.5% for Hispanic and 7.5% for non-Hispanic Asian adults.

With respect to the Medicare population, the 2012 Medicare Current Beneficiary Survey examining racial and ethnic differences in self-reported measures on access to care, propensity to seek care, self-care knowledge and behaviors, diabetes management and complications among Medicare beneficiaries ages 65 and older, the prevalence of diabetes among Black (37%) and Hispanic (38%) beneficiaries was higher than among their White counterparts (25%). Minority beneficiaries with diabetes also are more likely to receive lower quality care and have diabetes-related complications, such as end-stage renal disease, chronic kidney disease and amputations. In addition, genetic predisposition, higher rates of obesity earlier onset, poor blood sugar control, diet and lack of exercise all have been shown to contribute to these racial and ethnic disparities.

There are race/ethnic differences in the epidemiology of diabetes, prediabetes and diabetes complications and mortality in the United States and globally. In addition to biological contributions to diabetes and metabolic syndrome disparities, studies show that other contributors to disparities include behavioral, social, environmental and health system factors.

Modifiable social determinants of health (mSDOHs), as distinguished from structural social determinants of health, are lifestyle risk factors recognized by healthcare team organizations, such as the International Diabetes Federation, and government organizations, such as the National Institutes of Health. mSDOHs include health behavior and other non-biological factors contributing to race/ethnic disparities in diabetes. Physical activity and smoking are well-recognized risk factors for developing diabetes. Non-Hispanic Blacks, Native Americans and Alaska Natives are reported to be less physically active compared to Non-Hispanic Whites, and Mexican American women are reported to have lower levels of physical activity compared to Non-Hispanic Whites and Non-Hispanic Blacks. Data on physical activity in Asian Americans are very limited.

Non-Hispanic Blacks and Non-Hispanic Whites have been reported to have similar smoking rates, whereas Native Americans and Alaska Natives have higher smoking rates compared to Non-Hispanic Blacks and Non-Hispanic Whites. Mexican Americans have the lowest smoking rates. In the Asian population, there is great variability in the rates of smoking with the highest rates among Korean men and the lowest among the Asian Indian men. Higher smoking rates among Native Americans may explain the higher prevalence of diabetes and peripheral arterial disease in that population.

Self-monitoring of blood glucose is recognized as an important behavior contributing to achieving glycemic control, reducing hypoglycemic events and reducing the risk of diabetes complications. Although some studies have shown that there is no difference in self-monitoring of blood glucose between race/ethnic groups, several other studies have shown decreased rates of self-monitoring of blood glucose among Non-Hispanic Blacks, Hispanic Americans and Asian Americans compared to Non-Hispanic Whites, while two studies found no difference in self-monitoring of blood glucose between Native Americans and Non-Hispanic Whites.

Depression also is a well-recognized comorbidity of diabetes, and diabetic patients with depression have poorer adherence to self-management behaviors compared with those without depression. Minorities are more likely to suffer from depression, and Native Americans and Alaska Natives have high prevalence rates of depression. Non-Hispanic Blacks also are more likely to underreport their depressive Symptoms, raising concerns that the presence of depression in Non-Hispanic Blacks may be under diagnosed and undertreated. Minorities also have been found to have a poorer adherence to medications and less frequent preventive health screening, which may result in more advanced disease at presentation.

Social and environmental factors are contributors to disparities in diabetes. Minorities often live in neighborhood environments having significant disparities with respect to access to healthy food sources, places to exercise or crime related safety. Such structural social determinants of health are documented regularly in the HHS-required reports of hospitals on their community needs assessments. Lack of healthy food stores, lack of places to exercise and increased psychosocial stressors related to crime or limited social cohesion have been linked to poor health outcomes. Poor access to supermarkets has been associated with increased body mass index (BMI) and neighborhoods with increased walkability have been associated with lower BMI. Evidence from the Multi-Ethnic Study of Atherosclerosis found that “better” neighborhoods were associated with improved insulin sensitivity and decreased risk of diabetes. “Inferior” neighborhoods also have been associated with increased smoking, physical inactivity and poorer control of blood pressure, which can contribute to the development of diabetes and its complications. Management of chronic diseases can also be more difficult in low socioeconomic areas. Price differences are greater in poorer compared to wealthier neighborhoods, low-income communities have fewer pharmacies, groceries stores and supermarkets, and consequently, access to medications and healthier foods is limited in low income and minority neighborhoods.

Further evidence of racial/ethnic disparities is reflected in the difference in values for diabetes screening based on waist circumference and ethnicity: U.S. American [men≥102 cm; women≥88 cm]; European [men≥94 cm, ≥80 cm]; South Asian [men≥90, women≥80 cm]; Chinese [men≥90 cm, women≥80 cm]; Japanese [men≥90 cm, women≥80 cm]; Native South And Central American [men≥90, women≥80 cm]; Sub-Saharan African [men≥94 cm, women≥80 cm]; Eastern Mediterranean and Middle Eastern [Arab] [men≥94 cm, ≥80 cm].

Health care access and health insurance are important factors that allow patients with diabetes to receive adequate medical care. Compared to Non-Hispanic Whites, minorities with diabetes often lack health insurance. Uninsured patients with diabetes have less frequent foot and ophthalmological examinations and are less likely to receive other preventive health care services. This population has higher odds of developing diabetic eye disease and having poor glycemic control. Among Hispanic patients with diabetes, the lack of insurance has been associated with higher rates of microvascular complications. Studies also have shown that the quality of care in disadvantaged patients with diabetes is inferior compared with more affluent individuals. Minorities with diabetes were less likely to have a dilated ophthalmological examination and a lipid profile, compared to Non-Hispanic Whites. Even in countries with universal health care, studies have shown that racial/ethnic minorities receive inferior quality of health care.

Health disparities in diabetes and metabolic syndrome and their co-morbidities and complications exist worldwide. It is estimated that 1 out of 3 adults could have diabetes by 2050, due primarily to expansion of the elderly and minority populations that are high risk for diabetes. Regional data indicate that certain areas of the world such as Middle East and North Africa will continue to bear the public health burden of diabetes. In the U.S., minority children are more likely to develop type 2 diabetes than type 1 diabetes, which has economic, public health and health care system implications for these young individuals who develop a chronic condition at such an early age. Minorities in the U.S. are more likely to develop microvascular complications of diabetes and lower limb amputations, a complication from diabetes which contributes to disability.

Diabetes and metabolic syndrome treatment methods commonly focus on their metabolic element, through a combination of medications, self-care and lifestyle changes. Medication commonly consist of insulin therapy, dietary supplements, hormone supplements, anticoagulants and statins. Self-care commonly comprises insulin monitoring, medical nutrition (including nutrition education and counseling and food selection), physical exercise and weight loss, informed by family, social and genetic histories and clinical information. Lifestyle changes commonly consist of quitting smoking, reducing alcohol intake and changing risky sexual practices, as well as medical nutrition and physical exercise, all informed by family, social and genetic histories and clinical information.

Screening & Prevention

An estimated 88 million adult Americans have what is known as prediabetes, which when left unmanaged will most likely become diabetics sooner or later. This number equates to more than 33% of the adult population in the U.S. In the US, in 2009, estimates were that 40% of people with diabetes remained undiagnosed. In less-developed regions of the world, the proportion of people with undiagnosed diabetes is considered to be 50% or higher.

The natural progression or history of diabetes includes a phase comprised of prediabetes and preclinical diabetes. The estimated annual relative risk of progression from prediabetes to diabetes is 4.7%-12%, compared to 0.7% among the normoglycemic population. In the Diabetes and Aging study, the seven-year cumulative incidence of complete remission was 0.14%.

Prediabetes is most often, but not always, asymptomatic. The duration of this latency period could be as long as 9-12 years. Several studies have shown that up to 50% of people with newly diagnosed or screen-detected diabetes already exhibit diabetes-related macrovascular complications (such as ischemic heart disease or myocardial infarction) and microvascular complications (such as retinopathy, chronic kidney disease and neuropathy).

There is a strong rationale for undertaking screening for prediabetes and undiagnosed diabetes among high-risk people in clinical settings. There is no direct evidence from a randomized controlled trial or observational trial evidence on the cost-effectiveness of screening. However, economic modeling studies have suggested that targeted opportunistic screening for prediabetes, as well as diabetes, would be cost effective. Intensive programs of lifestyle modification (particularly diet, exercise and behavior) do reduce the incidence of diabetes among screen-detected people in the US Diabetes Prevention Program. In addition, there is robust evidence on the beneficial effects of early treatment of prediabetes and similar evidence is accumulating on early treatment for undiagnosed diabetes.

Screening tests for diabetes include risk scoring tools. Various tools based on known metabolic risk factors for diabetes have been developed to identify people at high risk of prediabetes or diabetes. In the U.S., the most widely validated and simple-to-use risk screening tool is the American Diabetes Association (ADA) risk questionnaire. This tool combines information on certain structural (but not person-centric modifiable) social determinants of health—age, body mass index, ethnicity, history of hypertension, family history of diabetes and history of gestational diabetes mellitus—to estimate the risk of prediabetes or diabetes. There is no tool for identifying people at high risk of prediabetes or diabetes based on lifestyle modification by changing or altering mSDOH, including mSDOH attributable to patient persona, personal preferences and other patient-centric characteristics.

Lifestyle Modification

Lifestyle modification, including weight loss, healthy diet and increased physical activity, is the cornerstone of therapy for diabetes and metabolic syndrome, as well as for their common component risk factors.

The metabolic aspects of the metabolic syndrome extend to lifestyle, social and environmental factors. In the Standards of Medical Care in Diabetes—2019, the ADA recognized the association between social and environmental factors and the prevention and treatment of diabetes. Such standards further recognized lifestyle modifications and interventions as treatment methods for diabetes. Such modifications and interventions include: diabetes self-management education and support, nutrition therapy (such as individualized assessment of eating patterns, preferences and metabolic goals), physical activity and smoking cessation. The ADA issued a call for research that seeks to better understand how such social determinants influence behaviors and how the relationships between such variables might be modified for the prevention and management of diabetes. Such standards also note that, notwithstanding such modifications and interventions, additional study is needed in the area of lifestyle modification.

Any risk factor for diabetes and metabolic syndrome requires attention, including lifestyle modification. Physical activity and diet have been identified as two core modifiable risk factors that impact onset or progression of diabetes and metabolic syndrome. The scope of lifestyle modification best practices includes birth control, consumer health, fitness, nutrition and healthy eating, smoking cessation, sexual health, stress management, weight loss and resiliency. However, it remains unclear how effective interventions are in modifying risk factors and which chronic diseases would benefit.

Treating diabetes, its comorbidities and metabolic syndrome can prevent or ameliorate diabetes and CVD, as well as many coexisting chronic diseases. Diabetes, its comorbidities and metabolic syndrome, their risk factors and long-term complications have certain commonalities of management. There is a relationship among: insulin resistance, diabetes and CVD; prevention of diabetes; metabolic syndrome and cardiovascular complications; and the goals of care and prevention aimed at prevention and mitigation of diabetes complications. Lifestyle modification is a core prevention and management approach, particularly individualized, systematic and intensive lifestyle interventions, including dietary changes, increased physical activity and weight loss. Lifestyle modifications are the most effective means of prevention of diabetes in general high-risk populations.

### SUMMARY

Methods and systems are provided comprising predicting the likelihood of patient motivation, adherence, compliance, and cessation with respect to therapy programs, promoting and improving therapy programs, patient achievement in the performance of therapy programs, patient preferences and choices in performing therapy programs, and patient retention in therapy programs, taking into consideration the patterns-of-life of the patient reflected in the social determinants of health correlated with the patient and the impact of such determinants on the design and improvement of therapy programs.

Methods and systems are described and directed to identifying, collecting, analyzing, synthesizing, and quantifying the social determinants of health and to operationalizing the resulting insights. The methods and systems are applied to structured, unstructured, disaggregated, and other data to generate insights into the patient's point of view toward adherence and compliance with therapy programs. The insights are operationalized through therapy programs for patient outreach, intervention, engagement, and retention. Statistical techniques, predictive modeling processes, therapy program components, communication methods and channels, and other methods and systems are described.

In general, the methods and systems may include: methods of identifying patients who are at risk of non-adherence, noncompliance, or likelihood of cessation, with a therapy program; predicting a basis attributable to the social determinants of health for such non-adherence, noncompliance, or likelihood of cession; and targeting interventions directed to patients who have been identified as likely to be non-compliant, wherein interventions take into consideration the predicted basis for noncompliance, particularly the impact on adherence and compliance of the social determinants of health as a basis for non-adherence and noncompliance. In addition, the methods and systems include methods of structuring therapy programs to increase the likelihood of adherence and/or compliance and to reduce the likelihood of cessation of therapy.

Assessment and measures may relate to assessing data points in every-day life, economic security and financial resources, livelihood security and employment opportunity, school readiness and educational attainment, environmental quality, civic involvement and political access, availability and utilization of quality healthcare services, adequate, affordable and safe housing, community safety and security, transportation, socioeconomic status, sociocultural status, psychosocial status, beliefs, attitudes, social support, intention to persist, health inequalities, inequities, justifiable inequalities and justifiable inequities, and other factors. The statistical methods and mathematic models applied may relate to the principles of decomposition, achievement, distribution, redistribution, dominance, curves, inequality, household mapping, poverty, welfare, and polarization, as well as factor analysis, cluster analysis, component analysis, cluster partitioning, multivariate analysis, univariate logistic and partial least square regressions, principal component analysis, and structural equation modelling.

The inventive subject matter and its basis in the social determinants of health may be used to address failures in adherence to and/or compliance with treatment, or medication, or preventive health therapy programs, may be used to increase the likelihood or probability that a patient will adhere to and/or comply with treatment, medication, or preventive health therapy, may be used for improving adherence and/or compliance with medication therapy and/or healthcare therapy that incudes medication therapy, may be used to improve compliance with and/or adherence to other wellness and/or healthcare programs, may be used to terminate nonperformance and return to performance of a prescribed or recommended compliance activity, may be used to diagnose illness and/or medical conditions, may be used to allocate resources, and may be used to design health benefit plans.

The unmet medical need addressed by the invention is the treatment of diabetes, including its prevention, diagnosis, delay in the onset, treatment and reduction in severity: (a) through the earlier identification of the risk of diabetes; (b) through lifestyle modification by the application of personal decision-making to change or alter mSDOH; (c) through the application of insights from such changes or alterations to inform lifestyle modification therapies and (d) through mSDOH-informed screening, health risk analysis, diagnosis, comprehensive care plan design/update, education, counseling, care plan outreach, engagement and retention and medication management.

The invention introduces a novel lifestyle modification instrument for the treatment of diabetes, including its prevention, diagnosis, delay in the onset, treatment management and reduction in severity and the coexisting lifestyle-management-aspects of diabetes' comorbid chronic diseases and diabetes' complications. The lifestyle modification instrument introduces novel changes or alterations by the patient of mSDOH, introduces novel operationalization by the healthcare team of the insights from such changes and introduces novel treatment processes, measures and outcomes informed by mSDOH. The lifestyle modification instrument informs the healthcare team in its: assessment of health risk; medical decision-making; comprehensive care plan development, updates and management; and patient outreach, engagement and retention in the care plan. Changed or altered mSDOH informing the healthcare team include: social determinants of health relevant to the patient and the basis for relevancy; care plan directives including intensity, frequency, duration, size, dosage and portion; performance and compliance with care plan directives; and the state and stage of diabetes. Such information is operationalized by the lifestyle modification instrument through the management of commonly-prescribed diabetes goals, education, counseling, training, nutrition and physical exercise. The lifestyle management instrument introduces a novel mode of healthcare delivery and feedback through remote, interactive, patient-reported-outcomes instruments informed by mSDOH and multidimension point-of-care-tests informed by mSDOH. Healthcare delivery and feedback measures, outcomes and analytics are based on nested domains and dimensions, endpoint families, a mSDOH-burden hazard index and dynamic interactive data cards based on such determinants.

The invention adds a new and improved dimension to lifestyle modification, namely changing or altering those social determinants of health that are a person's modifiable behaviors. The invention recognizes as part of the meaning and scope of modifiable behaviors a person's persona, including a person's value proposition and associated traits, conditions and habits, personal preferences and patient-centric characteristics. The invention operationalizes the patient's persona, together with changes or alterations therein during the patient's navigation of the pattern-of-life, to inform the healthcare team and patient in the diagnosis of diabetes and in the design, delivery and management of diabetes treatment, delay in the onset and prevention.

The invention addresses mSDOH as a new and improved type of healthcare information and treatment method. The invention uniquely recognizes a person's persona, as a predictable pattern of Signs and Symptoms or physical findings that are risk factors to controlling or significantly influencing diabetes and its risk factors coexisting with metabolic syndrome. The invention identifies, changes or alters the mSDOH of a person and interprets and applies the resulting insights to inform the health risk, diagnosis, treatment, delay in the onset and prevention of diabetes.

The health-related efficacy of the invention's treatment interventions and behavioral outcomes are recognized by the National Institutes of Health in their definition of a clinical trial. The invention's treatment method evaluates the effects of interventions that change or alter mSDOH on health-related biomedical and behavioral processes, endpoints, outcomes and a person's quality of life. As a new or improved treatment method, the invention intervenes and manipulates the patient's mSDOH and the mSDOH in the patient's environment, such as treatment strategies, prevention strategies and implementation of care plans and diagnostic strategies and procedures.

As a new or improved treatment method, the invention evaluates the outcome or effect of interventions that change one or more mSDOH on a patient's biomedical and behavioral status or quality of life, such as: positive or negative changes to disease processes; positive or negative changes to health-related behaviors; positive or negative changes to quality of life; and positive or negative changes to psychological or neurodevelopmental parameters, such as: mood management intervention for smokers, reading comprehension and information retention.

Lifestyle modification as practiced by the invention is not solely intensive management, diabetic education or a diet supplement, but instead is comprised of (a) the modifiable risk factors of prescribed (i) weight loss, (ii) healthy diet, (iii) increased physical activity and (d) at least one mSDOH, together with at least one of (b) another lifestyle modification strategy, guideline or directive recommended by a diabetes professional organization or government (such as diabetes education, self-management education and support, counseling, individual or group therapy, stress reduction and smoking cessation) and (c) all delivered through a multidisciplined comprehensive care team approach.

The invention informs one or more components of the lifestyle modification of the patient's mSDOH and changes or alterations to the patient's mSDOH. The invention informs the care team in its health risk analysis and its design and updates of the comprehensive care plan based on changes or alterations to mSDOH, as well as other risk factors.

The invention reports delay, progression and recurrence of risk factors of diabetes, its coexisting CVD and other chronic diseases and metabolic syndrome. The invention also reports the progression, improvement and risk reduction measures of diabetes, its coexisting CVD and other chronic diseases and metabolic syndrome.

The invention uniquely combines and interprets mSDOH to inform diagnosis and treatment during the patient's navigation of the pattern-of-life. The mSDOH pattern-of-life data is obtained through remote patient-reported Signs, Symptoms, patient performance of care plan directives and outcomes. Remote reporting is through multiplexed point of care testing simultaneously quantifying a variety of mSDOH variables from a single patient encounter face-to-face with or external to the healthcare team. As such, while navigating the pattern-of-life by the patient, the invention provides continuous (as distinguished from episodic) mSDOH data and interpretations of such data for pro-active decision-making by patients, for medical decision-making by the comprehensive care team and pro-active decisioning by caregivers of the patient. The invention interprets such data to generate a unique mSDOH-informed patient profile and a unique mSDOH-informed patient risk score.

The unique mSDOH-informed patient profile and risk score are interpreted by the invention to enable the patient and the comprehensive care team to identify and modify or adjust: the patient's lifestyle value proposition; mSDOH; and the resulting impact on the existence, state, stage and progression of metabolic abnormalities of diabetes. The unique mSDOH-informed patient profile and risk score are interpreted by the invention: to evaluate compliance with the directives of the comprehensive care plan; and to alert the patient and the healthcare team to the patient's likelihood of cessation from the care plan.

The unique mSDOH-informed patient profile risk and score are interpreted by the invention to identify socioeconomic disparities impacting lifestyle and to change mSDOH to improve the design and updating of the comprehensive care plan and the patient's compliance with such plan. By identifying and changing or altering those mSDOH that impact, or are impacted by, socioeconomic disparities, the invention designs and operationalizes lifestyle modifications that control progression from prediabetes to diabetes, including the risk factor cluster correlating with diabetes, CVD, metabolic syndrome and other coexisting comorbidities.

Changes or alterations to patient persona, personal preferences and other patient-centric characteristics, specific components of lifestyle modifications and the composition of the care team contribute to better outcomes. As a result, the invention improves: the early discovery and diagnosis of diabetes in asymptomatic patients; the delay in the onset of diabetes in prediabetic patients; the treatment of diagnosed diabetics; the patient's quality of life; and reduction in premature death, healthcare cost to the patient and providers and the patient's utilization of the healthcare system.

### Terms; Concepts; Definitions

Throughout this description of the invention, several terms and related concepts and definitions are used to aid the understanding of certain concepts pertaining to the associated treatment methods. These terms, concepts and definitions are intended to help provide an easy methodology of communicating the ideas expressed herein and are not necessarily meant to limit the scope of embodiments of the invention. In addition, where terms, concepts and definitions are utilized herein that have been used inconsistently in the literature, the list of these terms and their integrated meanings, concepts and definitions will be adopted as follows:

Concept or Clinical Concept: Generally, health-related continuous variables, attributes, features, models, functions, equations or information that have been or are capable of being converted or partitioned into nominal or discretized counterparts capable of being encoded. The Concept includes the specific measurement goal (that is, the thing that is to be measured by a health assessment, whether objective or subjective, including a patient-reported-outcome and a xPOCT) to measure the effect of a medical intervention on one or more Concepts.

A Clinical Concept may include Clinical Variables, Clinical Values, Clinical Information Elements, Proxies, Proxy Variables and ensembles of such variables, values and information elements. For example, a Clinical Variable having a Clinical Value may be encoded as a single code representing at least one of the Clinical Variable and the Clinical Value or as a single code representing a combination or ensemble of one or more Clinical Variables and one or more Clinical Values.

Condition or Clinical Condition: Diabetes or a co-existing or related disease, illness, diagnosis, medical issue or medical event for a patient, wherein an event may include an epoch or series of epochs.

Information Element: A piece or component of health-related information for a patient used for medical decision-making to help make patient care decisions, such as for example a social determinant of health, a mSDOH, health risk, social history, family history, medical history, a lab result, finding, test, or study or other component element of clinical information.

Value or Clinical Value or Measure: Patient-specific value associated with a Clinical Variable, such as for example: 132 lbs. for the Clinical Value weight; 32 years for the Clinical Value age; 120 for the Clinical Value systolic blood pressure; age, gender, race/ethnicity for the Clinical Value demographics; moderate, acute or chronic for the Clinical Value diagnosis; and low, moderate or high for the Clinical Value health risk analysis.

Variable/Attribute or Clinical Variable/Attribute: A category or type of clinical information about a patient used for medical decision-making to help make patient care decisions, such as for example social determinants of health (such as for example social history [including marital status and living arrangements, current employment status, occupational history, the usage status of drugs, alcohol or tobacco, level of education, sexual history and other relevant social factors], family history, medical history) and mSDOH, as well as health risk state or status, Signs, Symptoms, organ system assessment, blood pressure, respiratory rate, body weight, blood glucose, sex, age, condition(s), diagnoses and other types of clinical information.

Program or Condition Program: A program, such as for example a course, algorithm, rule(s), routine, guideline, care plan or goal, for determining a patient's likelihood of having or developing a Clinical Condition and prescribed or recommended by the healthcare professional responsible for the patient.

Risk Factors: A set of Clinical Variables and associated values for a patient that are determined to be relevant to a Clinical Condition, including a medical decision-making supporting event, and that are used for determining a patient's likelihood for having or developing a Clinical Condition. In some embodiments and scenarios, Risk Factors may include other Clinical Conditions such as for example coexisting comorbidities of a primary chronic Clinical Condition.

Risk Score: A mathematical or other expression of the likelihood or probability that a patient has or will develop a Clinical Condition or the likelihood its state or status will be static or will change for the positive or negative. Where appropriate, a degree or intensity or frequency of the patient's condition (such as for example mild, severe or acute; worse, some, better; on a scale of 0-10 such as where “0” is best and “10” is worst) is also considered in assessing such likelihood or probability.

Domain: A sub-Concept represented by a score of a PRO Instrument or xPOCT that measures a larger Concept comprised of multiple Domains. For example, Domains include treatment sites, comorbidities, medical histories, care plans/medical decision-making, family histories, extended histories, social histories, patient personalities and repeat measures.

Essentialities: Evidence of the patient's unique social and personal competencies, particularized to the dimensions, preferences and factors valuable, as well as harmful, that the patient utilizes in navigating the patient's pattern-of-life and situated within and expressed by the patient's pattern-of-life, comprised of perceived HRQoL components further comprising demographics and compliance Domains where compliance Domains are further comprised of: (a) physical health and capacity (comprising Symptoms or Signs of at least one of pain, discomfort, energy, fatigue, sleep and rest); (b) psychological (comprising Symptoms or Signs of at least one of positive feelings, thinking, learning, memory, concentration, self-esteem, bodily image, appearance and negative feelings); (c) level of independence (comprising Symptoms or Signs of at least one of mobility, activities of daily living, dependence on medication or treatments and work capacity); (d) social relationships (comprising Symptoms or Signs of at least one of personal relationships, social support, sexual activity, clinical family history, clinical social history and pros and cons of how the patient functions, feels, survives); (e) environment (comprising Symptoms or Signs of at least one of physical safety, security, home environment, financial resources, healthcare accessibility, healthcare quality, social care accessibility, social care quality, opportunities to acquire new or improved information and skills, opportunities for recreation activities, opportunities for leisure activities, participation in recreation activities, participation in leisure activities, pollution physical environment, traffic physical environment, climate physical environment and transport); (f) spirituality-religion-personal beliefs (comprising Symptoms or Signs of at least one of comfort, well-belonging; security; sense of belonging, purpose, strength, intensity; capacity; frequency; evaluation of states and evaluation of behaviors).

Provider, Healthcare Team, Healthcare Entity: a medical organization, a care team, a healthcare individual or a self-reporting patient or a related entity including caregivers, health care administrators, insurance providers and patients, that reports or otherwise provides patient health information pursuant to government requirements or otherwise for quality assurance or quality improvement purposes or is otherwise associated with a local user of such information or the patient;

HRQoL: Health-related quality of life is an individual's satisfaction or happiness with Domains of life insofar as they affect or are affected by “health”. The World Health Organization defines health as a concept that incorporates notions of well-being or wellness in all areas of life (physical, mental, emotional, social, spiritual), and “not merely the absence of disease or infirmity.” Accordingly, health is a broad concept that subsumes the related concepts of disease, illness, wellness and other Clinical Conditions. Generally, assessment of HRQoL represents an attempt to determine how Clinical Concepts and other variables within the dimension of health relate to particular dimensions of life that have been determined to be valuable or important to people in general (generic HRQoL) or to people who have a specific disease (condition-specific HRQoL). Conceptualizations of HRQoL included in the Concept Domain of HRQoL emphasize the effects of Clinical Conditions on physical, social/role, psychological/emotional and cognitive functioning, as well as Symptoms, Signs, health perceptions and overall quality of life. HRQoL is distinguished from quality of life in that HRQoL concerns itself primarily with those factors that fall under the purview of healthcare providers, healthcare entities, healthcare systems and healthcare interventions. Quality of life is an individual's satisfaction or happiness with life in Domains the individual considers valuable or important. Historically known as “life satisfaction” or “subjective well-being,” quality of life also is known as “overall quality of life” or “global quality of life” to distinguish quality of life from “health-related quality of life.”

Item, Element: An individual question, statement or task that is evaluated or assessed by the patient, or the patient's Proxy, to address a Concept. Items include individual mSDOH Clinical Variables.

mSDOH: Modifiable social determinants of health, being those pattern-of-life factors that impact health, that are relevant to the patient's pattern-of-life, that are alterable by changing one or more of such factors or patterns and that, when altered as directed in a care plan, improve the health of the patient, or when not altered as directed in a care plan, harm the health of the patient.

PRO Concepts: Patient-reported-outcomes or expressions of how the patient functions or feels with respect to a Clinical Condition or a Treatment Risk Or Benefit, where the expression is an assessment made directly the by the patient, or in some cases when appropriate, by a Proxy for the patient.

PRO Data: Patient-reported-outcome measurement of Signs, Symptoms, patient behaviors and changes therein over time based on a report that comes directly from the patient, or in some cases when appropriate, by a Proxy for the patient, and that assesses the state or status of the patient's health condition without amendment or interpretation of the patient's response by a clinician or anyone else.

PRO Instrument Concept: Patient-reported-outcome aspects of how the patient feels or functions with respect to treatment risk or benefits.

PRO Instrument: An interactive personal digital assistant used by the patient to capture, assess and report PRO Data and to measure the change over time in how the patient feels or functions.

Proxy or Proxy Variable: A measurement required to stand-in for or operationalize variables that are not directly relevant or that cannot be directly measured, but that serve in place of an unobservable or immeasurable variable, where the Proxy or Proxy Variable has a close correlation with the variable of interest, relates to an unobserved variable, correlates with disturbance and does not correlate with regressors once disturbance is controlled for. The close correlation may be positive or negative and need not necessarily be linear. Examples of Proxies or Proxy Variables include: a clinical trial, where indices, scores and decompositions are created or applied as Proxies for evaluating the effects of medical interventions for the purpose of modifying health-related behavioral processes and endpoints, including trials defined by the National Institutes of Health that measure positive or negative changes to disease processes, to health-related behaviors, to quality of life and to health-related quality of life; Body Mass Index (BMI) as a Proxy for true body fat percentage; changes in height over a fixed time as a Proxy for hormone levels in blood; years of education and/or GPA as a Proxy for cognitive ability; per-capita GDP as a Proxy for measures of standard of living or quality of life; country of origin or birthplace as a Proxy for race, or vice versa; widths of tree rings as a Proxy for historical environmental conditions; satellite images of ocean surface color as a Proxy for depth that light penetrates into the ocean over large areas.

Responder Definitions, Response Outcomes: The change, as reported by a PRO Instrument or xPOCT, in one or more component factors comprising a Condition Risk Score for a patient over a predetermined time period that is interpreted as a Treatment Risk Or Benefit. Measurement of such change may be quantitative (such as for example by number or percent of reductions or improvement or other measure) and qualitative (such as for example worse, some, better; etc.).

Sign: Any objective evidence of a disease, health condition or treatment-related effect that is not a Symptom.

Symptom: Any subjective evidence of a disease, health condition or treatment-related effect that can be noticed and known only by the patient.

Treatment Benefit: How the patient survives, feels or functions.

Treatment Risk Or Benefit: The effect of treatment on how a patient survives, feels or functions.

In the following description, for purposes of explanation, numerous specific details of various objects, features, aspects, and advantages of the inventive subject matter are set forth in order to provide a thorough understanding of example embodiments. It will be evident, however, to one of ordinary skill in the art that embodiments of the invention may be practiced without these specific details.

Unless otherwise defined, all technical and scientific terms used herein have the same meaning as commonly understood by one of ordinary skill in the art to which the inventive subject matter belongs. Although methods and materials similar or equivalent to those described herein can be used in the practice or testing of the present invention, suitable methods and materials are described below. All publications, patent applications, patents, and other references mentioned herein are incorporated by reference. In case of conflict, the present specification, including definitions, will control. In addition, the materials, methods, systems, and examples are illustrative only and not intended to be limiting. Other features, objects, and advantages of the invention will be apparent from the description and drawings and from the claims.

### BRIEF DESCRIPTION OF THE DRAWINGS

FIG. 1 is a block diagram of an example system, according to an example embodiment;

FIG. 2 is a block diagram of an example data stream storing, mining, and synthesizing sub-system deployed in the system of FIG. 1, according to an example embodiment;

FIG. 2A is a block diagram of a method of predictive model building;

FIG. 3 is a block diagram of an example prediction modeling sub-system deployed in the system of FIG. 1, according to an example embodiment;

FIG. 3A illustrates an example prediction module that may be deployed in a the data-modeling sub-system of FIG. 3, according to an example embodiment;

FIG. 3B is a block diagram of a flowchart illustrating a method of predicting likelihood of cessation of a therapy program according to an example embodiment;

FIG. 3C is a block diagram of a flowchart illustrating a method of predicting compliance with a therapy program, according to an example embodiment;

FIG. 3D is a block diagram of a flowchart illustrating a method of predicting adherence to a therapy program, according to an example embodiment;

FIG. 3E is a block diagram of a flowchart illustrating a method for therapy program implementation, according to an example embodiment;

FIG. 3F is a block diagram of a flowchart illustrating a method for predicting adherence to, compliance with, or cessation of a therapy program, according to an example embodiment;

FIG. 3G is a block diagram of a flowchart illustrating a method for activating the adherence, compliance, and cessation predictions in the design and/or improvement of a therapy program, according to an example embodiment;

FIG. 3H is a block diagram of a compliance model according to an example embodiment;

FIG. 3i is a block diagram of a method by which of the inventive subject matter may be operationalized by an electronic medical records system;

FIG. 3J shows a block diagram of a method by which the inventive subject matter may be operationalized for patient targeting and outreach, according to an example embodiment;

FIG. 4 is a block diagram of an example prediction activation sub-system deployed in the system of FIG. 1, according to an example embodiment;

FIG. 4A is an example illustration of a therapy strategy surveillance and monitoring and reinforcement subsystem, according to an example embodiment;

FIG. 4B and an example illustration of a therapy compliance measurement subsystem, according to an example embodiment;

FIG. 4C is a block diagram of a method by which the inventive subject matter may be operationalized by patient intervention and engagement, according to an example embodiment;

FIG. 5 is a block diagram of an example communication device for a healthcare professional deployed in the system of FIG. 1, according to an example embodiment;

FIG. 6 is a block diagram of an example communication device for a patient deployed in the system of FIG. 1, according to an example embodiment; and

FIG. 7 is a block diagram of a machine in the example form of a computer system within which a set of instructions for causing the machine to perform any one or more of the methodologies discussed herein may be executed.

FIG. 8 depicts a Summary: Diabetes Treatment Method Informed By mSDOH.

FIG. 9 depicts a Summary: Diabetes Treatment Method Informed By mSDOH; Comprehensive Care Plan System.

FIG. 10 depicts a Summary: Diabetes Treatment Method Informed By mSDOH: mSDOH Operating Systems.

FIG. 11 depicts a diagram of a Comprehensive Care Team.

FIG. 12 depicts a diagram of a Patient Profile.

FIG. 13 depicts a diagram of a Hazard Assessment Process.

FIG. 14 depicts a diagram of a mSDOH-Informed Multiplexed Point-Of-Care Diabetes Hazard Assessment System Measures.

FIG. 15 depicts a diagram of a mSDOH-Informed Multiplexed Point-Of-Care Diabetes Hazard Assessment System Diagnostic Test: mSDOH Hazard Calculator.

FIG. 16 depicts a diagram of a mSDOH-Informed Multiplexed Point-Of-Care Diabetes Hazard Assessment System Diagnostic Test: Hazard Calculation Adjustments For mSDOH.

FIG. 17 depicts a diagram of a mSDOH-Informed Multiplexed Point-Of-Care Diabetes Hazard Assessment System mSDOH-Informed Prediabetes Comprehensive Care Plan.

FIG. 18 depicts a diagram of a Patient Pattern-Of-Life Profile.

FIG. 19A depicts a diagram of a mSDOH Translation System: Patient Preference Machine.

FIG. 19B depicts a diagram of a msDOH Interpretation System.

FIG. 20 depicts a diagram of a mSDOH Translation System: Clinical Reported Outcomes Machine Module.

FIG. 21 depicts a diagram of a mSDOH Translation System: mSDOH Reported Outcomes Machine Module; Nested Domain Structure.

FIG. 22 depicts a diagram of a mSDOH Translation System: mSDOH Reported Outcomes Machine Module; Primary End-Point Families Proxies; Nested Domain Structure.

FIG. 23 depicts a diagram of a mSDOH Translation System: Patient Reported Outcomes Machine Module; Nested Domain Structure.

FIG. 24 depicts a diagram of a mSDOH Medical Decision-Making System: Diabetes Pattern-Of-Life Knowledge Machine Module.

FIG. 25 depicts a diagram of a mSDOH Medical Decision-Making System: Diabetes Pattern-Of-Life Knowledge Machine Module; Patient Reported Outcomes Modifiable Behavior Domains.

FIG. 26 depicts a diagram of a mSDOH Medical Decision-Making System: Diabetes Pattern-Of-Life Knowledge Machine Module; Patient Reported Social & Economic Circumstances Domain.

FIG. 27 depicts a diagram of a mSDOH Medical Decision-Making System: Diabetes Pattern-Of-Life Knowledge Machine Module; Patient Reported Outcomes Socio Needs Domains.

FIG. 28A depicts a diagram of a mSDOH Medical Decision-Making System: Diabetes Pattern-Of-Life Knowledge Machine Module; Health Risk Calculator.

FIG. 28B depicts a diagram of a mSDOH Medical Decision-Making System: Diabetes Pattern-Of-Life Knowledge Machine Module; Statistical Considerations—Nested Domain Structure.

FIG. 29 depicts a diagram of a mSDOH Medical Decision-Making System: Diabetes Knowledge Feedback And Evaluation Machine Module; Health Risk Evaluator.

FIG. 30 depicts a diagram of a mSDOH Medical Decision-Making System: Diabetes Optimized Therapy Program; Lifestyle Modification Instrument Components.

FIG. 31 depicts a diagram of a mSDOH Medical Decision-Making System: Diabetes Optimized Therapy Program; Lifestyle Modification Instrument Comprehensive Care Plan Requirements.

FIG. 32 depicts a diagram of a mSDOH Medical Decision-Making System: Diabetes Optimized Therapy Program; Lifestyle Modification Instrument Manager.

FIG. 33 depicts a diagram of a mSDOH Medical Decision-Making System: Diabetes Optimized Therapy Program; Lifestyle Modification Instrument Program Components.

FIG. 34 depicts a diagram of a mSDOH Medical Decision-Making System: Diabetes Optimized Therapy Program; Lifestyle Modification Instrument Program Components (cont.).

FIG. 35 depicts a diagram of a mSDOH Diabetes Pattern-Of-Life Navigation System: Pattern-Of-Life Navigation Services Module; Patient Outreach And Engagement.

FIG. 36 depicts a diagram of a mSDOH Diabetes Pattern-Of-Life Navigation System: Pattern-Of-Life Navigation Services Module; Patient Outreach And Engagement—Patient Reported Outcomes Instrument.

FIG. 37 depicts a diagram of a mSDOH Diabetes Pattern-Of-Life Navigation System; Pattern-Of-Life Navigation Services Module; Conceptual Framework Of The PRO Instrument.

FIG. 38 depicts a diagram of a mSDOH Diabetes Pattern-Of-Life Navigation System: Community Assets Utilization Services Module.

FIG. 39 depicts a diagram of a mSDOH Diabetes Pattern-Of-Life Navigation System: Celebration Management Services Module.

### DETAILED DESCRIPTION

The term “Social Determinants Of Health,” including the respective Components Of Health of such determinants and the respective Indicators Of Health of such components, as used with respect to the inventive subject matter shall mean the conditions in which people are born, grow up, live, work, and age, including the heath system, including without limitation: (a) the circumstances and/or patterns of daily life, including differential exposure to influences that cause disease in early life, social and physical environments, and work associated with social stratification; (b) the circumstances, patterns, and/or conditions of daily life that influence a person's opportunity to be healthy, a person's risk of illness, and/or a person's life expectancy; (c) healthcare responses to health promotion, disease prevention, and/or treatment of illness; (d) the structural drivers that address the nature and degree of social stratification in society; (e) the norms and values of society; (f) global, national, and local economic and social policies; and (g) national and local governance processes. For convenience with respect to the inventive subject matter, the term Social Determinants Of Health may be used interchangeably with the Components Of Health and the Indicators Of Health as the context of the inventive subject matter may require.

The term “data” when used in correlation, attribution, or otherwise associated with the inventive subject matter, the Social Determinants Of Health, a patient and/or a population (including an illness population and/or a medical condition population) shall include, without limitation and without regard as to the source, collection method, format, or delivery channel of such data: consumer data, demographic data, deprivation data, healthcare access data, healthcare supply data, healthcare use data, life-stage data, lifestyle data, livelihood strategy data, pattern-of-life data, personal health data associated with an individual, population health data, poverty data, psychosocial data (including psychosocial status), public health and personal health data associated with a population, social exclusion data, social inclusion data, sociocultural (including sociocultural status) data, socioeconomic (including socioeconomic status) data, and vulnerability data.

Before the inventive subjective matter is described in further detail, it is to be understood that such subject matter is not limited to the particular aspects or embodiments described, as such may, of course, vary. It is also to be understood that the terminology used herein is for the purpose of describing particular aspects and embodiments only, and is not intended to be limiting, since the scope of the present inventive subject matter will be limited only by the appended claims.

Where a range of values is provided, it is to be understood that each intervening value (to the tenth of the unit of the lower limit unless the context clearly dictates otherwise) between the upper and lower limit of that range and any other stated or intervening value in that stated range is encompassed within the inventive subject matter. The upper and lower limits of these smaller ranges independently may be included in the smaller ranges and also are encompassed within the invention, subject to any specifically excluded limit in the stated range. Where the stated range includes one or both of the limits, ranges excluding either or both of those included limits are also included in the invention.

Unless defined otherwise, all technical and scientific terms used herein are to be understood to have the same meaning as commonly understood by one of ordinary skill in the art to which the present invention belongs. Although any methods and systems similar or equivalent to those described herein also can be used in the practice or testing of the present invention, a limited number of the exemplary methods and systems are described herein.

It is noted and to be understood that, as used herein and in the appended claims, the singular forms “a,” “an,” and “the” are to include plural referents unless the context clearly dictates otherwise.

All publications mentioned herein are incorporated herein by reference to disclose or describe the methods and systems, in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Example methods and systems for creating new or improving, or for managing therapy adherence, compliance, and cessation are described. In the following descriptions, for purposes of explanation, numerous specific details are set forth in order to provide a thorough understanding of example aspects and embodiments. It will be evident, however, to one of ordinary skill in the art that aspects and embodiments of the inventive subject matter may be practiced without these specific details.

In an aspect of an embodiment, a module may be directed to operationalizing adherence, compliance, and/or the cessation of adherence or compliance as the degree to which a prescribed or recommended therapy program is followed expressed as a coefficient, index, score, or other value representing or corresponding to the degree to which two or more variables are similar, where such value measures, analyzes, and/or explains and synthesizes multiple, daily, pattern-of-life activities. and other complex social phenomena, determined through one or a plurality of statistical techniques, predictive modeling methods and/or other techniques and/or methods. In this embodiment, a module may be directed to processing a coefficient or a set of coefficients, such as a form of the Gini coefficient or a plurality of forms of the Gini coefficient, which coefficient is readily known to persons of ordinary skill in the art as an inequality index in applied statistical analysis, together with extensions, modifications, and adjustments, by way of additive decomposition of such coefficient through population subgroups where the concentration of societal measures of compliant or deprivation are expressed as one or a plurality of Indicators Of Health, Components Of Health, and/or Social Determinants Of Health having a direct algebraic link to the Gini coefficient, together with re-writing new variable derivatives of such coefficient expressing in a single summary statistic, such as an index, or score, or a sub-index, or sub-score, one Indicator Of Health or a plurality of Indicators Of Health, and/or one Component Of Health or a plurality of Components Of Health, and/or one Social Determinant Of Health or a plurality of Social Determinants Of Health.

An aspect of the invention may include some embodiments having a module or a plurality of modules directed to identifying, analyzing, adjusting, and/or comparing health inequality and well-being across distributions of living standards using data from the Indicators Of Health and other numerical and non-numerical disaggregated data, each data point representing a quantitative value indicative of one Indicator Of Health or a plurality of Indicators Of Health.

An embodiment may be directed to decomposing an Indicator Of Health or a plurality of Indicators Of Health represented by health inequalities and inequities into justifiable inequalities (such as, differences, variations, and disparities in the health achievements of individuals and groups of people such as, age, gender, etc.) and justifiable inequities (such as, a difference or disparity in health outcomes that is systematic, avoidable, and unjust such education, income, place of residence, etc.), wherein health inequality is measured by a disproportionate concentration expressed as a contribution index. In this embodiment, a module may be directed to quantifying the contribution to inequity from one unjustifiable health determinant or a plurality of unjustifiable health determinants by linking through linear regression health outcomes (such as, asthma, overweight, hypertension) to inequalities and inequities and their respective components, so that the contribution for a health outcome is linearly related to the contributions of the inequalities and inequities, and their respective components, where the coefficients of the contributions are the elasticities of the health outcomes with respect to the inequalities and inequities and their respective components. A module in such embodiment may be directed to calculating concentration indices using health outcomes and health utilization variables including: living standards measuring continuous variables (such as, consumption, expenditure, income, asset index or score); weights and survey settings relating to sample design information (such as, sampling weight, cluster, strata); household identification; standardized variables for health outcomes (such as, asthma, overweight, hypertension); standardized variables for health care utilization (such as, age, gender, urban location, and health need); and control variables (such as, income, education, place of residence, and the presence/absence of health insurance).

An embodiment may utilize smart databases that apply rules engines to execute instructions to establish and/or to combine with weighted importance the quantitative value of an Indicator Of Health and/or the quantitative values of a plurality of Indicators Of Health. A rules engine may execute instructions to establish the weighted importance of one such quantitative value or a plurality of such quantitative values utilizing aggregative methods, stratification, inverse weighting, propensity scoring, principal components analysis, factor analysis, and/or other relative methods each of which methods is readily known to persons of ordinary skill in the art. A rules engine may execute instructions to perform indexing or scoring and to arrive at one or more indices or scores associating and/or correlating an index or score with an Indicator Of Health and/or a plurality of indices or scores with a plurality of Indicators Of Health. One index or score associated with an Indicator Of Health, and/or one index or score associated with a plurality of Indicators Of Health, and/or a plurality of indices or scores associated with a plurality of Indicators Of Health may represent a quantitative contribution to an overall Indicator Of Health index or score (“Indicator Index”). An Indicator Of Health and/or a plurality of Indicators Of Health may represent a sub-index or sub-score (“Indicator Sub-Index”).

An embodiment may be directed to weighting or scaling a Determinant of Health or a plurality of Determinants Of Health for relevancy, importance, appropriateness, or otherwise significant health or utilization, and to weighing or scaling at the high end as highly unequally distributed or otherwise disproportionately weighted or scaled, and/or to weighing or scaling at the low end as equally distributed or low equal distributed, and to accounting for a comparatively larger share or component of the inequality in health or utilization.

Some embodiments may have a module or a plurality of modules directed to identifying, analyzing, adjusting, and/or comparing health inequality and well-being across distributions of living standards using data from the Components Of Health and other numerical and non-numerical disaggregated data, each data point representing a quantitative value indicative of one Component Of Health or a plurality of Components Of Health.

An embodiment may be directed to decomposing Components Of Health represented by health inequalities and inequities into justifiable inequalities (such as, differences, variations, and disparities in the health achievements of individuals and groups of people such as, age, gender, etc.) and justifiable inequities (such as, a difference or disparity in health outcomes that is systematic, avoidable, and unjust such education, income, place of residence, etc.), wherein health inequality is measured by a contribution index. In this embodiment, a module may be directed to the contribution to inequity from one unjustifiable health determinant or a plurality of unjustifiable health determinants by linking through linear regression health outcomes (such as, asthma, overweight, hypertension) to inequalities and inequities and their respective components, so that the contribution for a health outcome is linearly related to the contributions of the inequalities and inequities and their respective components where the coefficients of the contributions are the elasticities of the health outcomes with respect to the inequalities and inequities and their respective components. A module in such embodiment may be directed to calculating concentration indices using health outcomes and health utilization variables including: living standards measuring continuous variables (such as, consumption, expenditure, income, asset index or score); weights and survey settings relating to sample design information (sampling weight, cluster, strata); household ID; standardized variables for health outcomes (such as, asthma, overweight, hypertension); standardized variables for health care utilization (such as, age, gender and health need); and control variables (such as, income, education, please of residence and health insurance).

An embodiment may utilize smart databases that apply rules engines to execute instructions to establish and/or to combine with weighted importance the quantitative value of a Component Of Health and/or the quantitative values of a plurality of Components Of Health. A rules engine may execute instructions to establish the weighted importance of one such quantitative value or a plurality of such quantitative values utilizing aggregative methods, stratification, inverse weighting, propensity scoring, principal components analysis, factor analysis, and/or other relative methods each of which methods is readily known to persons of ordinary skill in the art. A rules engine may execute instructions to perform indexing or scoring and arrive at one or more indices or scores associating and/or correlating an index or score with a Component Of Health and/or a plurality of indices or scores with a plurality of Components Of Health. One index or score associated with a Component Of Health, and/or one index or score associated with a plurality of Components Of Health, and/or a plurality of indices or scores associated with a plurality of Components Of Health may represent a quantitative contribution to an overall Component Of Health index or score (“Component Index”). A Component Of Health and/or a plurality of Components Of Health may represent a sub-index or sub-score (“Component Sub-Index”).

An embodiment may be directed to weighting or scaling a Component Of Health or a plurality of Components Of Health for relevancy, importance, appropriateness, or otherwise significant health or utilization, and to weighing or scaling at the high end as highly unequally distributed or otherwise disproportionately weighted or scaled, and/or to weighing or scaling at the low end as equally distributed or low equal distributed, and to accounting for a comparatively larger share or component of the inequality in health or utilization.

Some embodiments may have a module or a plurality of modules directed to identifying, analyzing, adjusting, and/or comparing health inequality and well-being across distributions of living standards using data from the Social Determinants Of Health and other numerical and non-numerical disaggregated data, each data point representing a quantitative value indicative of one Social Determinant Of Health or a plurality of Social Determinants Of Health.

An embodiment may be directed to decomposing Social Determinants Of Health represented by health inequalities and inequities into justifiable inequalities (such as, differences, variations, and disparities in the health achievements of individuals and groups of people such as, age, gender, etc.) and justifiable inequities (such as, a difference or disparity in health outcomes that is systematic, avoidable, and unjust such education, income, place of residence, etc.), wherein health inequality is measured by a contribution index. In this embodiment, a module may be directed to quantifying the contribution to inequity from one unjustifiable health determinant or a plurality of unjustifiable health determinants by linking through linear regression health outcomes (such as, asthma, overweight, hypertension) to inequalities and inequities and their respective components, so that the contribution for a health outcome is linearly related to the contributions of the inequalities and inequities and their respective components where the coefficients of the contributions are the elasticities of the health outcomes with respect to the inequalities and inequities and their respective components. A module in such embodiment may be directed to calculating concentration indices using health outcomes and health utilization variables including: living standards measuring continuous variables (such as, consumption, expenditure, income, asset index or score); weights and survey settings relating to sample design information (sampling weight, cluster, strata); household ID; standardized variables for health outcomes (such as, asthma, overweight, hypertension); standardized variables for health care utilization (such as, age, gender and health need); and control variables (such as, income, education, please of residence and health insurance).

An embodiment may utilize smart databases that apply rules engines to establish and/or to combine with weighted importance the quantitative value of a Social Determinant Of Health and/or the quantitative values of a plurality of Social Determinants Of Health. A rules engine may execute instructions to establish the weighted importance of one such quantitative value or a plurality of such quantitative values utilizing aggregative methods, stratification, inverse weighting, propensity scoring, principal components analysis, factor analysis, and/or other relative methods each of which methods is readily known to persons of ordinary skill in the art. A rules engine may execute instructions to perform indexing or scoring and to arrive at one or more indices or scores associating and/or correlating an index or score with a Social Determinant Of Health and/or a plurality of indices or scores with a plurality of Social Determinants Of Health. One index or score associated with a Social Determinant Of Health, and/or one index or score associated with a plurality of Social Determinants Of Health, and/or a plurality of indices or scores associated with a plurality of Social Determinants Of Health may represent a quantitative contribution to an overall Social Determinant Of Health index or score (“Social Determinant Index”).

An embodiment may be directed to determining the concentration index and the related concentration curve associated or correlated with an Indicator Of Health, a Component Of Health, a Social Determinant, and/or a plurality of Indicators Of Health, Components Of Health and/or Social Determinants Of Health, where the concentration index and related concentration curve provide a means of quantifying the degree of income-related inequality in a specific health variable. The concentration index and the concentrated curve are readily known to persons of ordinary skill in the art. The concentration index is defined with reference to the concentration curve.

In such embodiment, the two key variables underlying the concentration curve are the health variable, the distribution of which is the subject of interest, and a variable capturing the living standards against which the distribution is to be assessed. The health variable is measured in units that can be aggregated across individuals. This is not necessary for the living standards measure. The data may be at the individual level (e.g., raw household survey data), in which case values of both the health variable and the living standards variable are available for each observation. Alternatively, the data may be grouped, in which case, for each living-standard group (e.g., income quintile), the mean value of the health variable is observed. The ranking of the groups (which group is poorest, which group is second poorest, and so on) and the percentage of the sample falling into each group may be known or unknown. In the case of grouped data, the advantage of the concentration curve over a table of group means is that such curve gives a graphical representation of the data.

Computing the concentration index, C, from grouped data may be performed in a spreadsheet program using the following formula:

C=(p1L2−p2L1)+(p2L3−p3L2)+ . . . +(pT−1LT−pTLT−1)

where p is the cumulative percent of the sample ranked by economic status, L(p) is the corresponding concentration curve ordinate, and T is the number of socioeconomic groups. Adjustments may be made, such as for example, for computing a standard error, for applications where the standard errors of the group means are known or unknown and for dominance of sampling variability.

Computing the concentration index from individual-level data on both the health variable and socioeconomic ranking variable may be performed in a spreadsheet program by making use of the convenient covariance result using the following formula:

C=2 cov(yi,Ri)/□

where y is the health variable whose inequality is being measured, □, is its mean, Ri is the ith individual's fractional rank in the socioeconomic distribution (e.g. the person's rank in the income distribution), and cov(.,.) is the covariance. Where the data are weighted, a weighted covariance is computed, and a weighted fractional rank is generated.

The STATA command GLCURVE can be used to generate the fractional rank in the distribution of income or whatever measure of living standards is being used. This can be used for weighted data. The COR command (weighted if necessary), along with the means and covariance options, then can be used to obtain the mean of the health variable and the covariance between it and the fractional rank variable. The SPSS command RANK can be used to generate the fractional rank variable. The CORRELATION command with the covariance option can be used to obtain the covariance between the health variable and the fractional rank variable. The DESCRIPTIVES command then can be used to calculate the mean of the health variable.

The concentration curve plots shares of the health variable against quantiles of the living standards variable, that is, such curve plots the cumulative percentage of the health variable (y-axis) against the cumulative percentage of the population, ranked by living standards, beginning with the poorest and ending with the richest (x-axis). Graphing concentration curves in Stata can be done using the command glcurve or using the two way command.

The predictive modeling techniques may include variable clustering. For example, the SAS procedure VARCLUS with centroid option may be used to cluster variables. General claims data and prescription drug data are unlikely to distinguish among the adherence problems (or categories of non-adherent healthcare plan members). However, the predictive modeling of the methods and systems discussed below may allow classifications not otherwise possible, and may promote better and more targeted interventions.

The predictive modeling techniques may include variable clustering and level, hierarchical, or multilevel, and/or logistic regression models. Multilevel models may be used to provide an alternative type of analysis for univariate or multivariate analysis of repeated measures.

An embodiment may be directed to the reduction of determinants, their components, their indicators and other dimensions. The richness of the input variable set necessitates a method for dimension reduction. Variable clustering, using PROC VARCLUS, may reduce input dimensions by approximately 40%. The SAS procedure VARCLUS with centroid option may be used to cluster variables. Briefly, this procedure collects into clusters variables that are highly correlated (parametrically, and non-parametrically via Spearman's and Pearson's correlation coefficients) with each other yet oblique (but not fully orthogonal as in principal component) to other clusters. For each variable in each cluster a ratio (usually referred to as the R-square ratio) is computed as:

R square ratio=(1−R.sup.2 own)/(1−R.sup.2 nearest)

where R.sup.2 own is the fraction of the in-cluster variation explained by the variable, and R.sup.2 nearest is the fraction of the nearest (not own) cluster variation explained by the variable. Selection may be made from each cluster of a representative that simultaneously represents its own cluster well (by explaining a large share of its own cluster variation), and is as orthogonal to other clusters as possible (by explaining only a small fraction of the variation of the nearest cluster). Such a candidate will have a small R square ratio.

Principal component analysis, and other methods also may be considered in dimension reduction. Correspondence analysis, a multivariate statistical technique conceptually similar to principal component analysis, may be applied to categorical data. In a similar manner to principal component analysis, correspondence analysis provides a means of displaying or summarizing a set of data in two-dimensional graphical form. However, variable clustering also may be used, as variable clustering may reduce the dimensionality of the model fit, and scoring problem, thus simplifying the scoring method, as each dimension selected by variable clustering may be used to represent a unique input variable from the original data set. This may ensure that the final scoring method has an intuitive description I explanation. The impact of each individual input may be ascertained directly without the need to interpret weighted combinations for principal components.

Selection of a candidate also may be weighed in non-tangible factors such as: how intuitive is the candidate? How computationally expensive is it to compute? In general, these factors typically may be used only as tiebreakers, and for the most part, the variable with the lowest R square ratio may be selected as the cluster's representative. Due to the importance (and number) of therapy class variables, separate clustering exercises may be performed for concurrent therapy class, and all other input variables.

To focus on the most promising input variables, bivariate screening may be performed on the remaining input variable set. The method may compute Spearman's rank correlation coefficient and Hoeffding's dependence coefficient (D-Statistic) for each remaining input variable against percent compliant. For each input variable, the method may plot the rank order of the Spearman correlation statistic against the rank order of the Hoeffding's D statistic. Variables at the upper-right corner of the plot may be eliminated from the input variable list. The rationale for this is that these variables have the least impact on percent compliance.

This plot also may be used to investigate non-monotonic associations. A high Spearman rank, together with a low Hoeffding's D rank, suggests that the relationship between input, and percent compliant is not monotonic. These variables may be further explored using empirical logic plots. The Spearman and Hoeffding ranks are familiar to one of ordinary skill in the art.

Empirical logic plots may be recursively generated for each non-monotonic variable, and bins adjusted at each step, until the plots confirm that any non-linearity had been neutralized. A binned version of the variable may be created using the final bins. The binned variable may be given a b_prefix to differentiate it from its unbinned source. The benchmark model may be selected using PROC REG with SiEPWISE option. Questionable variables may be removed from the model specification, and predictive power and robustness may be reassessed. In cases where the impact of removal may be marginal, the offending variables may be permanently removed.

An embodiment may be directed to weighting or scaling a Social Determinant Of Health or a plurality of Social Determinants Of Health for relevancy, importance, appropriateness, or otherwise significant health or utilization, and/or to weighing or scaling at the high end as highly unequally distributed or otherwise disproportionately weighted or scaled, and/or to weighing or scaling at the low end as equally distributed or low equal distributed, and/or to accounting for a comparatively larger or smaller share or component of the inequality in health or utilization.

An embodiment may be directed to accessing, cleaning, storing, extracting, retrieving, and converting a type or plurality of types of data including by way of example, but not limited to, the following: an Indicator Of Health, a Component Of Health, and/or a Social Determinant Of Health, or a plurality of Indicators Of Health, Components Of Health, and/or Social Determinants Of Health, and/or other pattern-of-life or every-day life data, familiar to one of ordinary experience in the art, such as including, but not limited to: social determinants and/or indicators of health; the social determinants of obesity; diagnosis codes found in Medicare and Medicaid inpatient, outpatient, and physician claims files; Condition Categories found in the Hierarchical Condition Category grouper; demographics, including age and gender; whether a spouse or domestic partner is present in the home; level of education; level of income (estimated or actual); place of residence; prescription information including intonation about the prescribing physician; consumer daily-life activity segmentation attributes such as NE66 segment; life stage grouping; prescription history, (such as, whether the patient is a participant in concurrent therapy); past medication adherence daily-life activity, including whether the patient has been compliant on other medications, whether the patient has been compliant on other therapy regimes, prior average compliance ratio, prescription data, including the particular therapy class at issue, the patient's copay, whether the patient participates in home delivery; and surveys.

An embodiment may be directed to collecting, categorizing, and processing structured, unstructured, mixed structured-unstructured, and disaggregated data, as such types of data are readily known to those skilled in the art, are collected and maintained in a database. The embodiment may include the patient's medical interview and related exit survey data, including the patient's health goals, health preferences, health status, therapy decision-plans, therapy family interaction plans, and Social Determinants Of Health data associated with the patient. Structured data may include Social Determinants Of Health population information collected from government, commercial, and other studies, and other health-related data (such as administrative, health insurance claims, clinical, population health data), as well as consumer, socioeconomic, sociocultural, and similar data evidencing patients' and consumers' patterns of life. Data collected as unstructured information may include books, journals, documents, metadata, health records, audio data, video data, analog data, images, files, and unstructured text such as the body of an e-mail message, Web page, word-processor documents, handwritten information, data collected from social media websites, data originating from mobile devices, data originating from monitoring and surveillance devices, and other information that does not have a pre-defined data model or is not organized in a pre-defined manner.

Data collection may include the patient's pattern-of-life habits, routines, activities, and circumstances, data evidencing the patient's performance of compliance activities of the patient's therapy program, data on the beginning-state and then-current state of the patient's health status and compliance performance activities, data on the period of time during which the patient utilizes or is engaged with a prescribed real-world or virtual-world therapy domain, and data on the patient's illnesses or medical condition.

Unstructured data may be analyzed and understood through the application of commercial software solutions. Such solutions are available from companies such as NetOwl, LogRhythm, ZL Technologies, Brainspace, SAS, Provalis Research, Inxight, ORKASH and IBM's SPSS or Watson. For analyzing unstructured social media data, specialized offerings are available such as People Pattern, Attensity, Megaputer Intelligence, Clarabridge, and Sysomos. Other vendors such as Smartlogic or IRI (CoSort) reportedly can find and structure data in unstructured sources, then integrate and transform the data along with structured data for business intelligence and analytic purposes.

Adjustments to data types may include converting to an acceptable format, such as STATA or SPSS, merging of files to create meaningful distributions, such as population segments, and sampling weights to be regionally representative.

Some embodiments may be directed to accessing and obtaining data from a plurality of other sources including by way of example: Alcohol, and Public Health databases; American Community Survey; American Human Development index; Behavioral Risk Factor Surveillance System (BRFSS); Bureau of Justice Statistics; CDC WONDER; CDC: Arthritis Data, and Statistics; CDC's Behavioral Risk Factor Surveillance System; CDC's Heart Disease, and Stroke Prevention Program; Charlson comorbidity calculator; Current Population Survey; Data Set Directory of Social determinants of health at the Local Level; diagnosis codes found in Medicare inpatient, outpatient, and physician claims files; Diagnostic Cost Groups; Elixhauser comorbidity measures; Health Utility Index Mark 3 system; Healthcare Cost, and Utilization index; Healthcare Cost, and Utilization Project; Health-Related Quality of Life; Index of Disparity; Inter-University Consortium for Political, and Social Research; Lexus Nexus; McKesson; National Center for Education Statistics; National Center for Health Statistics Data Warehouse; National Criminal Justice Reference Service; National Diabetes Surveillance System; National Oral Health Surveillance System; National Program of Cancer Registries; NCI SEER; Office of Applied Studies, Substance Abuse, and Mental Health Statistics; Social determinants of health Maps; State Cancer Profiles; State Tobacco Activities Tracking, and Evaluation (STATE) System; UNESCO Institute for Statistics; U.S. Department of Health & Human Services; U.S. Census Current Population Survey; U.S. National Health Interview Survey; U.S. Census Bureau; U.S. Census Community Health Status Indicators; U.S. National Health, and Nutrition Examination Survey; United States Renal Data System (USRDS); USDA Food Desert Locator Map; World Health Organization Statistical Information System; and Youth Risk Behavioral Surveillance System (YRBSS).

In an aspect of some embodiments, a module may be directed to applying predictive analytic modeling and other techniques to: modeling approaches (such as, baseline, vendor, and/or internal proprietary methodologies; explore all data strategies; multiple techniques including segmentation techniques [clustering techniques, neural networks, visualization], concept descriptions [rule induction methods, conceptual clustering], classifications [discriminant analysis, rule induction methods, decision tree learning, neural networks, K nearest neighbor, case-based reasoning, genetic algorithms], prediction [regression analysis, regression trees, neural networks, K nearest neighbor, Box-Jenkins methods, genetic algorithms], and dependency analysis [correlation analysis; regression analysis; association rules; Bayesian networks; inductive logic programming; visualization]); analytics data understanding processes (such as, data collection, data description, data exploration, data quality verification); data preparation processes (such as, data selection, data cleaning, data construction, data integration, data formatting); data modeling processes (such as, modeling methodologies plan, modeling techniques plan, variables plan, scoring plan [records; frequency; dynamic data], generating test design, model build, model assessment); model evaluation processes; model development processes (such as, deployment plan, monitoring and maintenance plan, final reporting, project review); and/or model review, adjustment, and updating processes.

An embodiment may be directed to applying SAS, STAT, Cognos, and other software tools, readily known to persons of ordinary skill in the art, to facilitate the analysis, adjustments, and comparisons of inequality and well-being and the development of concentration indices across distributions of living standards using numerical and non-numerical disaggregated data. Inequality data and ordering outputs from such software tools may be visualized and compared through Lorenz curves or other concentration curves, a graphical tool readily known to persons of ordinary skill in the art. An aspect of some embodiments may include a module directed to performing on various types of data predictive analytic or other modeling, statistical techniques and other techniques: to data infrastructure functions, data warehouse functions, and data mart functions for new predictive or other analytic models and for adjusted pre-existing or updated predictive or other analytic models; to performing predictive and other analytic modeling; to containing and managing a predictive or other modeling environment hosting application vendors (such as, SAS, SPSS, R. Pythos, Java, Cognos), document components (such as, data directories, mining schema, transformation directories, analytic models), algorithms for predictive modeling and for therapy programs, and predictive model portfolios; to deploy, distribute, and scale data and processing in runtime environments (Hadoop streaming; Mapper; Python; Reducer; running totals); to performing health status change analysis (such as, base-line time-series database management of populations and Indicators Of Health, baseline-change estimates, and actual or real time baseline-changes including time-based change-detection of status updates for patient patterns-of-life events embracing the number of events in an event category's previous time period graphically represented with feature vectors in an event loop tracking patient pattern-of-life events such as, “take next event,” “update one or more associated feature vectors,” and “rescore updated feature vectors to compute alerts”); health status change module management (such as, analytic models for Indicators Of Health, Components Of Health, and Social Determinants Of Health) and multidimensional data analysis environments for one or a plurality of data types, such as, Indicators Of Health, Components Of Health, and Social Determinants Of Health, and management of indices/scores, patient and healthcare provider actions, and measures; to performing deployments of predictive and other analytic models, physiology and other physical activity surveillance and monitoring, and other sensing and measurement utilizing dashboards and other patient and healthcare provider communication and compliance surveillance, monitoring, and performance activities; to embedding one or more predictive models in prescribed or recommended therapy programs.

Some embodiments may be directed to predicting whether a patient is likely to be adherent or compliance or cession with a therapy program. Health factors, determinants, components, indicators, interventions, various demographic, and other data have been identified, as well as patients' historical adherence and/or compliance records, taking into consideration the Social Determinants Of Health and other data which may be used to predict whether a particular patient is likely to be adherent or compliant or cessation with a therapy program. Such factors, determinants, components, indicators, interventions, demographic, and other data and historical adherence and compliance records are included in a predictive tool, wherein the tool is used to predict the likelihood or probability of therapy adherence or non-adherence, or therapy compliance or non-compliance, or cessation of adherence or compliance, taking into consideration among other things the Social Determinants Of Health and the application of one or a plurality of indices or scores or sub-indices or sub-scores derived from the analysis, synthesis, and other application of the Social Determinants Of Health.

In some embodiments, an aspect may be directed to predicting a patient's likelihood of therapy adherence, and/or non-adherence, compliance, and/or non-compliance, and/or the cause of cessation of adherence or compliance where there may include data points related to Indicators Of Health, Components Of Health, and/or Social Determinants Of Health.

Embodiments of the inventive subject matter and its methods and systems may be directed to methods and systems that identify patients who are at risk of non-adherence or non-compliance and/or at risk of the likelihood or probability of cessation with a therapy program, predicting a basis for such non-adherence, or non-compliance, or likelihood or probability of cessation, and targeting outreach, intervention, and engagement directed to patients who have been identified as likely to be non-adherent, or non-compliant, or likely to cease compliance.

Adherence is based on a combination of a patient's likelihood of continuing therapy and a patient's likelihood of complying with therapy.

In one example embodiment, a separate subsystem is used based on whether a patient is new to therapy (inexperienced) or continuing therapy (experienced). In another example embodiment, the same subsystem is provided for both inexperienced and experienced patients.

In one example embodiment, a separate subsystem is provided based on the disease state, health condition, or health risk of the patient. For example, one subsystem may be provided for a patient with hypertension, another subsystem may be provided for a patient with diabetes, and still another subsystem may be provided for a patient with asthma. In one example embodiment, the subsystem is not dependent upon the patient's disease state. In an example embodiment in which separate subsystems are provided for both disease states and inexperienced and experienced patients, then the prediction method may predict adherence, compliance, and/or cessation for patients with hypertension, diabetes, and asthma and may include an inexperienced hypertension subsystem, an experienced hypertension subsystem, an inexperienced diabetes subsystem, an experienced diabetes subsystem, an inexperienced asthma disease subsystem, and an experienced asthma disease subsystem.

In an aspect, predictive modeling techniques may be directed to applying to data points such as Indicators Of Health, Components Of Health, and Social Determinants Of Health rating a particular patient's risk of non-adherence or non-compliance; prioritizing patient outreach based on predicted risk; prioritizing patient intervention and engagement therapies based on predicted risk; diagnosing adherence and/or compliance problems (such as, failure to understand or accept the prescribed or recommended therapy, crime and safety in the patient's neighborhood, the patient's lack of mobility, the patient's residing in an urban food desert, or the patient's lack of proximity or access to care) based on predicted risk; and intervening as appropriate at the patient level.

Indicator Sub-Indices, Component Sub-Indices, Determinants Sub-Indices, a Social Determinants Index and/or a similarly-derived index for a given community, individual patient and/or other population segment may be directed to comparing across communities and individual patients and correlating with: data on the design of therapy programs; data on the algorithms and elements of prescribed or recommended therapy program; data on patient performance of therapy programs; data on specific health outcomes across communities and/or individual patients; data managing adherence, compliance, and cessation of prescribed or recommended therapies; data identifying health goals; data on health outcomes; data on health disparities; and/or data on preventive health programs and wellness programs.

Where the methods, and systems utilize a raw score, rather than an adherence index, the SAS STAT software version 14.1 and HLM hierarchical data modeling (Scientific Software International) may be applied in performing many of the analyses of the Social Determinants Of Health.

Embodiments of the inventive subject matter and its methods and systems also may be directed to assessing and detecting a primary cause of non-adherence, or non-compliance, or cessation for a patient identified as likely to be non-adherent or non-compliant, or identified as likely to cease adherence or compliance. Such assessment and detection also may occur simultaneously with the identification of a primary cause of non-adherence, noncompliance, or cessation.

Embodiments of the inventive subject matter and its methods and systems also may be directed to improving therapy adherence or compliance, such as the promotion of preventive health and the utilization of internet-based, mobile-device-based and other channels remote from the traditional bricks-and-mortar healthcare provider for delivering prevention promotion therapies and patient outreach, interventions, and engagement with such therapies.

Embodiments of the inventive subject matter and its methods and systems may be directed to creating or designing benefit plans or programs that deliver and/or promote adherence to and/or compliance with therapy programs comprising of: identifying members of such plans who are at risk of non-adherence and/or noncompliance with a medically-supervised therapy program and/or at risk of the likelihood or probability of cessation of adherence or compliance; predicting a basis for such non-adherence, or non-compliance, or cessation; structuring the benefit plan or program offering for such member to increase the likelihood or probability of compliance, and/or adherence, and/or to reduce the likelihood or probability of cessation of the therapy program; structuring benefit plans to promote preventive health; and utilizing communication-based channels remote from traditional bricks-and-mortar healthcare providers for delivering to benefit plan members and managing prevention promotion therapies and interventions and sustained engagement with such therapies and interventions.

Embodiments of the inventive subject matter and its methods and systems may directed to designing therapy programs to increase adherence or compliance, or to terminate cessation of compliance.

Embodiments of the inventive subjective matter may be directed to patient targeting for purposes of outreach, intervention, engagement and retention. In targeting, intervention may be directed to a patient's primary cause of non-adherence or primary cause of non-compliance.

In some embodiments, patient outreach may be directed to initially, or on a recurring basis, contacting or communicating with a patient initiated by a healthcare professional. An example would include offering the patient a prescribed or recommended therapy program based on the patient's disease or condition, the health status of the patient, the compliance performance requirements associated with the patient, and the patient's predicted motivation, compliance, and cessation scores. Other examples would include a reminder delivered through a patient notification device (such as, a text message or phone call utilizing a mobile phone), a mailing, or a preventive health advertisement in electronic media (such as, a social media network, Internet, television, or radio), a comment to a patient's account in the patient-accessible record of electronic medical records of a healthcare provider, receipt of medication refill through a home delivery service, or a face-to-face consultation with the healthcare professional.

Outreach involving offering the patient a therapy program may be completed where the patient formally accepts the program agreeing to adhere to the program's aims, goals, performance measures, and other requirements. Patient acceptance of the therapy offer may be by way of the patient's enrollment in a therapy program or agreement to accept and perform those benefits of a healthcare plan that cover the prescribed or recommended therapy program.

In an embodiment, a potentially non-compliant patient may be further identified as associated with forgetfulness as a primary cause of non-motivation, non-adherence, non-compliance or cessation of compliance. The forgetful patient may be classified as “Occasional Neglectors,” “Energetic Circumspectors,” and “Obedient Delayors”. Occasional Neglectors may be those patients who have a positive perceived value of therapy and are not intentional in their adherence or compliance through their daily-life activity. They periodically neglect to perform the prescribed or recommended therapeutic activities (such as, maintain a nutrition diary or take their medications in managing diabetes), and as a result of occasional neglect, such patients occasionally are not adherent or compliant. Energetic Circumspectors do not, for a variety of reasons, place a positive value on their therapy. This could be because they believe the prescribed or recommended therapy activities are not effective, are experiencing side effects, don't like being “prescribed to,” or do not believe the therapy offers sufficient benefit relative to the cost. As a result, Energetic Circumspectors actively choose not to perform the therapeutic activities as prescribed or recommended. Obedient Delayors do a good job performing the recommended or prescribed therapeutic activities, as long as the activities are convenient for these patients, such as performance at a convenient time or place. However, as Obedient Delayors get out of the routine of performing such activities, such patients put off performance all together. As a result, Obedient Delayors may experience a gap in care. This daily-life activity is less intentional than Energetic Circumspectors and does not reflect a negative view of therapy itself.

In some embodiments, interventions may be directed, by way of example, at: letters combining authority (for example, signed by chief medical officer and a physician) and loss aversion (pointing out risks of non-adherence rather than benefits of adherence); reduction of co-payments; reminder systems (particularly effective for Occasional Neglectors); auto-refill programs (particularly effective for prescription Obedient Delayors); financial assistance (particularly effective for Energetic Circumspectors); discussion with a healthcare professional (particularly effective for Energetic Circumspectors); and encounters or engagement in social media and other electronic domains with and support from a group of persons who also have the same chronic or other disease or condition and comorbidities as that of the patient or those who have the same or similar therapy regime as that of the patient.

Interventions also may be directed to encountering patients in traditional bricks-and-mortar healthcare environments, such as acute care hospitals and outpatient facilities, and in environments or domains external to and remote from such traditional environments. External remote environments may be directed to Internet, mobile-devices, virtual, social media, telehealth, and other domains and environments.

Intervention directed to patient retention in an enrolled or other benefit plan may be directed to initiatives from the healthcare provider for a patient enrolled in a healthcare plan that seek to have the patient re-enroll or change over from another plan. Retention intervention also may be directed to preventive health initiatives that are permissible under health marketing laws.

Interventions also may be directed to targeting patients enrolled in a health benefits plan who are identified as likely to be non-adherent as identified through predictive modeling of the Social Determinants Of Health and disparities of health may lead to greater therapy adherence. By targeting such factors, their Components Of Health and/or their Indicators Of Health as manifested in healthcare gamification domains, for example, for those patients deemed more likely to be non-compliant and/or non-adherent based on daily-life activity characteristics of those patients, the resources of a social intervention delivered through the healthcare gamification domain may be more effectively and efficiently used. Plan benefits may be designed to incorporate healthcare gamification domains for such a targeted group of members. Thus, in one embodiment, predictive modeling techniques are applied to data points such as those listed above to: (1) rate a particular patient's risk of non-adherence; (2) prioritize outreach based on predicted risk, and the deferred or delayed performance of a prescribed or recommended preventive therapy; (3) diagnose adherence problem (such as, Energetic Circumspector, Occasional Neglector, or Obedient Delayor); and (4) intervene as appropriate at the patient level.

In one embodiment, patients more likely to benefit from an intervention may be identified and/or targeted based on a health condition. Such health condition may be known (such as, affirmatively included in patent data available to an attending healthcare provider) or predicted (such as, based on available data regarding the Social Determinants Of Health).

For example, patients requiring therapy for diabetes, asthma, obesity, or hypertension may be particularly benefited by use of the methods and systems for improving therapy adherence. The applications of predictive modeling of the methods and systems also may be used to identify a window of time in which intervention is most likely to be effective. For example, an Occasional Neglector may benefit from periodic reminders whereas an Obedient Delayor may benefit most from an intervention that occurs at or near the expiration of an existing prescription fill. Early interventions may be most effective for an Energetic Circumspector.

Having identified a patient as at risk of non-adherence or non-compliance and/or having identified such patient's primary non-adherence or non-compliance cause according to one embodiment, the methods and systems further may be directed to targeting such patient for intervention. In targeting, intervention takes into consideration such patient's primary cause of non-adherence or primary cause of non-compliance. Interventions may include, by way of example: letters combining authority (for example, signed by a chief medical officer and/or a supervising physician), and loss aversion (pointing out risks of non-adherence rather than benefits of adherence or compliance); reduction of copayments; reminder systems (particularly effective for Occasional Neglectors); auto-refill programs (particularly effective for prescription Obedient Delayors); financial assistance (particularly effective for Energetic Circumspectors); and discussion with a clinician (particularly effective for Energetic Circumspectors).

An embodiment may be directed to utilizing rules-based engines and mathematic and statistical methodologies and techniques for application of actionable insights derived from determinants, components, indicators, dimensions, and disparities data to improve adherence to, compliance with and/or cessation of treatment therapies through patient interventions and engagement domains. The methods and systems may predict the likelihood of adherence, compliance and/or cessation by decomposing one or more coefficients or sets of coefficients that express one or more social or other determinants, components, indicators, dimensions. or disparities of health. Such coefficients, expressed as indices or scores, may be scaled, may correlate the indices or scores with actionable insights applied to prescribed or recommended therapy programs, and may be correlated with a portfolio of therapy domains and a portfolio of patient engagement domains or engagement assets.

In some embodiments, the method may be directed to handling adherence or compliance as not one daily-life activity, but as two or a plurality of sequential or non-sequential daily-life activities that are independent of one another. First, the patient may either stay or not stay in compliance, and conditioned on staying in compliance, the patient will either do a good job or will do a bad job. By separating those daily-life activities, that separation of daily-life activities allows the method to be more robust. The method may include a process where it goes through to look at the disease state and how long the patient has been in therapy, and then there are more particular factors that the method may look at when making the adherence prediction. The weighting that the method uses for those factors may be unique.

In some embodiments, the predictions that the method produces may be directed to processing the likelihood of continuing or ceasing therapy by decomposition of a coefficient or set of coefficients that expresses the likelihood of continuing adherence or compliance by multiplying the likelihood or probability of continuing or ceasing times a Social Determinant Of Health Index (or a related Component Index or Indicator Index). In this instance, multiplication is the Indicator Index, Component Index, Social Determinant Index, or respective sub-index—a cessation or continuation index. On a periodic basis, for example a weekly basis, the method may have a daemon that wakes up, goes to all the patients that are in the scored segment, processes the algorithm, and populates the result back to the database.

The Indicator Index, Component Index, and the Determinant Index, and/or their respective sub-indices or scores, may be determined from a model or a plurality of models. The structure of the model may be determined by examining many variables in the data sources above mentioned (such as, social 1 Determinants Of Health, patient demographics, prescription drug history, and past therapy or medication daily-life activity such as cessation of other treatments and/or adherence on other treatment or medication therapies), and determining patterns. Probabilities of adherence, compliance and/or cessation may be determined by using such patterns.

Additional adjustments to the coefficient or plurality of coefficients, where their use is as the primary indicator, may be made for age, gender, and comorbidities. Further adjustments may be made for State-level characteristics such as population level measures of income, and education, and the supply of healthcare resources. Adjustments also may be made for measures of socioeconomic status at the patient level, and for geographic variations. The use of multilevel modeling methods may distinguish compositional from contextual factors. Risk of adherence may be influenced by such factors as the strength of a patient's social network, and support systems, and the patient's capacity for managing their own care, including obtaining follow-up care, adhering to prescribed or recommended treatment therapies, adhering to complex medication regimes and/or complying with other post-discharge instructions.

Once the basic models are obtained, real time data may be applied from current patients to further refine the model. There may be a probability variable in a cessation model that is indicative of the disease type or therapy type. The cessation model may be further divided based on the likelihood or probability of a patient to stop performing a prescribed or recommended treatment activity. The cessation model also may be divided by a new patient and an experienced patient.

The models may be used to target high risk patients for outreach. The model may determine likelihood or probability of cessation. If the probability of cessation is sufficiently low, the model may determine what the likelihood or probability of adherence or compliance is developing an adherence or compliance index by calculating a Determinant Index, a Component Index and/or an Indicator Index and/or the corresponding respective sub-indices or scores. The model may predict the probability that the patient is not suitably motivated for adherence. Accordingly, the model may have an adherence model, a compliance model, a cessation model, and a motivation model. Each model may be further divided into sub-models by a new patient model, and an experienced patient model, and consequently, resulting in six models.

Some embodiment may be directed to predictive modeling techniques based on the Social Determinants Of Health, together with inference analysis. This approach may provide a comprehensive set of predictive attributes for use in assessing the impact on the design or improvement of therapy programs by identifying, developing, optimizing, and targeting treatment and/or preventive health interventions. This approach also may provide outreach to and engage with the patient in regular, sustained, long-term intervention domains that support the self-management of chronic and other illnesses and medical conditions.

An aspect of these embodiments may apply hierarchical logistic regression models to estimate the association between the Social Determinants Of Health measured at different levels, together with the patient's risk of adherence or compliance, while sequentially controlling for socioeconomic, sociocultural, psychosocial, and other characteristics. The Gini coefficient for income inequality may be deconstructed in the measurement of health determinants, indicators, dimensions, and disparities. Clustering may be performed of the determinants and disparities data, and on data on patient progression toward health improvement, together with disease clustering, and disease progression data. Clustering may be adjusted for adherence or compliance probabilities based on several factors, such as for example, instantiated variables around attributes meaningful to predictive adherence and confounding. Adherence or compliance performance scores may be generated using an adherence or compliance index and/or raw scores encompassing Social Determinants Of Health and disparities scores, patient intervention or engagement scores, patient pattern-of-life activity scores, adherence impact scores, data-conflict scores, adherence and compliance cost scores.

Adherence or compliance performance scores, which also may be referred to as Risk Scores, indicative of the patient's adherence or compliance may be communicated to the patient's healthcare professional and to the patient, together with recommended opportunities for intervention based on predicted treatment therapies, predicted intervention therapies and/or patient preference. Adherence or compliance performance scores also may be used as a basis for recognizing and awarding pattern-of-life activity-based incentives and rewards.

An embodiment may be directed to mapping a gap index or gap score that tracks, links, or correlates one or more Social Determinant Of Health and/or a health disparity. The indexed or scored determinants or disparities may be compiled and applied through a Module that predicts the likelihood or probability that a patient will adhere or comply with a therapy program, that a patient will cease adherence or compliance, and that the patient is not suitably motivated for adherence and compliance. The indices or scores also may be directed to valuing patient interventions or engagements with health therapy domains external to the clinician's environment. The values may be used to award monetary and non-monetary recognition to patients for achieving health therapy goals by engaging with such domains.

To develop the Determinants Index or Determinants Score, an embodiment may be directed to decomposing one or more coefficients or sets of coefficients of Social Determinants Of Health. Such coefficients, expressed as indices or scores, may be scaled and correlated with a portfolio of therapy programs, including patient intervention or engagement domains external to the healthcare professional's facilities.

A Module, according to an aspect of an embodiment of the inventive subject matter in an embodiment may be directed to designing or improving therapy domains based on the recommended treatment algorithms and treatment guidelines of public health agencies, healthcare professional associations and/or evidence-based best research and practices. The effectiveness of the treatment algorithms may be measured periodically with respect to each patient. The module may measure effectiveness by utilizing a portfolio of algorithms maintained by a smart database managed by a rules engine. Each treatment algorithm may be measured for its success in achieving the goals of the patient's therapy program. Measurement may be performed by establishing the baseline goals of the patient's therapy program, with an input value assigned to each articulated factor or context that determines or influences the prescribed or recommended performance mechanics of such program. A determination or assessment may be made periodically of the progress toward such goals. The difference between the baseline assessment and the current status of progress toward the plan's goals may be referred to as a treatment gap index or treatment gap score. The components of the treatment gap index or treatment gap score may include the symptoms, and characteristics of the specific patient's illness or medical condition, the symptoms and characteristics of the illness' or condition's comorbidities, the profile of diseased population of which the patient is a member, the Social Determinants Of Health and health disparities associated with the patient, and the therapy intervention domains prescribed by the therapy program, together with a prediction of adherence or compliance or cessation with the therapy. Each component of the treatment gap index or treatment gap score may be measured by weight and scale. Measurement of the Social Determinants Of Health and health disparities may be made by decomposition, a statistical method known to those skilled in the art. Periodic redetermination of the treatment gap index or treatment gap score may be made from time-to-time, such as during each time a medical interview is administered to the patient. The aggregate measurement may be converted to the treatment gap index or treatment gap score.

An embodiment may be directed to establishing the gap index or gap score using disaggregated data. A number of indices may be utilized. Distributive comparisons and adjustments may be made for asymptotic standard errors to enable statistical inference. The range of statistical analysis and techniques familiar to one of ordinary skill in the art include, for example: Decomposition (where statistical techniques include: FGT Decomposition by Groups, by Sources, by Growth & Redistribution, by Transient & Chronic, S-Gini Decomposition); inequality (where statistical techniques include: Anderson Index, I S-Gini coefficient, Entropy Index, Income-Component Proportionate Growth); Polarization (where statistical techniques include: Wolfson Index, Ducios, Esteban, and Ray Index); Poverty (where statistical techniques include: FGT Index, Watts Index, S-Gini coefficient, CHU Index, Impact of Price Change, Inequality-neutral Targeting, FGT Elasticity, Impact of Demographic Change); Dominance (where statistical techniques include: Poverty Dominance, Inequality Dominance, Indirect Tax Dominance); Welfare (where statistical techniques include: Atkinson-Gini coefficient, ATK-Impact of Price Change, ATK Impact of Income-Component Growth); Distribution (where statistical techniques include: Density Function, Joint Density Function, Distribution Function, Joint Distribution Function, Plat-Scott_XY, Non-Parametric Derivative, Conditional Standard Deviation, Descriptive Statistics, Group Information); Redistribution (where statistical techniques include: Tax/Transfer, Transfer vs. Tax, Horizontal Inequality, Redistribution, Coefficient Of Concentration, HI: Duclos & Lambert_1999; HI: Duclos, Jalbert & Araar_2003); and Curves (where statistical techniques include: Lorenz, Generalized Lorenz, Concentration, Generalization Concentration, Quantile, Normalized Quantile, Poverty Gap, Pro-Poor, C-Dominance, Bi-Polarization, Relative Deprivation). Many of such statistical techniques may be performed with DAD (Distributive Analysis/Analyze Distributive) and SAS.

In general the adherence index or compliance index may be determined by factoring the likelihood or probability of adherence or compliance developed from the Social Determinants Of Health and pattern-of-life data. Initially, a predictive model may be built from general population historical demographic data, and predicted health determinants data. The general population data also may be further segmented by other factors, such as by a particular community or a population within a certain geographic region or organization (such as, a business enterprise) or a population having a certain category of disease (such as COPD). Individual patient demographics and other individual patient data (such as, individual patient therapy performance records or medication possession ratios) may be applied against the model to further refine the model. The refinements may be made by evaluating differences between actual and predicted adherence or compliance and evaluating related patterns. Once actual data is gathered, an actual past gap index or score may be considered to further refine the prediction model.

An aspect of the inventive subject matter may be directed to normalizing data by a rules engine that converts data to a predetermined format, processing disparate data from the databases into normalized data formatted by the engine's rules. The rules engine may define each field of the data and converts each field to a corresponding field in the predetermined format. The rules engine also may define how the normalized data should relate to each other pursuant to predetermined instructions.

Some embodiments may be directed to generating the gap index or score as an integer. It may be generated by adding several values, each of which represents the patient's risk of non-adherence or non-compliance with the therapy program or risk of cessation in the therapy program. For example, a first component of such index or score may be a value or a range of values that represents Economic Security & Financial Resources as a Social Determinant Of Health with the average number of vehicles in a household as a data point. The second component may be a value or range of values that represents Livelihood Security & Employment Opportunity as a Social Determinant Of Health with the unemployed as a percent of the civilian labor force and by the labor force participation for adult males as data points. The third component of such embodiment may be a value or range of values that represents School Readiness & Educational Attainment as a Social Determinant Of Health with the percent of students enrolled in special education in high school, by the percent of students eligible for free or reduced price meals in elementary school as data points. A fourth component may be a value or range of values that represents Environmental Quality as a Social Determinant Of Health with the number of chronic diseases, asthma-related emergency room visits without admissions, and asthma-related hospitalizations for children as data points. A fifth component may be a value or range of values that represents the Availability and Utilization Of Quality Healthcare Services as a Social Determinant Of Health with the rate of outpatient visits without admission for all ages and all causes and the rate of emergency room visits without admission for all ages and all causes as data points. A sixth component may be a value or range of values that represents Adequate, Affordable & Safe Housing as a Social Determinant Of Health with the percent of housing in units that are owner-occupied, the rental vacancy rate, the percent of households paying more than 25% of their household income for rent, and the percent of households paying more that 25% of their household income for mortgage as data points. The following table provides an example of how values or a range of values representing the foregoing components may be indicated:

| SOCIAL DISPARITY | DIMENSIONS OR INDICATORS | RANGE OF VALUES |
|---|---|---|
| 1) Economic Security & | Average number of vehicles in household | 0 = I point 2 = 2 points |
| Financial Resources | | 2+ = 3 points |
| 2) Livelihood Security & | Unemployed as a percent of civilian labor | 0 = 1 point |
| Employment Opportunity | force | 1 − 2 = 2 points |
| | | 3+ = 3 points |
| | Labor force participation for adult males | 0 = 1 point |
| | | 1 = 2 points 2 − 3 points |
| 3) School Readiness & | Percent of students enrolled in special | 0 = 1 point |
| Educational Attainment | education-high school | 1 = 2 points |
| | | 2 = 3 points |
| | Percent of students eligible for | 0 = 1 point I = 2 points |
| | free/reduced price meals-elementary | 2 = 3 points |
| 4) Environmental Quality | Count of chronic diseases | Diseases/conditions = |
| | | 1 point 1 disease/ |
| | | condition = 2 points |
| | | 2+ diseases/conditions = |
| | | 3 points |
| | Asthana-related emergency room visits | 0-2 ER visits = 1 point |
| | without admission | 3 ER visits; ; ; ; ; ; 2 points |
| | | 4+ ER visits = 3 points |
| | Asthma-related hospitalizations: children- | admits = 1 point |
| | annual asthma hospitalization rate per | 1 admit = 2 points |
| | person | 2 admits = 3 points |
| | | 3+ admits = 4 points |
| 5) Availability & Utilization | Rate of outpatient visits without admission | visits = I point |
| Of Quality | (all ages, and all causes per persons) | I visits = 2 points |
| Healthcare Services | | 2 visits = 3 points |
| | | 3+ visits = 4 points |
| | Rate of emergency room visits without | visits = I point I |
| | admission per persons, (all ages, all causes | visits = 2 points |
| | per persons) | 2 visits = 3 points |
| | | 3+ visits = 4 points |
| 6) Adequate, Affordable & | Percent of housing units that are owner- | 0 = I point |
| Safe Housing | occupied ; Rental vacancy rate | 1-2 = 2 points |
| | | 3-4 = 3 points |
| | Percent of households paying more than | 0 = 1 point |
| | 25% of their household income for rent | 1-2 = 2 points |
| | | 3-4 = 3 points |
| | Percent of households paying more than | 0 = I point |
| | 25% of their household income for | 1-2 = 2 points |
| | mortgage | 3-4 = 3 points |

Although the above values and components may used in an example embodiment, it will be readily obvious to persons skilled in the art that different components and/or values could be used to compute the gap index or gap score. By way of further example, fractional or decimal values instead of integers could be used. Likewise, rather than merely adding component values, the index or score could be determined based on a weighted matrix in which certain components are weighted more heavily or less through the use of an appropriate scaling factor. Moreover, persons of ordinary skill in the art may determine that other components may be useful in determining the action score. Biographical data, such as the patient's age and/or related medical data, such as the patient's likelihood of developing a particular medical condition, could also be used in determining an action score. The foregoing, therefore, should not be interpreted as a strict protocol upon which a gap index or score is determined. Instead, the above table is provided merely as an example of an algorithm for determining such indices or scores.

Other embodiments that also are directed to evaluating through predictive models patterns from Social Determinants Of Health data may analyze predictive adherence and compliance data for a given disease type or for a type of drug treatment. From these probabilities, a basic model may be developed. The basic model may be refined by correlating individual patient demographics and actual patient pattern-of-life activity with the basic model and updating the model as appropriate. For example, such data may include age, income bracket, severity of disease or disease type, number of concurrent medications, symptomatic disease, type of drug treatment, partner status, and partner adherence. The model may capture and statistically evaluate patterns in data that at first glance may appear unrelated to adherence and correlate the pattern to adherence. For example, the model may capture and statistically evaluate lifestyle patterns that have statistical significance to adherence. For instance, the model may look at whether a patient consistently smokes or uses alcohol or lives in a high-crime area with education challenges or pays a high portion of household income for rent, and correlate one or more of such factors to the patient's likelihood or probability of adherence.

The adherence index also may identify the patient as high risk. A likely barrier to adherence also may be determined such as forgetfulness, prescription refill delays or medication cost. From this information, the model may develop an individual patient intervention program, which may include membership in a support group, a portfolio of relevant incentives and rewards, reminder tools for regular eating, calorie-in/calorie-out, appointments with the Provider, an annual wellness assessment, prescription refills by mail order subscriptions and/or increased dosages requiring medication to be taken less often. The patient intervention program may be based on what the model determines would be the most effective program for the given patient, which may be based on historical modeling. Once an intervention program is implemented, the patient may continue to be modeled, and data may be collected and related to the patient's adherence. The adherence index or score may be continuously or periodically updated including the new information being gathered.

Improvements in the adherence index may be tracked and correlated in various ways. For example, the model may determine under what parameters a particular healthcare intervention domain or reminder device is most effective or in other words determining an optimum point or combination of factors or qualities for a particular intervention program. This continued refinement of the optimum point may improve the model's ability to more efficiently target patients who will be most likely to be impacted by intervention and to determine a most effective intervention program for a given patient.

An embodiment may be directed to operationalizing the coefficient or score from one or a plurality of the Social Determinants Of Health, their Components Of Health, and their Indicators Of Health by further applying the principles of the decomposed Gini coefficient, the concentration curve, and the concentration index, the extended concentration index, and the achievement index. The resulting coefficient or score may be presented to a healthcare professional or patient as a gap index or gap score. The gap index or gap score may be applied by embodiments of the inventive subject matter to enable or support measuring, tracking of adherence to and/or compliance with therapy programs, by surveilling, monitoring, evaluating, translating, weighing, and reporting to healthcare professionals and/or patients the dimensions, parameters, comparisons, and other factors generated by daily-living activities and represented by social and other determinants, indicators, dimensions, and disparities of health, and the root causes of chronic and other disease. Such reporting may include: notification through a Risk Score or risk indicator that a patient is above, below, or in a targeted or otherwise acceptable range of performance of prescribed compliance activities; recommended remedial activities, such as for example “lose weight,” “take medication,” or “exercise” where the patient is below the targeted performance range; or seek assistance from a professional healthcare or other care provider. Such reporting may be made through Internet, mobile phone, text, social network, telephone, or other communication channel.

The methods of the inventive subject matter may map the gap index or gap score from the Social Determinants Of Health, their Components Of Health, and their Indicators Of Health. Such indexed or scored data may be compiled and applied through statistical techniques that predict the likelihood or probability that a patient will adhere to or comply with a prescribed or recommended therapy, that predict the likelihood or probability that a patient will cease adherence or compliance, and that predict the likelihood or the probability that the patient is not suitably motivated for adherence or compliance. The indices or scores also may be applied through rules engines: that quantify and assign values for patient encounters or engagements with therapy domains operating inside or outside of the clinician's environment; that award non-monetary, non-negotiable credits, currencies, advantages, and other recognition to patients for achieving therapy and compliance goals through engagement with such domains; and that convert the non-monetary recognitions, credits, currencies, and advantages to valuable, negotiable, monetary credits and currencies.

Adherence and compliance may be operationalized by applying the gap index or gap score to give clear directions for assessment and intervention. Such directions may be based on intervention factors, intervention themes, and patient engagement with supportive and self-managed patient intervention domains, group intervention domains, Risk Scores, and feedback reporting.

Some embodiments may be directed to a patient's medical interview, readily known to those skilled in the art, conducted by the healthcare professional in an effort to determine the extent to which the Social Determinants Of Health affect the design of a therapy program for the patient or the improvement of an existing therapy program prescribed for the patient. In seeking to establish or maintain rapport with the patient, diagnose the patient's health condition, review the patient's perception of the patient's health status, obtain information about limitations on the patient in daily work and other pattern-of-life activities, and motivate the patient to adhere and comply with the therapy program, the patient may exchange with the healthcare professional data about the those Social Determinants Of Health associated with the patient, active in the patient's pattern-of-life, impacting the patient's quality of life, and otherwise relevant to the patient.

As part of collecting such information, the healthcare professional may observe the patient and record the patient's responses. Patient data observation and data recording may occur during a face-to-face encounter with the patient, or occur remotely such as in an Internet telehealth video encounter. Proxy responses by a third party on behalf of the patient may be allowed where the patient has a physical or mental condition prohibiting the patient from responding. Such observing and recording also may occur through surveillance or monitoring devices that collect the patient's biometric and physical activity. A data point for data collected by such devices might be, for example, a wearable device or weight scale equipped with data transmission capabilities, such as by way of radio frequency identification from the device to a nearby mobile phone, which then transmits and forwards the data to the healthcare professional's data center.

The medical interview may include a survey of the patient comprised of a series of questions and answers. The survey may include open-end questions designed to encourage the patient to speak openly and candidly. The survey also include closed-end questions where a simple “yes” or “no” might be more appropriate. Complete thoughts of the patient may are recorded by the healthcare professional. The survey questions, answers, and complete thoughts may be grouped into categories, and the questions, answers, and categories may be coded in the same manner or a similar manner as done by healthcare professionals using video or audio devices as part of an interactive communication system. Such systems known by those of ordinary skill in the art include the Roter Interaction Analysis System (“RIAS”), the Maastricht History-Taking And Advice Checklist, the Medical Communication Behavior System, the Patient-Centered Method, and other patient data recording systems. By way of example, the RIAS reportedly records by audio device complete thoughts during the interview and subsequently analyzes each thought. The analysis categorizes the thoughts into approximately 30 categories. Several of those categories may be applicable to the Social Determinants Of Health. Examples of the RIAS categories whose scope include the Social 25 Determinants Of Health are illustrative in the following table.

General RIAS Communication Codes

| Name | Description | Example | codes |
|---|---|---|---|
| Social talk | All aspects of social | So, you managed to get | Personal remarks, |
| | conversation, personal | here Personal in time | social conversation, |
| | remarks, laughter, and | despite the heavy | laughs, tells jokes |
| | compliments, not related | conversation, laughs, | |
| | to the patient’s health status. | tells snow this morning— | |
| | | jokes exhausting, eh? | |
| Questions about | All questions regarding | Any progress on your | Asks questions (open |
| lifestyle and | psychological, lifestyle, | weight-loss program so | or closed ended) |
| psychosocial | social or other non- | far? | lifestyle |
| issues | biomedical issues. | | psychosocial |
| | | | other |
| Information about | Facts and figures, advice, | You see, the strain on | Gives information/ |
| lifestyle and | opinions and suggestions | your lower back gets | counsels |
| psychosocial | regarding psychological, | more intense the more | lifestyle |
| issues | lifestyle, social, or other | you gain weight. | psychosocial |
| | non-biomedical issues. | | other |
| Empathy | An emotionally laden | Oh yeah, that must be | Empathy |
| | supportive utterance or | really painful | Reassurance, optimism |
| | comment to patient's | | Legitimizes |
| | speech confirm | | Partnership |
| | | | Shows approval |

Data points captured and grouped or categorized through the medical interview that include the Social Determinants Of Health may be: economic security and financial resources; livelihood security and employment opportunity; school readiness and educational attainment; environment quality; civil involvement and political access; availability and utilization of quality healthcare services; adequate, affordable and safe housing; community safety and security.

Within each such category, embodiments may be directed to analyzing data points that relate to socioeconomic, sociocultural, psychosocial and other life-contextual factors, including for example: economic security and financial resources (such as, whether the patient is facing financial distress that prevents the purchase and taking of medications in amounts or as frequently as prescribed); livelihood security and employment opportunity (such as, where such financial distress is the result of being laid off by the patient's employer); school readiness and educational attainment (such as, where the patient fully understands the prescribed regime for medicating frequency or dosage amounts or fears self-administering medication); environmental quality (such as, where there is a smoker in the household of an asthma patient); civil involvement and political access (such as, where the patient is angry or depressed or feeling hopeless arising from the failure of the city to plow the snow on the patient's street causing the patient to miss time from work and be docked in pay); availability and utilization of quality healthcare services (such as, where the patient frequently misses scheduled follow-up appointments with the healthcare professional because the patient has no car or no convenient access to public transportation); adequate, affordable and safe housing (such as where the patient is “house poor” with a disproportionate amount of household income devoted to rent or mortgage payments resulting in insufficient funds to buy or maintain a prescribed medical device); community safety and security (such as, where the patient is afraid to perform prescribed outdoor activities or walk to the grocery or drug store because of high neighborhood crime); the relationship among the patient and the patient's family, family caregivers, and social caregivers (such as, where there is a single-parent home with a grandparent as the head of the household); the relationship among the patient and the patient's co-workers (such as, where the co-workers and the patient are part of a group that exercises together as part of a rewards program of the employees' benefit plan); the patient's expectations toward the therapy program (such as, “I know this hospital has such a good reputation that I don't have to do all the hard things in my therapy program); and the patient's attitude toward the patient's health condition (such as, my mother and her sisters were obese, so my obesity is genetic, my mother and her sisters have lived with it for 60+ years, and I probably will live at least 60+ years—anyway, I can't do a lot about it).

Some embodiments may be directed to analyzing data points that relate to the healthcare professional's personally observation of the patient for verbal and non-verbal health clues. These data points may relate to data such as care-oriented and cure-oriented clues, and socioeconomic, sociocultural, psychosocial and other pattern-of-life factors associated with the patient's health status or limitations on mobility. Such clues and factors may include such subjects as the patient's ideas about the patient's health problem, the patient's thoughts, worries, feelings, and expectations for treatment success, as well as family influences on the patient's health and the performance on the therapy program and how the patient's health condition affects the patient's life.

Some embodiments may be directed to analyzing data points that relate to the patient's preferences and healthy choices made during the performance of a therapy program, as well as data points that relate to the influences impacting such preferences and choices. In such embodiments, the patient's healthy choices may be viewed as not only predictive of morbidity and mortality, but also as related systematically to the patient's socioeconomic status, sociocultural status, and psychosocial status. These statuses of the patient may be viewed by such embodiments as predictors of health preference and health choices, as such, may be considered as being impacted by the structure of the social conditions that shape the life experiences of the patient. Data points for such life-shaping experiences may be grouped or categorized by the patient's socioeconomic status, sociocultural status, and psychosocial status within the patient's social structure. Methods and systems for assessing such statuses of the patient may analyze the links between social stratification and health outcomes, on one hand, and the patterned response of the patient's social group to the realities and constraints of the group's external environment, where the group environment is personalized to the patient by the application of group-level pattern response factors to patient-level pattern response factors relevant to the patient.

Some embodiments may be directed to grouping or subgrouping the pattern response factors of the patient into data points and/or categories, such as for example, income, education, occupation. An aspect of an embodiment may analyze categories such as category importance, category relationship, and category combination. The background context of the data points and categories for the patient may be the Social Determinants Of Health associated with the patient and relevant to the patient's socioeconomic status, sociocultural status, and psychosocial status.

Embodiments directed to identifying and classifying pattern response factors relevant to the patient may include data points related to: poor health practices and behaviors, attitudinal orientations toward health, (such as, beliefs about personal control), stressful environments (such as, high rates of crime, unemployment, residential mobility, and marital instability, and stress in family and in occupation), stressful life events (such as, unemployment, marital difficulties, divorce, and adult and infant morbidity and mortality), chronic role-related stress (such as, occupations, marital, parental, financial), and daily irritations and hassles, social ties, integration and support (such as, family, friends, and workplace networks), perceptions of mastery and control and disproportionate exposure to experiences that lead to a sense of powerlessness and a loss of control in one's life (such as, income, occupational status, education, high-status jobs, and subjective ratings of social class, where a sense of powerlessness is demoralizing in itself and reduces the will and motivation to cope actively with problems), lack of or reduced physical mobility, the presence of obesity, age patterns (such as, the high prevalence of traumatic and accidental death for those under 35, while chronic disease becomes the important factor in the middle years and beyond), risk factors for distant health outcomes compared to basic survival strategies of immediate day-to-day existence (such as, delayed response, delayed gratification, or deferred gratification; the ability to resist the temptation for a smaller immediate reward in order to receive a larger or more enduring reward later, linking the ability to delay gratification to a host of other positive outcomes, including physical health, psychological health, and social competence), the impact of being a racial minority (such as, higher rates of some stressors [such as unemployment], exposure to both poverty and discrimination, exposure to occupational carcinogens and other occupational hazards, limited access to social support and stable community ties (such as, low frequency of contact with friends and relatives, low levels of organizational involvement and church attendance), high divorce rate, less emotional support from spouse, lack of a husband-father presence, less happiness in the marriage, wives less likely to turn to their husbands as confidants, stressful informal networks of mutual aid, the presence of health risk factors (such as, elevated serum cholesterol, smoking, elevated blood pressure, and sedentary lifestyle), exposure to physical hazards (such as, air and water pollutants, accidents, hazardous waste, pesticides, and industrial chemicals), nutritional behavior, seat belt use, breast self-examination, and drug use.

Data on the patient's patterned response factors may be collected by the healthcare professional during the medical interview, as well as during surveys of the patient performed during or upon exiting an interview or as part of other encounters with the healthcare professional.

In some embodiments, predictive modeling techniques may be directed to socioeconomic, sociocultural, psychosocial, and Social Determinants Of Health data points such as those listed above to: predict the patient's health preferences and health choices based on the patient's socioeconomic status, sociocultural status, and/or psychosocial status; diagnose therapy motivation, adherence, compliance, and cessation problems; rate the patient's risk of non-adherence to, non-compliance with, and cessation of the therapy program; intervene as appropriate; prioritize patient outreach; prioritize patient therapy performance and engagement activities; design and improve therapy programs; design and improve healthcare benefit plans; design and improve strategies and activities for patient enrollment in healthcare benefit plans; and design and improve patient retention in therapy programs and healthcare benefit plans.

Some embodiments may be directed to maintaining a database of the patient's pattern-of-life variables generated by the questions and answers in the medical interview, including the clues and factors, statuses, Social Determinants Of Health associated with the patient, and the categories of such data. The variables may be scaled or weighted, with each variable or category of variables having a proportional weight. Each scale or weight may be adjusted up/down over time to reflect experience with the patient's compliance against the compliance of a group such as an enrolled population or disease population of which the patient is a member. A rules engine may execute instructions to establish the weighted importance of one such variable or a plurality of such variables utilizing aggregative methods, stratification, inverse weighting, propensity scoring, principal components analysis, factor analysis, and/or other relative methods. Each of such methods is readily known to persons of ordinary skill in the art. A rules engine may execute instructions to perform indexing or scoring and to arrive at one or more indices or scores associating and/or correlating an index or score with one or a plurality of such variables. Such index or score may act as a sub-index or sub-score representing a quantitative contribution to a larger or an overall index or score.

Embodiments may be directed to mapping the patient's data associated with the Social Determinants Of Health, including the statuses, to key health themes associated with health-related quality of life.

Some embodiments may be directed maintaining a database that may include third-party survey data from national health surveys. Such data may be collected and mapped to health-related quality of life including key health themes evaluated in such surveys. Examples of such national health surveys include the National Health Interview Survey (“NHIS”) in the United States, the European Community Household Panel, the World Bank's Living Standards Measurement Study, and the Demographic and Health Surveys performed in developing countries.

The third-party survey data may include data points relative to personal-level, family-level, and household-level demographic, socioeconomic, and health utilization data. The third-party survey data points may represent health variables and respondent-level information associated with health-related quality of life, such as for example, respondent characteristics, health conditions and risk factors, psychological well-being, perceived discrimination, socioeconomic status, and data on the census tract in which survey respondents resided. The third-party survey data also may be categorized and scaled or weighted on a basis similar to the manner in which the patient's data obtained through the medical interview is categorized and scaled or weighted.

With respect to the NHIS, for example, data points for survey questions relevant to the Social Determinants Of Health may include those found in the NHIS at: Household-Level File, the Family-Level File, and the Person-Level File. Additional data points relevant to the Personal-Level file may include sections on Health Status and Limitation of Activity, Health Care Access and Utilization, Socio-Demographics, and Income and Assets. In addition, the Sample Adult section may include data points relevant to many of the subject areas included in the Family Core but gather more detailed data, such as: Adult Socio-Demographics, Adult Conditions, Adult Health Status and Limitation of Activity, Adult Health Behaviors, Adult Health Care Access and Utilization, and Adult Internet and Email Usage. Data points related to variable names and labels and their associated question numbers in the NHIS may be found at the survey's In-house files and Public-use files.

An embodiment may be directed to merging data from the medical interview survey and the third-party surveys, as well as other data.

An embodiment may be directed to measuring the outcomes of the patient's therapy program. Outcome measurements may include the impairment on the patient's quality of life, limitations on the patient's activity, and the socioeconomic, sociocultural, and psychosocial status and effects attributable to Social Determinants Of Health associated with the patient. Such measurement may be performed by application of the instrument Health and Activities Limitation Index (HALex), which is known to those of ordinary skill in the art. HALex scoring may represent an assessment of health-related quality of life based on a patient's perceived health status and activity limitation. The items that comprise HALex may be part of the core questions in the NHIS where the survey respondent data contain features similar to the Social Determinants Of Health for those survey respondents prospectively measured by the NHIS. An embodiment may be directed to applying the HALex operation to one or a plurality of components or health dimensions, such as for example, the patient's perceptions of the patient overall health status and the patient's functional status. With respect to the patients' perceptions of the patient's overall health status, in the HALex scoring system, there may be, for example, five levels of perceived health status ranging from excellent (scored 1) to poor (scored 0). With respect to the patients' functional status, patients with the most limited function require assistance with basic daily life functions may be assigned a single attribute score of 0. Patients who may be completely independent and report no limitation of activities may be assigned a single attribute score of 1. For the HALex, there may be six levels of functional capacity. A matrix may be created of the five levels of perceived health status and the six levels of functional limitations. Each unique combination of these 30 possible health states may be assigned an index value from the matrix that serves as the HALex quality of life index or score.

An embodiment may be directed to scaling numerically the patient's perceived health status (“PHS”) with questions from the medical interview and/or questions from the NHIS. With respect to the NHIS, an aspect of the embodiment may scale numerically the patient's PHS with, for example, the NHIS-question PHSTAT. For this questions, survey respondents were asked: “Would you say (your) health in general is excellent (score_1), very good (score_2), good (score_3), fair (score_4), or poor (score_5)?” For calculation of the utility index, a response of excellent may be assigned a PHS coefficient of 1.0, very good_0.85, good_0.7, fair_0.3, and poor_0. Below is a summary of illustrative NHIS questions, variables containing the responses to these questions, and the single attribute score assigned for affirmative answers to each question.

National Health Interview Survey:
Family Core Health Status and Limitation of Activities Questions Regarding Quality Of Life

| | Survey | HALex |
|---|---|---|
| Survey Questions | Variable | Coefficient |
| Because of a physical, mental, or emotional problem [do you] | PLAADL | 0.0 |
| need the help of other persons with PERSONAL CARE NEEDS, | | |
| such as eating, bathing, dressing, or getting around inside [the] | | |
| home? | | |
| Because of a physical, mental, or emotional problem [do you] | PLAIADL | 0.2 |
| need the help of other persons in handling ROUTINE NEEDS, | | |
| such as everyday household chores, doing necessary business, | | |
| shopping, or getting around for other purposes? | | |
| Does a physical, mental, or emotional problem NOW keep [you] | PLAWKNOW | 0.4 |
| from working at a job or business? | | |
| Are [you] limited in the kind OR amount of work [you] do | PLAWKLIM | 0.65 |
| because of physical, mental, or emotional problems? | | |
| Are [you] LIMITED IN ANY WAY in any activities because of | PLEVIANY | 0.75 |
| physical, mental, or emotional problem? | | |
| NONE of the above limitations | | 1.0 |

An aspect of the embodiment may calculate the utility index from the formula set forth in the Statistical Notes. 1995: 7:10-4 in the Technical Notes of Healthy People 2000. The utility index=0.10+(0.90×M), where M=(0.41×PHS)+(0.41×SAS)+(0.18×PHS×SAS). PHS is the coefficient assigned in response to the perceived health status question in the NHIS PHSTAT. SAS is the coefficient associated with the single attribute score, based on the responses in the NHIS to the questions PLAADL, PLAIADL, PLAWKNOW, PLAWKLIM, and PLIMANY.

Statistical calculations may be made with the SAS Package version 8.1 (SAS Inc., Cary, N.C.). The NHIS recommends that users of the survey data utilize computer software that provides the capability of variance estimation and hypothesis testing for complex sample designs. The survey uses SUDAAN software (Research Triangle Institute 2008) with Taylor series linearization methods for survey variance estimation. The survey provides SUDAAN code and a description of its use to compute standard errors of means, percentages, and totals with the survey database. The survey also provides example code for SPSS, Stata, R, SAS survey procedures, and VPLX.

Embodiments may be directed to determining the effect various conditions have on the relationship between the utility index and the Social Determinant Of Health by analysis of covariance. A general linear model may be used, with the utility index serving as the dependent variable, the relevant Social Determinants Of Health associated with the patient as the independent variable, and the presence or absence of the health condition as the covariate (class variable).

Embodiments may be directed to assessing the medical interview and/or the NHIS. The interview and the NHIS may contain a series of questions regarding activity limitations. These questions may be assessed by taking the mean response score for the answer to each question and categorizing the scores by Social Determinant Of Health. Linear or logistic regression may be used where appropriate to determine if there is a significant effect between the presence or the degree or intensity of the presence of relevant Social Determinants Of Health and diminished quality of life, as indicated in the patient's or the survey respondents' answers to specific health-status questions. The presence or the degree or intensity of the Social Determinants Of Health are determined by a determinant's range and the patient's self-reported score for the determinant within the mid-portion of the range.

An embodiment may be directed to assessing the therapy program by taking into account the patient's household. The healthcare professional in performing the medical interview may collect from the patient and record and monitor information on the relationship among the patient, the patient's household, and the illnesses or health conditions of the patient and household members who are care givers to the patient. The inventive subject matter may be applied, for example, where a Social Determinant Of Health impacts obesity, obesity is a comorbidity of an illness or condition, the illness or condition is a variable in the NHIS, the utility index is established for the relationship between Body Mass Index (“BMI”) and a Social Determent Of Health, and the presence, change, or absence of BMI on the illness or condition is measured by the change in the utility index. This interrelationship may be illustrated in a report to the healthcare professional or to the patient accounting for, describing, or discussing, for example, the following information.

EFFECT OF MEDICAL CONDITIONS ON THE UTILITY INDICES-BMI INTERACTION:
Therapy Achievement Range

| Variable In The | | | Utility | | Variance; |
|---|---|---|---|---|---|
| NHIS or | | Social Determinant Of | Index | Utility Index | Change In |
| Medical | | Health Category (see the | With The | Without The | Health |
| Interview | Medical Condition | Legend below) | Condition | Condition | Status |
| STREV | Stroke | 1; 2; 3—diet; inactivity | 0.52 | 0.85 | 0.33 |
| CHDEV | Coronary artery | 1; 2; 3—diet; inactivity | 0.58 | 0.85 | 0.27 |
| | disease | | | | |
| ANGEV | Angina | 1; 2; 3—diet; inactivity | 0.6 | 0.85 | 0.25 |
| PAINLEG | Leg pain | 2; 4; 6; 8—reading skill; | 0.66 | 0.81 | 0.15 |
| | | access to transportation; | | | |
| | | fear of neighborhood | | | |
| | | violence if walking to | | | |
| | | healthcare provider | | | |
| HYPEV | Hypertension | 2; 3—workplace stress | 0.73 | 0.87 | 0.14 |
| PAINECK | Neck pain | 2; 3;—workplace injury | 0.74 | 0.86 | 0.12 |
| | | & inadequate employee | | | |
| | | benefit plan | | | |
| JNTYR | Joint pain | 2; 3;—workplace injury | 0.75 | 0.88 | 0.13 |
| | | & inadequate employee | | | |
| | | benefit plan | | | |
| AASMEV | Asthma | 4; 2; 7—plant and | 0.76 | 0.85 | 0.09 |
| | | household allergies; | | | |
| | | ignorance of symptoms | | | |
| | | and therapies | | | |
| PAINLB | Low back pain | 2; 3;—workplace injury | 0.76 | 0.87 | 0.11 |
| | | & inadequate employee | | | |
| | | benefit plan | | | |
| SINYR | Sinusitis | 4; 2; 7—plant and | 0.79 | 0.85 | 0.06 |
| | | household allergies; | | | |
| | | ignorance of symptoms | | | |
| | | and therapies | | | |
| AHAYFYR | Hay fever | 4; 2; 7—plant and | 0.81 | 0.84 | 0.03 |
| | | household allergies; | | | |
| | | ignorance of symptoms | | | |
| | | and therapies | | | |

### Legend:

1=economic security and financial resources; 2=livelihood security and employment opportunity; 3=school readiness and educational attainment; 4=environment quality; 5=civil involvement and political access; 6=availability and utilization of quality healthcare services; 7=adequate, affordable and safe housing; 8=community safety and security

Some embodiments may be directed to analyzing BMI as a proxy for overweight or obesity. Such embodiments may apply a quality of life HALex-utility index method in making health status, health risk, and other determinations associated with comorbidities of overweight and obesity, such as for example, hypertension, elevated cholesterol, and elevated blood pressure.

The application of a HALex-utility index method also may be directed to assessing the patient's perceptions and/or the healthcare professional's observations of the patient's overall health status. The data points in the NHIS may be proxies for the data points related to the patient's socioeconomic status, sociocultural status, and psychosocial status. The statuses data points and the statuses survey questions may be equivalent to or reasonably similar to the questions in the medical interview and/or the core questions in the NHIS. The NHIS variables associated with the NHIS core questions may be proxies for socioeconomic status, sociocultural status, and psychosocial status and for other Social Determinants Of Health. Examples of NHIS variables that may be proxies for statuses data points are scheduled in the following table.

Examples Of NHIS Variables That May Be
Statuses Data Points

| | | |
|---|---|---|
| NHIS Question No. | NHIS Variable | NHIS Question |

Socioeconomic Status Indicator: Income/Stress

| | | |
|---|---|---|
| ACN.125_000.270 | ASTRESYR | Frequently stressed past 12 months |
| ACN.412_05.074 | HREMTP05 | Stress reduction relaxation method |
| ALT.036_87.000 | ACUCND87 | Used acupuncture for stress |
| ALT.112_87.000 | BIOCND87 | Used biofeedback for stress |
| ALT.810_87.000 | DITCND87 | Used special diet for stress |
| ALT.916_00.000 | RELU_STR | Used stress management class past 12 months |

Socioeconomic Status Indicator: Income/Workplace Conditions

| | | |
|---|---|---|
| FSD.070_00.000 | FWRKLWCT | Number of family members working full time last week |
| FSD.050_00.000.R01 | FDGLWCT1 | Number of family members looking for work last week |
| QOL.445_01.000 | P_ANX_4A | Feelings caused by type/amount of work I do |
| ACN.100_00.030 | AWZMSWK | Number of work days missed due to asthma |
| ALT.068_05.000 | ACUNNT05 | Never used acupuncture because I don't believe in it |
| MFM.000_00.000 | FM_EDUC1 | Education of adult with highest education in family |

Socioeconomic Status Indicator: Income

| | | |
|---|---|---|
| FHI.290_08.000 | HISTOP8 | Loss Medicaid/new job/increase in income |
| FIN RECODE | INCGRPI2 | Total combined family income |
| FIN RECODE | POVRATI3 | Ratio of family income to poverty threshold |
| FIN.040_00.000 | FSALCT | Number of family members receiving income from |
| | | wages |
| CAU.130_00.000 | CHCAFYR | Can't afford prescription medicine past 12 months |
| CAU.135_03.000 | CHCAFYR3 | Can't afford dental care past 12 months |
| CAU.135_04.000 | CHCAFYR4 | Can't afford eyeglasses past 12 months |

Socioeconomic Status Indicator: Education

| | | |
|---|---|---|
| FID.360_01.000.RO3 | MOD_ED | Education of father |
| FID.360_01.000.RO2 | MOM_ED | Education of mother |
| QOL.590_00.003 | QOL_2C | Going to school or achieving your education goals |

Sociocultural Status Indicator: Environment

| | | |
|---|---|---|
| ASI.150_00.00 | ASITENUM | Length of time living in neighborhood |
| ASI.160_00.000 | ASINHELP | Agree/disagree people in neighborhood help each other |
| ASI.170_00.000 | ASINCNTO | People I count on in the neighborhood |
| ASI.180_00.000 | ASINTRU | People in the neighborhood can be trusted |
| ASI.190_00.000 | ASINKNT | Close knit neighborhood |

Sociocultural Status Indicator: Social Network

| | | |
|---|---|---|
| ALT.830 00.000 | DIT_FFC | Used a special diet recommended by family, friends, etc. |
| ASD.210 00.180 | WORKWFAM | Compatibility of work and family responsibilities |
| QOL.590_00.009 | QOL_21 | Participating in community gatherings |
| AHB.135_00.010 | DSHFAC | Access to health club/fitness facility |
| NAE.025_04.000 | FE1B 4 | Used help/support from friends/family to stop smoking |
| QOL.590_00.005 | QOL_2E | Getting out with friends/family |
| ACD.120_00.000 | VSLLGFAM | Family, friends, associates had trouble understanding |
| | | what you say |
| BCK.130_07.000 | LCATION7 | Location of interview-in a home of neighbor, friend, |
| | | relative |
| CMH.030_01.000 | RSCL3_P2 | At least one good friend |

Psychosocial Status Indicator: Quality Of Life

| | | |
|---|---|---|
| NAI.100_00.000 | QOL | Reported quality of life |
| QOL.200_00.006 | MOB_3F | Use someone else's assistance |
| AFD.590_00.007 | QOL_2G | Using transportation to get to places you want to go |
| AHB.136_5.050 | DISHFLO6 | Inadequate transportation |
| AOH.080_04.000 | ONODEN_4 | No transportation to dentist past 12 months |
| CAU.080_05.000 | CHCDLYR5 | No transportation past 12 months |

To assist the patient and the healthcare provider manage the patient's achievements under the therapy program, an embodiment may be directed to applying predictive models to establish a the likelihood or probability of the patient's motivation toward the therapy program, the patient's adherence to and compliance with the therapy program, and the patient's cessation from the therapy program, based on the relevant Social Determinants Of Health data and statuses data collected by the patient's health survey during the medical interview and the third-party survey data.

Some embodiments may be directed to predicting therapy adherence, compliance. and/or cessation by evaluating the Social Determinants Of Health to establish to what degree, if any, the patient at the individual level (as distinguished from the household level) is challenged to perform the therapy program as prescribed or recommended based on the impact on the patient of social exclusion, a circumstance known to those skilled in the art, and the resulting impact on the patient's quality of life.

An examination of the impact of social exclusion may identify primary dimensions such as, for example, the health vulnerabilities and the health disparities of the patient. Information on the health vulnerability of the patient may be collected from such sources as, for example, during a medical interview, in the Social Determinants Of Health records associated with the patient and maintained by the healthcare professional responsible for the patient, and/or part of data in a health benefit plan in which the patient may be enrolled. Other sources of data on an individual patient's social exclusion

Indicators of health vulnerability may include, for example: whether and to what degree the patient has economic assets (such as, for example, wage, steady wage, full-time or part-time wages, access to credit or social support networks, saving accounts, home equity in the patient's name, cash value of life insurance that the patient can draw upon, ownership of the equity in an automobile, few assets that can be sold or pawned); whether and to what degree the financial burden on the patient is large in relation to the assets available to the patient (such as, for example, tuition debt incurred under a non-cancellable government education program, the number of children, elderly, other adults, and extended family the patient is supporting); and the composition of the household the patient is supporting (such as, for example, the presence elderly members, the number of pre-teen children, the number of teenage children, an individual with a disability, the regular receipt of alimony); whether and to what degree the patient has diversified income (such as, for example, full-time or part-time employment, underemployment, social security income, retirement income, disability income); whether and to what extent the patient can suffer an economic shock (such as, for example, an automobile accident that keeps the patient from earning living, a severe illness requiring hospitalization, natural disaster, and/or one of such instances for a family member supported by the patient); and whether the patient has a chronic or other long-term illness or medical condition.

Information on the health disparity of the patient may be identified in the same manner as information is identified on the health vulnerability of the patient. After gender and age, indicators of health disparity may include whether and to what degree, for example: the patient is educated (such as, the ability to read, write, and do number calculations at a level for employment compensated by a salary or by hourly wages); the patient has adequate shelter (such as good protection from the weather, separate sleeping areas from kitchen and living areas, privacy, a floor that is easy to keep clean); the patient rents or owns shelter (such as, unpredictable levels of rent increases, unwillingness of landlord to make repairs required for safe, decent, and sanitary living); the patient has sexual autonomy (such as, for example, control over whether, with whom, and how often the patient has sexual relations for social or economic reasons); the patient has freedom from violence (such as, subject to physical, sexual, or emotional violence, in the home or outside it, and has reason to fear violence); the patient is free from the disruptive behavior of other people (such as, gambling, drug abuse, alcohol abuse, and prostitution having a major impact on the patient's quality of life); the patient feels comfortable in their body (such as, reliable access to personal care products such as soap, sanitary products, toothpaste, toothbrush); the patient has free time (such as, time to relax with family and friends, pursue hobbies, without being consumed with housework, taking care of family, and work); the patient has access to information and communication (such as, no or limited access to television, radio, telephone, or other sources of information); the patient has cooperative family relationships (such as, cooperative and supportive family members, participation in major family decisions, joint budgeting decisions); the patient has a voice in the community (such as, participation and influence on decisions that affect the patient's community, feeling of disempowerment in decisions that affect the patient's community); the patient has control of decision-making and personal support (such as, whether the patient will go out of the house into the community, with whom will the patient associate outside of the household, or when and from whom the patient will seek health care); and the patient is able to participate in major community functions (such as, religious, wedding, and funeral ceremonies, where the patient lacks suitable clothing, personal care, low social standing, lacks means to purchase gifts).

Some embodiments may be directed to quantifying the indicators of health vulnerability and health disparity specific to the patient. Quantification may include categorization of the vulnerabilities and disparities. Categories may include, for example: education attainment; economic (such as, indicators of household well-being, food and non-food consumption or expenditure, and income, and non-monetary proxies of household well-being such as ownership of productive assets or durables); demographic (such as, gender and age); poverty; social (such as, nonmonetary indicators of household well-being such as quality and access to education, health, other basic services, nutrition and social capital); race; urban development; and vulnerability (such as, the level of household exposure to shocks that can affect poverty status, for example, environmental endowment and hazard, physical insecurity, political change, the diversification and riskiness of alternative livelihood strategies, household size and composition, asset liquidity, and income diversification).

The scope of quantification also may include weighting or scaling each indicator and category. Weighting decisions may include scaling where, for example, all factor loadings are considered relatively equal. Under this approach, gender, education, and income, for example, may be deemed to be equal in their impact on the patient's adherence or compliance with a therapy program.

More complex and precise weighting or scaling decisions may include ranking by ordinal each indicator and/or each category. An ordinal ranking may be placed into an interval scale. Weighting also may be performed by applying multivariate statistical techniques, principal components, factor analysis and ordinary least squares, each of which is known to persons of ordinary skill in the art. The principal components statistical technique reduces a given number of variables by extracting linear combinations that best describe the variables, and then transforming them into one index. The first principal component, the linear combination capturing the greatest variance, may be converted into factor scores that serve as weights. The index may be crossed with spatially based criteria, such as the patient's neighborhood or city and the patient's access to health, school, and social service infrastructures.

An embodiment may be directed to spatially oriented variables associated with the specific individual patient and patients in the neighborhood or other relatively close or otherwise relevant living environment with the patient, particularly when seeking to individualize the patient's variables out of a community-set of variables. Similar in approach to small-area estimation, a statistical method known to those skilled in the art, the relevant databases for comparison with the patient may include zip code level data collected by the National Health Interview Survey. The small-area estimation approach may be applied in some embodiments that combine survey and census data to estimate indicators for disaggregated geographical units such as municipalities or rural communities, where small-area estimation parameters from a predictive model are applied to identical variables in a census or auxiliary database. A principal assumption under this approach is that the relationship defined by the predictive model holds for the larger population as well as the original sample.

Other weighting approaches may include aggregative methods, stratification, inverse weighting, propensity scoring, principal components analysis, factor analysis, and/or other relative methods. Each of such methods is readily known to persons of ordinary skill in the art.

The weighting for an individual patient may be adjusted up or down based on the experience with the patient in the performance of the therapy program.

In establishing the social exclusion index, an embodiment may be directed to examining the patient's livelihood strategies, wherein evaluation is made of the combination of activities that a patient chooses to undertake in order to achieve the patient's livelihood goals. These goals may include, for example, increased income, reduced vulnerability, reduced dependency, and increased well-being. Evaluation factors may include, for example, the patient's productive activities, investment strategies, and reproductive choices; the patient's access to assets; the Social Determinants Of Health associated with the patient and their impact on the context of the patient's pattern-of-life; the influence of such context on the patient's healthy preferences and healthy choices; reinforcement of the positive aspects of these strategies; and mitigation against the constraints on these strategies. These factors may be weighted or scaled using any one or a plurality of weighting methods discussed above. The most variable of the factors may be the base in the weighting, with the other factor weights comprising the elements of indices or scores representing each variable. Multivariate analysis may establish a combined index reflecting the livelihood strategy. The livelihood strategy itself may be a factor in establishing the social exclusion index. In addition, the livelihood strategy may be a sub-component to the health vulnerability index and/or the health deprivation index as sub-indices of the social exclusion index.

Some embodiments may be directed to analyzing the patient's social exclusion index by focusing on the patient's livelihood strategy and the patient's expected livelihood outcomes. Evaluation may be made of the manner in which the patient arranges options available to the patient—capabilities, assets (including material and social resources) and activities—as applied by the patient to patient cope with and withstand economic shocks. Based on the strategies in such arrangements, the patient's expected livelihood outcomes may be the goals to which the patient aspires and the successes from of pursuing the livelihood strategies.

Some embodiments may be directed to motivating the patient's compliance with the therapy program by issuing to the patient a recognition, incentive, reward, advantage, benefit, or intervention domain (collectively referred to herein as a “motivation instrument”) in consideration for the patient's performance of the prescribed activity. These motivation instruments may be tangible, such as badges, pins, flags, and other insignia, branded cups and writing instruments, rings, experiences such as dinning and travel, reductions to the patient's insurance premium, and cash. Motivation instruments also may be intangible, such as points, miles, credits, and services. Intangible motivation instruments may be converted to tangible instruments, and vice versa. In addition, motivation instruments may be issued in consideration for the patient utilizing a health-related intervention domain. Such domains may be wide in scope ranging from the scheduled follow-up appointment with the healthcare professional where the professional's encounter utilizes the professional's office or telehealth facilities, to the patient's shopping at a grocery store or other merchant that has health-related merchandise that participates in the therapy program.

An embodiment may be directed to measuring the patient's performance of a therapy compliance activity. An activity transaction that causes the issuance of a motivation instrument may be measured by a performance indicator sent to the healthcare professional's therapy performance management service. The performance indicator may compare and register a performance transaction and its relationship to the degree of prescribed performance. For example, the transaction may be registered as of one of “high,” “medium,” or “low”. Each indicator may be assigned a performance value, such as for example, a “high” performance may be awarded a value of “10,” a “medium” performance may be awarded a value of “5,” and a “low” performance may be awarded a value of “1”. The performance indicators may be totaled and the balance allocated to the patient's account at the healthcare professional's therapy performance management service. The patient may access such account by such means as the Internet, a mobile communication device, telephone to the patient service center maintained by the healthcare professional's therapy performance management service, or visiting a merchant that participates in the therapy program having facilities that access such performance management service.

An aspect of some embodiments may be directed to delivering to the patient notice that the patient has earned a motivation instrument or that it has or will be issued, and to crediting the value of the motivation instrument to the patient's account in the system that manages the patient's performance of therapy program. The patient may redeem the virtual currency represented by the motivation instrument. The redemption process may include options for the patient to convert non-tangible motivation instruments to tangible, to convert or exchange like-kind motivation instruments, and to allocate excess or non-distributable values of motivation instruments to transactions representing non-earned therapy performance activities.

Some embodiments of the inventive subject matter may be directed to sponsoring or underwriting the motivation instruments. Sponsors may be the healthcare professional, the professional's employer, and/or one or more third parties. A sponsor may include a for-profit organization, a not-for-profit organization, and a government organization, as well as a healthcare provider, education institution, merchant, community organization, civic organization, and government entity. These sponsors or their brands may have affinities with the patient's illness or medical condition, as well as with the Social Determinants Of Health associated with the patient.

An embodiment may be directed to establishing by the sponsor the value for the motivation instrument. Valuation may utilize a perceived value proposition. The proposition may be a promise or expectation to the patient that the therapy program will give the patient the best possible or the desired health outcome. The proposition may establish or seek to establish a point at which the value to the patient of the motivation instrument is sufficient to cause the patient to perform a specified requirement, reach a goal, or maintain a status or condition of the therapy program. An aspect of the embodiment may identify and calculate motivation instruments and other benefits, tangible and intangible, along with the value that will be assigned and allocated to the patient for those benefits that patients will receive when performing one or more elements of the therapy program. An aspect may calculate the patient's perception of the perceived value based on patient insights derived from a predictive model. An aspect of the embodiment may calculate changes in patient's health status, so that as the patient's health status changes, the perceived value proposition of the therapy program to the patient will change.

The perceived value proposition may utilize a scorecard as a method to help communicate the value proposition in a way that the patient can understand. The scorecard may include graphics that map collected data to a visual representation of the therapy program's objectives, goals, and other therapy measurement factors to the patient's performance status, so that the therapy program may become more approachable and performance measures may become more clear to the patient and the healthcare professional.

In addition, the perceived value proposition may help create a more personal and honest relationship between the healthcare professional and the patient, and accordingly, help achieve an objective of the medical interview. The proposition gives the patient another reason to adhere and comply with the therapy program. The proposition also creates a perception or understanding by the patient that the professional is delivering something of value that is relevant to the context of the patient's pattern-of-life and, thus, genuinely has a priority for the patient's health and welfare.

An embodiment may be directed to delivering to the patient opportunities to perform therapy performance activities underwritten, directly or indirectly, by the sponsor. Performance transactions may include prescribed or recommended cure-related activities, such as stress reduction therapies, or care-related activities such as the patient's activities utilizing the decision-making plan and the family interaction plan.

An embodiment may be directed to implementing the sponsor's perceived value proposition through hierarchies or tiers of patient performance and earned motivation instruments. For example, a tier may utilize stages of performance goals, wherein Tier I may have a weight-loss goal, a time value during which the goal is to be achieved, and an assigned value for motivation instruments for reaching that goal; Tier II may have a greater weight-loss goal, the same or greater time value for achieving the goal, and a larger value for the motivation instrument. The values of such motivation instruments may range in value to include experiences with health and wellness affinities, such as a weekend massage experience, to a higher-value week-long trip to a food preparation and hiking experience.

Such embodiment also may be directed to offering and delivering to the healthcare professional motivation instruments and to redeeming such instruments by the healthcare professional. These opportunities may provide an additional evidence of the professional's personal or professional investment in the patient's healthy outcome. These opportunities also may support the professional's employer's strategies for cost-savings and performance incentives.

Some embodiments may be directed to offering and delivering to the patient opportunities for outreach, engagement, and education encounters as part of a the implementation or reinforcement of a therapy program. Some of such embodiments also may enable opportunities for the healthcare professional to initiate outreach, engagement, and education encounters with the patient. Such opportunities may be exercised by the patient and/or the professional communicating with each other or a therapy environment through a channel remote from the traditional bricks-and-mortar real-world domain of such professional. These channels may include, for example, video-audio conferencing with the professional through the Internet or a mobile device, a mobile or land line telephone, and a device equipped to capture the patient's biometric or other sensory data, transmit the data to a relay transmitter such as a mobile phone for forwarding direct to the healthcare provider's therapy management service, or forward the data direct to such service.

Some embodiments may be directed to supporting the healthcare professional's management of therapy intervention themes, known to one of ordinary skill in the art, and associated with successful outcomes. Such themes may that take into account the Social Determinants Of Health and statuses and their impact on the therapy program. Such themes may include cognitive-behavioral therapy programs. These programs are well known to those of ordinary skill in the art. An active patient theme may promote patient self-care. In such situation, a social support theme may provide help to the patient in meeting illness-related or medical condition-related demands. A fear arousal theme may increase patient concern about the consequences of an illness or medical condition—possibly death. A patient instruction theme may involve the patient's reflection on the complexity of the therapy program. Self-care and social support themes may be associated with the strongest effects on treatment outcome.

Some embodiments may be directed to oversighting the healthcare professional in the management of the patient's self-management intervention themes. Self-management may be particularly critical or otherwise useful in the management of a patient with chronic disease, when over the long term, such patients must rely on unassisted effort and self-regulation to maintain adherence and compliance with the therapy program. Therapy programs or themes may be effective, at least in the short term, where the program known to one of ordinary skill in the art include: self-surveilling and/or self-monitoring; goal-setting; stimulus control; behavioral rehearsal; corrective feedback; behavioral contracting; commitment enhancement; creating social support; reinforcement; and relapse prevention. Data points associated with such programs or themes include the Social Determinants Of Health and statuses associated with the patient and the context of the patient's daily pattern-of-life. Such data may be collected by the patient's engagement with pattern-of-life activities, recorded by the patient, and forwarded to the healthcare professional, or collected and recorded by the professional as part of a medical interview.

Some embodiments may be directed to oversighting the healthcare professional in the management of the patient's adherence and compliance using a multiple strategy approach, particularly where no single intervention targeting patient activity may be effective. Multiple strategies known to one of ordinary skill in the art include: providing social support and other reinforcement for patients' efforts to change; providing feedback to the patient on progress; tailoring education to patients' needs and circumstances; teaching skills' continuity of care; increasing accessibility of services; and a collaborative treatment relationship; behavior skills; self-rewards (such as, taking a walk, taking a day off from work); social support; and personal communication (such as, telephone and electronic text) follow-up. Such embodiments may use components of multi-modal programs implemented in an individualized or tailored manner. Representative multi-strategy/multi-modal approaches include: providing social support and other reinforcement for patients' efforts to change; providing feedback on progress; tailoring education to patients' needs and circumstances; teaching skills' continuity of care; increasing accessibility of services; and a collaborative treatment relationship. All of such adherence and compliance strategies are impacted directly by the Social Determinants Of Health and statuses associated with patient and the context of the patient's daily pattern-of-life.

Other multi-modal approaches may include patient engagement or intervention domains. Such domains may be virtual-world environments (such as, health-themed social media domains), as well as real-world environments (such as areas within a building or within a campus of buildings associated with a treatment or preventive health therapy, work or education environments, acute care facilities, outpatient facilities, outpatient clinics, wellness clinics, health clubs, pharmacies, patients' homes, health-affinity retailers, and health-related government facilities). Such environments may be segmented by predicted intervention, predicted adherence, predicted compliance, and predicted cessation. The intervention domains may be segmented further by patient intervention components such as, for example: Determinants Of Health, Indicators Of Health, disease population, patient lifestyle, patient life-stage, and other components of patient daily-life activities. A patient intervention domain may be correlated with a portfolio of multimodal intervention programs. The components of the multimodal intervention programs may include standalone intervention modals and multicomponent intervention modals. Standalone intervention modals may include, for example, self-monitoring, goal-setting, stimulus control, preferred activity rehearsal, corrective feedback, preferred activity contracting, commitment enhancement, creating social support, reinforcement, and relapse prevention. Multimodal intervention modals also include multicomponent intervention modals, such as patient education, patient activity skills, social support, and personal communication follow-up. Multimodal intervention programs may include providing the patient social support and other reinforcement for patient daily-living change efforts, providing the patient feedback on the patient's progress toward defined change goals, tailoring education to the patient's needs and circumstances, teaching daily-living skills to the patient, providing the patient continuity of care with respect to the patient's personal care plan, increasing the patient's accessibility to healthcare services, and collaborative patient-medical team treatment relationships. Such embodiment may utilize smart databases that apply rules engines to collate with the patient a portfolio of enrolled therapy domains, segmentations, intervention modals, diseases and conditions, so that predicted interventions appropriate to a specific patient, based on the predicted or likelihood of adherence, compliance or cessation, may be mapped to the patient. The active-patient intervention modal may be measured for effects on therapy design and outcomes by weight and scale.

Patient engagement and intervention domains may include utilizing incentives, rewards, and achievements having value to the patient, by way of association with an intervention domain such as a gamification domain. Patient engagement and intervention domains may utilize data from healthcare gamification environments such as those based on one or more chronic diseases and their comorbidities, or based on employer environments and/or based on other social environments. Interventions utilizing such a domain may be based on strategies of delayed or deferred gratification and delay discounting. Delayed gratification may be associated with resisting a smaller but more immediate reward in order to receive a larger or more enduring reward later. Delay discounting may be associated with the patient's preference for smaller immediate rewards over larger but delayed rewards. Health gamification domains and related rewards therapies may link the ability to delay gratification to positive outcomes, including physical health, psychological health, social competence, and academic success. Such outcomes may be particularly important in managing obesity, and its comorbidities as factors associated with chronic diseases.

In addition, patient engagement and intervention domains may include performing prescribed or recommended activities at facilities of a healthcare provider, a retail healthcare site or at a web, mobile device, wearable technology or other electronic healthcare domain.

Patient engagement and intervention domains also may include the use of learning classifier systems. Classification problems may arise where there is inconsistent gameplay reward requiring multiple actions before a reward is obtained, where there is complex modeling of healthcare game strategy, where healthcare domain navigation is complex, and where modeling is required for complex, time-dependent, incentive/reward causal interrelationships impacting deferred gratification and delay discounting. Classification problems may include game analysis, pattern recognition, and Boolean function learning. Learning classifier systems may analyze Social Determinants Of Health and disease population classifications and their correlation with the root causes of an individual patient's pattern-of-life activities and the association with gameplay pattern-of-life activities in connection with predicting the likelihood or probability of adherence, compliance and/or cessation with the therapy program. Learning classifier systems also may be applied to analyze reinforcement learning problems in modeling adherence gameplay strategies and other problems attributable to the real time gameplay.

Some embodiments may be directed to intervention modals that utilize cognitive-behavioral therapy. Such therapy is known to those skilled in the art. Cognitive-behavioral therapies may be particularly suitable for aspects of such embodiments that recognize the Social Determinants Of Health and statuses as part of the context of a patient's health, where such therapies include resource management, problem solving skills, solution-focused techniques, and coping skills. An example of a coping skills therapy may be described in the medical interview as questions or comments regarding how the patient has managed to cope, not to give up, or not to break down completely. Responses from the healthcare professional may be, “How do you manage all this pain?” and “What kind of resources do you have that help you withstand the pain?” The therapy prescription in this case may be tailored to the situation. For example, the prescription for intense persistent lower back pain that flares up when walking may be to purchase a mobile wheel chair if the patient has the financial means or has an insurance benefit that will pay for the chair.

An example of a resource management therapy may be described in the medical interview as questions or comments from the patient regarding the healthcare professional's comments on what the professional might consider a positive asset or situational aspect of the patient's health status. Responses from the professional may be, “Well, it is nice to have your grandchildren around! That must be nice, don't you agree?” Solution-focused therapy techniques may be an intervention where questions and comments explore exceptions to the pressure of the symptom, where the symptoms are less or even gone. An example may be, “Are there any circumstances under which the lower-back pain is less or even gone?” Another solution-focused therapy technique may be a situation where the healthcare professional alone or in discussion with the patient defines homework to be performed by the patient prior to the next session. An example may be, “Are there any issues you would like to explore a bit more until we meet next Friday?”

Other intervention modals that an embodiment may be directed to include those established under healthcare guidelines issued by the Institute for Clinical Systems Improvement and other healthcare professional associations, guidelines and treatment algorithms issued by the CDC and CMS, preventive health promotions approved by the Inspector General of the U.S. Department of Health and Human Services, prevention activities authorized by legislation such as the Affordable Care Act, and other medically-acceptable therapy programs.

Some embodiments may be directed to utilizing therapy reinforcement intervention from time to time. Reinforcement may be appropriate given the continuous or recurring impact over time of the Social Determinants Of Health and statuses on the patient's health and the context of the patient's pattern-of-life activities. Reinforcement interventions may consist of a motivation instrument. Reinforcement interventions also may include the healthcare professional's empathetic response to the patient, encouragement to the patient, assistance to the patient, and guidance to the patient, particularly where they are furnished pursuant to a health decision-making plan and/or a family interaction plan. Reinforcement utilizing a family interaction plan may include a family-member's response the patient's care requirements, encouragement to the patient, assistance to the patient, and guidance to the patient pursuant to the guidelines of the plan.

Some embodiments may be directed to extracting data from the databases and applying the data to predictive models to establish the likelihood or probability of patient motivation, adherence, compliance, and/or cessation with respect to diagnosing the health condition of the patient, designing a newly-created therapy program, or managing a continuing therapy program. Therapy programs may include the performance of prescribed remedial or curative activities, as well as the utilization of health-related assets, such as follow-up visits to healthcare professionals, use of healthcare specialists, use of exercise facilities, use of family, social, and workplace networks as prescribed in therapy decision-making and family-interaction plans, and shopping for health-related goods and services at establishments having affinities to health and wellness.

An embodiment may perform the calculations of the predictive models and present their calculations through weighted or scaled results referenced to a prediction range. The prediction ranges may be, for example, ranges of motivation, adherence, compliance, and cessation with the therapy program, as well as ranges of performance of required compliance activities, ranges of choice related to expected quality of life, ranges of the likelihood of survival, ranges of the impact on the patient of those Social Determinants Of Health associated with the patient. Such compliance performance ranges may be the patient's making prescribed choices between beneficial or harmful effects, particularly where the further-most harmful effect may be a severe medical condition, hospitalization, an operating procedure, or even death. A compliance performance range, for example, may be “0” for low or no compliance with the prescribed element of the therapy program and “1.0” for high or full compliance. The resting point within the mid-portion or the upper or lower limits of the prediction range may be the indicator of the predicted subject matter, such that for example, a resting point of “6.5” may indicate the level of performance or achievement toward a blood pressure goal, a weight goal, or a glucose goal. The positon within the predicted range may be an indicator of the patient's then-current status or achievement of therapy goals, outcomes, performance requirements, and other measures of adherence and compliance established by the therapy program. Prediction scores or indicators also may be combined into an overall health or wellness score, goal, or indicator.

The position within the predicted range also may be an indicator of whether the patient is entitled to receive motivation instruments, such as for example, recognitions, incentives, rewards, advantages, and benefits, where the motivation instruments may be available to the patient through a benefit plan associated with a healthcare professional or a third-party benefit plan that is not associated with the professional. The third-party benefit plan may or may not have an affinity with health.

An aspect of the embodiment may calculate, prior to the issue to the patient of a motivation instrument, the difference in value of the motivation instrument in consideration for performance of a requirement of a therapy program and the future healthcare costs reasonably expected to be avoided as a result of preventive care. Such calculation may use one or more predictive models. Such calculation may take into account perceived value propositions to establish a point at which the value to the patient of the motivation instrument is sufficient to cause the patient to perform a specified requirement, reach a goal, or maintain a status or condition of a therapy program. Where the difference in value of the motivation instrument exceeds such costs, the excess value of the motivation instrument may not be issued to the patient where the healthcare professional is to be reimbursed under the Medicare or Medicaid programs for servicing the transaction in which the motivation instrument is issued in connection with the patient's performance of the prescribed activity of the therapy program. In some aspects of the embodiment, such excess value may be deferred, stored, and issued in consideration for the performance of non-earned compliance activities, where such activities are not connected with a commercial transaction in which there is a payment by cash, cash equivalent, or other consideration of value including credit, debit, or gift card, or an exchange of property or services. Non-earned compliance activities may include, for example, those preventive care services listed in the U.S. Preventive Services Task Force's Guide to Clinical Preventive Services and preventive health assessments and screenings permitted under the Affordable Care Act.

The patient's eligibility to receive a motivation instrument may be based on the total or net balance of the values of the predicted indicator scores. Such balance may reflect net values of non-negotiable and negotiable motivation instruments. Such balance may be maintained in an account administered by the benefit plan associated with the therapy program. The patient may access information in such account through such channels as, for example, telephone call to the patient or customer care service center managed by the benefit plan, Internet telehealth system managed by the benefit plan, and direct access by Internet and mobile communication devices.

Some embodiments may be directed to reporting the predicted indicator scores to the patient and to the healthcare professional. The report may include a graphic and/or a numerical presentation of one or more of such scores. The report also may include an analysis of such scores. The analysis may include the then-current health status of the patient benchmarked against the beginning health status established during the initial medical interview and the goals of the therapy program. The report may include scenarios where the patient must make decisions choosing between expected beneficial or harmful effects. Such scenarios may include, for example, “Mary! You are a diabetic who must carefully watch your weight. It is reaching a dangerous level. You may be in danger of losing your toes or feet if you have pain or numbness in these areas while your weight is at this current level. Please immediately call Dr. Jones at 312.555.5555 for an appointment.” The report may include one or more therapy reinforcements to the patient, such as, “Great job! We will post to your therapy peer group that you received a gold star in recognition of your achievement. You might achieve more of your goals if you increase your walking exercise from three to five times a week.” or “Caution! You did not report that you took your insulin shot last night. Failure to take this medication might lead to severe complications and consequences including your loss of mobility, hospitalization, or death. Please immediately call Dr. Jones at 312-555-5555.” The report may be made through several communication channels, such as for example, phone call, text message, Internet communication, telehealth video conference, and letter delivered by postal service.

In response to the report to the healthcare professional, some embodiments may be directed to adjusting the therapy program. Adjustments may include, for example, increased or decreased medication dosages, follow-up appointments, and visits by nurses or social workers to the patient's home. Adjustments also may include, for example, recommendations to the patient to change therapy goals or the time periods within which to reach goals, to change location of a healthcare facility used by the patient in the patient's neighborhood to a location in a different neighborhood, and to change healthcare specialists and outpatient services based on an increase or decrease in the patient's health status or change in the circumstances of the Social Determinants Of Health associated with the patient.

Some embodiments may be directed to designing or improving intervention and treatment therapy programs. Factors that may be considered and applied in the design or improvement methods may include, for example: the patient's knowledge and skills about the patient's health status and health care, (including by way of illustration: the patient's health problem, patient self-regulation of the prescribed or recommended compliance activities contained in the patient's therapy program, the mechanics of the prescribed or recommended compliance activities, and the importance of adherence and compliance); the patient's beliefs and attitudes about health and healthcare, (including by way of illustration: the patient's perceived severity and susceptibility of the illness, the patient's self-efficacy, self-esteem, or self-worth, the patient's expected outcomes, and the patient's expected cost to adhere or comply); the patient's motivation assessed, (including by way of illustration: by the patient's association of successful outcomes from adherence or compliance with the therapy program as a reinforcement factor for continuation with the therapy, and assessed, for example, by the patient's association of negative outcomes from adherence or compliance as a basis for reflection on, and modification of lifestyle activities [not failure]); and the patient's action, (including by way of example as stimulated by relevant cues, as driven by information recall, evaluation, and selection of patient activity options, and as limited or enabled by financial resources available to the patient, social support, and other resources).

Data points for the design or improvement of intervention and therapy programs may be obtained from a variety of relevant databases, including for example, databases containing: the Social Determinants Of Health and statuses, the individual patient's personal profile, the medical interview surveys of the patient and from other third-party surveys, notes from the healthcare team supporting the healthcare professional, and from disease population databases. Intervention and/or compliance data point may be measured by weight, scale, and other factors. The aggregate measurement may be converted to an Intervention Index or Intervention Score. The weighting, scaling, and scoring may be performed with SAS.

Some embodiments may be directed to applying intervention themes in the therapy design or improvement methods utilized by the inventive subject matter for treatment therapies and domains and for intervention and engagement domains. Intervention themes may include, for example: an active patient theme, which may promote self-care or self-management; a social support theme, which may promote help to the patient in meeting prescribed or recommended compliance activities; a fear arousal theme, which may reflect a patient's increase in concern about the consequences of the disease; and a patient instruction theme, which may reflect the complexity of the prescribed or recommended therapy.

Data points for the design or improvement of the intervention and therapy programs may be obtained from a variety of relevant databases, including for example, databases containing: the Social Determinants Of Health and statuses, the individual patient's personal profile, the medical interview surveys of the patient and from other third-party surveys, notes from the healthcare team supporting the healthcare professional, and from disease population databases. Intervention themes may be measured for effects on treatment outcomes by weight, scale, and other factors. The strongest weight, scale, and scoring may be allocated to the active patient theme and the social support theme. The basis for allocation by strength of theme is known to those skilled in the art. The aggregate measurement may be converted to an Intervention Index or Intervention Score. The weighting, scaling, and scoring may be performed with SAS.

Embodiments may be directed to managing data challenges associated with interventions that seek to design or improve adherence and compliance. Data challenges may include identifying and collecting data points associated with the many root causes that may impact the design and performance of the therapy program. The multitude of such data points and the social context in which such data points reside may result in there being no single intervention that solves adherence and compliance problems for many patients. Other challenges may include the time associated with analyzing the data points associated with the patient—perhaps several months—to reliably identify the non-adherent patients. This may mean that non-adherent patients are identified after the fact. In order to provide the level of detail needed to determine the root cause of non-adherence at the individual patient level, additional challenges may include the need for micro data points that originate deep in the Components Of Health and the Indicators Of Health associated with the patient, as well as the patient's trust in the healthcare professional, trust in the privacy of the data, and trust and acceptance by the patient in the privacy of the data collection/intrusion process. These challenges may make it difficult to know which intervention is the most relevant to and needed for an individual patient. Notwithstanding such challenges, the inventive subject matter based in the Social Determinants Of Health uniquely may be suitable for managing such challenges.

Directing the identification and collection of data supporting the design or improvement of therapy programs may include communication channels relevant to the patient's pattern-of-life. For example, a message to a patient's physician may be most effective for an Energetic Circumspector, whereas a text message to the patient may be most effective for an Occasional Neglector. Others communications may include the type of pre-commitment to the therapy program made by the patient, benchmarking and reporting to the patient of compliance by the patient against other patients at an aggregated interventions level, issuance of rewards to patients who comply with preventive health therapies, entrance into lotteries for patients who take their medications each day, for patients identified as at-higher risk of non-compliance, communication channel may escalate more quickly through flashing, beeping, and phone call reminders, telephone (land line or mobile device), Internet, web-, mobile-, and other multimedia-based channels for the delivery of healthcare instruction, daily-life activity counseling, and engagement with persons in the patient's social network who have similar chronic illnesses, and therapy performance incentives, and rewards.

Directing the identification and collection of data supporting the design or improvement of therapy programs may include the use of biometric data from wireless devices as scales, glucose meters, and blood-pressure monitors and/or data acquired from a software application delivered by an Internet or mobile device. MapMyRun, a software application that specializes in fitness, and exercise, records a patient's running routes, distances traveled, and finish times, and enables the patient to set goals, and establish a training schedule. This software application may be connected to Apple's Health app, which enables the patient's runs to be added to Appel Health's daily distance traveled tally. Other apps may analyze a patient's runs and other physical activities in different ways.

Directing the identification and collection of data supporting the design or improvement of therapy programs may include daily activity data acquired from a software application. The Lark software application reportedly pulls a patient's fitness, sleep, and nutrition data from other software applications and breaks down the patient's daily progress in a conversational tone. Lark sends text-like messages explaining these health details. Lark reads sleep, fitness, and nutrition info, and then provide the patient a daily status in a conversation. Lark also may remind the patient to get up, and take a walk when the patient has been sitting at a desk for too long. A patient may check Lark throughout the day. Lark reportedly can compare a patient's stats to those of previous days and a patient's typical averages to check progress. In another example of an online application, an intervention using a preemptive-activity evaluation technique may be used for managing diabetes, by utilizing nutrition data. Reportedly, MyFitnessPal is a software application for tracking meals. It has a database of common grocery store items that may be searched and has tabs for saving information on foods prescribed or recommended wider a preventive health therapy program, as well as foods the patient frequently likes to eat, favorite meals, and recipes. The software application also enables the patient to scan an item's barcode to log that item into the patient's food diary. The software application enables the patient to view the micronutrient breakdown of the logged food. The software application converts a patient's steps-to-calories-burned and includes that data in the patient's daily calorie-in versus calorie-out goal.

Directing the identification and collection of data supporting the design or improvement of therapy programs may include patient daily activity data collected by a wearable technology such as clothing and devices. The Apple Watch wearable device is designed to give a patient a more complete picture of the patient's all-day physical activity by measuring the quantity of the patient's movement, such as the number of steps taken, by measuring the quality, frequency, and progress, and by motivation to sit less, move more, and get exercise. Over time, the Apple Watch is designed to use what it learns about the way one moves to suggest personalized daily fitness goals and encourage achievement so as to live a better day and a healthier life.

Directing the identification and collection of data supporting the design or improvement of therapy programs may include consumer marketing data providing inferred daily-life activity, lifestyle, and attitudinal information on consumers from their demographic data, retail purchasing history, and credit history.

Some embodiments may be directed to assessing the success of the therapy program from the patient's point of view. Such an assessment may include factors for the patient's comments on and evaluation of the therapy program's therapy aim, goal, performance requirements, performance measures, health outcome goal, and health outcome measure. The assessment may be made with consideration of factors for the impact on the therapy adherence and compliance performance in light of the impact of Social Determinants Of Health associated with the patient. The assessment also may include factors for the patient's evaluation of the success of the therapy program's action plan for the patient's management of the program's implementation.

Assessment of the success of therapy program from the patient's point of view may include factors for the program's decision-making plan. Such factors may include changes in a diagnosis and a prognosis, changes in an improvement or a decline in health status, a request for medical information and healthcare support, a change in, a reduction of, and/or an addition of healthcare support, a change in medical evident and interpretation of medical evidence, a contact for a clinician and a caregiver, a plan and the implementation success of such plan for care coordination among a discharging healthcare professional and a receiving healthcare professional, an indicator from the patient of the patient's trust of the healthcare professional, an indicator from the patient of the value to the patient of the patient's expected benefits and harms from the therapy program, an indicator from the patient of the patient's expected health outcomes from the therapy program, and where appropriate, a written advance care directive.

Assessment of the success of therapy program from the patient's point of view may include factors for the program's family interaction plan. Such factors may include a presence and/or an absence of a plan for communicating among the patient and a family representative. Such factors may include identifying and addressing patient and family concerns about the patient's disease or condition. Additional factors in such plan may identify the patient's, and/or one or both of the parents of the patient, and/or one more of the children of the patient, for healthcare treatment preferences and for managing barriers to the implementation of treatment. Other factors may include the process of developing treatment goals, as well as the goals per se, together with active patient-assessment and patient management of the patient's disease or condition.

Assessment of the success of therapy program from the patient's point of view may include factors relevant to the patient and internal or subjective to the patient. These internal factors are complex and may, for example, include: the patient's attitude toward the condition being treated, and the patient's fear or avoidance of a medical crisis. An internal factor implicit in the patient's point of view is the patient's belief in a right of intelligent non-compliance based on the impacts on the patient of the factors and patterns of everyday life. The patient often may decide that the therapy program is all well and good, but in practice, “I'll do what I can as long as it's not too painful, too expensive, or too inconvenient.” Other factors include the patient's perception of health risks of the illness or condition and the relative benefits of the therapy program. These factors reflect the patient's dread, anxiety, or apprehension that may be revealed in compliance with self-medication, a complex therapy regime requiring one or more procedures, activities, or steps, treatment over a long-term (particularly in the case of a chronic illness) where patterns-of-life may interfere with compliance (such as the illness of a child or patent), actual or perceived complex self-medication or self-performing therapy, self-administration of medication on a time-line protocol. Additional factors may include requirements for the establishment, entry, and maintenance of an activity log at multiple recording intervals, the entry into a log or device of the patient's biometric and other sensory data, the mechanics of communicating remotely the patient's sensory data to a healthcare professional and concerns about the privacy of the communication channel, and the performance of a remedial physical activity on a repetitive time-base protocol.

The complexity of the patient's internal factors in assessing the success of the therapy program also include evaluation factors reflecting: the patient's perceived remedial effect of the treatment and medication, the patient's experiences with undisclosed side-effects, the patient's awareness of a change in the disease or condition, misdiagnosis of the patient's disease or condition by the healthcare provider, inappropriate prescribing by the healthcare provider, the patient's belief of the importance of cues and mechanisms for patient action, the patient's belief toward at least one of the patient's perceived severity of the disease or condition, the patient's perceived susceptibility to the disease or condition, and the patient's perceived relevance of the disease or condition to the patient, the patient's concerns about side effects, the patient's control of the symptoms of the disease or condition, the patient's enjoyment of a quality lifestyle that may be adversely affected by the therapy program (such as the prescribed use of a walker medical device), the patient's expectations of outcomes, the patient's knowledge and skill required to perform, develop, and/or acquire in order to comply with the therapy program, the patient's maintenance of financial comfort during the conduct of the therapy performance specifications, the patient's perception of good health (such as, for example, “I feel good, so I won't do all this stuff until I feel bad”), the patient's perception of the therapy performance specifications as being manageable, tolerable, or effective, the patient's progress toward the stated goals of the therapy program, the patient's assessment of and comfort with the healthcare provider, the patient's family, friends, and workplace support structure, the patient's understanding of the therapy program's aims, performance requirements, and performance measures, and the patient's understanding of how the Social Determinants Of Health affect the disease or condition being treated.

Assessment of the success of therapy program from the patient's point of view may include factors relevant to the patient and impacting the patient from the external environment. Such factors may include: the patient's activity limitation (evidenced by, for example, unassisted effort and self-regulation), the patient's follow-up therapy reinforcement strategies utilizing face-to-face, telephone, computer, mobile device, Internet, video conference, letter, text message, a sensory device, the patient's frequency of communication with at least one of a healthcare worker, family member, friends, and social network. External factors also may include the patient's receipt of healthcare education, behavior skills, independent living skills, and self-medication skills tailored to the patient's disease or condition and needs and circumstances. Other external factors may include the patient's receipt of continuity of care between hospital discharge and outpatient care and home care, the patient's receipt of feedback on progress in improving health status or in performing the therapy program, the patient's receipt of social support from family, friends, workplace, and social networks, the patient's stimulation to comply with the therapy program motivated by relevant cues driven by the patient's recall of information and the patient's evaluation of the recalled information, and the patients eligibility for and receipt of incentives, rewards, advantages, recognitions, credits, currencies, and other consideration for achieving therapy and compliance goals.

Some embodiments are directed to communicating to the patient and the healthcare professional the results of the measurements and determinations by the patient of the therapy program's success. Communication may be provided by several channels, such as for example, face-to-face conference (such as in a medical interview), letter, text message, telephone, local area networks, the Internet, and communication-enabled medical device.

As one can readily see from the above challenge-management strategies, the inventive subject matter and its basis in the Social Determinants Of Health uniquely may be suitable for managing the challenges associated with identifying and collecting data associated with a patient's Social Determinants Of Health and their integral Components Of Health and Indicators Of Health. The inventive subject matter may eliminate or reduce patient disruption since only patients predicted to be or identified as being at risk or high risk of non-adherence or noncompliance may receive an outreach or intervention from the healthcare professional. In addition, tailored interventions may be offered, versus a one-size-fits-all solution. Moreover, by predicting in advance which patients are at elevated risk, methods and systems may promote proactive action rather than reactive action to therapy adherence and compliance challenges.

As can be appreciated by one skilled in the art, a computer system with an associated computer-readable medium containing instructions for controlling the computer system may be utilized to implement the exemplary embodiments that are disclosed herein. The computer system may include at least one computer such as a microprocessor, a cluster of micro-processors, a mainframe, and networked workstations.

Thus it is seen that methods for predicting a patient's adherence to a therapy program, and optimizing the therapy elements are provided. While the foregoing written description of the inventive subject matter enables one of ordinary skill in the art to make and use what presently is considered to be the best mode thereof, those of ordinary skill in the art will understand and appreciate the existence of variations, combinations, and equivalents of the specific embodiments, methods, and examples herein, and will appreciate that the present inventive subject matter can be practiced by other than the described embodiments, which are presented for purposes of illustration, and not of limitation. The inventive subject matter should therefore not be limited by the above described embodiments, methods, and examples, but by all embodiments, and methods within the scope, and spirit of the inventive subject matter, and the present inventive subject matter is limited only by the claims which follow. Moreover, in interpreting the disclosure, all terms should be interpreted in the broadest possible manner consistent with the context. In particular, the terms “comprises”, and “comprising” should be interpreted as referring to elements, components, or steps in a non-exclusive manner, indicating that the referenced elements, components, or steps may be present, or utilized, or combined with other elements, components, or steps that are not expressly referenced.

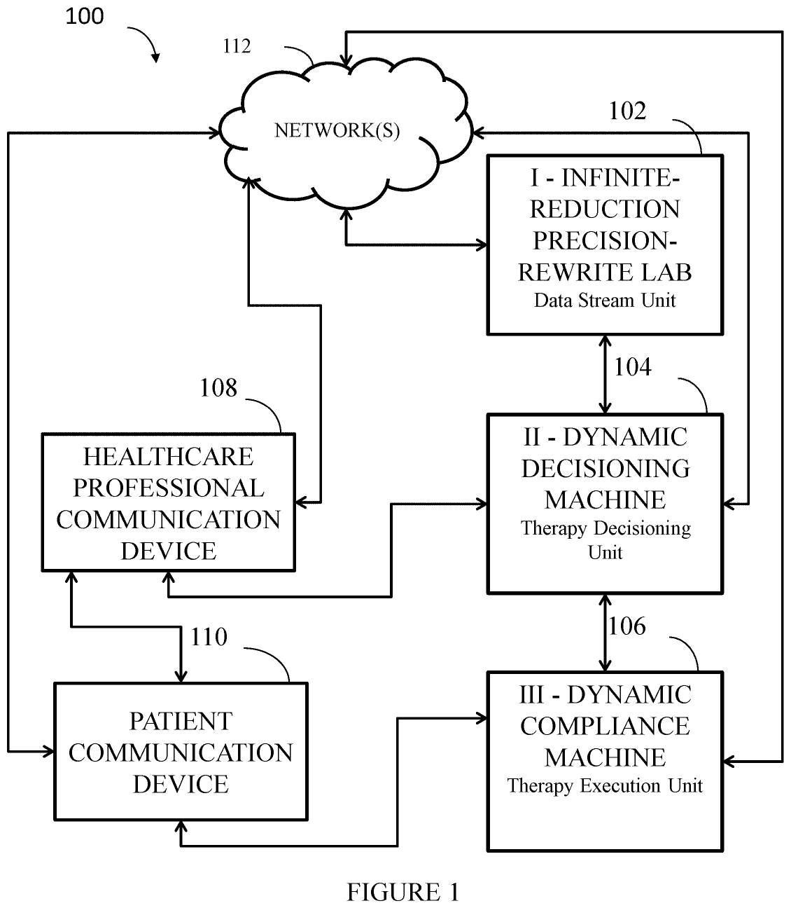

FIG. 1 shows a block diagram of an example system 100, according to an example embodiment. The system 100 is an example environment in which a patient's adherence to and compliance with a medically-supervised and prescribed or recommended therapy program may be improved. The system 100 includes an infinite-reduction precision-rewrite lab 102 subsystem, according to an example embodiment, a dynamic decisioning machine 102 subsystem, according to an example embodiment, and a dynamic compliance machine 106 subsystem, according to an example embodiment. More or less subsystems may be used. Each of such subsystems 102, 102, and 106, may be in communication with each other, each of such subsystems 102, 102, and 106, also may be in communication with a healthcare professional communication device 108 and a patient communication device 110, and each of such subsystems 102, 102, and 106, and communication devices 108 and 110 may be in communication with the system 100 over a network 112.

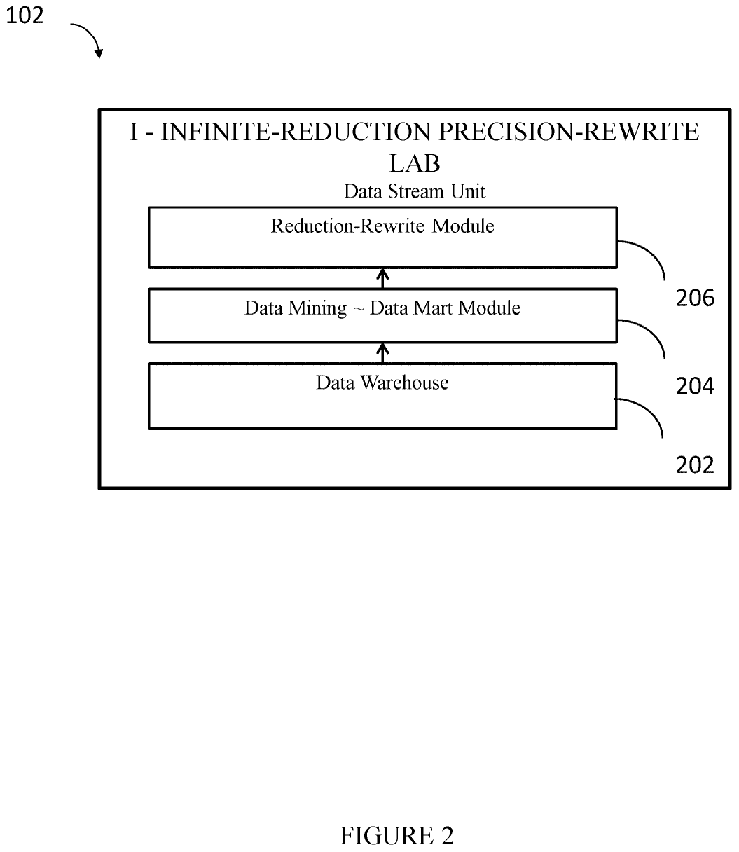

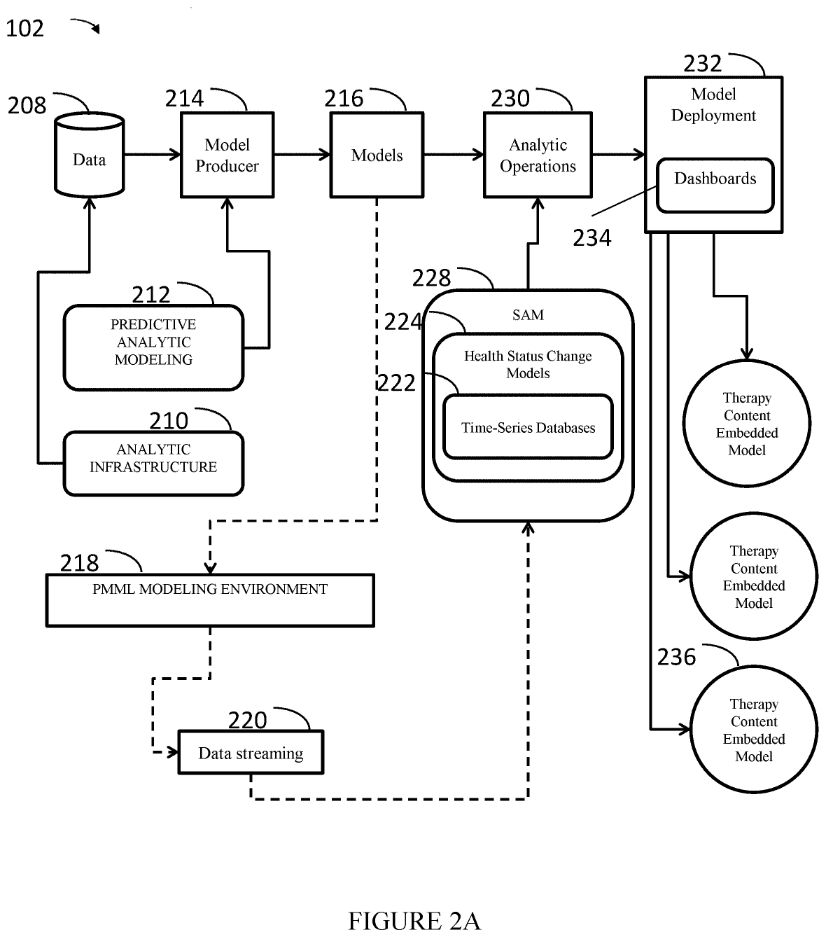

FIG. 2 shows a block diagram of the infinite-reduction precision-rewrite lab 102 subsystem of system 100, according to an example embodiment. The infinite-reduction precision-rewrite lab 102 subsystem may be comprised of data warehouse 202, data mining-data mart module 204, and reduction-rewrite module 206. The infinite-reduction precision-rewrite lab 102 subsystem collects, mines, indexes or scores, and warehouses the stream of Social Determinants Of Health data correlated with a patient, as well as such data correlated with populations having illnesses and medical conditions correlated with the patient and other populations.

The module 202 of infinite-reduction precision-rewrite lab 102 subsystem may collect data. Data collected by module 202 may include Social Determinants Of Health data correlated with the patient, such as for example, data from the patient's medical interviews and social exclusion data such as data on the patient's health vulnerability, health disparity, and livelihood system. Social Determinants Of Health correlated with populations may include survey data (such as, for example, household surveys, census, participatory assessments, sectoral, and spatial surveys), secondary data (such as administrative data), guided group discussion data, key informant interview data, and consumer data. Patient-specific data may be collected from the patient by the healthcare professional responsible for the patient and from other patient-supplied data sources specific to the patient, such as, for example, data from a healthcare benefit plan in which the patient has enrolled, data from biometric sensory devices, data transmitted from mobile phones and other mobile devices, data from by social media websites, and data from virtual therapy domains. Such data may be static, and also may be dynamic realtime or near-realtime. Such data may be structured (such as, for example, databases), unstructured (such as, for example, video and audio), a hybrid structured-unstructured (such as, for example, web-based interactive audio-video-text), and other formats. Such data collection functions may be performed in a module of the infinite-reduction precision-rewrite lab 102 subsystem. After data is extracted from module 202, processed, and activated in therapy programs through other subsystems of the system 100, the data in its then-current state is stored in module 202 for future use in the management of the patient's therapy program.

The data mining-data mart module 204 of infinite-reduction precision-rewrite lab 102 subsystem may perform data cleaning, sorting, aggregation, segmentation. When these processes are completed, the data may be extracted by reduction-rewrite module 206 of the infinite-reduction precision-rewrite lab subsystem.

The reduction-rewrite module 206 of the infinite-reduction precision-rewrite lab subsystem may analyze and synthesize the data, as well as perform various statistical analyses on the data, such as for example, decomposition, inequality, poverty, distribution, redistribution, dominance, polarization, welfare, and curves. Such analysis and synthesis may be performed with respect to populations who have the same or similar illnesses or medical conditions, a population of individual patients who have the same or similar illnesses or medical conditions, and the individual patient having the same or similar illnesses or medical conditions. The illnesses or medical conditions may include comorbidities and chronic illnesses or medical conditions. Results of such analysis and synthesis may include indexing or scores correlated with Social Determinants Of Health, patient populations, and specific patients.

The dynamic decisioning machine 102 of system 100 may be comprised of dynamic prediction machine module 302, dynamic feedback scoring machine module 304, and dynamic therapy optimization machine module 306.

FIG. 2A shows a block diagram of a method of predictive model building that may be employed by reduction-rewrite module 206 of infinite-reduction precision-rewrite lab 102 of system 100, according to an example embodiment. Databases 208 may be built upon analytics infrastructure 210, including a data warehouse, operational data, and an analytics data mart. Predictive analytic modeling 212 method may include cleaned data extracted from the data mart, exploratory analysis of the extracted data, construction of candidate analytic models, and validation of the candidate models. Validated models may be stored in model producer 214.

The validated models may be processed in modeling environment 218, which may include application vendors (such as for example, SAS, SPSS, R, Python, Java, Cognos), document components (such as for example, data directories, mining schema, transformation directories, model components [segments, ensembles, etc.), model verification, univariate statistics, optional extensions), models (such as for example, cubes, trees, associations, neural nets, naïve Bayes, sequences, text models, support vector machines, rulesets, polynomial regression, logistic regression, enter based clusters, density based clusters), statistical techniques (such as for example, segmentation, concept description, classification, prediction, dependency, inequality poverty, decomposition, redistribution), and algorithms (such as hybrid aggregate/detection event processes, treatment therapy portfolios).

Analytic operations 230 may encompass several processes. Data streamed 220 from modeling environment 218 may be processed by a time-series databases 222 module. These databases may include a baseline for patients, for general population, for illness-specific and/or medical condition specific populations, as well as and for “driver” indicators per dimensions per cube, estimated baseline changes, and detected changes from the baseline. Processed time-series databases 222 may further process through health status change models 224. These models may include models for each Social Determinant Of Health, their respective Components Of Health, and/or their respective Indicators Of Health using multidimensional data cubes (star/snowflake schema) for one or more of such determinants, components, and/or indictors. The data stream may further process through SAM (scores, actions/alerts, measures) 228 module.

Model deployment 232 may encompass the operationalization of one or more analytic models. Operationalization may include embedding one or more models into therapy content 236, where the insights from the models would be applied by healthcare professionals in the design, improvement, and/or optimization of therapy programs. Surveillance and monitoring of the patient's compliance activities by the embedded models, and the feedback loop, may be presented in interactive dashboards. The dashboards may be accessed by healthcare professionals.

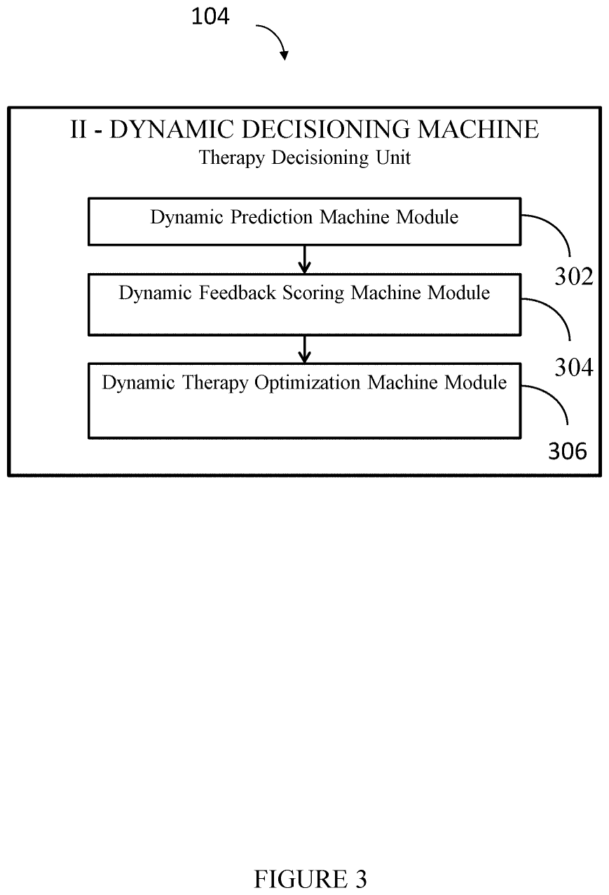

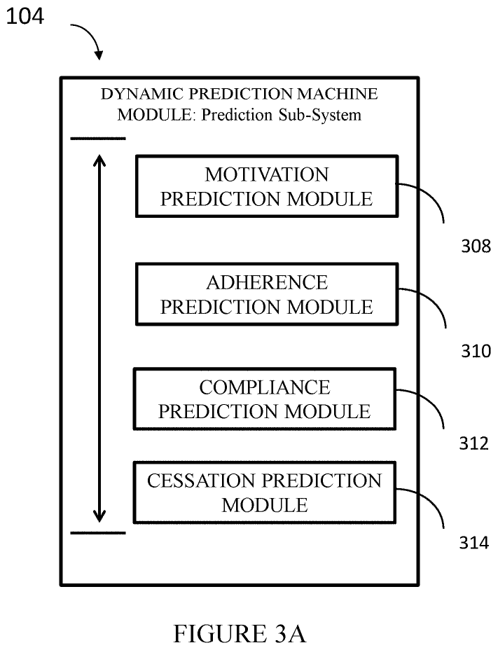

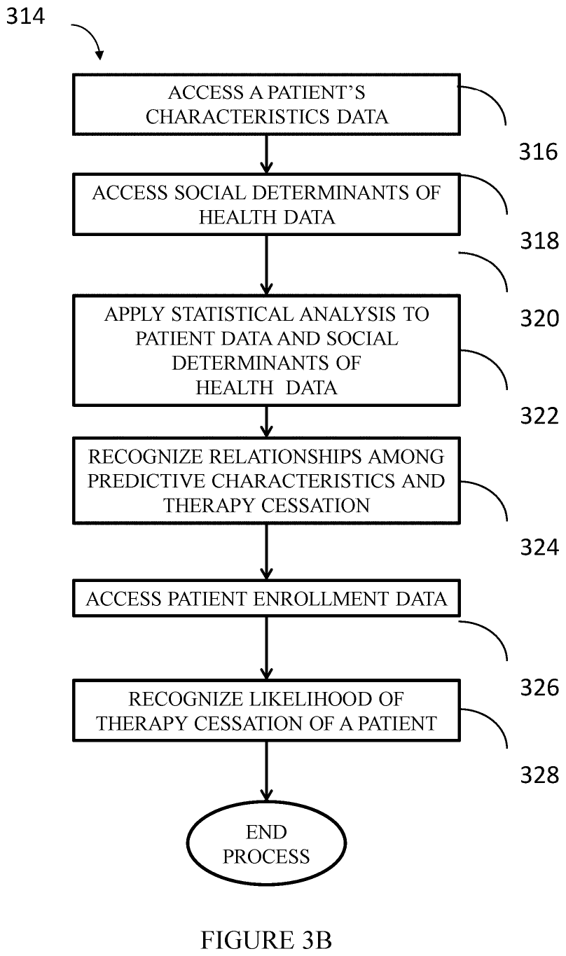

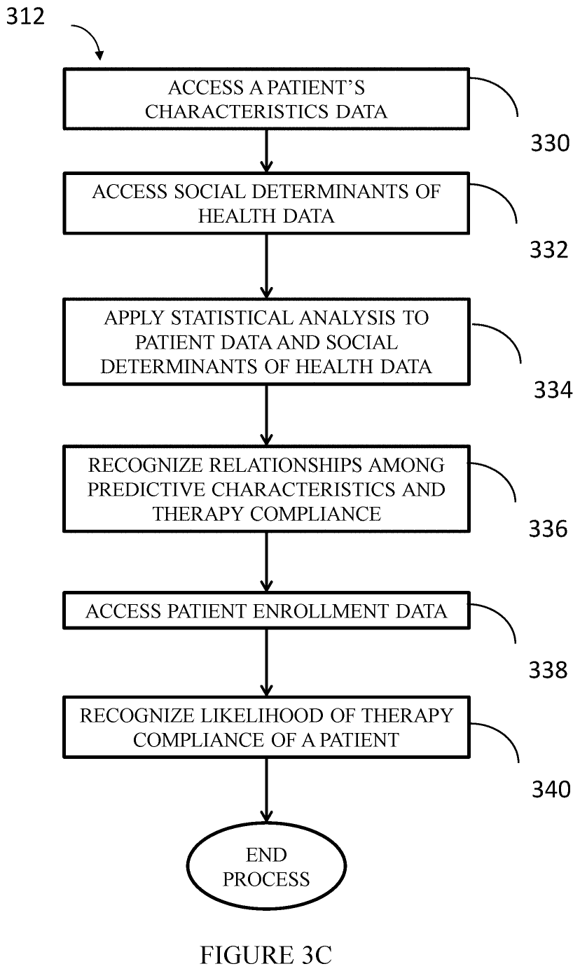

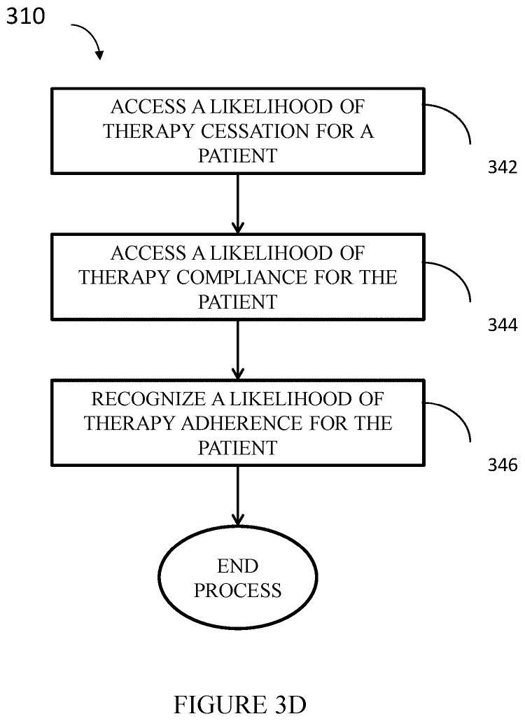

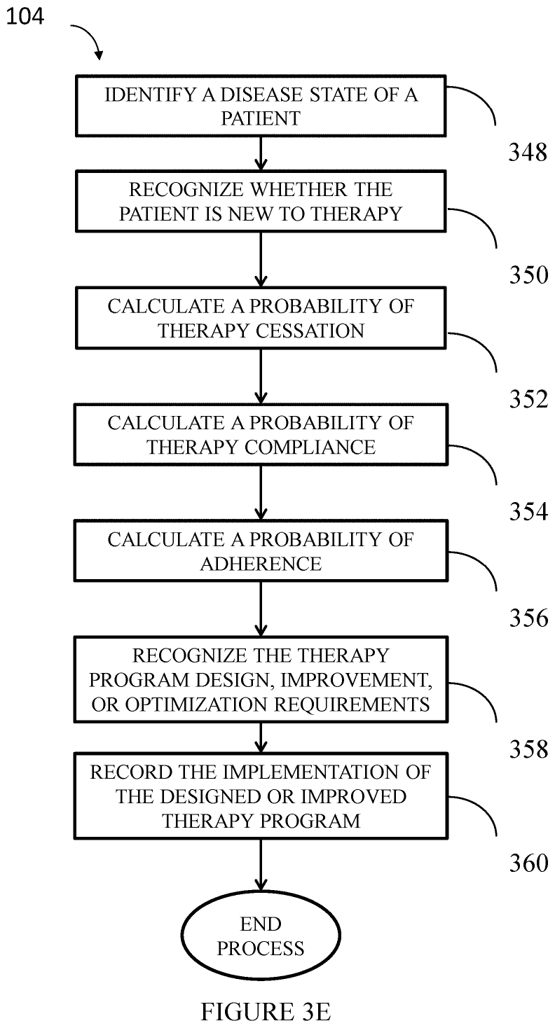

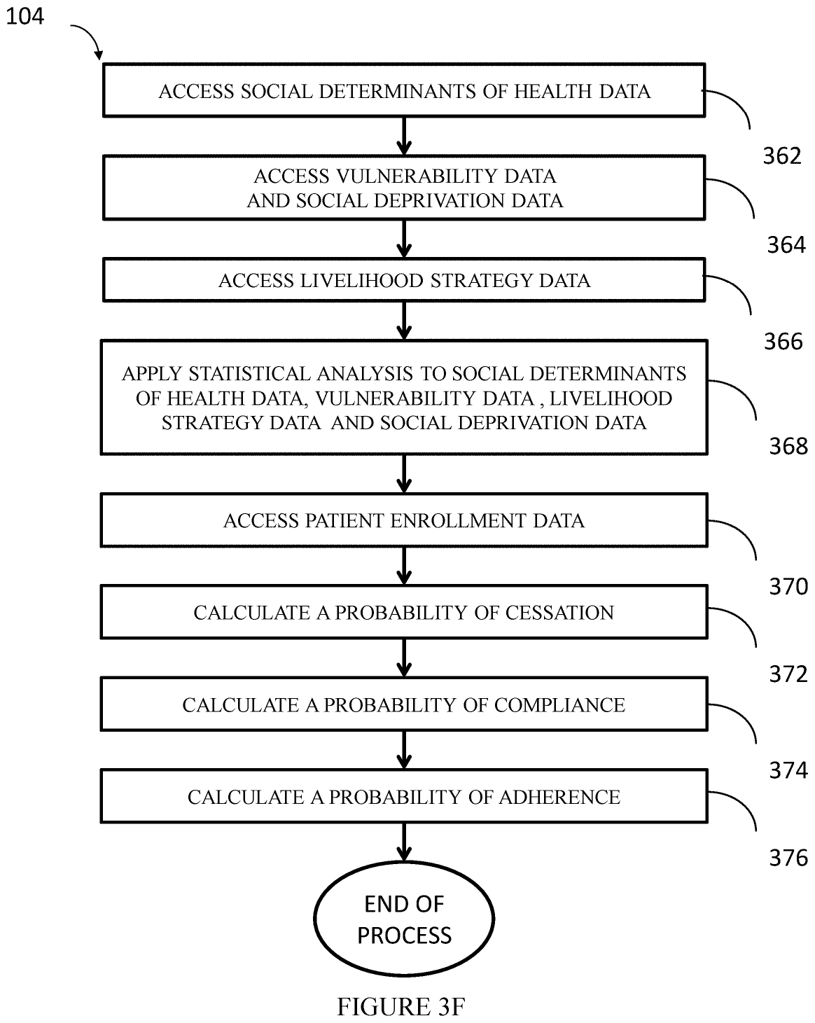

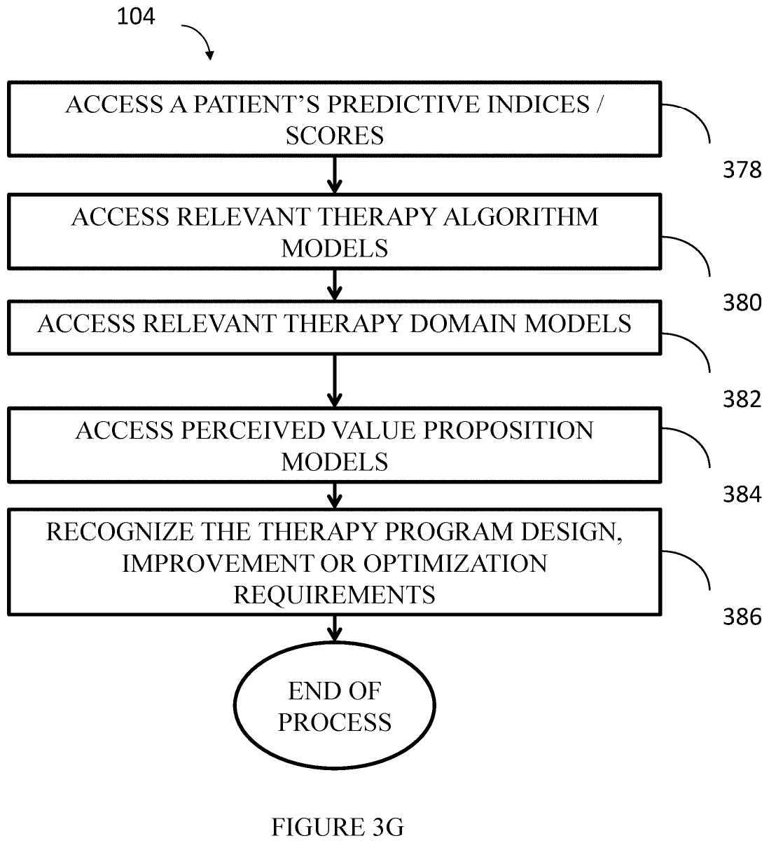

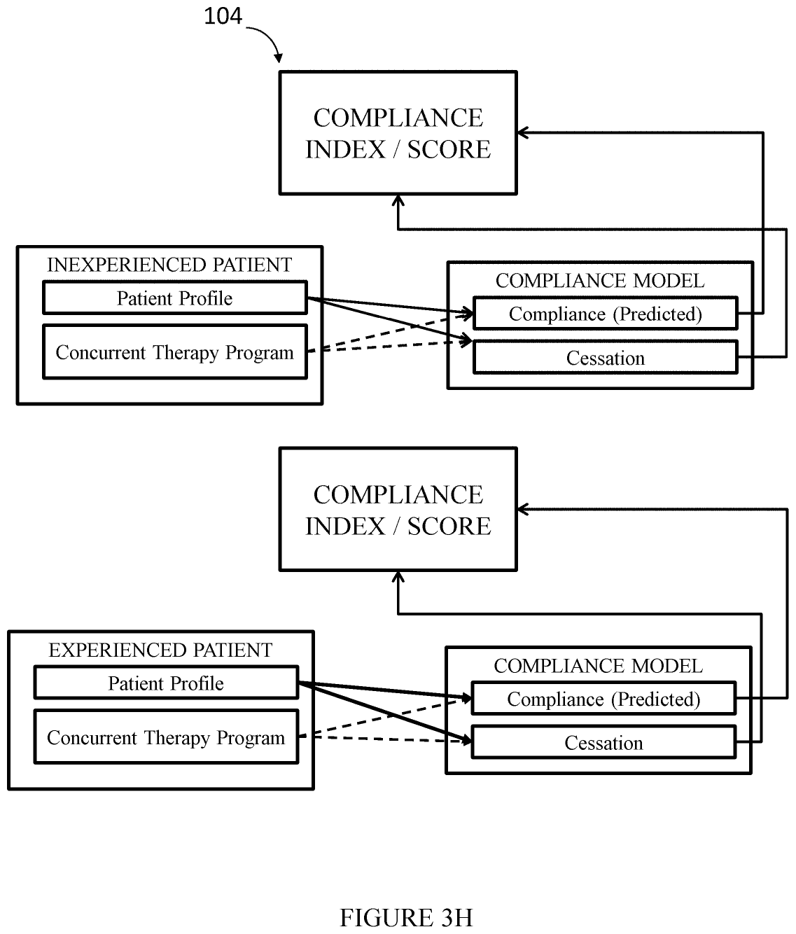

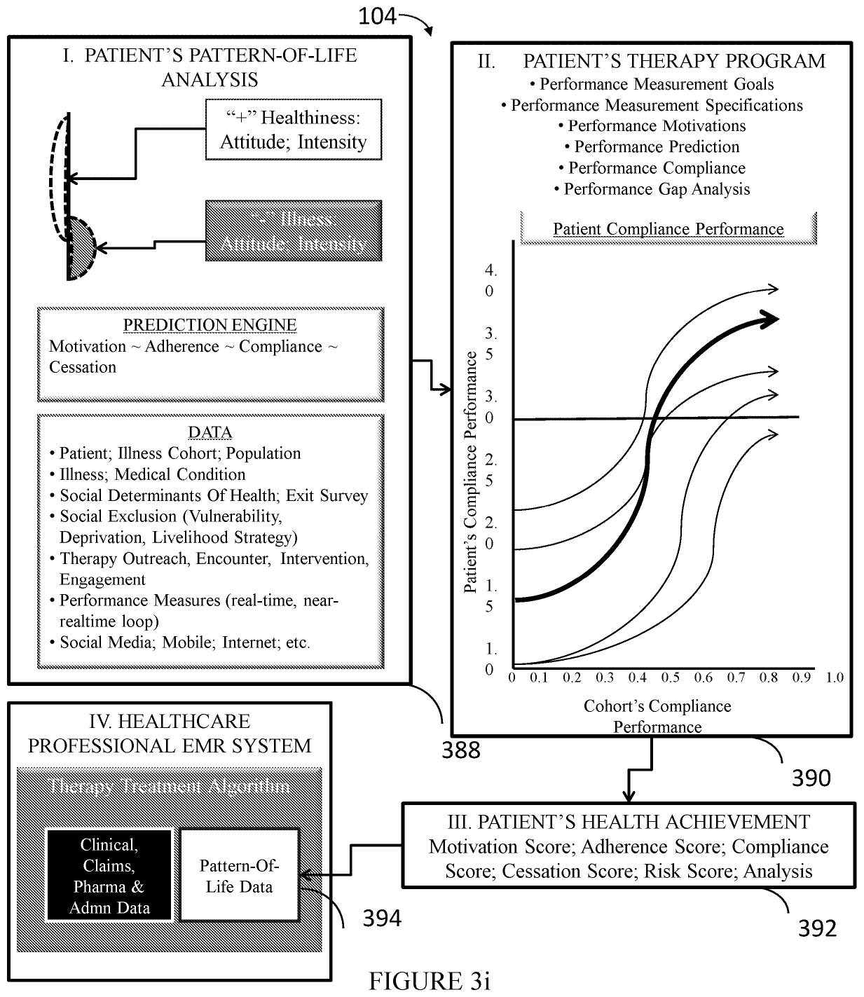

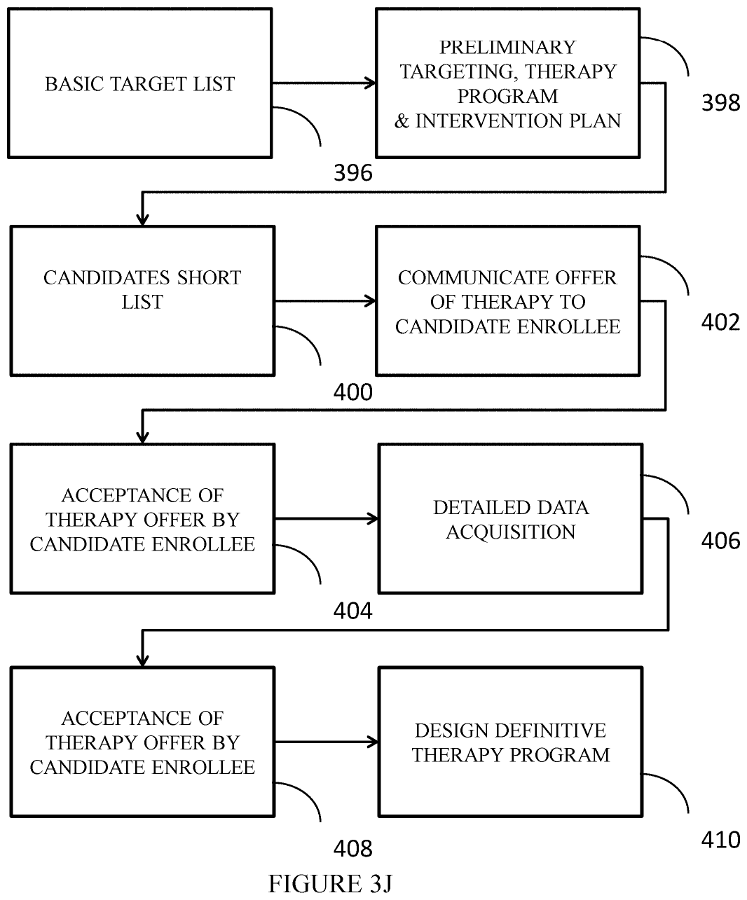

FIG. 3 shows a block diagram of the dynamic decisioning machine 102 subsystem of system 100, according to an example embodiment. This therapy decisioning unit may include predictive models, such as for example, motivation prediction module 308, adherence prediction module 310, compliance prediction module 312, and cessation prediction module 314. Therapy programs that may be more likely to be effective in improving therapy adherence and/or compliance of patients with a particular likelihood of therapy adherence and/or compliance may be identified based on a score and/or range of scores.

FIG. 3B illustrates a method 316-328 of predicting by recognizing the likelihood of the patient's cessation from the therapy program. This method accesses a patient's characteristics data at block 316, accesses Social Determinants Of Health correlated with the patient at block 318, applies statistical analysis to the patient data and the Social Determinants Of Health data at block 318, recognizes the relationships among predictive characteristics and therapy cessation at block 324, accesses the patient's enrollment data at block 326, and recognizes a likelihood of the patient's cessation from the therapy program at block 328. Enrollment data is access to recognize that the patient has a healthcare cost reimbursement plan and to perform the billing code linking and healthcare service reporting requirements. In some embodiments, the method 316-328 may be performed by the cessation prediction module 314 (see FIG. 3A) or by a cessation prediction tool.

FIG. 3C illustrates a method 330-340 of predicting by recognizing the likelihood of the patient's compliance with the therapy program. The method accesses the patient's characteristics data at block 330, accesses the Social Determinants Of Health data at block 332 correlated with the patient, applies statistical analysis to the patient's data and the Social Determinants Of Health data at block 334, recognizes relationships among predictive characteristics and therapy compliance 336, accesses patient enrollment data at block 338, and recognizes a likelihood of the patient's compliance with the therapy program at block 340. Enrollment data is access to recognize that the patient has a healthcare cost reimbursement plan and to perform the billing code linking and healthcare service reporting requirements. In some embodiments, the method 330-340 may be performed by the compliance prediction module 312 (see FIG. 3A) or by a cessation prediction tool.

FIG. 3D illustrates a method 342-346 of predicting by recognizing the likelihood of the patient's adherence to the therapy program. The method accesses the likelihood of therapy cessation for the patient at block 342, accesses the likelihood of therapy compliance for the patient at block 344 and recognizes a likelihood of the patient's adherence to the therapy program at block 346. In some embodiments, the method 342-346 may be performed by the cessation prediction module 310 (see FIG. 3A) or by an adherence prediction tool.

FIG. 3E illustrates the method 348-360 of applying the predicted likelihood of motivation, cessation, adherence, and compliance to the design or improvement of a therapy program. The method identifies a disease state of the patient at block 348, determines whether the patient is new to therapy at block 350, calculates a probability of therapy cessation at block 352, calculates a probability therapy compliance at block 354, calculates a probability of adherence at block 356, recognizes the results of such calculations to design or improve a therapy program at block 358, and records the implementation of the designed or improved therapy program at block 360. In some embodiments, the method 348-360 may be performed by the dynamic prediction machine 102 subsystem (see FIG. 3A) or by a therapy design, improvement and/or optimization tool.

FIG. 3F illustrates the method 362-376 of recognizing the predicted likelihood of the patient's cessation, compliance, and adherence correlated with the social exclusion of the patient. The method accesses Social Determinants Of Health data at block 362 correlated with the patient, vulnerability data and social deprivation data at block 364 correlated with the patient, livelihood strategy data at block 366 correlated with the patient, applies statistical analysis to Social Determinants Of Health data, vulnerability data, social depreciation data, and livelihood strategy data at block 368, accesses patient enrollment data at block 370, calculates a probability of cessation at block 372, calculates a probability of compliance at block 374, and calculates a probability of adherence at block 376. Enrollment data is access to recognize that the patient has a healthcare cost reimbursement plan and to perform the billing code linking and healthcare service reporting requirements. In some embodiments, the method 362-376 may be performed by the dynamic prediction machine 102 subsystem (see FIG. 3A) or by a therapy design, improvement, and/or optimization tool.

FIG. 3G illustrates the method 378-386 of applying the predictive indices to the design and/or the improvement of the therapy program. The method accesses the patient's predictive indices or scores 378, accesses relevant therapy algorithm models 380 (such as self-management interventions, peer group interventions, and social exclusion interventions), accesses relevant therapy domain models 382 (such as bricks-and-mortar intervention domains and virtual intervention domains), accesses perceived value proposition models 384 (such as the patient's perceived value [monetary or non-monetary] to the patient of therapy models, bricks-and-mortar intervention domains, virtual intervention domains, and recognitions, incentives, rewards, advantages, and benefits), and correlates such indices or scores, algorithm models, domain models, and perceived value proposition models with the design and/or the improvement or optimization of the therapy program 386. In some embodiments, the method 378-386 may be performed by the dynamic prediction machine 102 subsystem (see FIG. 3A) or by a therapy design, improvement, and/or optimization tool.

With regard to the design, improvement or optimization of therapy programs pursuant to the predictive methods performed by dynamic decisioning machine subsystem 102 of system 100, through method 348-360 (see FIG. 3E) and/or method 378-386 (see FIG. 3G), these methods may support the healthcare professional in the identification and correlation of a therapy compliance and/or adherence program for the patient, based on the patient's likelihood of therapy compliance or adherence. The therapy program design, improvement, or optimization may be a no-program or no-intervention decision where, for example, no program has been identified as likely to increase therapy compliance or adherence or if the patient is identified as so likely to be adherent that an intervention is unnecessary. The method 348-360 and/or the method 378-386 also may be used to identify those patients and correlated therapy programs that may be most likely to improve therapy compliance and/or adherence, and thereby, enable healthcare resources to be targeted where they are relatively more likely to have a significant impact on therapy compliance or adherence.

Dynamic decisioning machine 102 subsystem of system 100 may include dynamic feedback scoring machine module 304, which may compile and correlate motivation scores, adherence scores, compliance scores, and cessation scores, which may calculate and store patient predictive models, illness or medical condition predictive models, population predictive models, and other predictive models. Such scores and predictive models may include beginning-stages, adjustments to therapy programs reflecting feedback from patient performance of therapy programs, and then-current stages.

Dynamic decisioning machine 102 subsystem of system 100 may include dynamic therapy optimization machine module 306 may include information on the patient's baseline condition, such as for example, whether the patient is new to therapy, the feedback on the patient's performance of the therapy program, and the optimizations of the therapy program, such as for example, frequency, dosage, time, record keeping, goal-progress, incentives, therapy domain relevancy and changes, decision-making plan, family interaction plan, and livelihood strategy changes.

FIG. 3H shows a block diagram of a predictive cessation model, according to an example embodiment. The model illustrates at a high level the predictive processes for a patient new-to-therapy program and an experienced patient currently participating in a therapy program. As discussed herein, the adherence or compliance prediction model can be separated into two components, a new enrollee component and an experienced or continuous enrollee component. Each of the components may perform a statistical analysis of the demographic data and then-current therapy data. Further, each component may include a compliance module and a cessation module, as well as an adherence module and an adherence cessation module. In either case, a predicted compliance or adherence can be calculated based on the patient profile. The predicted compliance and/or adherence can be factored in to refine the compliance module or the adherence module. Once current patient profile data has been collected and actual past patient profile data can be factored in with the current patient profile data and the then-current therapy in the case of the experienced enrollee component. As more and more data on the patient's performance of the therapy program is collected, the most recent patient profile data can continue to refine the model. Further, as actual data is collected, a future likelihood of compliance and adherence can be predicted and potentially utilized to factor in with the compliance module and the adherence module.

Accordingly, there can be 18 underlying predictive models—(1) the new enrollee compliance model, (2) the new enrollee predicted compliance model, (3) the new enrollee refined predicted compliance model, (4) the new enrollee adherence model, (5) the new enrollee predicted adherence model, (6) the new enrollee refined predicted adherence model, (7) the new enrollee cessation model, (8) the new enrollee predicted cessation model, (9) the new enrollee refined predicted cessation model, (10) the experienced enrollee compliance model, (11) the experienced enrollee predicted compliance model, (12) the experienced enrollee refined predicted compliance model, (13) the experienced enrollee adherence model, (14) the experienced enrollee predicted adherence model, (15) the experienced enrollee refined predicted adherence model, (16) the experienced enrollee cessation model, (17) the experienced enrollee predicted cessation model, and (18) the experienced enrollee refined predicted cessation model.

These underlying models can be further refined based on type of illness, type of medical condition and/or type of medication therapy, together with the correlated Social Determinants Of Health data, which may correlate to a particular set of characteristics for why people may or may not be compliant or adherent. For example, behavior data may reflect what appears to be a totally obscure factor that appears to have nothing to do with the illness state, but shows up as a statistically significant event and is repeatable and can be validated. The inventive subject matter can also factor in this statistically significant event. In addition, the validation of the model and the statistically significant event is a continuous refinement. The model can capture patterns of behavior that may be related to illness type, or medical condition type, or comorbidities, or medication therapy, or other factor and can extract things that are common. If a pattern is identified as statistically significant, then such factor may be one of the factors used for the prediction of future compliance.

FIG. 3i shows a block diagram of method 388-394, according to an example embodiment, of dynamic decisioning machine 104 subsystem of system 100, by which of the inventive subject matter may be operationalized by being processed from the patient's pattern-of-life analysis 388, and may be further processed to the patient's therapy program 390, and may be further processed to the patient's health achievement 392, and may be further processed through communication to the healthcare professional of the patient's pattern-of-life data 394 representing the predicted insights based on the Social Determinants Of Health correlated with the patient. Patient's pattern-of-life analysis 388 may collect data from a variety of sources and on a variety of data points relevant to the patient's Social Determinants Of Health and correlated, according to the inventive subject matter, with the patient's illness and medical condition. A prediction engine component of patient's pattern-of-life analysis 388 may recognize, according to the inventive subject matter, the patient's predicted motivation, adherence, compliance and cessation. A report may be generated indicating a measure of the patient's compliance performance 390 of the therapy program. This compliance performance measure may begin at start or base line of the patient's health status at the commencement or preliminary analysis of the patient's risk for adherence, and may include the then-current status of performance.

Compliance performance measures may include the patient's then-current motivation score, adherence score, compliance score, cessation score, illness or medical condition risk score and a comparative analysis. The scope of the report may include a comparison of the patient's status with the status of other patients in the same illness or medical condition cohort as that of the patient. The patient's health achievement 392 may be reported to the patient through patient communication device 110 and to the healthcare professional through healthcare professional communication device 108. The report to the healthcare professional also may be integrated into the healthcare professional electronic medical records system 394 and utilized in the design, improvement and/or the optimization of the therapy program.

FIG. 3J shows a block diagram of method 396-410, according to an example embodiment, of dynamic decisioning machine 104 subsystem of system 100, by which the inventive subject matter may be operationalized for patient targeting and outreach. Basic target list 396 may identify candidate patients through the enrollment pool in health benefit plans and/or medical interviews. Preliminary targeting, therapy program and intervention plan 398 may apply the predictive methods of the inventive subject matter to the identified candidate enrollees' data and recognizes a preliminary analysis of the motivation, adherence, compliance, and cessation risk of each candidate enrollee, and a preliminary therapy program may be designed for each candidate. Candidates short list 400 may be generated from the preliminary predictive analysis of candidate enrollees, based on a threshold predictive score indicating each enrollee's likelihood of motivation, adherence, compliance, and/or cessation. An offer of therapy may be made by the healthcare professional to each short-listed enrollee through communicate offer of therapy to candidate enrollee 402. An acceptance of therapy offer by candidate enrollee 404 may be communicated to the healthcare professional. During one or more medical interviews with the accepting enrollees, detailed data acquisition 406 may be collected by the healthcare professional, including data on the Social Determinants Of Health correlated with the enrollee. Based on such data, therapy strategies and implementation approaches 408 are designed by the healthcare professional, discussed with the candidate enrollee, and decided upon with mutual agreement. After agreement, the design definitive therapy program 410 may be prescribed or recommended by the healthcare professional.

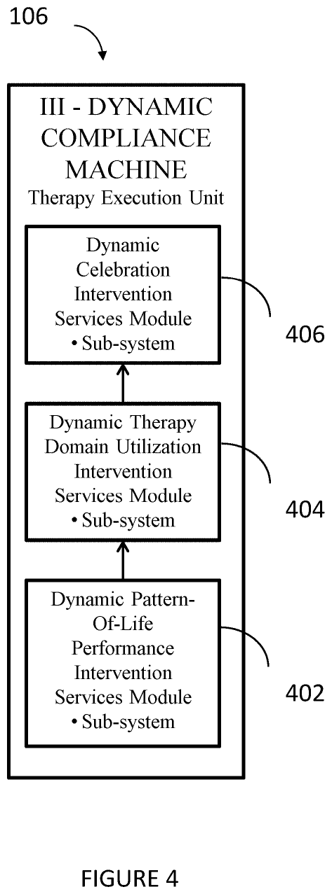

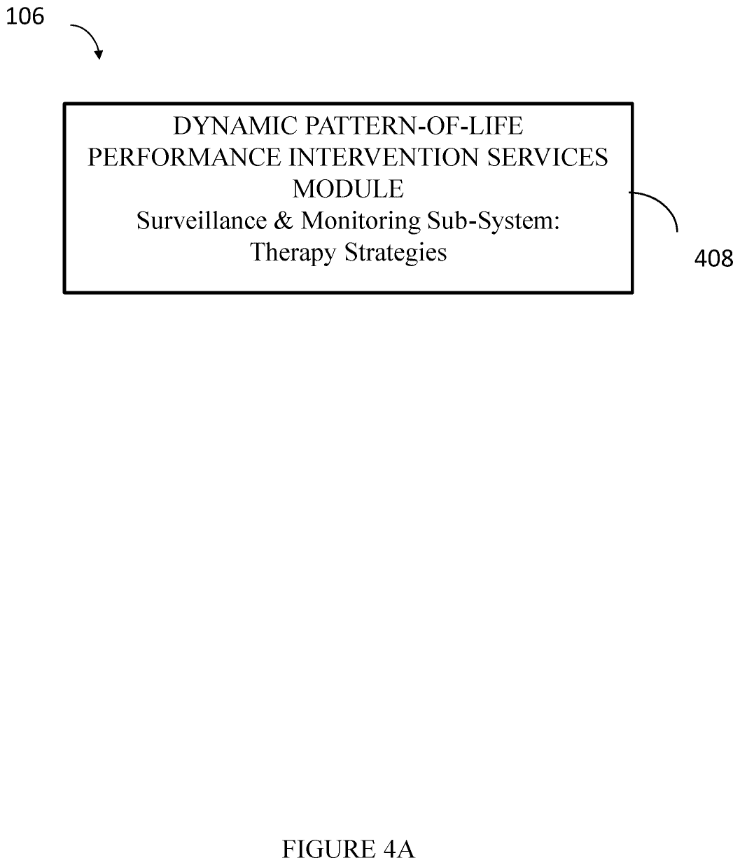

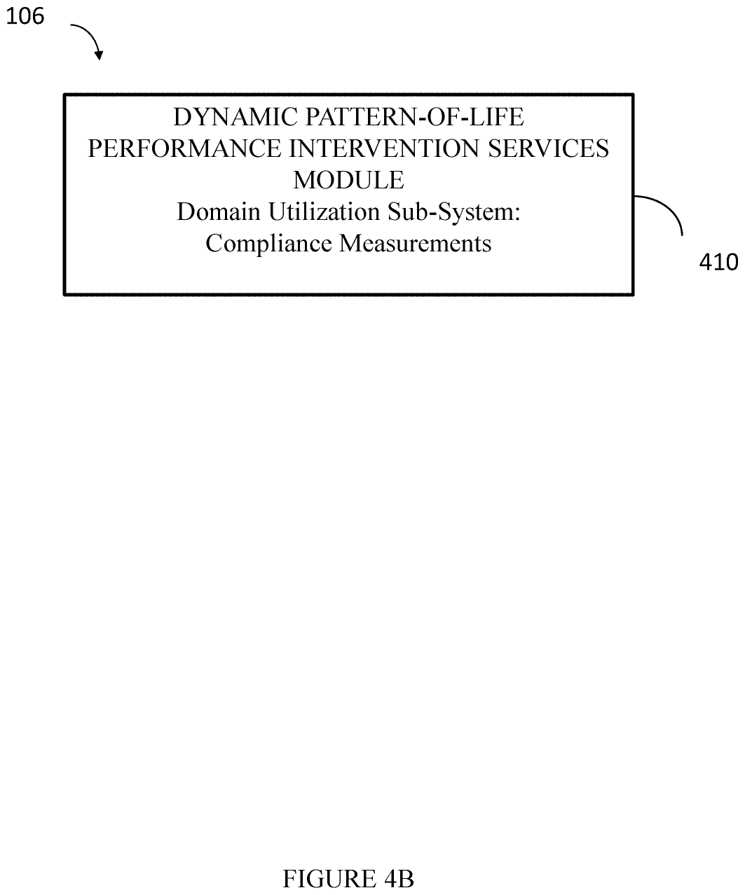

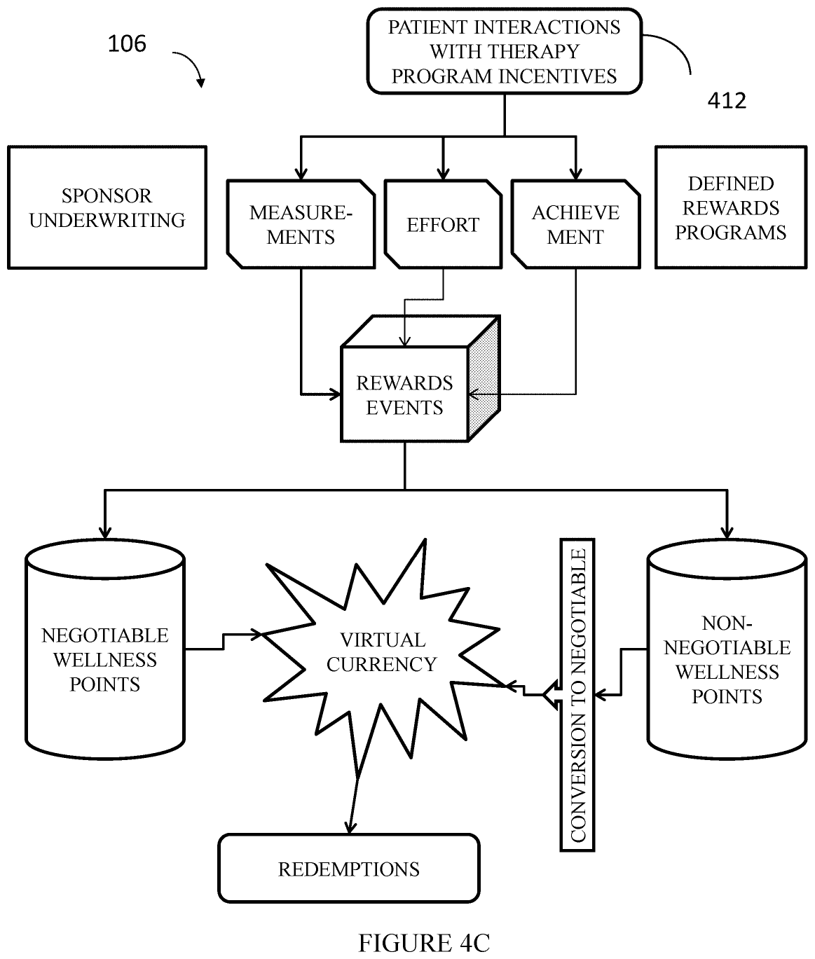

FIG. 4 shows a block diagram of the dynamic compliance machine 106 subsystem of system 100, according to an example embodiment. The therapy execution unit may include dynamic pattern-of-life performance intervention services module 402, according to an example embodiment, dynamic therapy domain utilization intervention services module 406, according to an example embodiment, and dynamic celebration intervention services module 410, according to an example embodiment.

FIG. 4A shows a block diagram of a therapy strategies performance surveillance and monitoring 404 module, according to an example embodiment, of dynamic pattern-of-life performance intervention services module 402 of subsystem 106 of system 100. Therapy performances by the patient surveilled and monitored may include the then-current status (as distinguished from a recent status) of therapy aims and goals, and patient performance requirements of the therapy program, such as for example, prescribed or recommended education, prescribed or recommended self-management, scheduled follow-ups, the social exclusion strategy, the livelihood strategy, the healthcare decision-making plan and the family interaction plan. Therapy strategies surveillance and monitoring also may include therapy reinforcements, such as for example, immediate interventions (such as realtime or near real-time), current interventions (such as daily or weekly), and/or less frequent interventions such as monthly. Immediate therapy reinforcement may utilize interventions, such as for example, sound, text, light and/or vibration when data is collected from the patient. Current therapy reinforcement may utilize interventions, such as for example, reports of activities by type and date, reports of team/group activities by type and date, reports of behavior invested by the patient, reports of patient investment required to achieve an incentive, and reports of health game advancement. Less frequent therapy reinforcement may utilize interventions, such as for example, feedback/reinforcement by a phone counselor or doctor, triggering of an encouragement phone message, a deposit to Health Savings Account or Flex Account, a reduction of a healthcare premium, a valuable incentive or reward if goal is attained, and notice of the award of a valuable incentive or reward if a therapy program goal is attained. FIG. 4B shows a block diagram of a compliance measurement of therapy domain utilization 408 module, according to an example embodiment, of dynamic therapy domain utilization intervention services module 406 of subsystem 106 of system 100. Compliance measurement of therapy domain utilization may include status (baseline, current [including updated data], and gap), frequency, duration of use, intensity of use, and medication usage with alerts, reminders, dosages, and re-fills. Domains may include bricks-and-mortar venues and virtual venues.

FIG. 4C shows a block diagram of method 412, according to an example embodiment, of dynamic compliance machine 106 subsystem of system 100, by which the inventive subject matter is operationalized for patient intervention and engagement through dynamic celebration intervention services module 406. According to an example embodiment, the patient's processes of interacting with and performing the therapy program may include for example: an incentives program correlated with the performance of the therapy program; the incentives program may enable the patient to earn health credits for performance of the therapy program; the degree of performance of the therapy program may be acknowledged through a notice to the patient that the patient has earned a recognition, incentive, reward, advantage, or benefit (collectively a “motivation instrument”) for compliance with the therapy program; acknowledgement to the patient may be accompanied with a health credit to the patient's therapy program account; the health credit may represent a value earned by the patient through a commercial transaction with a health-affinity domain or for a value unearned by the patient through a non-commercial transaction with a health-affinity domain; the health credit may be integrated with a financial sponsor; the health credit may represent a tangible or an intangible value correlated with bricks-and-mortar and/or virtual health-affinity domains or other domains such as a merchant; earned value in excess of a designated amount may be transferred and allocated to unearned value in the patient's account; the health credit may have a negotiable value or a non-negotiable value; a non-negotiable value may be converted to a negotiable value; the patient my redeem a negotiable motivation instrument at a participating health-affinity domain, such as for example, a primary care medical home, a health insurance carrier, or a merchant; and the therapy program incentives management service may be operated by the healthcare professional or a third party.

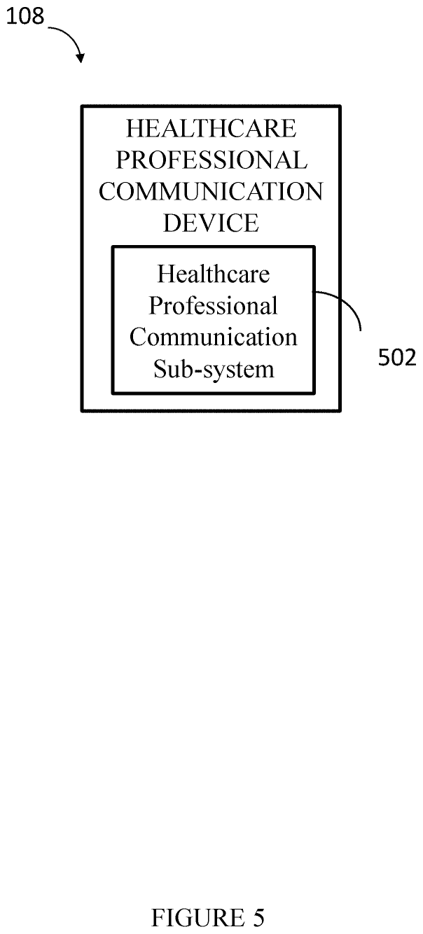

FIG. 5 shows a block diagram of healthcare professional communication device 108, according to an embodiment, of system 100. Communications with patients before and/or after identified therapy programs have been implemented may be facilitated at the healthcare professional communication device 108. For example, a healthcare professional may use the healthcare professional communication device 108 to facilitate communications with a patient to gather data points for patient data 330 stored in a database 202 that may be used to determine the patient's likelihood of therapy compliance 340. After a therapy program has been identified for a particular patient, the healthcare professional communication device 108 may be used to implement or aid in implementing the therapy program. Examples of healthcare professionals who may operate the healthcare professional communication device 108 include a nurse, physician, physician's assistant, pharmacist, and other health care providers and/or personnel trained to administer and/or implement a therapy adherence program. Communications through the healthcare professional communication device 108 may generate voice communications to a patient, may be used for automated phone counseling, to prescribe or recommend a change in a therapy procedure, to reschedule a follow-up appointment, to enroll and/or offer home delivery of medication to a patient, and the like.

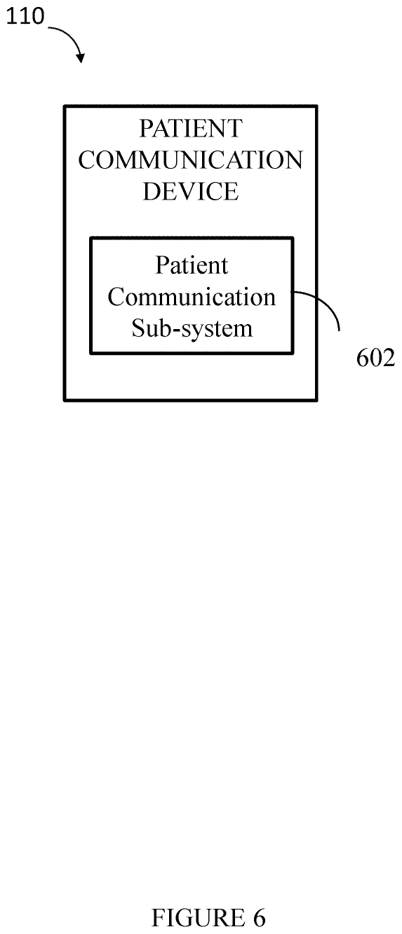

FIG. 6 shows a block diagram of patient communication device 110, according to an embodiment, of system 100. Patient communication device 110 may be computing or personal assistant devices for receiving messages and other communication to improve adherence and/or compliance. Special applications may be utilized on computing or personal assistant devices for management of therapy programs. Patient communications may include, for example, medical interview questionnaires, surveys, and therapy program compliance reports. Special applications for management of therapy programs may include, for example, electronic time clocks, mobile phones and other mobile devices, wearable RFID tags, and biometric sensor devises, weight scales, and pedometers equipped with RFID capabilities for transmitting data to a mobile phone that can forward the data to the healthcare professional.

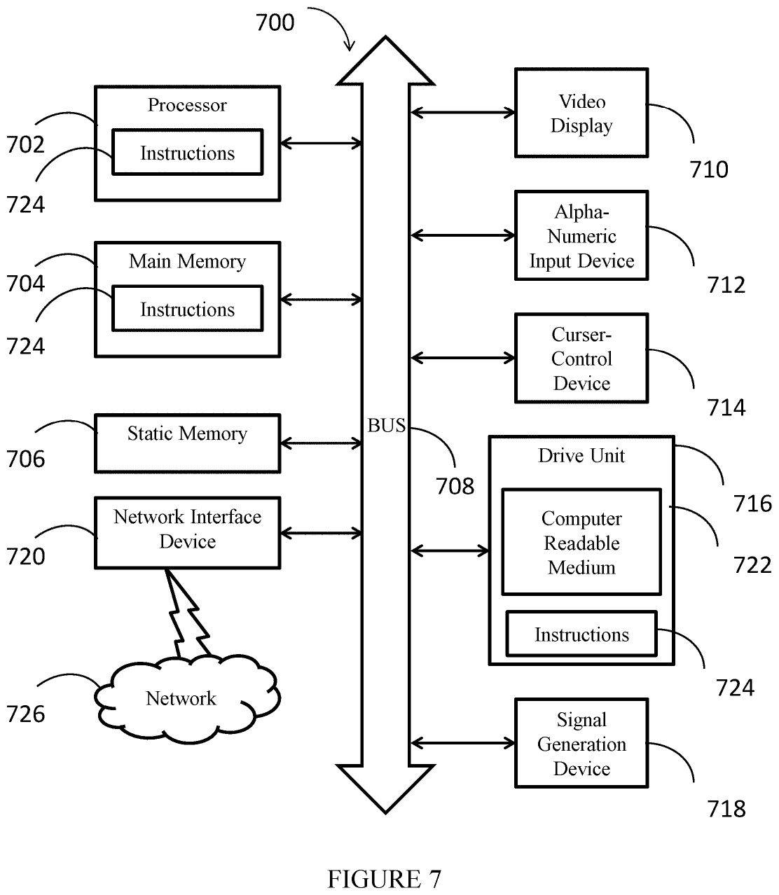

FIG. 7 shows a block diagram of a machine in the example form of a computer system 700 within which a set of instructions may be executed causing the machine to perform any one or more of the methods, processes, operations, or methodologies discussed herein. The infinite-reduction precision-rewrite lab 102, dynamic decisioning machine 102, dynamic compliance machine 106, healthcare professional communication device 108, and/or the patient communication device 110 may include the functionality of the one or more computer systems 700.

In an example embodiment, the machine operates as a standalone device or may be connected (e.g., networked) to other machines In a networked deployment, the machine may operate in the capacity of a server or a client machine in server-client network environment, or as a peer machine in a peer-to-peer (or distributed) network environment. The machine may be a server computer, a client computer, a personal computer (PC), a cloud computer, a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 700 includes a processor 702 (e.g., a central processing unit (CPU) a graphics processing unit (GPU) or both), a main memory 704 and a static memory 706, which communicate with each other via a bus 708. The computer system 700 may further include a video display unit 710 (such as for example, a liquid crystal display (LCD) or a cathode ray tube (CRT)). The computer system 700 also includes an alphanumeric input device 712 (such as for example, a keyboard), a cursor control device 714 (such as for example, a mouse), a drive unit 716, a signal generation device 718 (such as for example, a speaker) and a network interface device 720.

The drive unit 716 includes a computer-readable medium 722 on which is stored one or more sets of instructions (that is, software 724) embodying any one or more of the methodologies or functions described herein. The software 724 may also reside, completely or at least partially, within the main memory 704 and/or within the processor 702 during execution of the software by the computer system 700, the main memory 704 and the processor 702 also constituting computer-readable media.

The software 724 may be further transmitted or received over a network 726 via the network interface device 720.

While the computer-readable medium 722 is shown in an example embodiment to be a single medium, the term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing or encoding a set of instructions for execution by the machine and that cause the machine to perform any one or more of the methodologies of the inventive subject matter. The term “computer-readable medium” shall accordingly be taken to include, but not be limited to, solid-state memories, and optical media, and magnetic media.

The network 112 may include a Mobile Communications (GSM) network, a code division multiple access (CDMA) network, 3^{rd} Generation Partnership Project (3GPP), an Internet Protocol (IP) network, a Wireless Application Protocol (WAP) network, a WiFi network, or an IEEE 802.11 standards network, as well as various combinations thereof. Other conventional and/or later developed wired and wireless networks also may be used.

Overview of mSDOH Diabetes Treatment Method

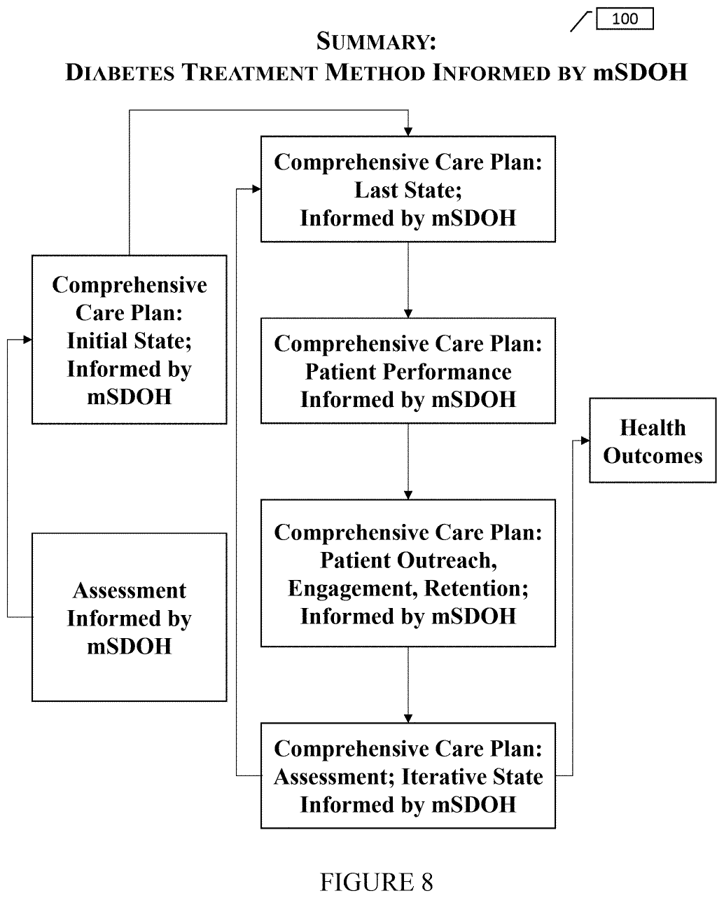

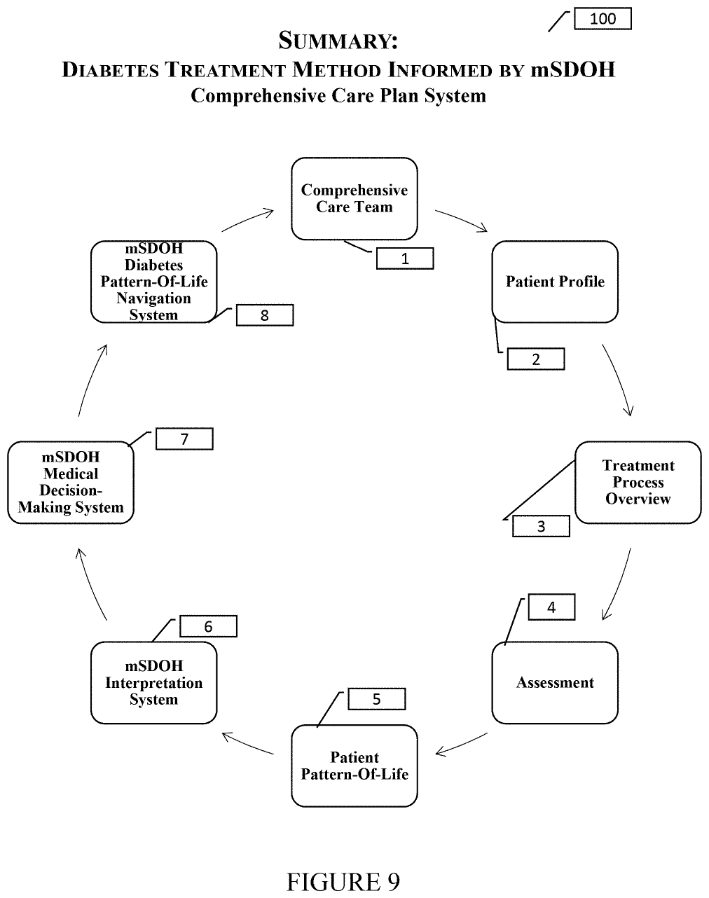

FIG. 8 presents the general treatment method and process of the invention. They assess the patient, develop a Comprehensive Care Plan based on the assessment, surveils and monitors the patient's compliance with the directives of the Comprehensive Care Plan, updates such plan over time with the changes, if any, in the patient's condition, objectives of the Comprehensive Care Plan and its directives and evaluates and reports such information. More particularly, the invention's treatment method operationalizes the system depicted in FIG. 9 comprised of (1) a comprehensive care team, (2) the introduction of a novel patient profile based on novel mSDOH and changes or alterations thereto, (3) the novel patient treatment process informed by the novel mSDOH and changes or alterations thereto, (4) assessments (baseline and follow-ups), (5) a novel profile of the patient's pattern-of-life evidencing the mSDOH relevant to the patient and changes or alterations thereto, (6) a novel mSDOH translation system, (7) a novel mSDOH-informed medical decision-making system and (8) a novel mSDOH diabetes pattern-of-life navigation system.

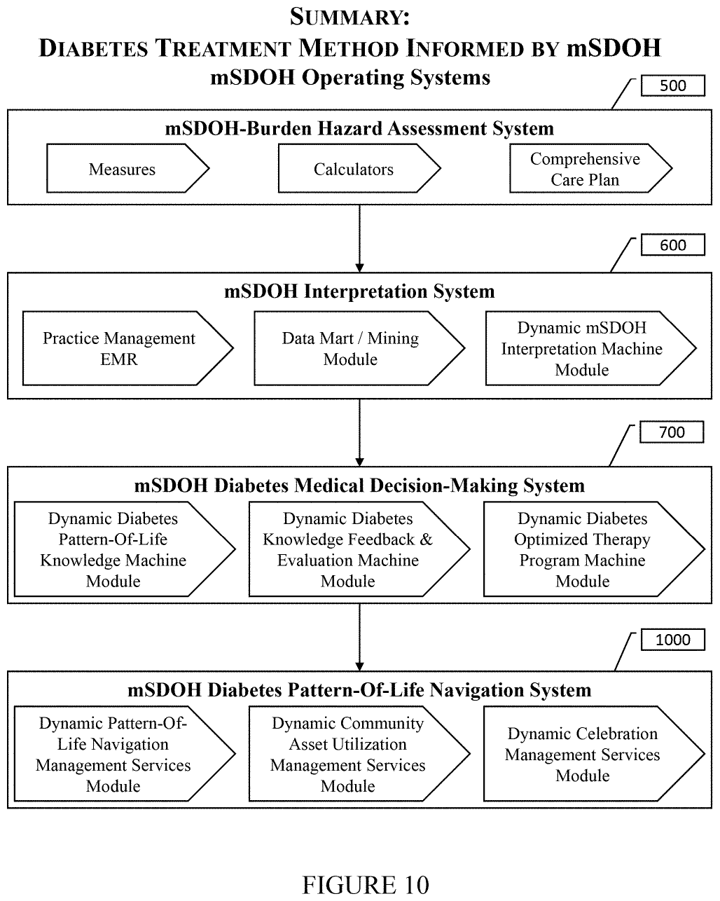

The invention's operating organization at FIG. 10 introduces a novel mSDOH Hazard Assessment System, a novel Dynamic mSDOH Translation System, a novel mSDOH Diabetes Medical Decision-Making System and a novel mSDOH Diabetes Pattern-Of-Life Navigation System. The mSDOH Hazard Assessment System module at FIG. 10 (500) is a diagnostic instrument informed of the impact of health disparities and mSDOH comprised of measures, dynamic calculators and guidance and reports directed to the development, updating and management of comprehensive care plans. The DOH Translation System at FIG. 10 (600) is comprised of three modules: the healthcare provider's practice management/electronic medical records (EMR) system, a data mining module and a dynamic mSDOH Translation Machine module. This module translates unstructured mSDOH data into structured data utilized by the EMR system and informs the EMR system and the healthcare provider of the insights from such translated data. The mSDOH Diabetes Medical Decision-Making System module at FIG. 10 (700) informs the healthcare team of the impact of mSDOH on the patient and further informs the team in their operationalization of the insights from mSDOH as the team develops, updates and manages the comprehensive care plan. The mSDOH Diabetes Pattern-Of-Life Navigation System module at FIG. 10 (1000) outreaches to and engages with the patient during the course of the patient's daily living routine, informing the healthcare team of patient-reported Signs, Symptoms and performance and compliance with care plan directives and analyzing and reporting pattern-of-life measures and outcomes to the team for evaluation, utilizing remote, interactive, patient-reported-outcomes instruments and multiplex point of care tests (xPOCT).

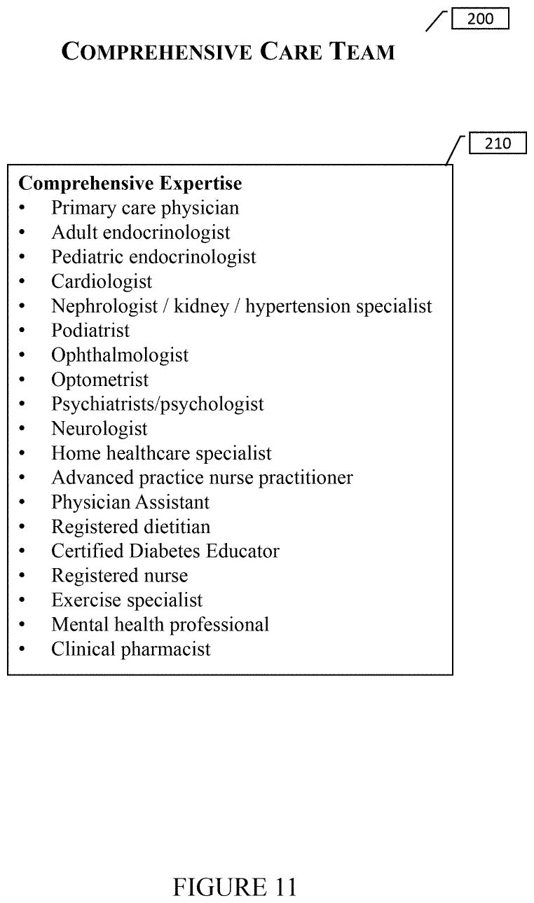

The invention utilizes a team-based comprehensive care approach to the treatment of diabetes. Team-based care recognizes and responds to the complexities of diabetes, including its coexisting chronic diseases, its complications and the impact on diabetes diagnosis and treatment of metabolic syndrome. FIG. 11 illustrates the required core healthcare team skill sets (210) commonly required to deliver comprehensive care to the diabetic patient.

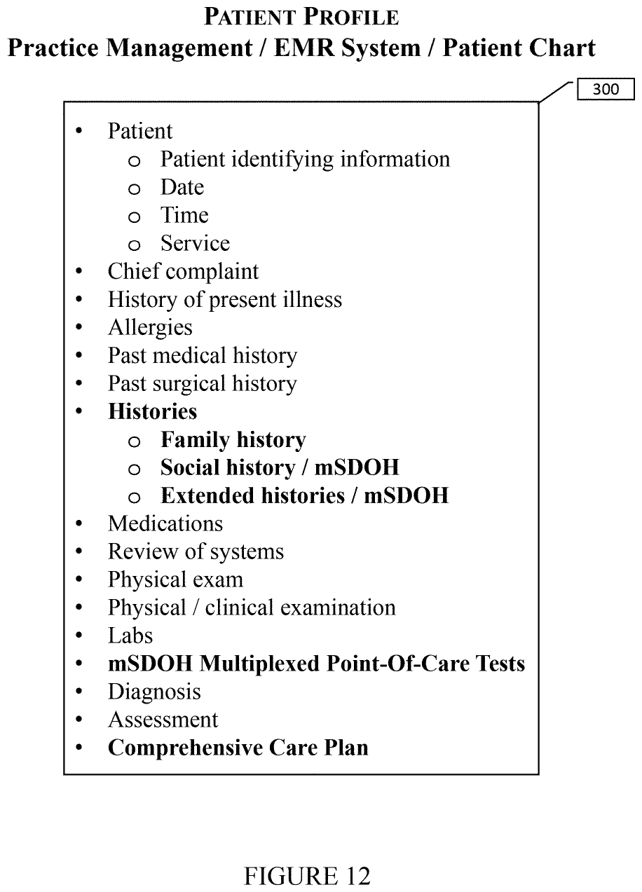

The invention informs the EMR system of the patient's mSDOH information, reporting to the patient chart managed by the EMR system. The reporting introduces at the common Care Plan section of the patient chart a novel mSDOH information category or class at the common Histories section and a novel mSDOH xPOCT section, as illustrated in FIG. 12.

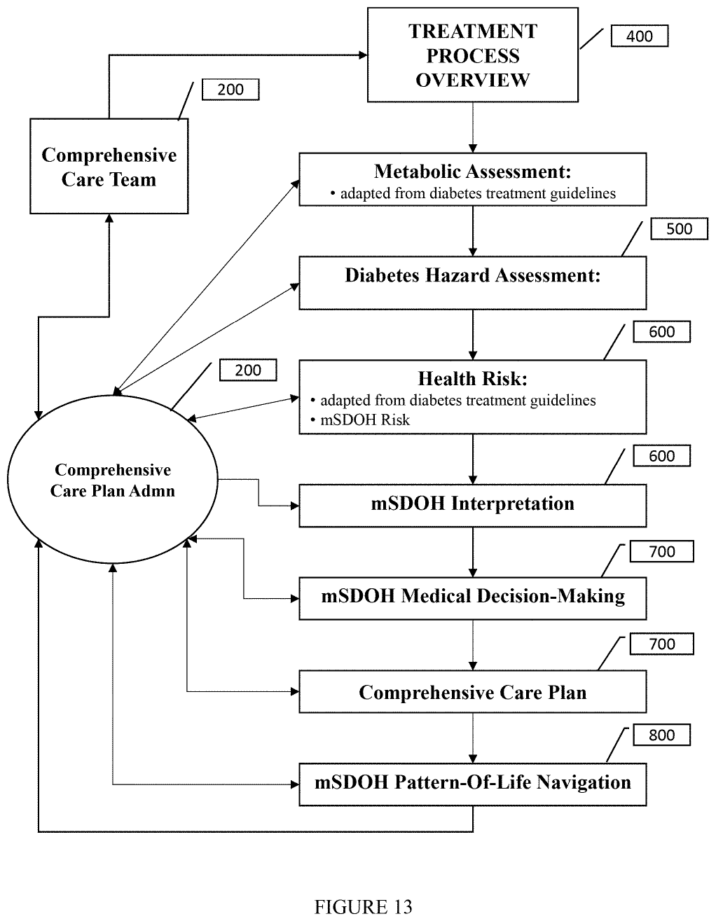

The administration of the treatment method and process of the invention is presented at FIG. 13. Along with the common metabolic assessment, FIG. 13 introduces the invention's overall treatment method with its novel diabetes hazard assessment (500), novel mSDOH-informed health risk analysis (600), novel mSDOH insights translation (600), novel medical decision-making (700) informed by changes or alterations to the patient's mSDOH, novel comprehensive care plan development, updating and management (700) similarly informed and novel mSDOH pattern-of-life navigation (800). Such processes are administered by the comprehensive care team at FIG. 11.

mSDOH-Burden Hazard Calculator

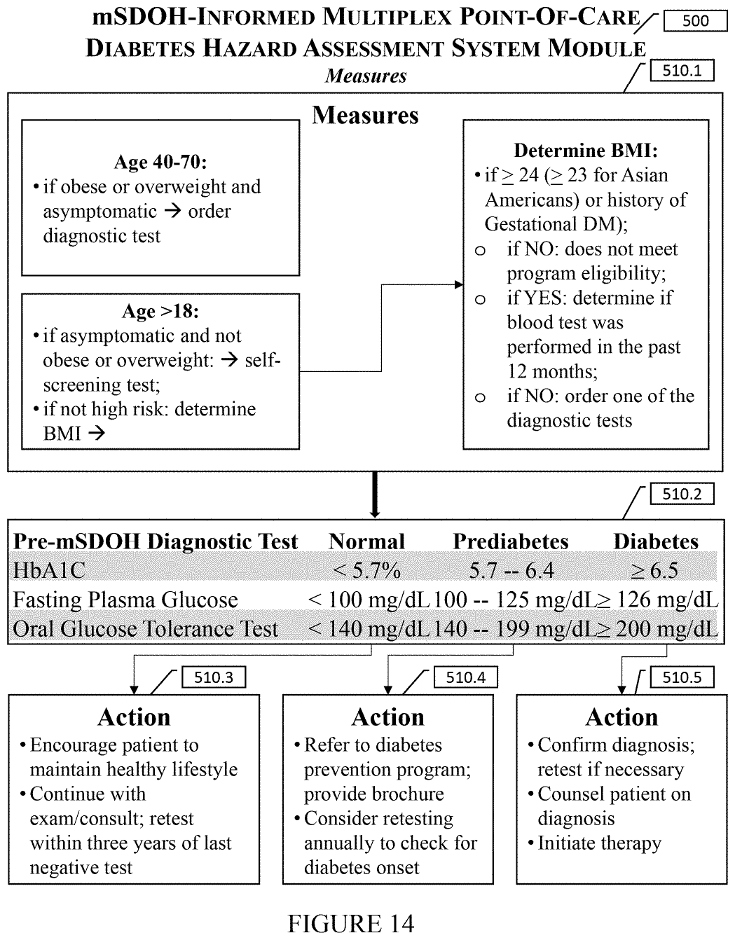

Commonly, cut point ratios support the assessment of the state or status of diabetes. A preferred embodiment introduces a novel method of improving the effectiveness of cut point ratios by increasing or decreasing the cut point by a ratio that is informed by mSDOH. In addition, a preferred embodiment introduces a novel method of predicting or improving the stage or status of diabetes by measuring a hazard ratio that is informed by mSDOH. FIG. 14 introduces the novel measures utilized by the novel diabetes hazard assessment system and its accounting for healthcare disparities and other mSDOH. In contrast, the common measures are age and BMI (510.1); the common determination of the disease status—normal, prediabetes, diabetes—uses common metabolic testing methods and their common cut points (510.2); the common medical decision-making produces an action plan based on whether the tests indicate the patient's status of normal (510.3), prediabetes (510.4) or diabetes (510.5); and the common action plans are those commonly recommended by the guidelines of the diabetes professional associations and government authorities.

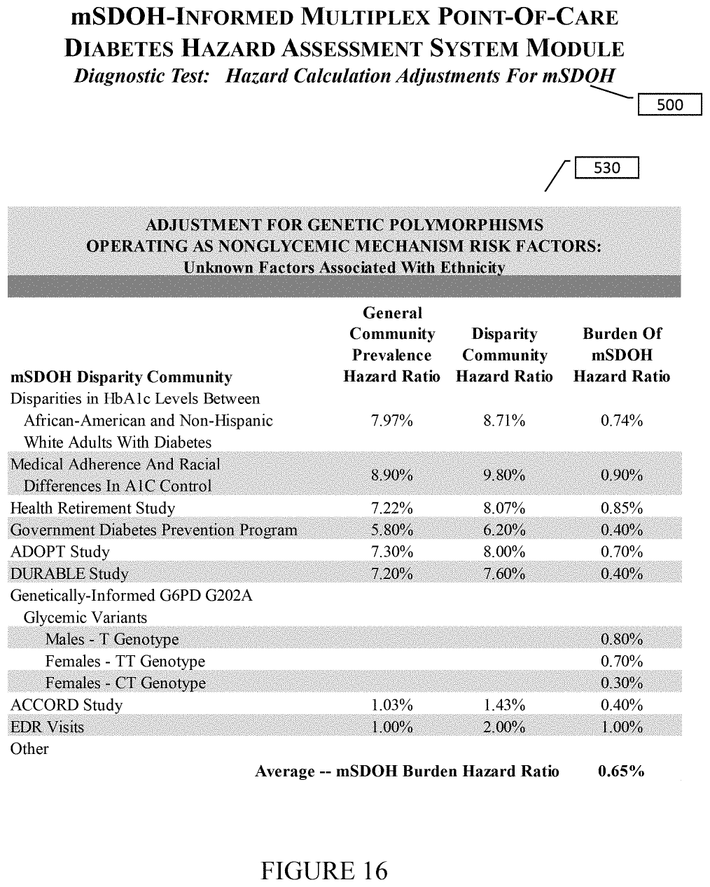

In addition to such common measures, the invention introduces novel new or improved cut points to the common cut points (520.1) used in such tests, by the use of a mSDOH hazard calculator. The calculator applies previously-unknown risk factors associated with race/ethnicity, by adjusting the cut points for genetic polymorphisms operating as nonglycemic mechanism risk factors through a process presented at FIG. 16 (530). The invention performs a novel process of recognizing and applying hazard ratios attributable to diabetes and other general disease prevalence in communities of health disparity, accounting for prevalence in the general community and accounting for prevalence in the disparity community. The difference between the hazard ratio of diabetes prevalence in the general community and the hazard ratio of diabetes prevalence in the disparity community is a novel indicator of the increased disease burden in the disparity community. This increased burden is recognized by the invention as the mSDOH-burden hazard ratio. The higher the ratio, the greater the hazard or risk of diabetes.

In a preferred embodiment, the hazard ratios of disparity communities introduce a novel data type identified and based upon the collective of community health disparities. In an embodiment, the hazard ratios of disparity communities are identified and analyzed by a government analysis. In yet another embodiment, the hazard ratios of disparity communities are identified and analyzed by a disease-specific analysis. Yet another embodiment may identify and analyze the hazard ratios of disparity communities in clinical operations or quality improvement analyses, while yet in still another embodiment hazard ratios may be based on other health-related or socioeconomic disparity analyses. Government analyses include for example the national Health Retirement Study and the Government Diabetes Prevention Program, as well as State and local analyses such as the neighborhoods represented in the Chicago Health Atlas. Disease-specific analyses include for example Disparities in HbA1C Levels Between African-American and Non-Hispanic White Adults with Diabetes. Clinical operations or quality improvement analyses include for example the number of physician or ER visits or preventable hospital stays of populations from disparity communities. The highest-value hazard ratio is that reflecting the most intimate relationship with or impact on the patient, such as a defined neighborhood or city- or county-wide disparity analysis.

In an embodiment of the invention, there is introduced novel indicators for such hazard ratio by weighting to maximize the correlation of the hazard ratio with quality improvement metrics established by governments and private organizations, such as quality of life, premature death rates and preventable hospital stays.

In other embodiment of the invention, the mSDOH hazard ratio introduces a novel articulation of multiple socioeconomic indicators (ranging for example from poverty to education, from access to poor outcomes and from retail expenditures to healthcare outcomes) or may combine multiple socioeconomic indicators into a single composite value. For example, ratios can range from zero to one or from one to 100, in each case the latter representing the highest socioeconomic need. A novel comparison of the disparity community to the general community is made by the mSDOH-burden hazard ratio. The higher the socioeconomic need, the higher the mSDOH-burden hazard ratio, the higher the diabetes hazard or risk and the more-rich is the novel mSDOH information for diabetes risk analysis, medical decision-making, care plan development, updating and management and patient outreach, engagement and care plan retention.

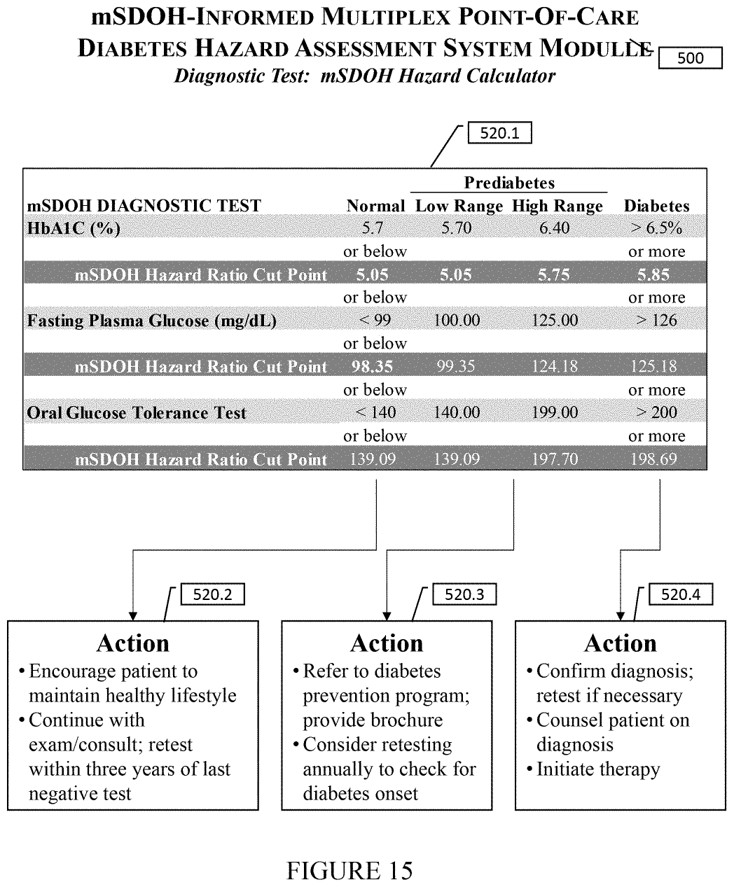

Having established a mSDOH-burden hazard ratio, the invention introduces a novel application of the hazard ratio by a novel application to the cut points for the common diabetes diagnostic tests. Using such ratio as a novel predictor or as a novel measure of disease state, status or intensity, the invention applies the mSDOH hazard ratio to reduce the range of blood glucose level scores utilized by the common tests. FIG. 15 (520.1) measures the reduction in the cut points for the common tests. For the normal status: in the case of the HbA1C test, the cut point is reduced from 5.7 to 5.05; in the case of the fasting plasma glucose test, the cut point reduction is from <99 to 98.35; and in the oral glucose tolerance test, the cut point is reduced from <140 to 139.09. For the prediabetes status: in the case of the HbA1C test, the cut point for the low range is reduced from 5.70 to 5.05, and from the high range the reduction is from 6.40 to 5.75; in the case of the fasting plasma glucose test, the cut point reduction for the low range is from 100.00 to 99.35, and from the high range the reduction is from 125.00 to 124.18; and in the case of the oral glucose tolerance test, the reduction is from 140.00 to 139.09 in the low range and from 199.00 to 197.70 in the high range. For the diabetes status: in the case of the HbA1C test, the cut point is reduced from 6.5 to 5.85; in the case of the fasting plasma glucose test, the cut point reduction is from >126 to 125.18; and in the oral glucose tolerance test, the cut point is reduced from >200 to 198.69.

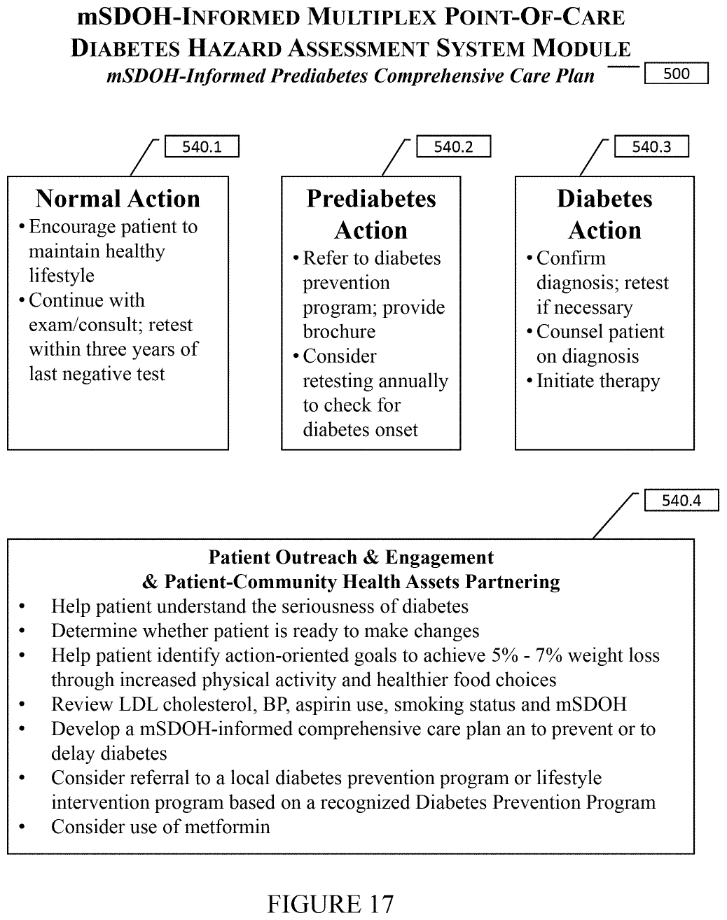

Based on the reduced cut points, FIG. 17 recites the common medical decision-making that then follows the actions for the normal patient (520.2), for the prediabetic patient (520.3) and for the diabetic patient (520.4). For the normal patient, the medical decision-making is to encourage the patient to maintain healthy lifestyle, continue with scheduled examinations and consultations and retest within three years of the last negative test. For the prediabetic patient, the medical decision-making is to refer to a diabetes prevention program, provide instructional information on diabetes and consider retesting annually to check for diabetes onset. For the diabetic patient, the medical decision-making is to confirm the diagnosis, retest if necessary, counsel patient on diagnosis and initiate therapy.

The effects of the invention's novel earlier cut point reductions are that they: inform an earlier prevention of diabetes, its comorbidities and their complications; inform a longer delay in the onset of diabetes its comorbidities and their complications; and inform an earlier treatment of diabetes. As a result, the diagnosis of diabetes is improved; the prevention of diabetes is improved; the treatment of diabetes, its comorbidities and their complexities is improved; diabetes treatment costs are reduced; and patients' HRQoL is improved.

FIG. 17 illustrates that in each case—the normal patient (540.1), the prediabetic patient (540.2) and the diabetic patient (540.3)—the invention's action plan is operationalized through a comprehensive care plan (540.4). The directives of such care plan include patient outreach and engagement and support in the performance of care plan directives that include the patient's utilization of community health assets and their partnering organizations. Developing and implementing such directives include a variety common, as well as of mSDOH-informed, lifestyle changes. In an embodiment of the invention, common lifestyle changes include: helping the patient understand the seriousness of diabetes; determining whether the patient is ready to make lifestyle changes; helping the patient identify action-oriented goals to achieve 5%-7% weight loss through increased physical activity, diet and nutrition choices; reviewing LDL, cholesterol, blood pressure, aspirin use and smoking status; considering referral to a local diabetes prevention program or lifestyle intervention program based on a recognized Diabetes Prevention Program; and considering the use of medication interventions. In contrast, the invention introduces novel mSDOH-informed lifestyle changes, such as changing or altering mSDOH and developing a mSDOH-informed comprehensive care plan to prevent or to delay the onset of diabetes, which novelties are presented at FIGS. 6 through 10 and their several sub-Figures.

mSDOH Translation System

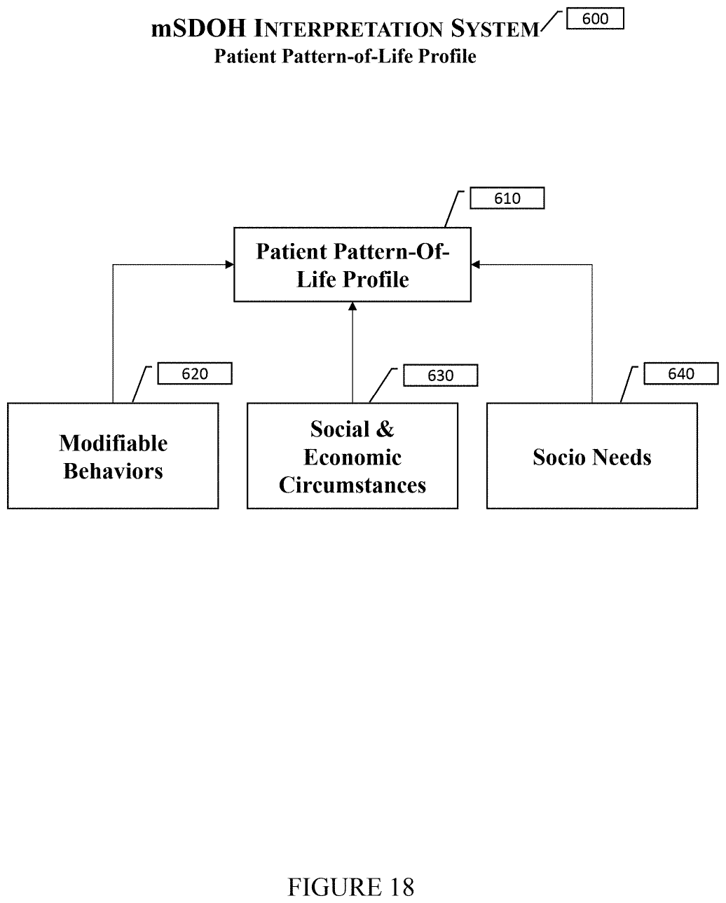

A component of the invention is a novel mSDOH Translation System summarily presented at FIG. 18. The overall function of this system is to convert or translate novel mSDOH data to a form of data that is acceptable to and processable by the EMR system. It operates with structured data manipulated in such a way as ultimately to conform to medical billing standards, as required by Federal statute. In contrast, mSDOH Translation System utilizes unstructured data attributable to human behavior and the management of human behavior, particularly data created by the patient while navigating the pattern-of-life. To do so, the invention introduces a novel patient pattern-of-life profile (610) and activates and manages the novel patient profile by changing or altering mSDOH and by operationalizing such changes or alterations through informing the healthcare team in its analysis of health risk, medical decision-making, the design, update and management of the patient's comprehensive care plan and patient outreach, engagement and retention in the care plan.

The invention introduces a diabetes treatment method based on a novel lifestyle modification, by changing or altering novel mSDOH to inform treatment methods and by a novel operationalization of the insights from such changes or alterations through navigation of the patient's pattern-of-life as a Proxy for lifestyle modification. Such novelties are a unique and advanced lifestyle modification therapies for the treatment of diabetes, as distinguished from the common diabetes lifestyle modification therapies of diet/nutrition, physical activity, education and counseling and medication management. The patient's novel pattern-of-life profile is comprised of modifiable behaviors (620), social and economic circumstances (630) and socio needs (640).

The invention introduces a novel health data category of modifiable behaviors and the further novelty of their being valuable behaviors (670) and harmful behaviors (689). Valuable behaviors are introduced as novel patient practices that the patient perceives as being helpful to the patient's self-image or to achieving the patient's goals. Harmful behaviors are introduced as novel patient practices that the patient perceives as being harmful to the patient's self-image or to achieving the patient's goals. Valuable/harmful behavior information is reported to the healthcare team by the patient through one or more of a common survey directly administered by a member of the team, a novel interactive, remote, patient-reported-outcomes instrument and a novel interactive, remote, patient-administered xPOCT.

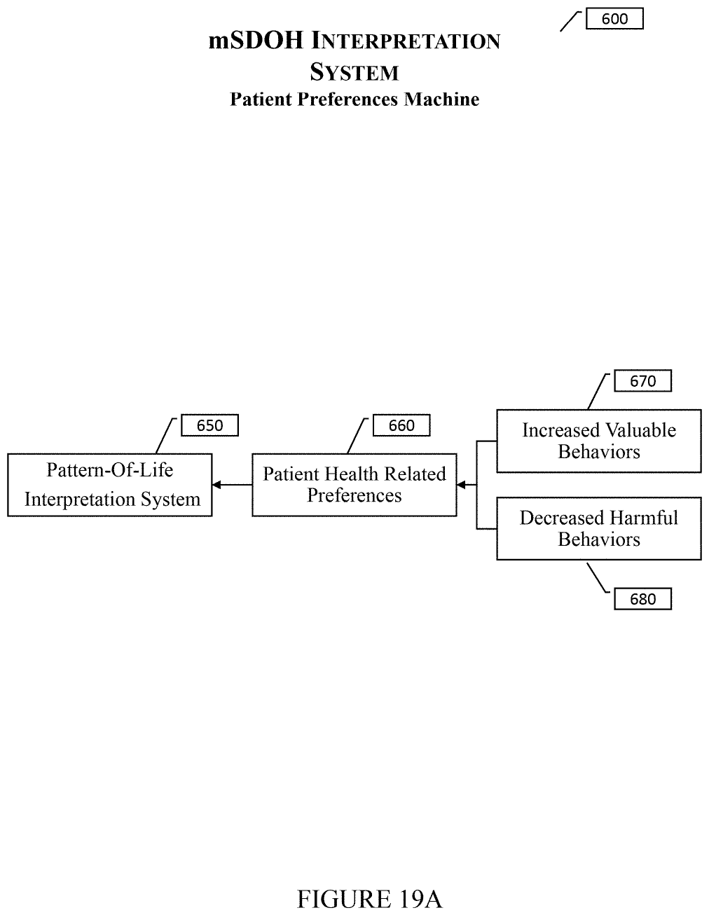

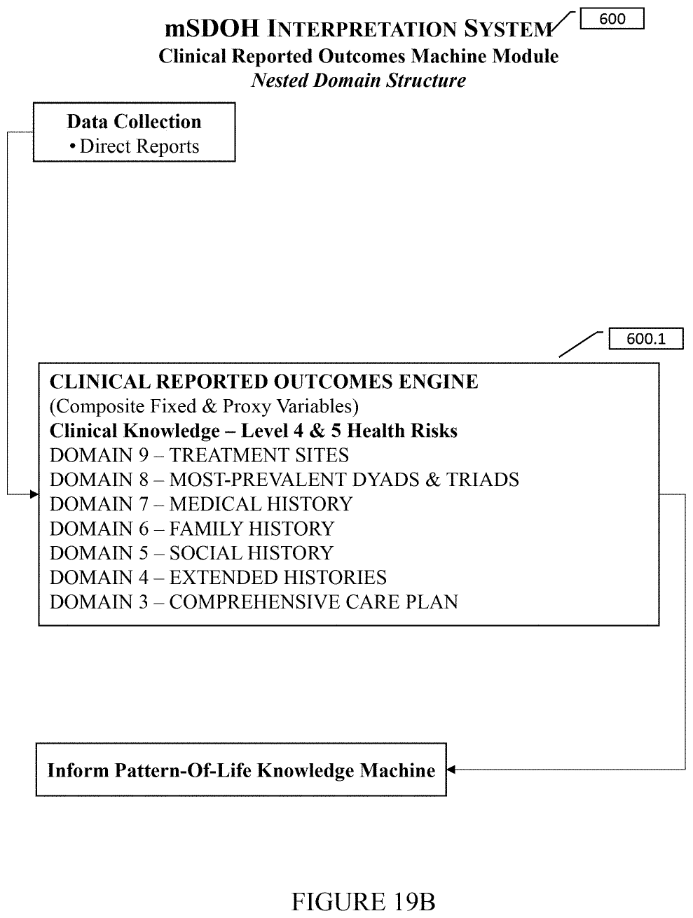

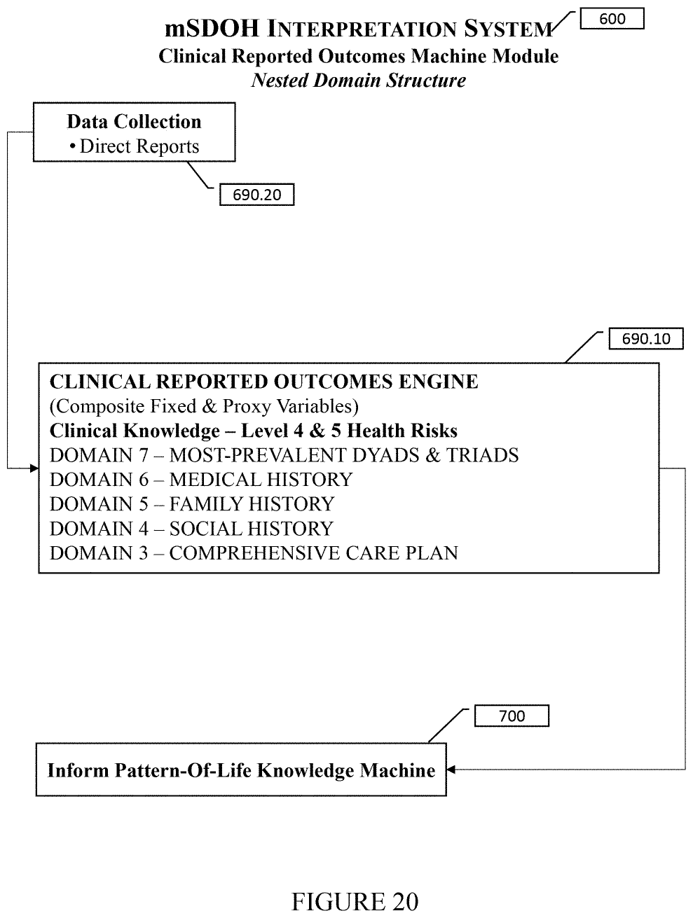

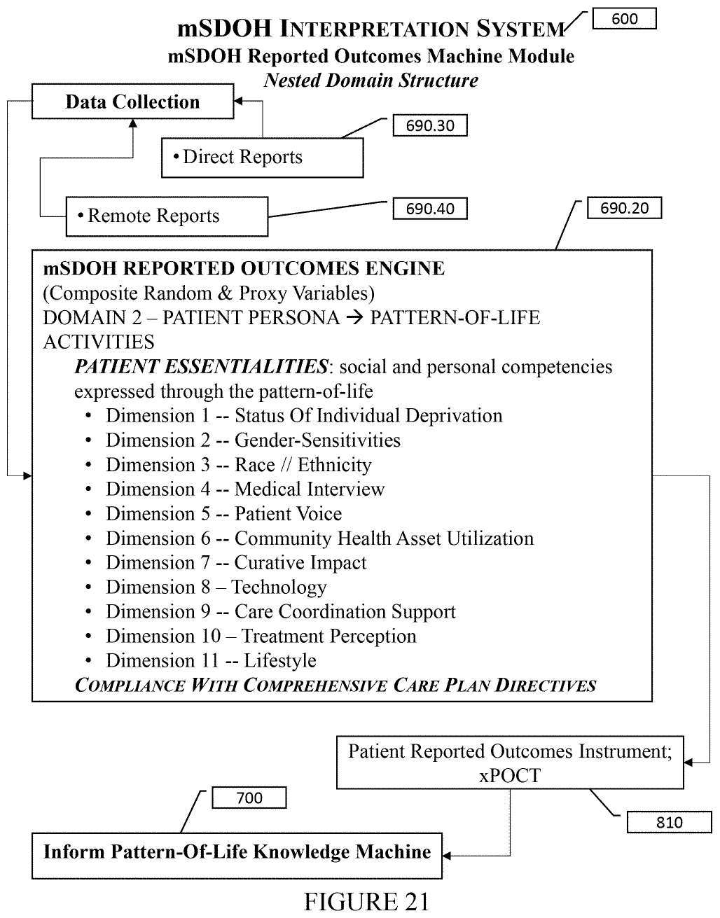

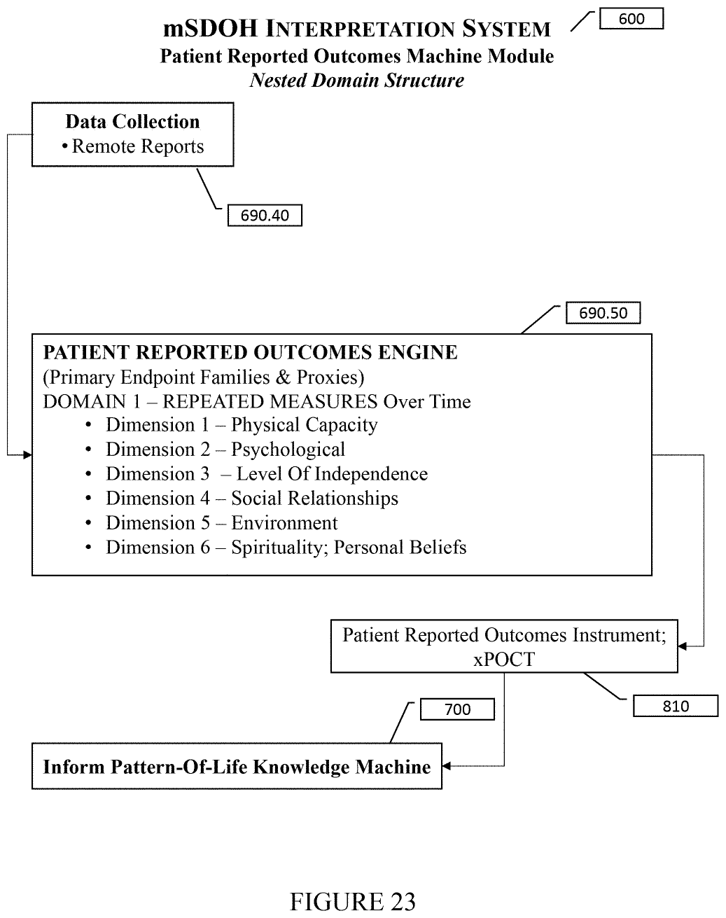

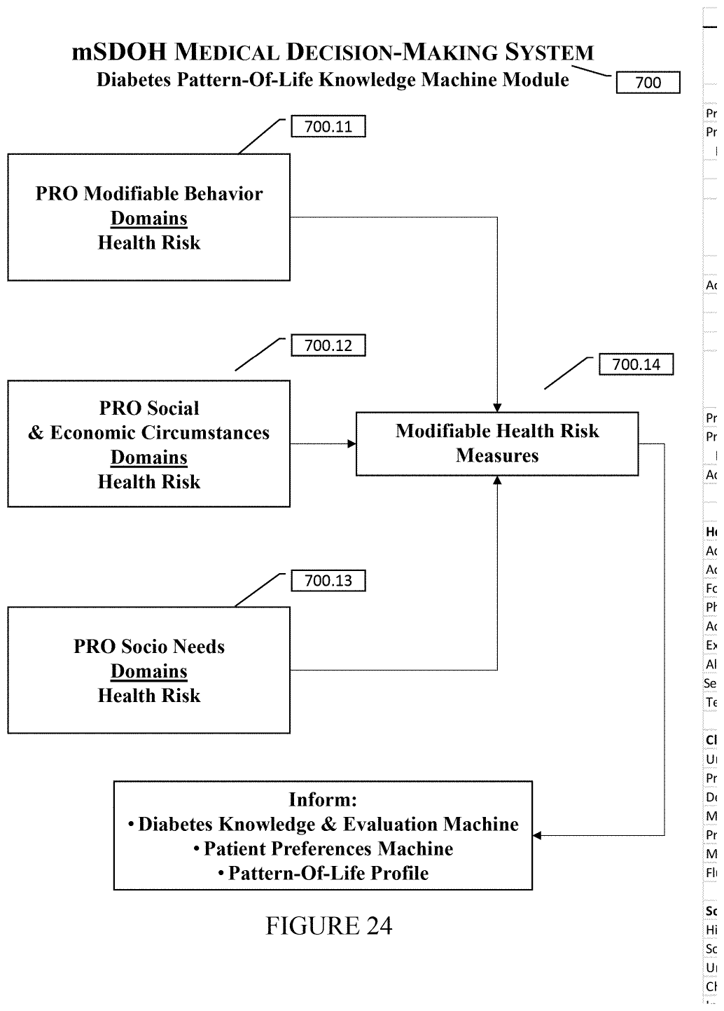

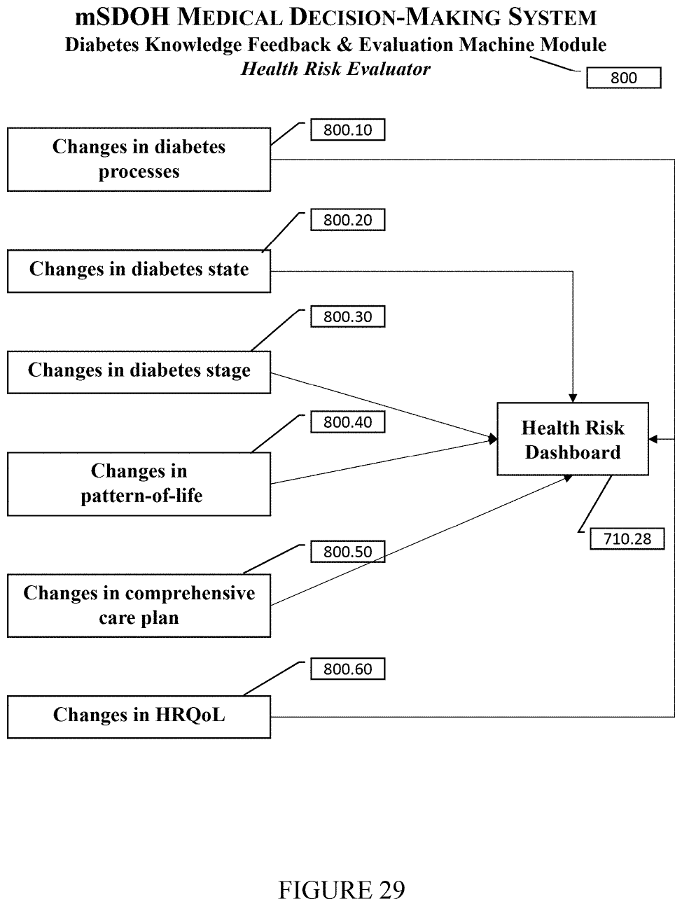

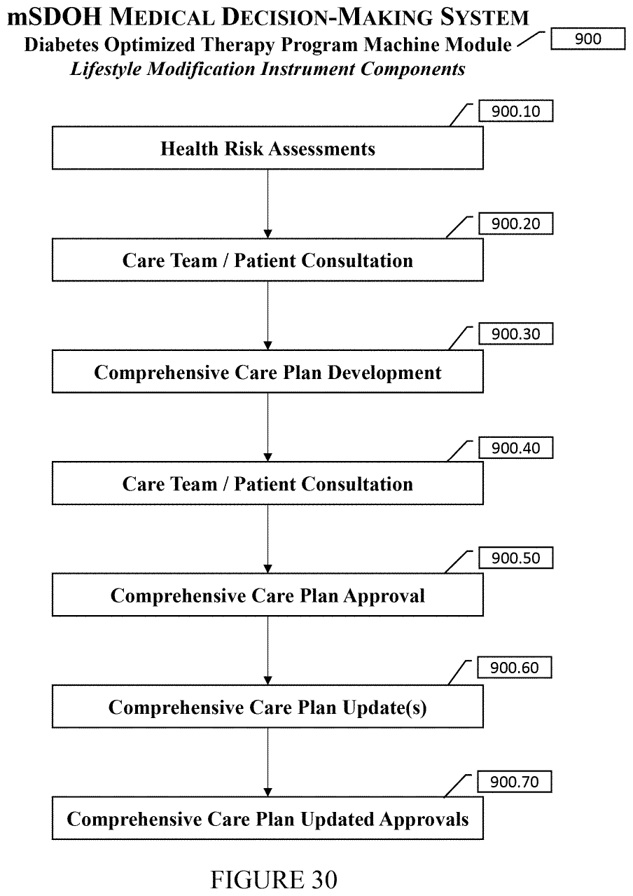

At FIG. 19A, patient-reported-outcomes are introduced as a novel correlation with the pattern-of-life of the patient. Pattern-of-life unstructured data variables are translated by the invention to structured variables (650). Such unstructured variables are comprised of novel preferred patient choices (660). Patient-choices of helpful or harmful behaviors are introduced as novel determinations by the invention of the patient's health-related preferences (660). To make the preferences determinations, the invention collects and analyzes information from the EMR system (FIG. 20), from novel patient pattern-of-life activities (FIG. 21) and from remote patient-reported outcomes (FIG. 23). Such information is evaluated to determine the impact on it by novel mSDOH measures and changes or alterations thereto (FIGS. 24-28B) and to apply such impact to inform the healthcare team in its medical decision-making (FIG. 29) and in its comprehensive care plan development, updating and compliance (FIG. 30 et seq.).

At FIG. 20 (690.10), clinically-reported EMR information includes the patient's chart reporting histories, the changes or alterations of novel mSDOH reported by the patient-reported-outcomes instrument or xPOCT and the care plan. The invention introduces a novel combination of such information, together with an evaluation of whether the patient's diabetes coexists with the most-prevalent coexisting chronic condition dyads and triads. These reports are weighted and the composite weight then is adjusted to account for the mSDOH-burden hazard ratio applicable to the patient and a composite score informs (700) the health risk calculator in the pattern-of-life knowledge machine (FIGS. 24-28B) as composite fixed and Proxy variables (700.14; 700.40; and 700.50) and diabetes treatment endpoint families (700.60).

Over and above the common repeated metabolic and related testing and treatment methods, the novel mSDOH repeated treatment measures correlate with a novel analysis of patient lifestyle through the patient's persona and pattern-of-life. A preferred embodiment of the invention introduces at FIGS. 14-21 a method for treating diabetes through lifestyle modification based on a novel unified Concept of the class or category of mSDOH by applying patient persona and patient Essentialities, as Proxies for lifestyle modification, together with insights from patient persona and patient Essentialities to inform the development, updates and compliance with the directives of the comprehensive care plan. Such Proxies resolve the personal choices and behaviors set of specific problems associated with lifestyle, including its basis in unstructured data and the significant scope, depth and subjectiveness of, and the extensive body of research on, lifestyle measures. Insights from such Proxies assist the healthcare team inform and are evaluated by the healthcare team (700.30.7), as the basis for determining health risk, medical decision-making, developing and updating the comprehensive care plan and managing patient outreach, engagement and care plan retention.

Patient Persona in a preferred embodiment introduces a novel composition of (a) common general demographic information, (b) a novel data class of patient Essentialities and (c) changes or alterations to mSDOH as novel measures of patient compliance with the directives of the comprehensive care plan. In addition to the clinical EMR data, a preferred embodiment collects and analyzes novel patient mSDOH behavior information (FIG. 21). Such information is collected from both direct reports of the EMR system evaluated by the healthcare team and remote patient-reported-outcomes and xPOCT reports (690.201). Novel patient mSDOH behavior information representing the patient's persona is expressed by the patient's social and personal competencies externally viewed through the patient's pattern-of-life.

Essentialities, a Dimension of patient persona, in the preferred embodiment are introduced to healthcare delivery as novel evidence of the patient's unique social and personal competencies, particularized to the Dimensions and factors based on their relevancy to the patient (that is, value or harm) to the patient and situated within the patient's pattern-of-life. See FIGS. 19A-19B and 18. Essentialities comprise the new or improved combination of 11 Dimensions shown at FIG. 21 and their factors evidencing the patient's unique social and personal competencies situated within and expressed by the patient's pattern-of-life. The Essentialities inform, influence and impact compliance with the comprehensive care plan directives. Essentialities are reported by the patient to and evaluated by the healthcare team (700.30.7), as the basis for determining health risk, medical decision-making, development and updating of the comprehensive care plan and managing patient outreach, engagement and care plan retention.

In a preferred embodiment of the invention, the novel Dimension of Essentiality (690) comprises one or more of: (a) status of individual deprivation (including social exclusion status, vulnerability status, socioeconomic status, sociocultural status, psychosocial status, behavior/lifestyle, social ties, chronic stress, health outcomes, livelihood strategies/occupation, education-achievement-competency, income, environment—social [distinguished from physical environment], core measures of HRQoL, obesity and poverty mapping); (b) gender-sensitive dimensions of status of individual deprivation (including food/nutrition, hunger, shelter, housing [materials and condition of the dwelling], homelessness, health/healthcare, health status, healthcare access, healthcare quality, education, competed schooling, competence [reading, writing, arithmetic], decision-making [authority/span, control over], personal support, personal care [clothing, presentation in public], protection from elements, freedom from violence, family planning, contraception [access to, use of], environment [physical environment problems], voice in the community [ability to participate in community decision-making; ability to change her community], time-use/labor burden [labor burden as percent of 24 hours; risk and respect; risk <paid and unpaid work>, status [paid and unpaid work]); (c) race or ethnicity; (d) patient perceptions of the medical interview (including pattern response factors, socioeconomic status, sociocultural status, psychosocial status, other life-contextual factors and healthcare team's personal observation—patient verbal and non-verbal health clues); (e) patient voice in the household (including preferences and healthy choices, influences impacting preferences and choices, health decision-making plan and family interaction plan); (f) community health asset utilization (including identification, location, reach/solicitation, enrollment, valuation, perceived value proposition, incentives/rewards portfolio, patient utilization, verification of patient engagement transactions and asset portfolio management); (g) the patient's perception of the efficacy of treatment (including, health outcomes [particularly those impacted by dyad and triad comorbidities], therapy program [improvement, cessation, self-management, group therapy], external environment impacts,); (g) the actual curative impact of treatment (including program valuation, performance utilization, surveillance, compliance and reporting); (h) technology use by the patient (personal-level [demographic, socioeconomic and community health asset utilization], family-level [demographic, socioeconomic and community health asset utilization]; household-level [demographic, socioeconomic and community health asset utilization]); (i) care coordination support (including patient valuation [transfer of care activities], coordination activities [patient utilization, analysis and reporting], community health asset utilization [portfolio management]); (j) mSDOH-burden hazard ratios; and (k) lifestyle.

The novel Essentialities inform, influence and impact compliance with the comprehensive care plan directives. In a preferred embodiment, compliance with such directives is comprised of: (a) adherence and understanding of the directives by the patient including education information with respect to the coexisting most-prevalent chronic diseases and self-management activities recommended in the comprehensive care plan, together with a prediction of adherence; (b) compliance with the prescribed or recommended self-care and management of conventional treatment (such as for example: strengthening; inhalers; oxygen therapy; medications; integration of treatment; smoking cessation), as prescribed or recommended by the comprehensive care plan, together with a prediction of compliance; (c) frequency, intensity and duration of patient-performed activities as prescribed or recommended by the comprehensive care plan, together with a prediction of motivation; and (e) deviations and causes of deviations from the patient-performed activities that have been prescribed or recommended by the comprehensive care plan, together with a prediction of cessation.

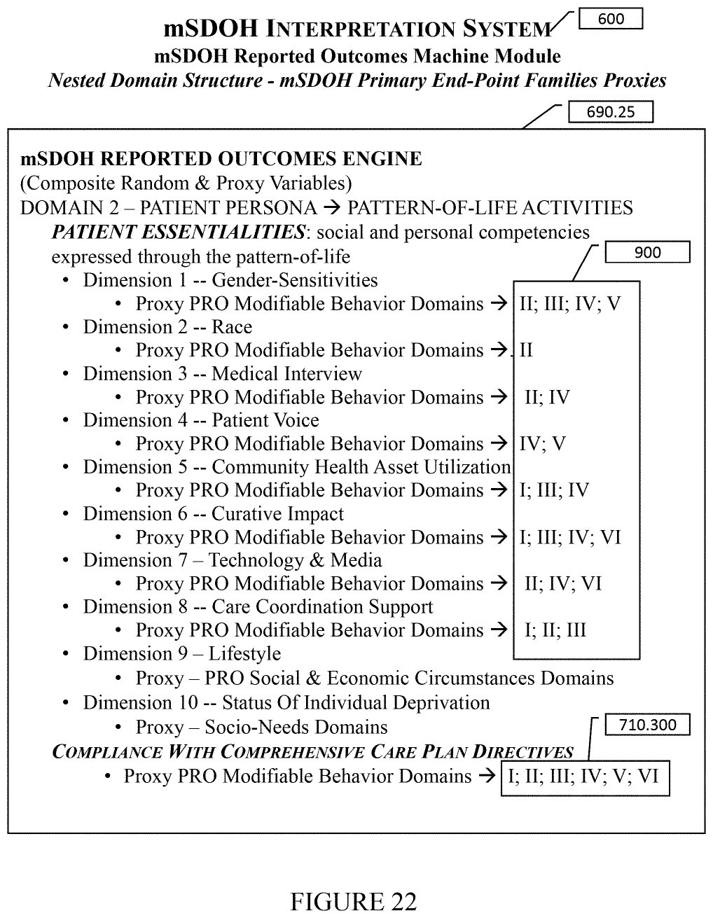

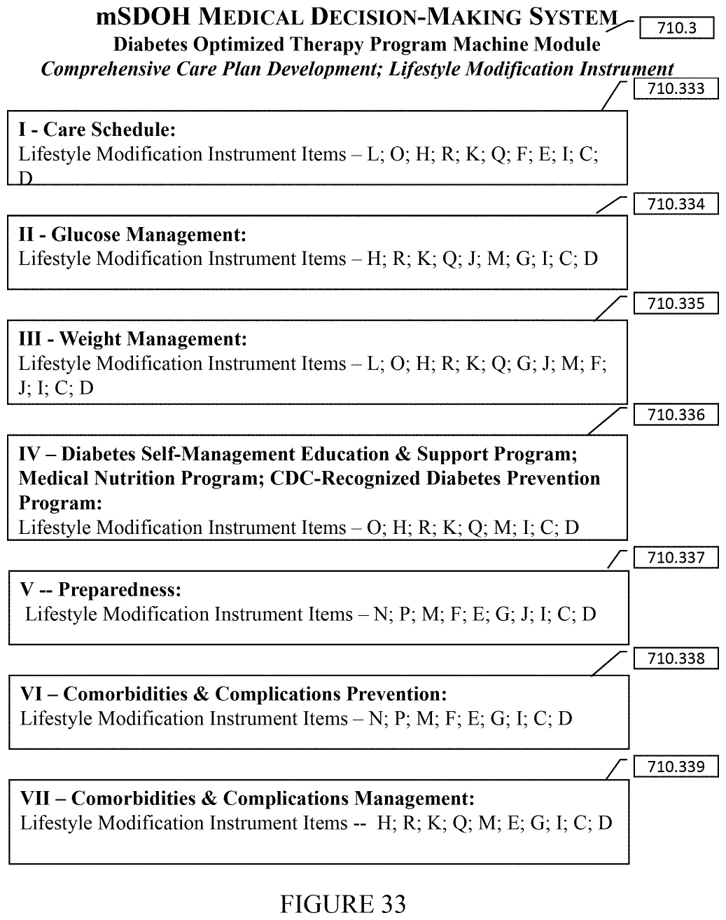

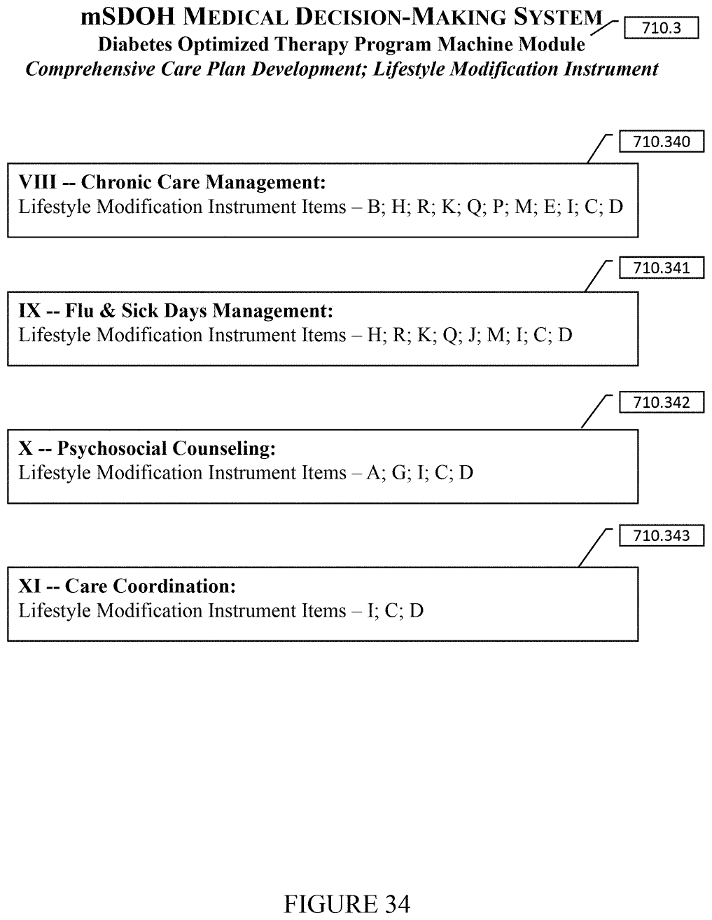

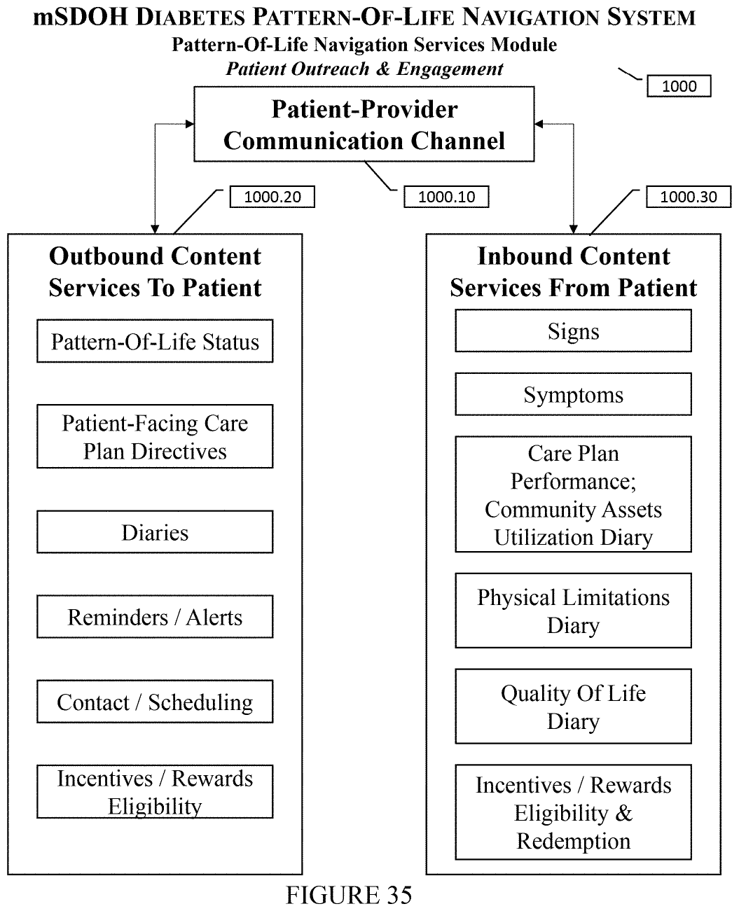

The operationalization by the invention of novel Essentialities as diabetes treatment methods by scoring each Essentiality, by applying the composite score as patient insights to inform the healthcare team through the mSDOH medical decision-making system (FIGS. 30-34) and the mSDOH pattern-of-life navigation system (FIGS. 35-39) and by applying the insights of each Essentiality (FIG. 22 (710.300)) to inform the healthcare team in its development, updating and management of compliance with the comprehensive care plan.

Essentialities are many in number and are common in the study of human behaviors and their impact on health. Accordingly, each Essentiality, except for lifestyle, is expressed by a score for the Essentiality ascribed by the patient from one to three, with three being the highest in importance as perceived by the patient. Lifestyle is disproportionately weighted at no less than a ratio the numerator of which is the mSDOH-burden hazard ratio applicable to the patient (such as city, county, State or national) and the denominator is the score of the sum of the other Essentialities. The composite score informs (700) the health risk calculator in the pattern-of-life knowledge machine (FIGS. 24-28B) through its composite random and Proxy variables (700.15).

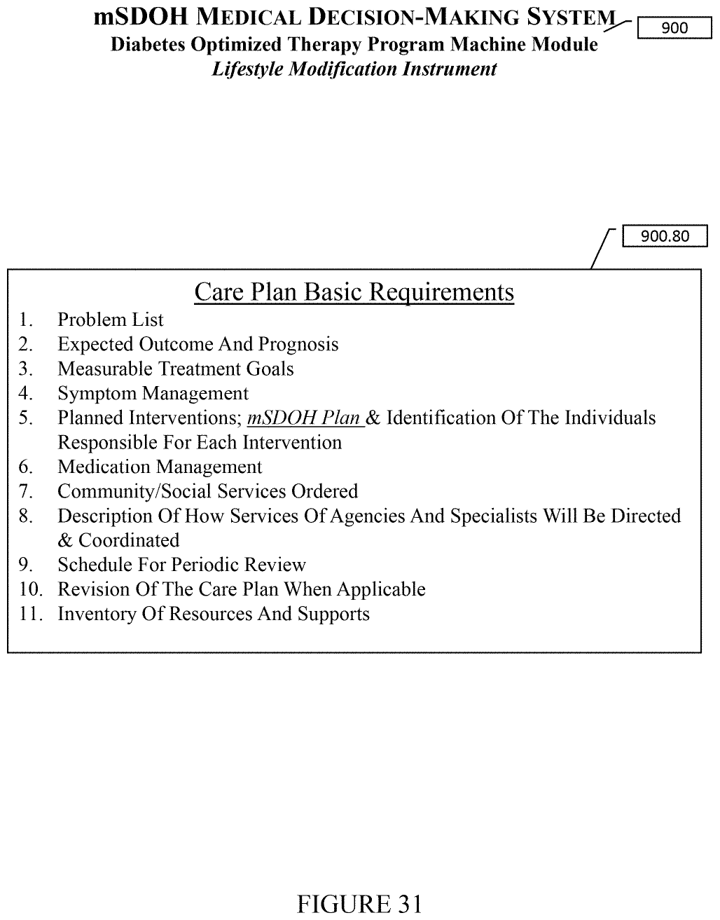

A preferred embodiment of the medical decision-making process introduces a novel mSDOH translation system, through its mSDOH reported outcomes machine shown in FIG. 21 where subjective patient behavior data is translated and incorporated into the structured data utilized by EMR systems. The preferred approach also introduces a novel process comprising changing or altering mSDOH as a method for assisted diabetes diagnosis of a patient, as well as a method for assisted development, updating and management of the patient's comprehensive care plan at the mSDOH medical decision-making system and its therapy management methods at FIGS. 31-34.

Further, at FIG. 23, a preferred embodiment introduces a novel means of patient navigation through the pattern-of-life by its Proxy, novel mSDOH repeated treatment measures over time, and by the novel patient-reported-outcomes assessment of novel mSDOH repetitive measures, together with common repetitive measures over time through a remote interactive patient-reported-outcomes instrument and a xPOCT (690.30). A preferred embodiment of the invention introduces a novel combination novel of mSDOH repeated measures together with common repeated measures: (a) such as novel mSDOH repeated measures of: (1) physical capacity (such as pain and discomfort, energy and fatigue and sleep and rest); (2) psychological factors (such as positive feelings, thinking, learning, memory, concentration, self-esteem, bodily image and appearance and negative feelings); (3) level of independence (such as mobility, activities of daily living, dependence on medication or treatments and work capacity); (4) social relationships (such as loneliness, personal relationships, social support, sexual activity, clinical family history, clinical social history and patient-reported-outcomes systems on how the patient functions, feels or survives); (5) environmental impacts (such as physical safety and security, home environment, financial resources, accessibility and quality of health and social care, opportunities to acquire new or improved information and skills, participation in and opportunities for recreation and leisure activities, physical environment [pollution, notice, traffic, climate] and adequate transportation); and (6) the patient's spirituality or personal beliefs (such as comfort, security, sense of belonging, purpose and strength, intensity, capacity, frequency, evaluation of states or behaviors) (690.50), together with the common repeated measures of: (7) the visit schedule; (8) pre-screening procedures (with the description of the visit procedures and assessment); (9) baseline visit (with the description of the visit procedures and assessment); (10) t-wave of periodic visits (such as month-to-month follow-on visits) together with the description of the visit procedures and assessment; (11) t-wave of periodic follow-on encounters including encounters remote from or external to the provider's direct supervision (with the description of the visit/encounter procedures and assessment); (12) patient's performance of the responsibilities under the treatment algorithm together with the compliance scale; and (13) final (end-of-intervention) visit together with the description of the visit procedures and assessment.

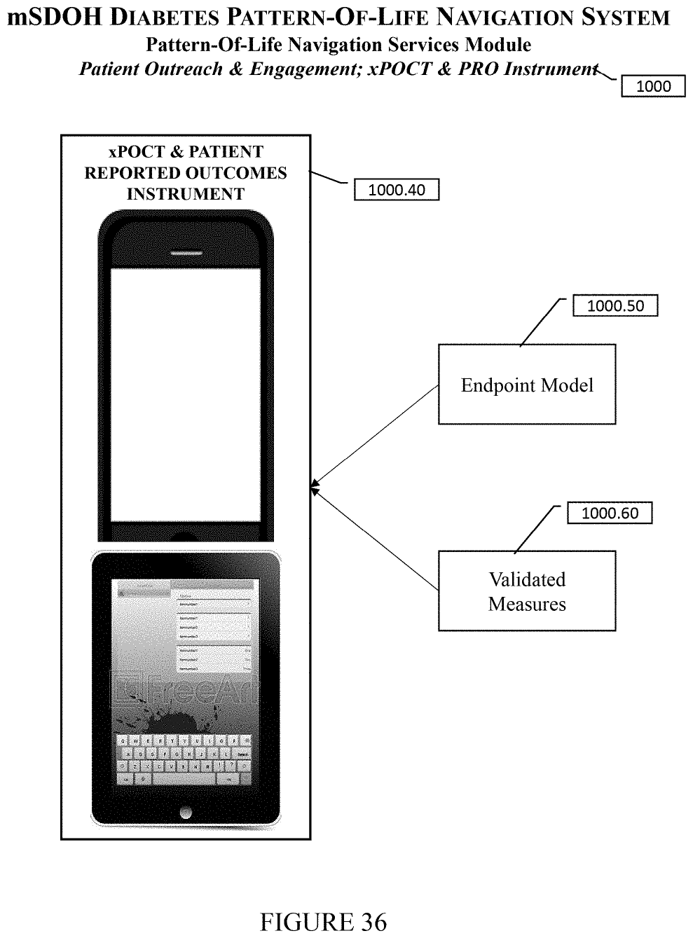

Diabetes Pattern-Of-Life Knowledge In a preferred embodiment (FIG. 36), the patient-reported-outcomes instrument and the xPOCT evaluate and inform such outcomes and the novel mSDOH repeated treatment measures through the use of common face-to-face survey tools, common remote interactive survey tools and common clinical measures tools, as well as novel, interactive, remote, mSDOH patient-reported-outcome Signs and Symptoms. Such common and new or improved tools, together, provide novel modifiable health risk measures that are grouped and managed by the novel diabetes pattern-of-life knowledge machine module at FIG. 24. The novel groups of measures at FIG. 24 are modifiable behavior Domains (700.11), social and economic circumstances Domains (700.12) and socio needs Domains (700.13). Each of such Domains is a novel patient-reported-outcome that details the patient novel pattern-of-life shown at FIG. 18 (610). The novel diabetes pattern-of-life knowledge machine module at FIG. 24 informs the diabetes knowledge feedback and evaluation machine at FIG. 29 et seq., the patient preferences machine at FIG. 19 and the pattern-of-life profile at FIG. 18, and all of which establish or update a novel basis for generating health risk scores used by the healthcare team as a novel basis for diagnosing the state and stage of diabetes and a novel basis for developing, updating and managing the comprehensive care plan and its related patient outreach, engagement and care plan retention.

The composite scores of each tool informs (700) the health risk calculator in the novel pattern-of-life knowledge machine (FIGS. 24-28B) through its novel patient reported outcomes primary endpoint families and Proxy variables (700.16). Each such individual score or composite scores, as may be necessary or desirable, are adjusted by the mSDOH-burden hazard ratio. Such outcomes assist the healthcare team diagnose the state and stage of diabetes and design, update and manage the comprehensive care plan. Such patient-reported-outcomes are reported to and evaluated by the healthcare team (700.30.7), as the basis for determining health risk, medical decision-making, development and updating of the comprehensive care plan and managing patient outreach, engagement and care plan retention.

In FIG. 24, a preferred embodiment introduces novel Domains of the mSDOH medical decision-making system's diabetes pattern-of-life knowledge machine module—the modifiable behaviors Domain, the social and economic circumstances Domain and the socio needs Domain. The three determinants are standardized, averaged and weighted to arrive at one composite index value. The index formula maximizes the correlation to the poor health outcomes of premature death and preventable hospitalizations, based on changes to novel mSDOH. Such changes to novel mSDOH are reported to and evaluated by the healthcare team (700.30.7), as the basis for determining health risk, medical decision-making, development and updating of the comprehensive care plan and managing patient outreach, engagement and care plan retention.

In a preferred shown in FIG. 24, a novel mSDOH health risk category is introduced comprised of modifiable health risk measures and their correlation with premature death and preventable hospital stays. Common health outcomes with respect to premature death and preventable hospital stays are ranked 50% and 5%, respectively, among a broad array of common health risks and outcomes. Commonly, the four health risk factor components of such two outcomes are ranked: health behaviors—30%; clinical care—5%; social and economic circumstances—25%; socio needs 21%. The novel mSDOH category results after adjustments to such two outcomes for their weighted average and is further adjusted for the components of the two outcomes to account for mSDOH, so that the two outcomes have a weighted average of 91% and 9%, respectively, and the four health risk components are ranked: by reducing the common health behaviors category 6% to 24%; by eliminating the common clinical care category; by reducing the common social and economic circumstances category 10.8% to 21%; by reducing the common socio needs category 9% to 12%; and by reducing the common socio needs category 9% to 12%. Such adjustments accommodate the novel mSDOH category. It is comprised of 43% of the outcomes representing 39.9% of premature death and 3.9% of preventable hospital stays.

The novel mSDOH category is accounted for: with respect to the common health behaviors category, by the elimination of the access to exercise opportunities factor (1%), the alcohol-impaired driving deaths factor (2.5%) and the teen births factor (2.5%); with respect to the common social and economic circumstances, by eliminating the children in poverty factor (7.5%), the single-parent households factor (2.5%), the violent crime factor (2.5%) and the injury deaths factor (2.5%); and with respect to the socio needs category, by substituting the category with the mSDOH-burden hazard ratio based on socioeconomic indicators such as zip codes.

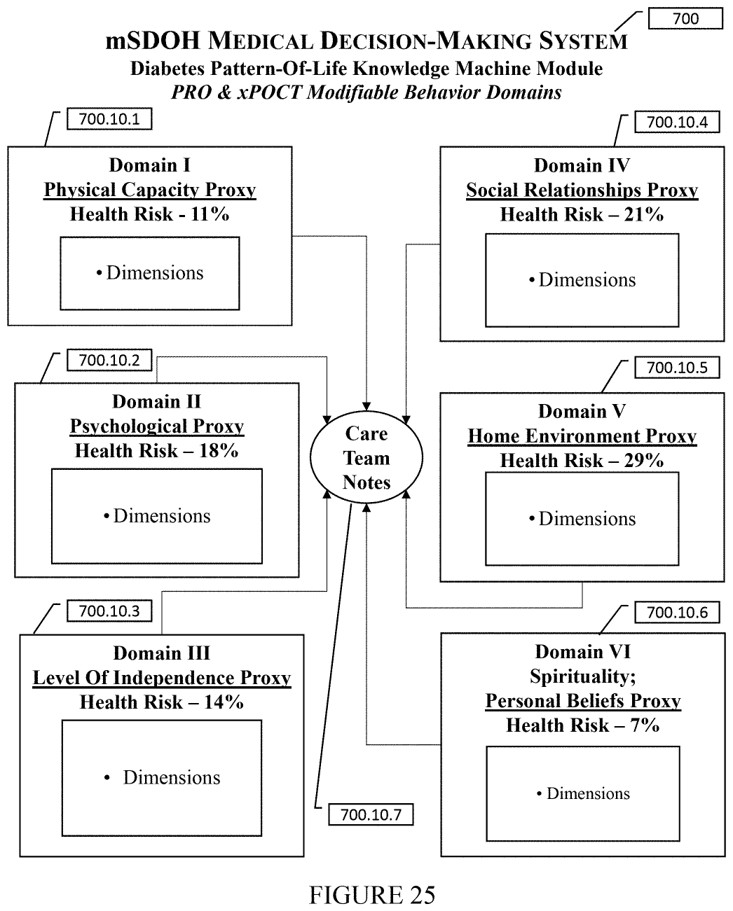

FIG. 25 introduces a novel patient-reported-outcomes component of the modifiable behavior Domains of the diabetes pattern-of-life knowledge machine module of the mSDOH medical decision-making system introduced by a preferred embodiment. This module has six Domains—physical capacity, psychological, level of independence, social relationships, home environment and spirituality. Each Domain is a Proxy for the Domain's composite Dimensions, and each Domain has a health risk score. The six Domains are standardized, averaged and weighted to arrive at one composite index value. The index formula maximizes the correlation to the poor health outcomes of premature death and preventable hospitalizations, based on changes to mSDOH and the assessment by the patient of the impact of changing or altering mSDOH on such outcomes. Such changes or alterations are evaluated by the healthcare team (700.30.7), as the basis for determining health risk, medical decision-making, development and updating of the comprehensive care plan and managing patient outreach, engagement and care plan retention.

In a preferred embodiment there is introduced a novel: a physical capacity Domain (700.10.1) which is weighted with a health risk score of 11% and introduces a novel Proxy for the patient-reported-outcomes composite of pain and discomfort, energy and fatigue and sleep and rest; a psychological Domain (700.10.2) which is weighted with a health risk score of 18% and introduces a novel Proxy for the patient-reported-outcomes composite of positive feelings, thinking-learning-memory and concentration, self-esteem, bodily image and appearance and negative feelings; a level of independence Domain (700.10.3) is weighted with a health risk score of 14% and introduces a novel Proxy for the patient-reported-outcomes composite of mobility, activities of daily living, dependence on medication or treatments and work capacity; a social relationships Domain (700.10.4) which is weighted with a health risk score of 21% and introduces a novel Proxy for the patient-reported-outcomes composite of personal relationships, social support, sexual activity, clinical family history, clinical social history and how the patient functions, feels and survives; a home environment Domain (700.10.5) which is weighted with a health risk score of 29% and introduces a novel Proxy for the patient-reported-outcomes composite of physical safety and security, home environment, financial resources, health and social care accessibility and quality, opportunities to acquire new or improved information and skills, opportunities for and participation in recreation and leisure activities and availability and adequacy of transportation; and a spirituality Domain (700.10.6) which is weighted with a health risk score of 7% and introduces a novel Proxy for the patient-reported-outcomes composite of comfort, well-being, security, sense of belonging, purpose and strength and their intensity, capacity and frequency, together with the evaluation of personal status. The evaluation of the patient-reported-outcomes in the above six Domains is made by the healthcare team (700.10.7), as the basis for determining health risk, medical decision-making, development and updating of the comprehensive care plan and managing patient outreach, engagement and care plan retention.

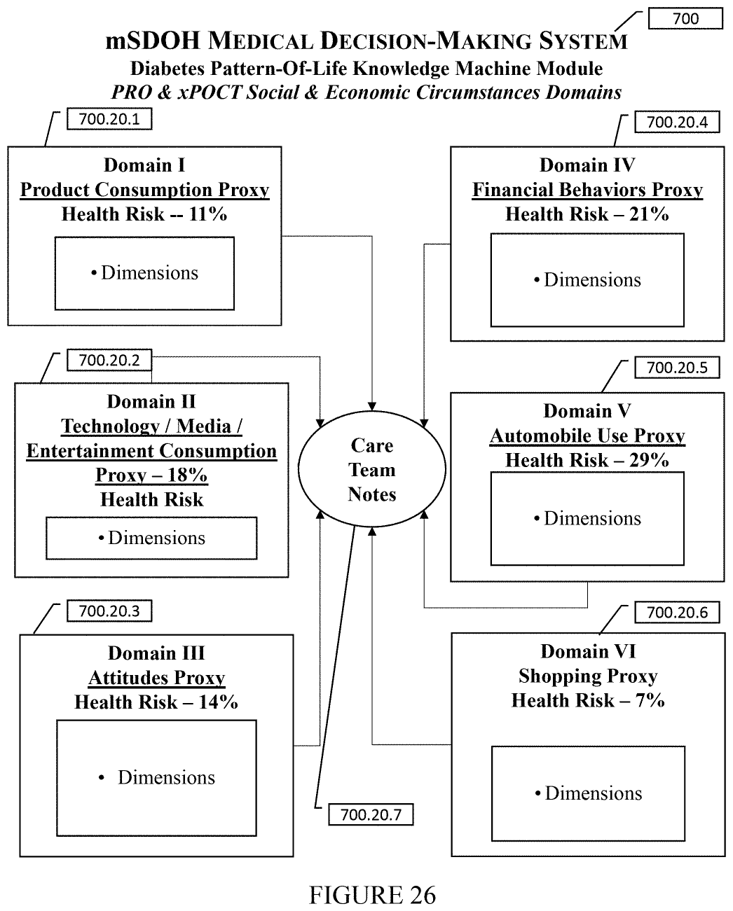

FIG. 26 shows a preferred embodiment introducing a novel patient-reported-outcomes social and economic circumstances Domains component of the novel diabetes pattern-of-life knowledge machine module of the novel mSDOH medical decision-making system. Common Domains of this module, and their respective health risk scores, are: product consumption—18%; attitudes—14%; financial behaviors—21%; automobile transportation—29%; and shopping—7%. Each Domain is a Proxy for the Domain's common composite Dimensions, as commonly used in the consumer marketing industry. Such Domains are standardized, averaged and weighted to arrive at one composite index value. In a preferred embodiment, the index formula introduces a novel correlation of poor health outcomes of premature death and preventable hospitalizations, based on changes to mSDOH. The evaluation of the patient-reported-outcomes in the above six Domains, adjusted for the novel correlation to mSDOH and the mSDOH risk hazard, is made by the healthcare team (700.20.7), as the basis for determining health risk, medical decision-making, development and updating of the comprehensive care plan and managing patient outreach, engagement and care plan retention.

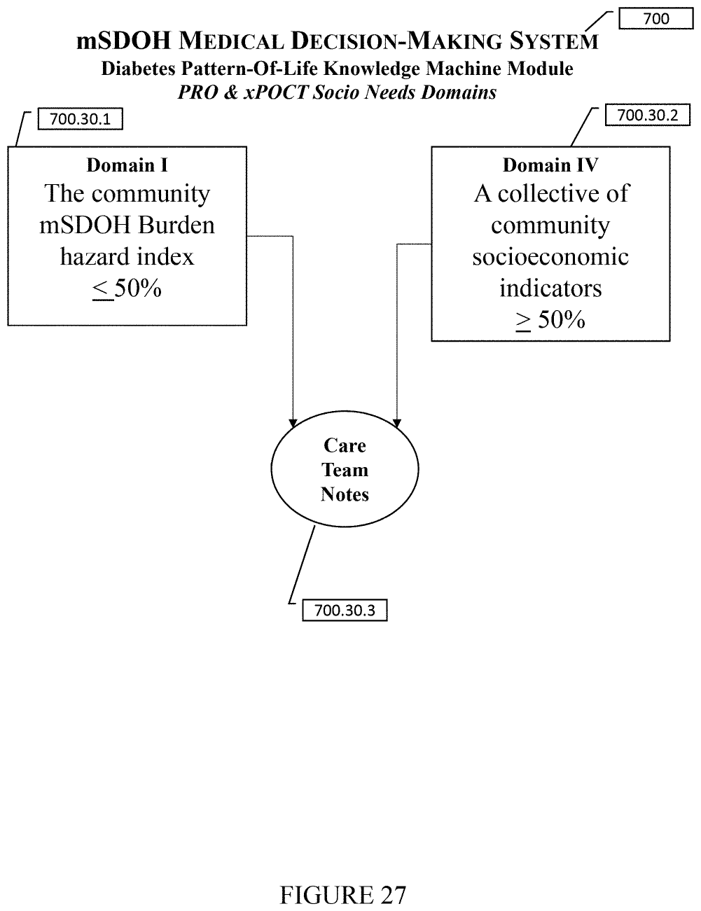

FIG. 27 shows a preferred embodiment introducing a novel patient-reported-outcomes socio needs Domains component of the novel diabetes pattern-of-life knowledge machine module of the novel mSDOH medical decision-making system. The Domains of this module, and their respective health risk scores, are the novel mSDOH-burden hazard ratio for the community in which the patient resides (700.30.1) and a collective of novel community socioeconomic indicators comprising poverty, income, unemployment, occupation, education application to career goals, language barriers and other SDOH indicators reported by one of the most-current community needs assessment of a hospital in the service area in which the patient resides, or other government-recognized geographic area such as zip codes, defined neighborhood or region, voting precinct and historical preservation district (700.30.2), as well as demographic definitions. The evaluation of the patient-reported-outcomes in the above six Domains is made by the healthcare team (700.30.3), as the basis for determining health risk, medical decision-making, development and updating of the comprehensive care plan and managing patient outreach, engagement and care plan retention.

Statistical Considerations

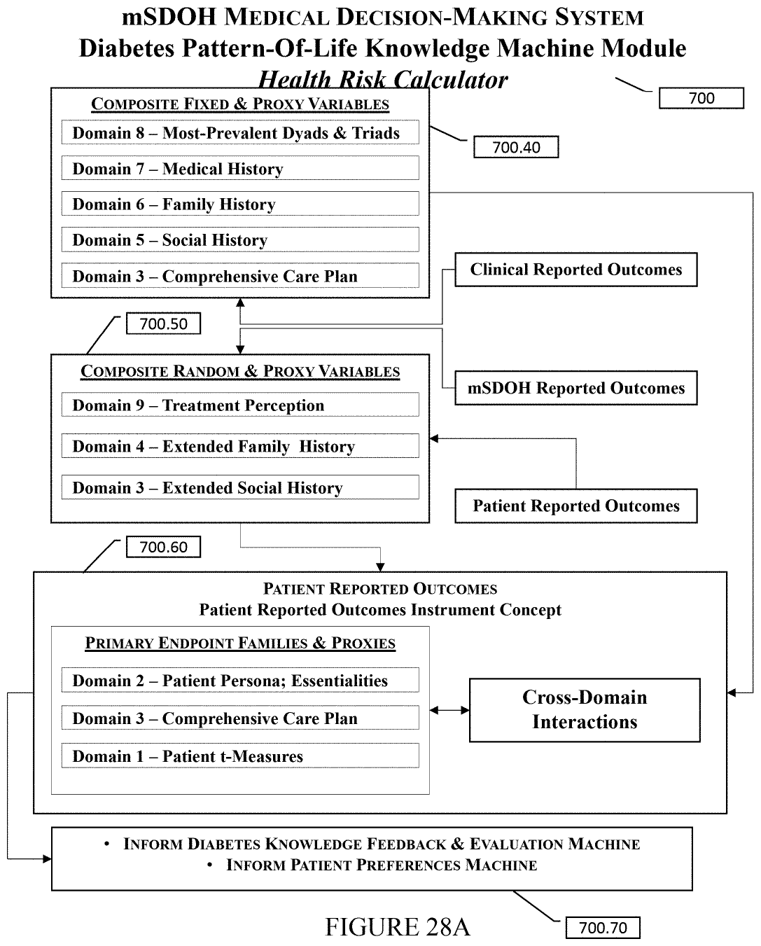

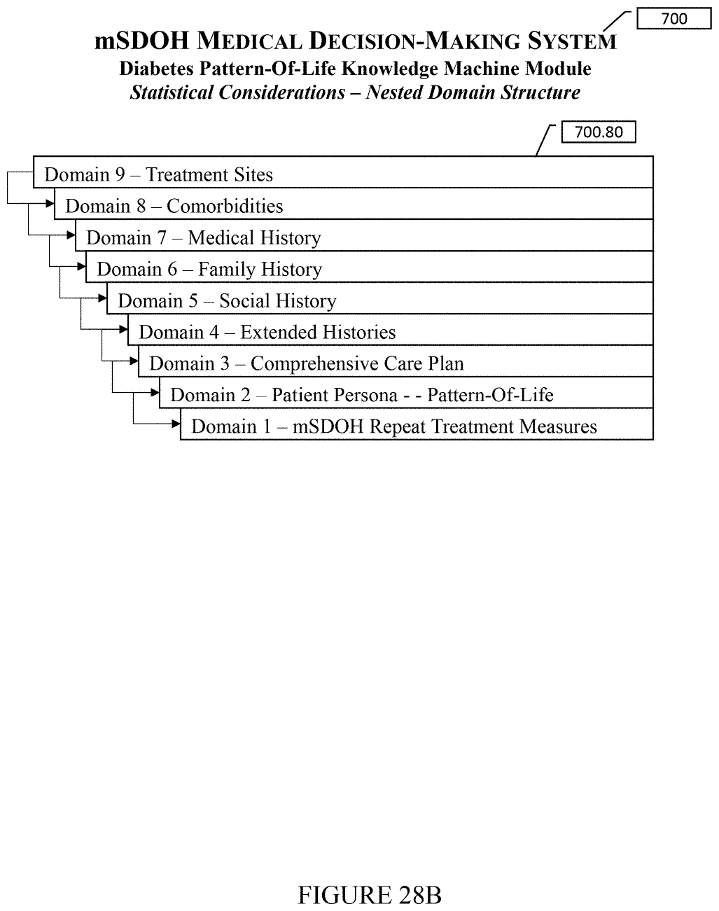

GENERAL STATISTICAL CONSIDERATIONS: Diabetes evidences disturbances in complex biological systems. As a result, the statistical approaches used by the health risk calculator at FIG. 28A must consider complex risk factors (such as those of diabetes, cardiovascular diseases and metabolic syndrome) and complex responses to interventions (such as diabetes treatment methods).

Finding diabetes treatments with the best or an improved risk/benefit ratio entails relating many variables to composite phenotypes, including lifestyle, its characteristics, its behavior and the products of its behavior. Special real-world problems arise when evaluating the impact of lifestyle on preventing, diagnosing, predicting or treating diabetes. Multiple measures of influences and outcomes must be considered. Further, most complex phenomena lack a physical scale to be “measured” in the traditional sense, as a single measure typically does not reflect all relevant aspects to be considered. In addition, a definitive measure may not even exist.

Moreover, when the definite measure is not easily obtained, surrogate or Proxy measures must be evaluated. While it is often reasonable to assume that “more data” is “worse” (for computational purposes) for each phenomenon, it is not be easy to determine, how much “more” is how much “worse”.

In comparison, under common linear scoring methods, most multivariate methods are either explicit (as in regression, factor, discriminant and cluster analysis) or implicit (as in neural networks). One scores each variable individually on a comparable scale, for example either present/absent, low/intermediate/high, 1 to 10, or z-transformation, and then defines a global score as a weighted average of these scores. Thus, data are interpreted as points in a Euclidian space. The number of dimensions is reduced by assuming them to be related by a function of known type (linear, exponential, etc.), allowing one to determine for each point the Euclidian distance from a model hyperspace. Such factors are relatively few parameters, when contrasted to the parameters of real world complex biological systems such as diabetes. In addition, lineal statistical methods lack computational efficiency in that the computational effort required could be prohibitive for the micro arrays with thousands of factors or variables for lifestyle/behavioral analysis.

The general statistical model of a preferred embodiment uses a longitudinal, multi-level, random-intercept, logic regression method. It enables: (a) analyzing of both clustered and longitudinal data to support multi-level or hierarchical versions of repeated nested measurements with the capability of simultaneously decomposing the overall variance into components related to within-subject effects and within-cluster effects; (b) modeling and comparing of longitudinal response patterns for continuous and categorical outcomes using a unified family of statistical models; and (c) estimating of person-specific trends.

The statistical approach shown at FIG. 28A utilizes common linear scoring methods for Domains 7, 6, 5, 4 and 3 as composite fixed and Proxy variables (700.40) and for Domains 9, 4 and 5 as composite random and Proxy variables (700.50). A multilevel, hierarchical, nested data model is used for the patient-reported-outcomes (700.60) for Domains 3, 2 and 1 and for cross Domain interactions.

A preferred embodiment introduces mSDOH, as a Proxy for patient preferences, lifestyle, its behavior and the products of its behavior, into the statistical analysis of diabetes. The scope of statistical analysis includes: identifying mSDOH relevant to the patient; identifying and predicting the health-related outcomes from changing or altering mSDOH; assessing such changing or alterations for their impact on the state, status, health risk and treatment of diabetes; informing methods for the prevention, delay in the onset, diagnosis and treatment of diabetes; and operationalizing the insights and analysis of mSDOH in determining health risk, making medical decisions, developing, updating and managing comprehensive care plans and patient outreach, engagement and care plan retention. The statistical analysis tests and informs the operation of the mSDOH-burden hazard ratio at FIG. 16 and the health risk calculator at FIG. 28A. The statistical analysis also tests and informs the operationalization of mSDOH insights through the mSDOH diabetes pattern-of-life navigation system and the operation of such system through patient outreach, engagement and care plan retention by the patient xPOCT and patient-reported-outcomes instrument at FIG. 36.

The general statistical model in a preferred embodiment is based on the nested domain structure at FIG. 28B. Such structure addresses nine nested levels, each a Domain. At such figure, the nine Domains are treatment sites, comorbidities, medical history, family history, social history, extended histories, comprehensive care plan, patient persona and patient reported outcomes. Other embodiments may use more or less than nine Domains. Each Domain represents a health risk category. Each category represents a correlation with a determinant of health and the determinant's associated cause of premature death as a hazard ratio of a disparity community or the general community. In some embodiments, the determinant of health may be associated with one or more other hazard ratios.

The data extracted by the healthcare team and the patient-reported-outcomes are scaled such that higher scores indicate higher HRQoL. The proportion of patients against the severity levels of diabetes and the six most prevalent coexisting chronic diseases, against the prevalence and severity levels of the mSDOH Domains, against the increase in comorbidities, against the rate of performance of or compliance with the comprehensive care plan and against the utilization of community health assets are described by severity, intensity and frequency. Relationships among such proportionalities are assessed for categorical variables. The distribution of baseline social, demographic and clinical values across the prevalence of mSDOH relevant to the patient are compared. Associations between patient characteristics, state and stage of diabetes and its comorbid chronic conditions, demographic and clinical parameters and the severity, intensity and frequency, as appropriate, of the mSDOH are assessed. The impact on the stage or stage of diabetes from changes or alterations in the mSDOH is established. Such impact informs diabetes risk analysis, medical decision-making, development and updating of comprehensive care plan and retention in the care plan through patient outreach and engagement.

The statistical models include the medically diagnosed presence or status and the severity or stage of diabetes and the most-prevalent chronic condition triads and dyads, the mSDOH relevant or valuable or harmful to the patient and relevant social and demographic variables, as well as the patient's clinical parameters (blood pressure, dietary intake, anthropometry, etc.). The health risk calculator at FIG. 28A tests and reports, through the outputs of the statistical models, whether abnormalities in such clinical parameters develop or change as a result of changes or alterations to mSDOH by way of compliance with changes or alterations directed or prescribed by the comprehensive care plan. Such reports inform the diabetes knowledge feedback and evaluation machine and the patient preferences machine.

The general statistical model at FIG. 28A, as well as its application by the xPOCT/patient-reported-outcomes instrument at FIG. 36, introduce a Data Card for each of the nine Domains. The data cards are discussed at FIG. 36.

EFFICACY ENDPOINTS: In a preferred embodiment, an endpoint of the treatment risks or benefits is the change from baseline to time T expressed as an index or score that represents the Concept of at least one or more of Symptom severity or intensity, Signs associated with presence or absence of diabetes or one or more of its comorbidities. Symptoms or Signs are at least one of the indicators of diabetes or the impact on the stage or state of diabetes by a change or alteration of a mSDOH as directed by the comprehensive care plan or the impact on an activity limitation (as a subset of HRQoL) associated with diabetes or the coexisting most-prevalent chronic diseases. Such change is measured and statistically compared over time to assess at least one of (a) the effect of changing or altering mSDOH on treatment risks or benefits and (b) the effect on treatment risks or benefits of changes or alterations to mSDOH in performing the directives of the comprehensive care plan.

As measures intended to reflect the effects of changing or altering mSDOH on a treatment risk or benefit, efficacy endpoints include assessments of patient Symptoms (such as for example, pain, dyspnea, depression), measures of function (such as for example, ability to walk or exercise), clinical events (such as for example, blood glucose level changes, stroke, pulmonary exacerbation, venous thromboembolism) and Proxies of these events or Symptoms, as well as the relationships among endpoints.

Efficacy endpoints include patient-reported-outcomes and non-patient-reported-outcomes. Non-patient-reported-outcomes endpoints include measures reported by the patient's Proxy (such as a caregiver where appropriate) and by clinician-reported measures.

The endpoint model of a preferred embodiment is expressed as the hierarchy of relationships among all endpoints, both patient-reported-outcomes and non-patient-reported-outcomes, that correspond to the embodiment's treatment method. The endpoint model includes as a Concept the measurement of the change in one or more or a group of Symptom-intensities, activity limitations or function-limitations of the patient. The endpoint measure is the change or alteration to mSDOH that inform, by way of general improvement, no reduction or negative increment or a reduction or negative increment, in the outcomes of treatment of the patient's diabetes or its coexisting most-prevalent chronic diseases and a composite score of the composite endpoints of (a) the changes or alterations of mSDOH as treatment benefits or risks and (b) the such changes or alterations as compliance treatment benefits or risks expressed through the patient-reported-outcomes of how the patient survives, feels or functions.

Supportive Concepts to the Concept are improvement in Symptoms and the delay in the onset or development of Symptoms. Supportive Concepts are operationalized through patient-reported-outcomes measures and non-patient-reported-outcomes measures including: a Symptom diary (particularly a patient-reported-outcomes assessment); a Signs diary (particularly a patient-reported-outcomes assessment); a physical performance diary (particularly a patient-reported-outcomes assessment); a community health asset utilization diary (including a patient-reported-outcomes assessment and a non-patient-reported-outcomes assessment); a medication diary (particularly a patient-reported-outcomes assessment); a related physical limitation diary (particularly a patient-reported-outcomes assessment); and a physical examination (a non-patient-reported-outcomes assessment). Changes in mSDOH data and outreach, engagement and retention intervention data are included with all of the diaries except the physical examination diary.

ENDPOINT FAMILIES: In a preferred embodiment, endpoints are grouped hierarchically according to the importance of the endpoints' contribution to treatment risk or benefit, and secondarily according to consideration of the expected frequency of the endpoint events and anticipated effects.

Primary endpoints are those that are essential to establish effectiveness of the treatment benefit. Secondary endpoints are those that demonstrate additional meaningful effects from the treatment risk or benefit. Exploratory endpoints are all endpoints that are not primary or secondary endpoints. Exploratory endpoints include endpoints that for other reasons are thought to be less likely to show an effect but are included to explore new hypotheses and important events that are expected to occur too infrequently to show a treatment effect.

Each of the several endpoints that support a conclusion of effectiveness are members of a Primary Endpoint Family. The Primary Endpoint Families are the mSDOH Primary Endpoint Family and the Compliance Primary Endpoint Family. The table below schedules each Primary Endpoint Family, together with its corresponding Domain, clinical importance and commonalities or reasonably-similar clinical importance.

Primary Endpoint Families

| Primary | | | Commonalities |
|---|---|---|---|
| Endpoint | | | Reasonably-Similar |
| Family | Domains | Clinical Importance | Clinical Importance |
| mSDOH | Domain 3 | Social Histories | HRQoL |
| mSDOH | Domain 2 | Patient Personalities/ | HRQoL |
| | | other Social Histories | |
| Compliance | Domain 1 | Directives of | Health goals/targets |
| | | Comprehensive | Objectives/how to |
| | | Care Plans | reach goals/targets |

PRIMARY ENDPOINT FAMILIES: In a preferred embodiment, Primary Endpoint Families consist of mSDOH and compliance. Domains 3, 2 and 1 are primary endpoint families. The clinical importance of Domain 3 is its social histories. The clinical importance of Domain 2 is its patient personalities (excluding social histories). The clinical importance of Domain 1 is its repetitive measures over time. The commonalities or reasonably-similar clinical importance of mSDOH Domain 3 are its patient-reported-outcomes interactive diaries, HRQoL, remote encounter settings and activity limitation. Similarly, the commonalities or reasonably-similar clinical importance of mSDOH Domain 2 are its patient-reported-outcomes interactive diaries, HRQoL, remote encounter settings and activity limitation. The commonalities or reasonably-similar clinical importance of mSDOH Domain 1 are its patient-reported-outcomes interactive diaries, health goals/targets, objectives (how to reach goals/targets), repetitive performance directives of comprehensive care plans, HRQoL, remote encounter settings and activity limitation.

SECONDARY ENDPOINT FAMILIES: Secondary endpoint families consist of Domain 4—Extended Histories and Domain 5—Family Histories.

EXPLORATORY ENDPOINT FAMILIES: Exploratory endpoint families consist of: Domain 9—Treatment Sites; Domain 8—most-prevalent coexisting chronic diseases; Domain 7—Medical Histories; Domain 6—comprehensive care plans/Medical Decision-Making; and Domain 5—Family Histories.

PRIMARY ENDPOINT FAMILIES—WEIGHTED IMPACTS OF SUCCESS: The coexisting most-prevalent chronic diseases have multiple sequelae, and consequently, have more than one clinical outcome. All outcomes are affected by the treatment risk or benefit. The scope and impact of such success are attributable to the commonalities or the reasonably-similar clinical importance of the composite endpoints of the Primary Endpoint Families. Accordingly, success on any one of the Primary Endpoint Families or their component composite endpoints will support a conclusion of treatment effectiveness.

PRIMARY ENDPOINT FAMILIES—COMMONALITIES/REASONABLY-SIMILAR CLINICAL IMPORTANCE: With respect to the mSDOH Primary Endpoint Family, its commonalities or reasonably-similar clinical importance are Social Histories and Patient Personalities/other Social Histories. The commonalities or the reasonably-similar clinical importance features of Social Histories and Patient Personalities/other Social Histories are their HRQoL, as well as remote encounter settings and activity limitation.

The HRQoL is a Proxy for the patient's pattern-of-life comprising behavior patterns and social circumstance, each of which is a cause of premature deaths in the U.S. The HRQoL are a multi-Domain Concept that represents the patient's general perception of the effect of illness and treatment on physical, psychological and social aspects of life. Claiming a statistical or meaningful improvement in HRQoL will imply: (1) that Domain 3—Social Histories and Domain 2—Patient Personalities are important to the patient for measuring and interpreting change in how the patient feels or functions, as a result of a change or alteration of a mSDOH and its impact on treatment benefit or risk with respect to diabetes or the coexisting most-prevalent chronic diseases; and that (2) there was at least one of: a demonstration of general improvement; a demonstration of no reduction or negative increment; or a demonstration of a reduction or negative increment as a treatment risk or benefit.

With respect to the Compliance Primary Endpoint Family, its clinical importance is the repetitive requirement to perform the directives of the comprehensive care plan and the repetitive compliance with the clinical-measures requirements.

COMPOSITE ENDPOINTS; INTERRELATIONSHIPS: In a preferred embodiment, the endpoints comprising each Domain combine into a composite endpoint for the Domain. Each Domain's composite endpoint constitutes a single variable. The variables representing the mSDOH Primary Endpoint Family and the Compliance Primary Endpoint Family are continuous random variables. The variables representing the secondary endpoint families and the exploratory endpoint families are fixed variables. The interrelationships between the mSDOH Primary Endpoint Family and the Compliance Primary Endpoint Family are continuous random variables. The interrelationships between and among the fixed-variable composite endpoints of Domains 9 through 4 are measured through the index or weight ascribed to each of the fixed variables.

DATA REDUCTION PLAN: The statistical model produces a significant effective number of variables. A preferred embodiment reduces the effective number of Items through the following method:
1. Assign a value to each predictor variable—Domains and Items.
2. Weight the predictor variables—Domains and Items—as sub-indices or scores, whereby:
   a. Domain 9—Treatment Sites—are designated as a fixed variable for each patient; this fixed variable is assigned a sub-index or score on the basis that they represent structured data attributable to public information.
      i. The Domain 9 sub-index or score is weighted 5% of the total Domain weight on the basis that environment is correlated with 5% (or the otherwise then-current percent) of the premature causes of death in the U.S.
   b. Domains 8, 7 and 6—Most-Prevalent Comorbid Chronic Conditions, comprehensive care plan/Medical Decision-Making and Medical Histories—are designated as fixed variables; these fixed variables are aggregated into a composite sub-index or score on the basis that they represent structured data attributable to claims, clinical and health administrative data.
      i. The Domain 8-7-6 sub-index or score is weighted 10% of the total Domains weight on the basis that claims, clinical and health administrative data characterize health care, which is correlated with 10% (or the otherwise then-current percent) of the premature causes of death in the U.S.
   c. Domains 5 and 4—Family Histories and Extended Histories—are designated as random variables; these random variables are aggregated into a composite sub-index or score on the basis that they represent a hybrid of non-modifiable SDOH and mSDOH as genetic predispositions and environment.
      i. The Domain 5-4 sub-index or score is weighted 12% of the total Domains weight on the basis that genetic predispositions represent 30% (or the otherwise then-current percent) of the premature causes of death in the U.S. and that 40% of the 30% (12%) of such premature cause will not account for the impacts on genetic predisposition of modifiable cultural inheritance, including non-additive genetic components, shared family environment and individual environment
   d. Domains 3, 2 and 1—Social Histories, Patient Personalities and Recurring Measures—are designated as random variables; these random variables are aggregated into a composite index or score on the basis that they represent mSDOH data as pattern-of-life behaviors, social circumstances and compliance.
      i. The Domain 3-2-1 sub-index or score is weighted 73% of the total Domains weight on the basis that pattern-of-life behaviors are correlated with 40% (or the otherwise then-current percent), social circumstances are correlated with 15% (or the otherwise then-current percent) of the premature causes of death in the U.S. and that 60% of the 30% (18%) of the genetic predisposition premature cause will account for the impacts on genetic predisposition of modifiable cultural inheritance, including non-additive genetic components, shared family environment and individual environment.
3. The cross-Domain interactions are limited to only Domains 3, 2 and 1; the interactions are random sub-indices or scores.
4. The composite sub-indices or scores are aggregated into a composite index or score. The composite index or score is adjusted so as to account for the impact of the community mSDOH-burden hazard ratio.
5. Reference is made to relevant portions of the Health & Retirement Survey where appropriate Proxies are applicable.
6. Additional appropriate Proxies are applied in place of gaps in the collected data and gaps in such survey.

In addition, U-statistics can be used as a non-parametric alternative for scoring multivariate ordinal data to improve scoring profiles that best correlate with complex risk factors and complex responses to an intervention.

The relationships and interactions among the number and types of Items and among the Domains adjusted pursuant to the Data Reduction Plan are illustrated in FIG. 28B.

Additional items to define mSDOH and disease severity levels also are developed, based on the hypothesized anatomy of mSDOH, interviews of community-based participatory researchers utilizing principles of community-based participatory research and the xPOCT of the patient. Scaling, using severity, frequency and intensity levels, of mSDOH are defined using nested domain categories encountered by the patient including: comorbidities, medical history, family history, social history, extended social history, medical decision-making as presented in comprehensive care plans and patient persona.

TREATMENT EFFECT: Each of the components of the composites in the two Primary Endpoint Families are of equal importance in the analysis of the composite. Consequently, the treatment effect on the composite rate is interpreted as characterizing the overall clinical effect. Accordingly, the occurrence-event of any one of the individual components is considered to be an endpoint event.

MULTIPLICITY: The component variables of the mSDOH Primary Endpoint Family and the Compliance Primary Endpoint Family are components of composite endpoints, are components that will have commonalities or reasonably-similar clinical importance and are of equal importance in the analysis of the composite. As a result, there are no multiplicity of variables.

TREATMENT EVENTS: The composite endpoints of the two Primary Endpoint Families have components that correspond to events. An event means (a) the first occurrence of any of the component events, (b) each subsequent occurrence of the component event and (c) each subsequence measurement of being event-free. The composite endpoints are analyzed using time-to-event analysis.

EVALUATING THE COMPONENTS OF COMPOSITE ENDPOINTS: The statistical model considers first-occurring events and all subsequent events. Results for each statistically significant individual components of a composite event in the Primary Endpoint Family—Domains 3, 2 and 1, Social Histories, Patient Personalities and Repeated Measures, respectively—are examined individually and are included in the intervention reports to the healthcare team.

In analyzing the contribution of each component of a composite endpoint, the approach to considering a patient who experiences more than one of the event-types is to consider the events of each type in the patient. With this method, each of the components also is treated as a distinct endpoint, irrespective of the order of occurrence.

MEASUREMENT OF TREATMENT COMPLIANCE: Treatment compliance for the patient is calculated as the number of days the therapy activities performed by the patient as prescribed by the comprehensive care plan invention were actually performed (i.e., number of days of compliance minus days of temporary discontinuation) divided by the number of days of follow-up, allowing for early permanent discontinuation of participation in the comprehensive care plan when applicable. Participation discontinuation may be defined as a minimum of 5 consecutive days without receiving a report of compliance from the patient.

Diabetes Knowledge Feedback & Evaluation

FIG. 29 shows a common health risk dashboard (800.70). In a preferred embodiment, there is introduced a novel dashboard that presents information to the healthcare team in its medical decision-making by marshaling and summarizing the mSDOH results of the health risk calculator at FIG. 28A. The health risk evaluator for the patient reports: changes in the processes of diabetes (800.10), including the identification and onset of comorbid chronic diseases and the onset of diabetes complications; changes in the state of diabetes (800.20), including movement or progression from asymptomatic, to prediabetes, to diabetes and progression to metabolic syndrome; changes in the stage of diabetes (800.30), including high-risk, very-high-risk and extreme-risk based on blood glucose tests, the mSDOH-burden hazard ratio and disparity community hazard ratio; changes in the patient's pattern-of-life (800.40), including the patient's adoption of prescribed valuable behaviors and reduction or elimination of noticed harmful behaviors; changes in the comprehensive care plan (800.50), including goals, achievement of goals, increases/decreases in prescribed dietary intake and physical activity; and changes in the HRQoL (800.60), including activity limitation, independence and overall well-being.

Diabetes Lifestyle Modification Instrument

FIG. 30 introduces the process of a novel diabetes lifestyle modification instrument. In a preferred embodiment, there is introduced to the lifestyle modification instrument a novel mSDOH plan. FIG. 31 (900.80). Such instrument and its mSDOH plan include: a health risk assessment (900.10) for new patients and existing patients which includes the common recurring blood glucose testing and the novel analysis of the mSDOH-burden risk ratio; a care team-patient consultation (900.20), where the patient is advised of the assessment's results and the outcome and prognosis strategy; the development by the healthcare team of the comprehensive care plan (900.30) for implementing such strategy; a care team-patient consultation (900.40), where the care plan is presented to and discussed with the patient where family and caregivers also may be included; the approval by the patient of the comprehensive care plan (900.50), where approval may be made at such consultation or later after the patient has had an opportunity to discuss the care plan with family and caregivers, and where the approval is recorded into the EMR system; updates to the comprehensive care plan (900.60), where over time the care plan needs to accommodate more or less stringent directives; and approvals by the patient and recordation into the EMR system of the updates.

The novel mSDOH plan (FIG. 31 (900.80)) of the lifestyle modification instrument assists the healthcare team by informing the team of the characteristics of the patient's mSDOH and their impacts on the patient's lifestyle, pattern-of-life and persona for the team's use in medical decision-making and in the design, updates and management of the comprehensive care plan. Such characteristics reveal or update lifestyle modification prescriptions and compliance, for example: the patient's need for transportation to the healthcare entity and whether the patient's healthcare plan has a transport benefit; the patient's need for wound care and whether the patient's caregiver is competent to deliver the required care; the patient's home is on two levels and whether the patient needs assistance to navigate up/down and throughout the home; the home has rugs and whether the patient needs a method of securing/removing the rugs to prevent slips/falls; the patient's need for dietary change and whether the patient is enrolled in a nutrition program (certified or non-certified) and whether it includes education and counseling on food shopping and food preparation.

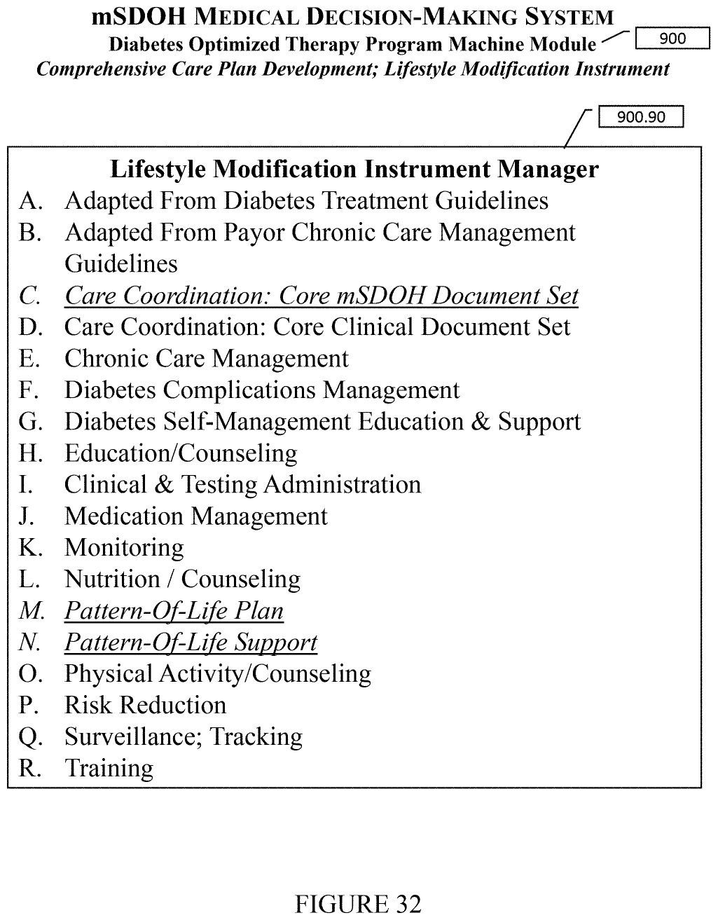

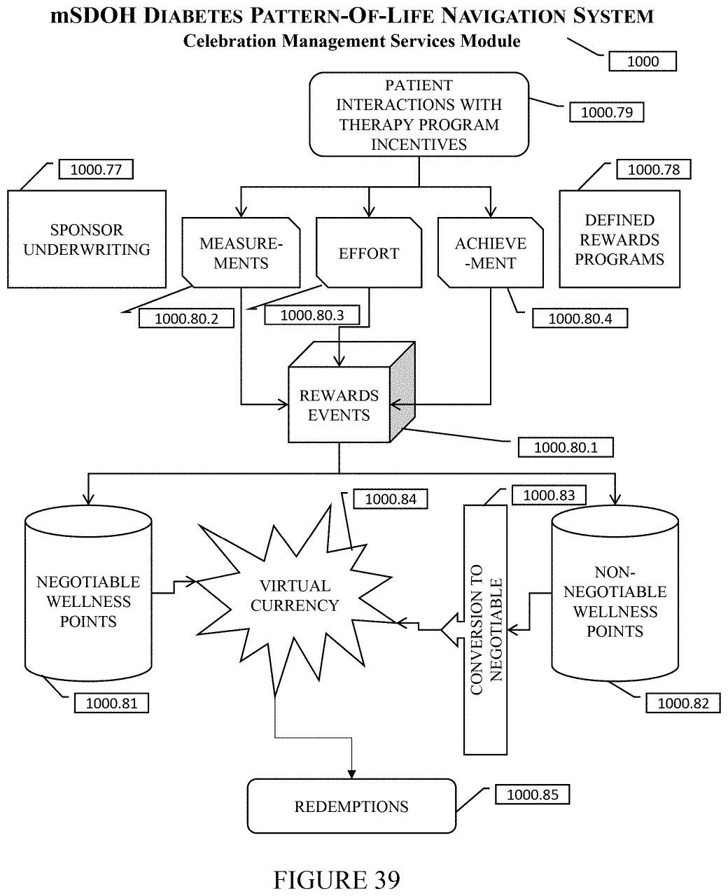

FIG. 32 introduces to the development of the novel lifestyle modification instrument a novel mSDOH medical decision-making manager system employing a lifestyle intervention instrument that improves lifestyle modification based on operationalizing the novel mSDOH plan, including the mSDOH plan's pattern-of-life component and the component for changes or alterations to modifiable risk factors (FIGS. 18-23). In a preferred embodiment, the lifestyle intervention instrument is comprised of: (a) no less than prescribed changes or alterations to modifiable risk factors of: (i) the prescribed novel mSDOH plan; (ii) weight loss, (iii) healthy diet, (iv) increased physical activity and (b) together with at least one of another prescribed lifestyle modification strategy, guideline or directive (900.90), such as: a common treatment method adopted from the guidelines of the ADA [portfolio Item A] or CMS chronic care management guidelines [portfolio Item B]; care coordination [portfolio Item C and D]; chronic care management [portfolio Item E]; diabetes complications management [portfolio Item F]; diabetes self-management education and support [portfolio Item G]; education/counseling [portfolio Item H]; clinical testing administration [portfolio Item I]; medication management [portfolio Item J]; monitoring [portfolio Item K]; nutrition counseling [portfolio Item L]; pattern-of-life plan only [portfolio Item M]; pattern-of-life support only [portfolio Item N]; physical activity/counseling [portfolio Item 0]; risk reduction only [portfolio Item P]; surveillance-tracking [portfolio Item Q]; training [portfolio Item R]; (c) care delivery through a multidisciplined comprehensive care team; (d) care duration no less than three months; (e) care follow-up assessment no less than six months after completing a goal or its prescribed timeline; and (f) regular and consistent reports to the healthcare team of (i) the delay of diabetes progression or occurrence in an asymptomatic patient and (ii) measures or Proxy measures of diabetes progression, improvement or risk reduction in a diabetic patient.
Common Characteristics Of Lifestyle Modification Intervention Instrument Component: usual/standard care; attention control (attention/education/materials/devices in addition to usual care; diet only; exercise only; medication therapy only; other (name and general description).
Novel Lifestyle Modification Intervention Instrument Operationalization Component: Activation of the lifestyle intervention instrument is introduced through a novel mSDOH-informed comprehensive care plan structure combining: (a) the novel mSDOH plan for changing the patient's pattern-of-life (FIGS. 18, 19) [patient-specific <individually tailored; regularly monitored>; self-directed <patient given a program to follow at home/in the pattern-of-life>; group focused); (b) together with the common: care schedule (900.910); blood glucose management (900.920); weight management (900.930); Diabetes Self-Management Education and Support, Medical Nutrition and CDC-Recognized Diabetes Prevention Program (900.940); program preparedness (900.950); comorbidities and complications prevention (900.960); comorbidities and complications management (900.970); chronic care management (900.980); flu and sick days management (900.990); psychosocial counseling (900.1000); and care coordination (900.1010) among the care team and specialists.
Novel Lifestyle Modification Intervention Instrument Goals Component: (a) application of the novel mSDOH plan and its patient-specific insights to inform the healthcare team in its health risk analysis and medical decision-making to establish the novel patient pattern-of-life (FIG. 18) including its modifiable behaviors to improve patient valuable behaviors and decrease patient harmful behaviors (FIG. 19); together with the common (b) reduction of risk factors for occurrence of diabetes; reduction of risk factors for progression of diabetes; reduction of risk factors for coronary heart/vascular disease; improved measures for metabolic variables; prevention of adverse clinical events due to diabetes or metabolic syndrome; improved weight loss; improved psychological wellbeing; improved self-sufficiency; increased physical activity and intensity; improved dietary behaviors.
Common Lifestyle Modification Instrument of Program Framework Component: one of—transtheoretical model (stages of readiness); social cognitive theory; cognitive behavioral theory; self-determination theory; other (name or general description).
Novel Lifestyle Modification Intervention Instrument mSDOH Plan Component: application of the novel mSDOH plan and its patient-specific insights to inform the healthcare team in its health risk analysis and medical decision-making through: the Pattern-Of-Life profile (FIG. 18 (610); the Patient Preferences; (FIG. 19 (660); the Patient Persona/Essentialities (FIG. 21 (690.20) and the Modifiable Behaviors (FIG. 25—delivery mode (including patient-reported-outcomes instrument; xPOCT; incentives and rewards (FIG. 39); the Social & Economic Circumstances (FIG. 24 (700.12) and FIG. 26)—delivery mode (including patient-reported-outcomes instrument; xPOCT; incentives and rewards (FIG. 39); the Socio Needs (FIG. 24 (700.13) and FIG. 27—delivery mode (including patient-reported-outcomes instrument; xPOCT; incentives and rewards (FIG. 39); and the Repeated Measures (FIG. 23 (690.50))—delivery mode (including patient-reported-outcomes instrument; xPOCT; incentives and rewards (FIG. 39);
Novel Lifestyle Modification Diet Intervention Instrument Component: (a) application of the novel mSDOH plan and its patient-specific insights to inform the healthcare team in its the design or update of the diet intervention based on the frequency, intensity, portion (including dosage, proportion, size), duration and the use of community health assets; together with (b) common dietary strategies, such as: weight loss; follow established guidelines; specific diet (vegan, low fat, high fruits and vegetables, high fish low educational material, high protein; general healthy eating—no specific program; other (name or general description); (c) novel delivery modes—follow-up/reinforcement devices (an interactive, remote, patient-reported-outcome instrument and an interactive, remote, xPOCT [FIG. 36]); and (d) common delivery modes, such as: diary; survey completion; telephone contact; personal interview <Internet, face-to-face>; progress reports; text messages; workbook; educational material; newsletter]; individual counseling/education with who, frequency and duration; group counseling/education with who, frequency and duration; self-directed change in eating habits only; materials/food provided.
Novel Lifestyle Modification Exercise Intervention Instrument Component: (a) application of the novel mSDOH plan and its patient-specific insights to inform the healthcare team in its design or update of the physical exercise intervention based on the frequency, intensity, duration and the use of community health assets; together with (b) common exercise strategies, such as aerobic/endurance activities; strength/resistance exercises; stretching; general increase in physical activity only; (c) novel delivery modes—follow-up/reinforcement devices (an interactive, remote, patient-reported-outcome instrument and an interactive, remote, xPOCT [FIG. 36]); and (d) common delivery mode, such as: individual counseling/education with who, frequency and duration; group counseling/education with who, frequency and duration; self-directed exercise only; materials/food provided; follow-up/reinforcement devices [interactive, remote, patient-reported-outcome instrument; interactive, remote, xPOCT; diary; survey completion; telephone contact; personal interview <Internet, face-to-face>; progress reports; text messages; workbook; educational material; newsletter].
Novel Other Lifestyle Modification Intervention Instrument Component: (a) application of the novel mSDOH plan and its patient-specific insights to inform the healthcare team in its design or update of other lifestyle modifications based on the frequency, intensity, duration and the use of community health assets for stress management, goal setting and monitoring, smoking cessation (method description); group discussions/support groups/education beyond diet and exercise; medication therapy (name); other (name and general description), together with (b) common interventions for: stress management; goal setting and monitoring; smoking cessation (method description); group discussions/support groups/education beyond diet and exercise; medication therapy (name); other (name and general description).
Novel Lifestyle Modification Intervention Instruments Measures Component Reporting of mSDOH measures and outcomes appears in the patient chart of the EMR system (FIG. 12). A preferred embodiment introduces the reporting of novel mSDOH information. In a preferred embodiment, novel mSDOH measures and outcomes reported are: (a) the mSDOH-burden hazard ratio and diabetes health risk calculator as novel elements of the common health risk assessment; (b) the mSDOH-informed diagnosis and prognosis elements of medical decision-making; (c) the mSDOH plan's strategy and periodic increases and decreases in the directives of the strategy; (d) the mSDOH-informed patient outreach, engagement and retention activities including the device or mode of the xPOCT/patient-reported-outcome instrument, any other outreach device/mode, the patient response rate, core compliance directives and the compliance rate; (e) changes in diabetes processes, changes in the diabetes state and stage and changes in the patient's pattern-of-life with the valuable and harmful factors indicated, and changes in the comprehensive care plan. The reporting includes measures at baseline, at the goal or reference time point of accomplishment in the comprehensive care plan, at the midpoint of such time point and at each last follow-up point of such time.

Pattern-of-Life Navigation—Patient Outreach; Engagement; Retention

Patient outreach and engagement is the method in a preferred embodiment for patient proactive participation in operationalizing the novel pattern-of-life information in the treatment of diabetes. FIG. 35 introduces a patient-provider communication channel (1000.10) the novelty of which is based on (a) the mSDOH plan and the interactive communication from the healthcare team to the patient (1000.20) of the status of the patient's pattern-of-life and the patient's eligibility for incentives and rewards associated with compliance with the directives of the comprehensive care plan and (b) the mSDOH plan and the interactive communication from the patient to the healthcare team (1000.30) of patient-reported-outcomes and xPOCT, including Signs, Symptoms, care plan performance (including the patient's utilization of community health assets), physical limitations, HRQoL and incentives and rewards eligibility status and redemption status associated with compliance with the directives of the comprehensive care plan.

A preferred embodiment introduces (1000.40) a novel patient-reported-outcomes instrument and xPOCT (jointly referred to as the PRO Instrument) based on assessment of the mSDOH plan informed by the endpoint model and its cross-domain interactions evaluation (FIG. 28A (700.60)) in establishing the patient's health risk and validated measures (1000.60).

Validated Measures; Patient Reported Outcomes; Data Cards

GENERAL: In a preferred embodiment, there is introduced at FIG. 36 a novel PRO Instrument and xPOCT that measures treatment risk or benefit based on patient reported outcomes (PRO) as the means to capture, assess and report PRO data—Symptoms, Signs and the pattern-of-life derived from changes or alterations to the patient's mSDOH made by the patient during the navigation of the pattern-of-life and from the patient's interaction, performance and compliance with the directives of the Comprehensive Care Plan. The PRO data is a measurement based on a report that comes directly from the patient and that assesses the status of the patient's health condition without amendment or interpretation of the patient's response by a clinician or anyone else. The PRO is measured by self-report (or by interview provided that the interviewer records only the patient's response).

PRO INSTRUMENT RELIABILITY: The PRO Instrument's reliability takes into account internal consistency with respect to the extent to which Items comprising a scale measure the same Concept, the intercorrelation of Items that contribute to a score and the internal uniformity and regularity of the data, particularly from the view of interclass correlation coefficients.

PRO INSTRUMENT VALIDITY: The PRO Instrument's validity takes into account content validity and construct validity. With respect to content validity, the PRO Instrument evidences that it measures the Concepts of interest, including evidence from qualitative studies that the Items and Domains of the PRO Instrument are appropriate and comprehensive relative to the PRO's measurement Concept, population and use.

The context of content validity includes treatment risk or benefit and outcomes measured in the environment of the patient's pattern-of-life impacted by mSDOH, including encounter Settings, encounter persons and encounter events. For each patient, the pattern-of-life encounter Settings is at least one of: (a) remote from and external to traditional healthcare bricks-and-mortar Setting; (b) face-to-face within traditional bricks-and-mortar Setting and (c) hybrid remote/face-to-face Settings. Remote Settings includes reality-based bricks-and-mortar encounter Settings and encounter Settings with the healthcare team. Remote Settings also includes electronic environment encounter Settings, such as for example health-related social media encounter Settings, mobile communication device encounter Settings, health-related gamification encounter Settings and health-related virtual reality encounter Settings. Gamification encounter Settings include the application of typical elements of game playing (e.g., point scoring, competition with others, rules of play) to health and wellness activities to encourage engagement with the patient's Comprehensive Care Plan, with healthcare services and with the utilization of community health assets.

The content validity of the PRO Instrument is based on validated instruments and research studies. Previously-existing validated instruments include analysis of mSDOH such as the following: challenges with quality of life; challenges with activities of daily living; the use of electronic personal assistant devices; mSDOH and other psychosocial subject matter such as for example attitudes, caregiving, chronic treatment, confusion-hubbub-disorder, experience sampling, gender, hedonic motivation, homelessness, living arrangements, mobile health, modified technology acceptance, new end-user computer information systems, organizational/social groups, pressures of patient adherence, self-esteem, social networking, social relationships, social support and stress-anxiety-depression.

With respect to construct validity, the PRO Instrument evidences that relationships among Items, Domains and Concepts conform to logical relationships that exist with measures of related Domains, Concepts or scores produced in the mSDOH plan.

PRO INSTRUMENT ABILITY TO DETECT CHANGE: The PRO Instrument's ability to detect change evidences that the instrument can identify differences in scores over time in individuals or groups who have changed with respect to the measurement Concept, including from the view of within-person change over time and effect-size change over time.

PRO INSTRUMENT CHARACTERISTICS: Characteristics of the PRO Instrument take into account the: (a) medical condition for intended use; (b) population for intended use; (c) Conceptual framework of the instrument; (d) Concepts being measured; (d) number of Items; (e) format; (f) administration mode; (g) data collection method; (h) response options; (i) recall period; (j) scoring; (k) weighting of Items and Domains; (l) respondent burden; (m) cultural competency/translation/cultural adaptation availability; and (n) operationalization.

THE MEDICAL CONDITION FOR INTENDED USE: The PRO Instrument is used to assess the impact of changes to mSDOH on Symptoms, Signs and treatment risks or benefits with respect to diabetes or two or more coexisting chronic diseases.

PRO INSTRUMENT POPULATION FOR INTENDED USE: The intended population that will utilize the PRO Instrument is the person diagnosed with diabetes, prediabetic persons and asymptomatic persons with a risk of diabetes established by common tests, as well as persons having metabolic syndrome or two or more coexisting chronic diseases.

PRO INSTRUMENT CONCEPT: The PRO Instrument Concept represents aspects of how the patient feels or functions with respect to treatment risk or benefits associated, directly or indirectly, with diabetes. The change over time in how the patient feels or functions is the thing or variable that is measured by the PRO Instrument.

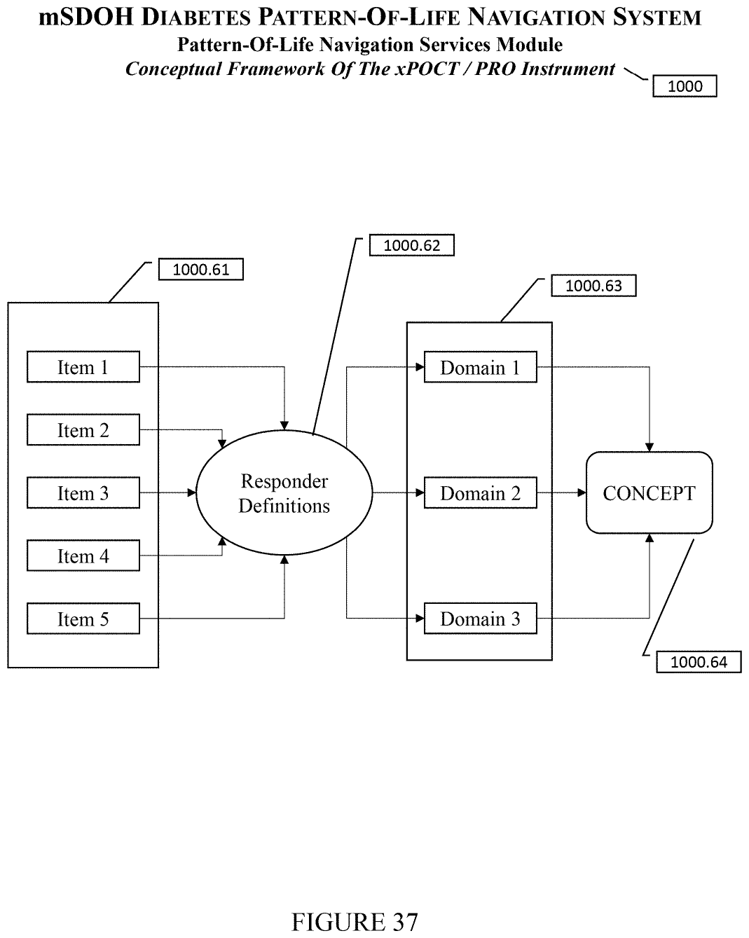

PRO INSTRUMENT CONCEPTUAL FRAMEWORK: The relationships between the Items in the PRO Instrument and the Concepts measured is introduced at FIG. 37 as a novel Conceptual framework of the PRO Instrument as informed by mSDOH. The framework is comprised of: Items (an individual question, statement or task that is evaluated or assessed by the patient to address a Concept. (1000.61) The Items are the mSDOH variables); Responder Definitions (the PRO Instrument score-change for an individual patient over a predetermined time period that is interpreted as a treatment risk or benefit. (1000.62) Measurement of the score-change is at least one of quantitative (by number or percent of reductions or improvement or other measure) and qualitative (worse, some, better; etc.). The Responder Definitions may be expressed as easy-to-manage icons, such as emoji; Domains (a sub-Concept represented by a score of the PRO Instrument that measures a larger Concept comprised of multiple Domains. (1000.63) The invention's nine Domains apply to the Concepts of diabetes, its most prevalent coexisting chronic diseases and metabolic syndrome); Concept (the specific measurement goal [i.e., the thing or variable that is to be measured by the PRO Instrument] to measure the effect of the mSDOH or other medical intervention on one or more Concepts. (1000.64) The PRO

Concepts of the mSDOH plan are how the patient functions or feels with respect to the health condition, treatment risk or benefit applicable to diabetes, its most prevalent coexisting chronic diseases and metabolic syndrome); and Treatment Benefit (how the patient survives, feels or functions).

PRO INSTRUMENT CONCEPTS BEING MEASURED: The treatment risk or benefit is the effect of treatment on how a patient survives, feels or functions. Treatment risk or benefit is demonstrated by the treatment effect. The treatment effect is measured as: the presence of a Symptom or health risk factor; the change in severity, intensity or value of a Symptom, Sign or health risk factor; the absence of a Symptom, Sign or health risk factor; the change in the value of the outcome; or an improvement or delay in the development of Symptoms, Signs or health risk factors. Measures that do not directly capture the treatment effect on how a patient survives, feels or functions are surrogate measures of treatment risk or benefit.

PRO INSTRUMENT NUMBER OF ITEMS: There may be well over 100 Items among the nine Domains. A representative Items-per Domain model is scheduled in the following table.

Number Of Items Model

| Domains | Items |
|---|---|
| Domain 9—Treatment Sites | 11 |
| Domain 8—Most-Prevalent Comorbid | 10 |
| Chronic Condition Dyads & Triads | |
| Domain 7—Comprehensive Care Plan/ | 4 |
| Medical Decision-Making | |
| Domain 6—Extended Histories | 8 |
| Domain 5—Medical Histories | 8 |
| Domain 4—Family Histories | 5 |
| Domain 3—Social Histories | 10 |
| Domain 2—Patient Personalities | 93 |
| Domain 1—Repeated Measures | 8 |
| Total Items | 157 |

PRO INSTRUMENT FORMAT: The PRO Instrument will have a long-form (vs wide form) format, wherein there is multiple rows for each patient capturing each variable and each time-point.

PRO INSTRUMENT ADMINISTRATION MODE: The Diary is interactive, enabling (a) face-to-face communication and administration of PRO data between the patient and the healthcare team and (b) remote communication of PRO data self-managed by the patient and, where appropriate, by a Proxy such as a caregiver for the patient. The communication is through electronic devices and channels, such as mobile phones, mobile tablets and Internet-connected desktop and mobile computers and devices.

PRO INSTRUMENT DATA COLLECTION METHOD: Collection of PRO data is performed by the patient's self-assessment of observations, experiences, educational and other activities prescribed in the patient's Comprehensive Care Plan to be performed by the patient. The patient's healthcare team or care giver will enter such data only when the patient is unable to do so, and as the patient's Proxy in entering such data, will not furnish information that evaluates the condition, circumstances and other information with respect to the patient.

PRO INSTRUMENT RESPONSE OPTIONS: The patient response options take into account the appropriateness of the intended patient and patient population and considers a variety of factors such as: clear and appropriate wording in responses; clear distinction between choices; adequate instructions for completing Items and selecting responses for the Items; justification of the number of response options such as by qualitative research, initial instrument testing and existing literature; appropriate ordering and intervals for responses; avoidance of potential response ceiling or floor effects so that fewer patients respond at the response continuum top or bottom (by introducing more responses to capture worsening or improvement); and bias in the weighted direction of responses.

The response options includes, as appropriate for each diary, at least one of: (a) recording of events as they occur, wherein specific events are recorded as they occur using an event log included in a patient diary or other reporting system (such as an interactive voice response system); (b) checklist, wherein the patient is provided a simple choice between a limited set of options, such as Yes, No and Don't know; or the patient is asked to place a mark in a space if the statement in the Item is true; (c) Likert scale, wherein an ordered set of discrete terms or statements from the patient are asked to choose the category that best describes their state or experience; the ends of rating scales are anchored with words but the categories are numbered rather than labeled with words; (d) rating scale, wherein a set of numerical categories from which the patient is asked to choose the category that best describes the patient's state or experience; the ends of the rating scale are anchored with words but the categories are numbered rather than labeled with words; (e) Visual analog scale (VAS), wherein a line of fixed length (usually 100 mm) with words that anchor the scale at the extreme ends and do words describing intermediate positions; the patient is instructed to indicate the place on the line corresponding to the patient's perceived state; the mark's position is measured as the score; (f) anchored or categorized VAS, wherein a vas has the addition of one or more intermediate marks positioned along the line with reference terms assigned to each mark to help the patient identify the location between the scale's end (such as half-way); and (g) pictorial scale, wherein a set of pictures, icons or emoji applied to any of the other response option types

PRO INSTRUMENT RECALL PERIOD: The recall period, the period of time during which the patient is asked to consider in responding to a PRO Instrument Item or question, is momentary (real time) or retrospective of varying lengths. The PRO Instrument encourages the recall period to be in real time or near real time, through instructions, reminders and incentives.

PRO INSTRUMENT SCORING: A score is a number or index derived from a patient's response to Items in the PRO Instrument. A score is computed based on a scoring algorithm used in statistical analyses. Scores where applicable are computed for individual Items, Domains and Concepts, as well as a summary of Items, Domains or Concepts. The patient's assessments are quantitatively scored, such as for example on a scale of 1 to 10, and qualitatively scored, such as for example on a scale of worse, better, best, no-change.

PRO INSTRUMENT WEIGHTING: Items, Domains and Concepts are nested and weighted. Weighting or scaling utilizes one or more systems of numbers or verbal anchors by which a value or score is derived for an Item, Domain or Concept.

PRO INSTRUMENT RESPONDENT BURDEN: The PRO Instrument is designed so as to avoid an undue physical, emotional and cognitive strain on patients. Such design takes into account factors that can contribute to respondent burden, such as for example: length of the PRO Instrument; formatting; font size too small to read easily; new instructions for each Item; requirement that patients consult records to complete responses; inadequate time to complete the PRO Instrument; literacy level too high for the mSDOH plan; questions that patients are unwilling to answer; perception by patients that the interviewer (healthcare team, caregiver or other appropriate patient Proxy) wants or expects a particular response; physical help in responding (such as for example assistance with a telephone or computer keyboard, turning pages, holding a pen or stylus, etc.).

PRO INSTRUMENT CULTURAL COMPETENCY; TRANSLATION/CULTURAL ADAPTATION: The PRO Instrument is culturally competent, taking into account racial, ethnic, language, community and other factors supporting patient outreach, engagement and retention. The PRO Instrument is in English with a patient-enabled option to translate questionnaires and tests into Spanish and other languages relevant to the patient.

PRO INSTRUMENT OPERATIONALIZATION: The PRO Instrument operates as a “smart” questionnaire capturing the patient's observations or assessment, together with the information and documentation that support the use of the instrument. As a “smart” questionnaire, the PRO Instrument functions as an electronic, interactive, multi-dimensional diary subject to the HIPPA requirements and integrating the patient's assessment, the directives of the patient's Comprehensive Care Plan and the patient's performance of such directives.

The diary is directed to Items and seven components: Symptoms, Signs, Physical Performance, Community Health Asset Utilization, Medication, Related Physical Limitation and Physical Exam (collectively, the “Component Diaries”). Although Signs generally are observed and interpreted by the clinician, in the case of the PRO Instrument, Signs are noticed, interpreted and reported by the patient. The Component Diaries is compiled and integrated, and as compiled and integrated, functions as parts of a Master Diary. The information recorded by the patient in each Component Diary is PRO data on the endpoints of the treatment risks or benefits.

Each Component Diary collects PRO data on the patient's subjective assessment of the treatment risks or benefits represented by such diary and their effect on diabetes, its most prevalent coexisting chronic diseases and the metabolic syndrome. The assessment for each Component Diary is sub-indexed or scored as the respective Component Diary index or score. Each Component Diary sub-index or score is reported electronically to the Master Diary. The sub-indices or scores of the Component Diaries are compiled into a composite index or score. The composite score is the Master Diary index or score. The Master Diary index or score represents the composite indices or scores of the composite endpoints of the treatment risks or benefits. The Master Diary index or score and the supporting information is reported to the comprehensive care plan and the patient's healthcare team.

In a preferred embodiment, the PRO Instrument and the xPOCT at FIG. 36, as well as the application by the general statistical model at FIG. 28A, introduce a novel Data Card for each of the nine Domains based on the mSDOH plan, where the Data Cards contains mSDOH information that informs the healthcare team in the management of health risk analysis, medical decision-making, development, updating and management of the comprehensive care plan and patient outreach, engagement and plan retention, as well as where the Data Cards contains information that informs the patient's navigation of the pattern-of-life and performance of the comprehensive care plan.
Domain 9 Data Card—Treatment Sites: In a preferred embodiment, the treatment sites commonly do not include mSDOH and are comprised of the face-to-face environments of the healthcare professionals, while in a novel embodiment there is introduced mSDOH sites remote from the face-to-face sites. Remote sites include the patient's home, community health assets and virtual sites such as an interactive electronic xPOCT or another patient-reported-outcome instrument.
Domain 8 Data Card—Comorbidities: In a preferred embodiment, mSDOH are introduced as novel data to the Comorbidities Data Card, which commonly includes the patient's: (a) comorbid chronic disease dyads; (b) the comorbid chronic disease triads; and (c) the any additional of the most prevalent comorbid chronic diseases the patient has. Such three groups of chronic conditions collectively are referred to as the “coexisting most-prevalent chronic diseases”. The coexisting most-prevalent chronic diseases mix is based on the latest CMS report presenting an analysis of the most prevalent chronic condition comorbidities with diabetes. A preferred embodiment utilizes the CMS Chronic Conditions among Medicare Beneficiaries, Chartbook, 2012 Edition. Baltimore, Md. 2012 or the then most-current similar report.
Domain 7 Data Card—Medical Histories: In a preferred embodiment, mSDOH are introduced as novel data to the Medical History data card, which commonly is comprised of the data collection requirements commonly articulated in the EMR system. For example, such requirements include lifestyle (which commonly does not include mSDOH and which commonly is known as smoking, consumption of alcoholic beverages, coffee, tea, drugs, etc.); hobbies/leisure time; pets/animals; lived outside the U.S.; stress factors (life-changes experienced by patient/family, unusual psychological stress, etc.); characterize diet/nutrition and any intolerances; exercise (regularly, how often, what type, limitations, etc.); and family medical history—distinguished from medical history—(status: living, deceased, age, major illness/cause of death).
Domain 6 Data Card—Family History: In a preferred embodiment, mSDOH are introduced as novel data to the Family History data card (preferably expressed as a genogram, otherwise in tabular form), which commonly is comprised of: health status/cause of death of parents, siblings, children; specific diseases related to problems identified in the chief complaint; diseases of family members which may be hereditary or place the patient at risk (up to three generations); ages and state of health of family members (for deceased family members, the age at the time of death and cause, if known); notation of the presence of diabetes and any of its coexisting most-prevalent dyads or triads.
Domain 5 Data Card—Social Histories: In a preferred embodiment, mSDOH are introduced as novel data to the Social Histories data care, which commonly is comprised of: marital status and/or living arrangements; current employment; occupational history; use of drugs, alcohol or tobacco; level of education; in a preferred embodiment, newly-introduced data on the application of educational attainment to livelihood strategy as measured on a scale, such as for example 1-to-5, worse-better; sexual history; born, raised, resides; current lifestyle (in a preferred embodiment, newly-introduced data on living situation, relationship support system, daily activities, leisure, cultural/spiritual beliefs, alternative health care practices, other); risk factors (health habits [nutrition, caffeine, exercise, sleep, safety, exposures, tattoos/piercings, etc.], tobacco, alcohol, recreational drugs, sexual risks, economic risks, stress, violence, advanced directives; other relevant social factors (see below at Essentialities).
Domain 3 Data Card—Extended Histories: In a preferred embodiment, mSDOH are introduced as novel data to the Extended History, which commonly is comprised of a profile of four elements of the history of the coexisting most-prevalent chronic diseases together with a report of the status of at least three active chronic conditions, encompassing: (1) location—the location of the chief complaint, such as for example ankle, chest, generalized, etc.); (2) quality—the way the patient describes their pain or Symptom, etc.; (3) severity—quantifies the Symptom, such as for example one or more of: on a scale of 1-10; mild, moderate, severe; etc.; (4) timing—the course of the patient's Symptoms, such as for example improving, worsening, constant, intermittent, etc.; duration—the total time the patient's Symptoms have been present (such as for example ×6 hrs, ×2 wks, ×25 min, since x years, life-time, etc.); (5) context—precipitating factors, risk factors, previous treatment and how the patient's present Symptoms fit in with prior related problems (including mSDOH relevant to the patient); (6) modifying factors—divided into exacerbating factors and mitigating factors (associated Signs & Symptoms); (7) problems relating to or accompanying the chief complaint; and (8) pertinent positives and negatives
Domain 2 Data Card—Comprehensive Care Plans/Medical Decision-Making: In a preferred embodiment, mSDOH are introduced as novel data to the comprehensive care plan, which commonly is comprised of the patient's health risk level data and Chief Complaint data. The comprehensive care plan commonly evidences the medical decision-making of the patient's healthcare team, based on a Health Risk Level. A preferred embodiment introduces mSDOH as a factor in determining the Health Risk Level. It correlates High Complexity Medical Decision-Making to the Health Risk Level. The expression of the Health Risk Level commonly is High Complexity Medical Decision-Making with respect to one of: (a) Moderate Risk [Risk Level 4] and (b) High Risk [Risk Level 5]. In a preferred embodiment, mSDOH are introduced to inform medical decision-making in the assessment of Moderate Risk and High Risk. The patient's Chief Complaint data commonly is comprised of: (a) a composite of the patient's coexisting most-prevalent chronic diseases and (b) the treatment algorithm prescribed or recommended by the patient's comprehensive care plan. In a preferred embodiment, mSDOH are introduced to inform the development, updating and management of the treatment algorithm.
Domain 1 Data Card—Patient Persona: In a preferred embodiment, mSDOH are introduced as novel data in the form of the patient's persona, personality or Essentialities data card. Such card commonly is not included in the EMR system or the general assessment of the patient, although SDOH commonly is an optional feature a healthcare provider can elect to include in an EMR System. The Patient Persona data card is a unifying concept for the class of mSDOH that includes the treatment of diabetes through lifestyle modification. Patient persona and Essentialities are introduced in a preferred embodiment as Proxies for lifestyle modification. As Proxies, patient persona and Essentialities resolve a set of specific problems associated with lifestyle, including its basis in unstructured data and the significant scope, depth and subjectiveness of, and the extensive body of research on, lifestyle measures.
Patient Persona at FIG. 21 is measured by common measures of general demographic information, together with measures of patient Essentialities and measures of patient compliance with the directives of the comprehensive care plan, which compliance measures may use measures commonly used by the consumer marketing industry.
Essentialities are many in number and are common in the study of human behaviors and their impact on health. Accordingly, each Essentiality, except for lifestyle, is expressed by a score for the Essentiality ascribed by the patient from one to three, with three being the highest in importance as perceived by the patient. Lifestyle is disproportionately weighted at no less than a ratio the numerator of which is the burden of mSDOH hazard ratio applicable to the patient (such as city, county, State or national) and the denominator is the score of the sum of the other Essentialities. The composite score informs (700) the health risk calculator in the pattern-of-life knowledge machine (FIGS. 24-28B) through its composite random and Proxy variables (700.15) and its primary endpoint families and Proxies (700.60).
A preferred embodiment introduces a health risk calculator at FIG. 28A based on mSDOH. Such basis is distinguished from evaluating compliance with directives of the comprehensive care plan using measures common to the consumer marketing industry including weighting, scoring, indexing comparing, correlating and other statistical methods to establish: (a) adherence and understanding of the directives by the patient, including education information with respect to the Coexisting most-prevalent chronic diseases and self-management activities recommended in the comprehensive care plan, together with a prediction of adherence; (b) compliance with the prescribed or recommended self-care and management of conventional treatment (such as for example: strengthening; inhalers; oxygen therapy; medications; integration of treatment; smoking cessation), as prescribed or recommended by the comprehensive care plan, together with a prediction of compliance; (c) frequency, intensity and duration of patient-performed activities as prescribed or recommended by the comprehensive care plan, together with a prediction of motivation; (d) deviations and causes of deviations from the patient-performed activities that have been prescribed or recommended by the comprehensive care plan, together with a prediction of cessation.
Domain 1 Data Card—Repeat Measures: In a preferred embodiment, mSDOH are introduced as novel data in the form of a Repeated Measures data card. Such card commonly is not included in the EMR system or the general assessment of the patient (although structural SDOH [as distinguished from mSDOH] commonly are an optional feature a healthcare provider can elect to include in an EMR system). Operation of the patient reported outcomes engine evaluates the capture and evaluation of the preferred embodiment's mSDOH repeated treatment measures to inform the determination of performance of the comprehensive care plan directives during the patient's navigation of the pattern of life.

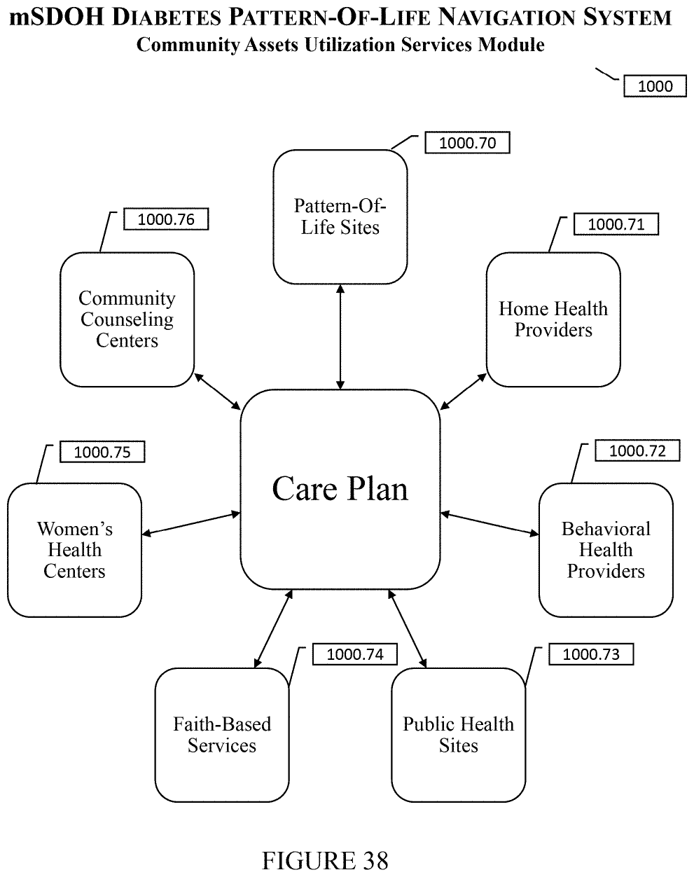

FIG. 38 introduces to the novel mSDOH diabetes pattern-of-life navigation system a novel community assets utilization services module based on the patient's performance of comprehensive care plan directives informed by mSDOH, such determinants comprising the utilization frequency, intensity and duration by the patient of community-based non-hospital health organizations and institutions including, in a preferred embodiment, pattern-of-life sites (1000.70), home health providers (1000.71), behavioral health providers (1000.72), public health sites (1000.73), faith-based and other trusted-community sites (1000.74), women's health sites (1000.75) and community counseling sites (1000.76).

Where the patient has met the goals and other performance and compliance requirements of the comprehensive care plan, a preferred embodiment at FIG. 39 introduces a novel celebration services module based on the mSDOH plan and its performance metrics in the lifestyle intervention instrument offering an incentives and rewards benefit to the patient. Such benefit commonly is underwritten by a third-party sponsor(s) (1000.77) through a defined rewards program (1000.78). Such program commonly is furnished by a third-party service provider. Celebration management services are initiated by patient outreach and engagement interactions with the diabetes therapy program incentive offerings (1000.79) communicated through the PRO Instrument (FIG. 36). Rewards events (100.80.1) are activated by the patient achieving one or more requirements or performing one or more directives of the mSDOH plan (1000.80.2), at a level of effort prescribed by the care plan (1000.80.3) so as to “achieve” (1000.80.4). Achievement is incentivized or rewarded with wellness points issued by a points bank. Wellness points may be negotiable in nature (1000.81) where at some point the points obtain a value that may be exchanged for products, services or money. Wellness points also may be non-negotiable (1000.82) such as badges, symbols and other recognitions of the patient's achievements, particularly where recognition improves the patient's wellbeing and advances the patient's esteem in the eyes of a peer group. Non-negotiable wellness points may be converted to negotiable points based on performance of the care plan (1000.83). Until wellness points are redeemed and exchanged for products, services or money, the points are a virtual currency (1000.84) and become actual valuable currency upon redemption (1000.85).

While preferred embodiments of the invention have been shown and described, it will be clear to those skilled in the art that various changes and modifications can be made without departing from the invention in its broader aspects as set forth in the claims provided hereinafter.

1. A method of treating diabetes comprising changing or altering mSDOH, further comprising:
(a) identifying mSDOH relevant to a patient;
(b) determining the patient's risk of diabetes informed by and based on mSDOH and their changes and alterations;
(c) medical decision-making informed by and based on the mSDOH and their changes and alterations;
(d) managing comprehensive care plans informed by and based on the mSDOH and their changes and alterations; and
(e) outreaching to and engaging with the patient informed by and based on the mSDOH and their changes or alterations.

2. The treatment method of claim 1 further comprising diabetes risk assessment comprising a mSDOH-burden hazard assessment and mSDOH interpretation system comprising:
(a) determining a difference between a prevalence of diabetes in
(i) a patient's general community and
(ii) a patient's affinity community;
wherein the general community prevalence is determined by an index established by a healthcare, health research, governmental health unit or similar authority for the geographic unit of government in which the patient resides and the affinity community prevalence is determined by an index established by a similar authority for the cultural community in which the patient identifies with or is attributed to,
wherein an affinity community prevalence greater than the general community prevalence is an mSDOH-burden hazard ratio;
(b) operationalizing the mSDOH-burden hazard ratio to prevent, delay the onset of, diagnose, manage or reduce the severity of diabetes by (i) adjusting the cut point of the diabetes-risk testing procedure in an amount where the cut point is earlier by the difference between the standard cut point and the mSDOH-burden hazard ratio, (ii) informing the risk score of such difference, (iii) reassessing the risk range of normal-range, low-range, high-range and diagnosed-diabetes informed by and based on the adjusted cut point and (iii) informing medical decision-making, the design of and updates to the directives of the comprehensive care plan and patient outreach and engagement, based on the risk factor adjusted to account for the mSDOH-burden hazard ratio, according to standard care guidelines for the normal-range, low-range, high-range and diagnosed diabetes; wherein an earlier cut-point is an alert for at least one of rising-risk, presence and severity of diabetes; and
establishing relevancy of the mSDOH to the patient comprising relevancy as (i) a Proxy for valuable patient pattern-of-life practices and Essentialities that the patient perceives, during the patient's navigation of the pattern-of-life, as being beneficial to the patient's self-image or to achieving the patient's goals or (ii) a Proxy for harmful patient pattern-of-life practices and Essentialities that the patient perceives during the patient's navigation of the pattern-of-life as being detrimental to the patient's self-image or to achieving the patient's goals; wherein valuable and harmful practices and Essentialities information is evaluated by the healthcare team after being reported by the patient through one or more of a survey, questionnaire, patient-reported-outcome instrument or patient-administered xPOCT.

3. The treatment method of claim 1, further comprising mSDOH diabetes medical decision-making comprising: (a) discovering pattern-of-life knowledge; (b) feeding back and assessing pattern-of-life knowledge; and (c) optimizing pattern-of-life knowledge;
wherein discovering pattern-of-life knowledge comprising: (a) identifying the pattern-of-life of the patient, (b) medically interpreting pattern-of-life knowledge as a health risk; (c) changing or altering mSDOH; and (d) lowering the health risk; wherein further:
identifying the patient's pattern-of-life comprising at least one of recognizing and establishing: (a) the patient's changeable or alterable relevant mSDOH, social and economic circumstances and socio needs; and (b) the patient's relevant valuable and harmful health-related preferences;
wherein further, the patient's changeable or alterable mSDOH, social and economic circumstances and socio needs comprising identifying pattern-of-life components (a) by remote evaluation and reporting from the patient to the healthcare professional through an interactive patient-reported-outcomes instrument mSDOH-outcome-forms of pattern-of-life knowledge components and (b) by direct evaluation and reporting from the patient to the healthcare professional clinical-outcome-forms of pattern-of-life knowledge components;
wherein further, the patient's changeable or alterable relevant mSDOH, social and economic circumstances and socio needs comprising clinical-outcome-forms of pattern-of-life components comprising clinical knowledge of level 4 and level 5 health risks composed of Domains representing a plurality of at least one or more of the patient's treatment sites, most-prevalent chronic disease triads or dyads coexisting with diabetes, medical history, family history, social history, extended histories and the comprehensive care plan; and wherein further informing the patient's records in the EMR system;
wherein further, the patient's valuable and harmful health-related preferences comprising relevant mSDOH-outcomes-forms of pattern-of-life components comprising the patient's Essentialities and persona Domain and repeat treatment measures, further comprising identifying, recognizing or establishing, and determining: (a) the patient's social and personal competencies; (b) dimensions representing a plurality of at least one or more of the patient's status of individual deprivation, gender-sensitivities, race/ethnicity, medical interview, patient voice, community heath asset utilization, curative impact, technology impact, care coordination support, treatment perception and lifestyle, (c) dimensions representing a plurality of at least one or more of physical capacity, psychological, level of independence, social relationships, environment and spirituality or personal beliefs; (d) social and personal competencies exercised through the patient's pattern-of-life; and (e) repeat time-based measures of patient performance of the comprehensive care plan; and
wherein further, the components of and changes in such Domains and dimensions (a) are measured, evaluated and reported by a plurality of at least one validated survey, validated questionnaire, multiplex point of care test (xPOCT), Signs and Symptoms and (b) inform the patient's records in the EMR system;
wherein further, medically interpreting of the pattern-of-life as a health risk comprising a health risk assessment wherein: (a) clinical-outcomes-forms of pattern-of-life components constitute composite fixed and random variables and their Proxies and are analyzed to establish an index; (b) mSDOH-outcomes-forms of pattern-of-life components constitute primary endpoint families and their Proxies and are analyzed to establish an index; (c) a composite index is calculated (i) where the aggregate mSDOH-outcomes-forms of pattern-of-life components do not exceed 92.7% or the sum of the percentages for tobacco, diet/activity patterns, alcohol, sexual behavior and illicit drug use reported in the most-recent issue of the National Research Council 2015. Measuring the Risks and Causes of Premature Death: Summary of Workshops. Washington, DC: The National Academies Press. https://doi.org/10.17226/21656 and (ii) where the aggregate clinical-outcomes-forms of pattern-of-life components do not exceed 7.3% or the sum of the percentage for medical errors so reported; and (d) the pattern-of-life health risk is an index the numerator of which is the mSDOH-outcomes-forms of pattern-of-life components score and the denominator is the sum of the mSDOH-outcomes-forms of pattern-of-life components score and the clinical-outcomes-forms of pattern-of-life components score;
changing or altering relevant mSDOH comprising: (a) diabetes risk assessment informed by mSDOH; (b) medical decision-making informed by mSDOH; (c) managing the development and updating of the comprehensive care plan informed by mSDOH; (d) patient outreach and engagement informed by mSDOH; and (e) patient self-management informed by mSDOH wherein:
diabetes risk assessment informed by mSDOH further comprising the treatment method of claim 2,
medical decision-making informed by mSDOH further comprising (a) assessing the patient's health risk and determining the risk level as one of normal-range, low-range risk, high-range risk or diagnosed-diabetic informed by and based on the mSDOH-burden hazard applied to at least one of a plurality of pattern-of-life components measured by mSDOH-outcomes-forms, together with (b) assessing whether one or more pattern-of-life components are relevant to the patient and whether an adjustment increasing or decreasing the relevancy to the patient will improve the health risk to the patient; wherein relevancy is (i) a Proxy for valuable patient pattern-of-life practices and Essentialities that the patient perceives, during the patient's navigation of the pattern-of-life, as being beneficial to the patient's self-image or to achieving the patient's goals or (ii) a Proxy for harmful patient pattern-of-life practices and Essentialities that the patient perceives during the patient's navigation of the pattern-of-life as being detrimental to the patient's self-image or to achieving the patient's goals; and
wherein further, valuable and harmful practices and Essentialities information is assessed by the healthcare team after being reported by the patient through one or more of a survey, questionnaire, patient-reported-outcome instrument or patient-administered xPOCT;
managing the development and updating of the comprehensive care plan informed by and based on mSDOH further comprising: (a) increasing, decreasing or otherwise changing at least one or more of the frequency, intensity, duration, size, dosage, proportion, community health asset utilization or other element of the directives of the comprehensive care plan informed by and based on changes or alterations to mSDOH; (b) testing for and assessing any change in the mSDOH-burden hazard informed by and based on changes or alterations to mSDOH; (c) testing for and assessing any change in the pattern-of-life of the patient informed by and based on changes or alterations to mSDOH; (d) testing for and assessing the rising-risk of diabetes and its state and stage informed by and based on changes or alterations to mSDOH; (e) identifying and measuring any deviations and causes of deviations from the directives of the comprehensive care plan informed by and based on changes or alterations to mSDOH; (f) predicting the risk of motivation to perform the comprehensive care plan informed by and based on changes or alterations to mSDOH; (g) predicting the risk of cessation to perform the comprehensive care plan informed by and based on changes or alterations to mSDOH; (h) monitoring performance of the comprehensive care plan directives informed by and based on changes or alterations to mSDOH; (i) tracking care management events eligible for chronic care management services informed by and based on changes or alterations to mSDOH; (j) referring the patient into care management specialty practices informed by and based on changes or alterations to mSDOH; (k) coordinating and documenting care management activities; (1) reconciling by the care management team patient clinical data, the comprehensive care plan and summary notes; and (m) reviewing and including the reconciliation in the patient's medical record;
patient outreach and engagement informed by mSDOH further comprising (a) designing and choosing campaigns that are appropriate informed by and based on the patient's pattern-of-life and changes or alteration to mSDOH; (b) optimizing the campaigns to include changes to message content and timing informed by and based on the patient's pattern-of-life and changes or alteration to mSDOH; (c) deploying multi-channel campaigns on a scheduled recurring basis informed by and based on the patient's pattern-of-life and changes or alteration to mSDOH; (d) managing patient contact, consent and appointment information informed by and based on the patient's pattern-of-life and changes or alteration to mSDOH; and (e) managing compliance with statutory requirements for placing or sending automated, prerecorded or artificial telephone calls, SMS messages or emails or other communication formats and media; and
patient self-management informed by mSDOH further comprising: (a) communicating among the patient and the care team and accessing the comprehensive care plan outside of appointments and between live contacts with the care team informed by and based on the patient's pattern-of-life and changes or alteration to mSDOH; (b) counseling and educating the patient with respect to the health risk of diabetes, the management of nutrition, the performance of exercise, the modification of lifestyle, the management of glucose level and glucose-level reporting equipment and the management of patient-reported-outcomes and xPOCT instruments informed by and based on changes or alteration to mSDOH; (c) assigning, tracking and monitoring compliance with comprehensive care plan directives and tasks sent by the care team to the patient, including diet suggestions, exercise activities, medication reminders and appointment instructions, informed by and based on the patient's pattern-of-life and changes or alterations to mSDOH; (d) designing and communicating tailored automated comprehensive care plans appropriate to the disease state, stage or progress for the rising-risk patient who does not require active care management, informed by and based on the patient's pattern-of-life and changes or alterations to mSDOH; and (e) monitoring, managing and following-up on patient-reported-outcomes, informed by and based on the patient's pattern-of-life and changes or alterations to mSDOH;
wherein communication between the patient and the care team includes a patient-facing mobile device, web browser or email application or other asynchronous communication channel for communicating patient-reported changes to the pattern-of-life, mSDOH and health outcomes;
wherein further, lowering the health risk comprising: (a) establishing the patient's health risk as indicated by the mSDOH-burden hazard by way of the treatment method of claim 1; (b) establishing the health risk at the time of the next follow-up encounter with the patient; and (c) comparing the health risk level at the time of the last previous encounter with the risk level at the time of the current encounter; wherein an improvement, decline or no-change in risk level is attributable to or associated or coexistent with having changed or altered the pattern-of-life, including having changed or altered at least one relevant mSDOH, evidencing an improvement, decline or no-change in the state or severity of diabetes;
wherein further, feeding back and assessing pattern-of-life knowledge comprising measuring mSDOH Domains consisting of patient-reported-outcomes of: (a) modifiable health-related behaviors; (b) social and economic circumstances; and (c) socio needs;
wherein further, modifiable health-related behavior Domains consist of Proxies for health risk comprising: (a) the physical capacity Proxy weighted to 11% of such Domain; (b) the psychological Proxy weighted to 18% of such Domain; (c) the level of independence Proxy weighted to 14% of such Domain; (d) the social relationships Proxy weighted to 21% of such Domain; (e) the home environment Proxy weighted to 29% of such Domain; and (f) the spirituality/personal beliefs Proxy weighted to 7% of such Domain; wherein such weights represent validated measures and such measures are calculated and assessed by the care team and entered into the care team notes in the EMR system;
wherein further, social and economic circumstances Domains consist of Proxies for health risk comprising: (a) the product consumption Proxy weighted to 11% of such Domain; (b) the technology/media/entertainment Domain Proxy weighted to 18% of such Domain; (c) the attitudes Proxy weighted to 14% of such Domain; (d) the financial behaviors Proxy weighted to 21% of such Domain; (e) the automobile-use Proxy weighted to 29% of such Domain; and (f) the shopping Proxy weighted to 7% of such Domain; wherein such weights represent validated measures and such measures are calculated and assessed by the care team and entered into the care team notes in the EMR system;
wherein further, socio needs Domains consist of Proxies for health risk comprising: (a) the mSDOH-burden hazard index weighted to no less than 50% of such Domain; and (b) the general community socioeconomic indicators weighted to no more than 50% of such Domain, wherein such measures are calculated and assessed by the care team and entered into the care team notes in the EMR system;
wherein further, such measures are evaluated by applying common statistical methods; and
the results of such evaluation are entered into and presented to the care team through a health risk dashboard presenting and reciting one or more of changes to the patient's mSDOH, the diabetes disease process, the diabetes state, the diabetes stage, the pattern-of-life, the comprehensive care plan and the patient's health-related quality of life;
wherein further, operationalizing pattern-of-life knowledge comprising a lifestyle modification instrument;
wherein further, the lifestyle modification instrument comprising comprehensive care plan management, pattern-of-life management and mSDOH management;
wherein further, the comprehensive care plan management comprising a plurality of: diabetes risk assessments; care team/patient consultations; treatment plan; care team/patient approvals; comprehensive care plan updates; approvals of comprehensive care plan updates; and plans for complications management, self-management education and support, disease, comorbidities and complications preparedness education and counseling, clinical and testing administration, nutrition education and counseling, plan cessation prediction and resumption management including flu and sick days management, psychosocial counseling, care transition, care coordination, pattern-of-life and mSDOH plans and supports, physical activity and counseling, risk reduction and technology-use training;
wherein further, the pattern-of-life plan comprising pattern-of-life change-goals and a plan documentation set including activities, measures, timelines, outcomes and risk reduction plan;
wherein further, the mSDOH plan comprising a plurality of components including at least: a pattern-of-life profile; patient preferences change-goals; patient persona change-goals; Essentialities change-goals; the mSDOH-burden risk; risk reduction plan; modifiable health-related behaviors; social and economic circumstances; socio needs and repeated measures; goals; program framework; diet intervention; exercise intervention; and inter-component measures of frequency, intensity, duration, size, dosage, proportion, and community health asset utilization; time-based directives; plan documentation; and calculation and assessment by the care team of the characteristics of the patient's mSDOH and their impacts on the patient's lifestyle, pattern-of-life and persona; and
wherein further, the comprehensive care plan, pattern-of-life plan and mSDOH plan each comprising: change-progress measures, notes and outcomes; scheduled interventions (direct and remote) with the healthcare team; scheduled interventions with community health assets including a description of how services of agencies and specialists will be directed and coordinated; identification of the individuals responsible for each intervention; an inventory of resources and supports; revisions to the plan; and education, training, and compliance monitoring, surveillance and tracking; and
wherein further, lowering the health risk comprising: (a) establishing the patient's health risk informed by and on the basis of the mSDOH-burden hazard by way of the treatment method of claim 1 and further informed by and on the basis of medical decision-making; (b) establishing the health risk at the time of the next follow-up encounter with the patient; and (c) comparing the health risk level at the time of the last previous encounter with the risk level at the time of current encounter; wherein an improvement, decline or no-change in risk level is attributable to or associated or coexistent with having changed or altered the pattern-of-life, including having changed or altered at least one relevant mSDOH, evidencing an improvement, decline or no-change in the state or severity of diabetes.

4. The treatment method of claim 1 further comprising outreaching and engaging with the patient comprising mSDOH pattern-of-life navigation through: (a) pattern-of-life navigation services; (b) community health asset utilization services; and (c) celebration services;
wherein patient outreaching and engaging comprises an interactive patient-provider communication channel comprising (a) outbound content services to the patient consisting of a plurality of at least pattern-of-life status, patient-facing comprehensive care plan directives, diaries, reminders and alerts, encounter contact scheduling and incentives and rewards eligibility and (b) and inbound content services from the patient consisting of a plurality of at least Signs, Symptoms, care plan performance diary, community health asset utilization diary, physical limitations diary, quality of life diary and incentives and rewards eligibility and redemption activity;
wherein further, patient-provider communication channel operations comprising patient-reported-outcomes and xPOCT informed by and based on validated measures and one or more statistical methods of evaluation, measures and reporting which may include nested domain structures and endpoint family modeling; and
wherein further, patient-provider communication channel measures comprising validated reliability, validity, ability to detect change, characteristics, concept, concepts being measured, number of items, format, administration mode, data collection method, response options, recall period, scoring, weighting, respondent burden, cultural competency, cultural adaptation and operationalization;
wherein further, pattern-of-life navigation services comprising a user interface front-end capturing the patient's entry of information reporting patient compliance with prescribed directives of the comprehensive care plan, including prescribed pattern-of-life management, mSDOH management, health, wellness and community health asset utilization directives;
wherein further, community health asset utilization services comprising electronic linkages to a plurality of at least one or more pattern-of-life sites, the healthcare team/care team, the home health provider, behavioral health provider, public health site, faith-based site, women's health center, community counseling center; wherein each such resource is specific to the patient's requirements;
wherein further, utilization comprising the frequency, intensity and duration of the patient's use of community-based health organizations;
wherein further, celebration services comprising a program recognizing the patient's compliance with directives of the comprehensive care plan, wherein such program further comprising a plurality of one or more of: virtual and real-life performance incentives and rewards; virtual currency; non-monetary, monetary and non-monetary-to-monetary conversion features; recognition of the patient, the patient's peer group, patient efforts and patient achievements; and subject to regulatory compliance; and
wherein further, lowering the health risk comprising: (a) establishing the patient's health risk informed by and on the basis of the mSDOH-burden hazard by way of the treatment method of claim 1 informed by and further informed by and on the basis of patient outreach and engagement; (b) establishing the health risk at the time of the next follow-up encounter with the patient; and (c) comparing the health risk level at the time of the last previous encounter with the risk level at the time of current encounter; wherein an improvement, decline or no-change in risk level is attributable to or associated or coexistent with having changed or altered the pattern-of-life, including having changed or altered at least one relevant mSDOH, evidencing an improvement, decline or no-change in the state or severity of diabetes.
